(19) United States
(12) Patent Application Publication
(10) Pub. No.: US 2024/0108662 A1
(43) Pub. Date: Apr. 4, 2024

(54) ENGINEERED CELLS FUNCTIONALIZED WITH IMMUNE CHECKPOINT MOLECULES AND USES THEREOF

(71) Applicant: The University of North Carolina at Chapel Hill, Chapel Hill, NC (US)

(72) Inventors: Andrew Wang, Chapel Hill, NC (US); Kin Man Au, Chapel Hill, NC (US)

(21) Appl. No.: 18/039,387

(22) PCT Filed: Nov. 23, 2021
(86) PCT No.: PCT/US21/60523

§ 371 Date: May 30, 2023

Related U.S. Application Data
(60) Provisional application No. 63/119,357, filed on Nov. 30, 2020.

(51) Int. Cl. A61K 35/39; A61K 9/00; A61K 39/00; A61K 45/06; A61P 3/10; A61P 37/04; C07K 14/47; C07K 14/705; C07K 17/00; C12N 5/071

(52) U.S. Cl. CPC A61K 35/39; A61K 9/0019; A61K 39/001111; A61K 45/06; A61P 3/10; A61P 37/04; C07K 14/4726; C07K 14/70532; C07K 17/00; C12N 5/0676

(57) ABSTRACT

Described herein are functionalized cells comprising an immune checkpoint molecule covalently attached to the cell surface or to a nanoparticle attached to the cell surface, and compositions comprising the functionalized cells. Also described are acellular pancreatic extracellular matrices comprising a functionalized cell(s) and decellularized pancreatic-derived protein(s). Also described are methods of treating disease by administering to subjects the functionalized cells and acellular pancreatic extracellular matrices. Also described are methods of making the functionalized cells and acellular pancreatic extracellular matrices described herein.

### CROSS-REFERENCE TO RELATED APPLICATIONS

This application claims priority to U.S. Provisional Patent Application Ser. No. 63/119,357, filed Nov. 30, 2020, which is incorporated herein by reference in its entirety.

### STATEMENT OF GOVERNMENT SUPPORT

This invention was made with government support under Grant No. CA198999 awarded by the National Institutes of Health. The government has certain rights in the invention.

### BACKGROUND

The immune system evolved robust immune responses against foreign antigens while tolerating self-antigens to avoid autoimmunity^{14, 15}. Regulatory T (T_{reg}) cells regulate homeostasis and maintain immunotolerance²⁸. Failure to maintain immunotolerance leads to the development of autoimmune disease^{14, 29, 30}. The ability to regulate autoreactive T cells without inducing systemic immunosuppression represents a major challenge to develop new strategies to treat autoimmune disease.

Immune checkpoints are key regulators in the immune system that help maintain self-tolerance.^{11-15, 37, 38} For example, cancer cells escape immune surveillance by stimulating co-inhibitory checkpoint molecules, such as programmed cell death protein 1 (PD-1), cytotoxic T-lymphocyte-associated protein 4 (CTLA-4), and T cell immunoglobulin mucin 3 (TIM-3) signaling in activated T cells.¹¹⁻¹⁵ Several studies have revealed that the deficiency of immune checkpoint molecules—such as PD-L1 (ligand for PD-1),^{5, 16, 17} CD86 (ligand for CTLA-4 in activated T cells),¹⁸ and galectin-9 (Gal-9, the ligand for TIM-3)¹⁹ in β cells is associated with the development of insulin-dependent diabetes mellitus (also known as type 1 diabetes, T1D). Further, studies have found that coinhibitory immune checkpoint pathways such as programmed death 1 (PD1)-PD ligand 1 (PD-L1)³⁷⁻³⁹, and cytotoxic T lymphocyte associated protein 4 (CTLA-4)-cluster of differentiation 86 (CD86)^{37, 38, 40} directly regulate the development and maintenance of myelin-specific induced T_{reg} cells⁴¹.

Recent studies have demonstrated that the systemic administration of PD-L1 genetic overexpressed β cells could reverse early-onset hyperglycemic nonobese diabetic (NOD) mice in vivo.^{5, 16} However, the use of genetically engineered β cells requires substantial genetic manipulation, which is not only expensive but also subject to considerable regulation.

In the case of multiple sclerosis (MS), autoreactive T cells attack the myelin in the central nervous system (CNS), causing the autoimmune neurological disorder multiple sclerosis (MS), which disrupts communication between the brain and peripheral system^{29, 31} At least 2.5 million people worldwide are affected by MS. Most patients initially experience episodes of reversible neurological deficits, followed by remission, before chronic neurological deterioration leads to severe, irreversible disabilities³¹. Unfortunately, MS cannot be completely cured, although available immunomodulatory therapies reduce the frequency and severity of MS relapses by inducing antigen-specific immunotolerance³²⁻³⁴ thus delaying the accumulation of disabilities. New treatment strategies involve the induction of antigen-specific T_{reg} cells^{35, 36} that suppress inflammatory pathogens and restore peripheral immunotolerance without causing systemic immunosuppression.

There remains a need for therapeutics to treat or delay the onset of an autoimmune diseases and to systemically administer immune checkpoint molecules.

### BRIEF SUMMARY

Compositions comprising a functionalized cell with an immune checkpoint molecule attached to the surface and methods of making and using the same are provided herein.

In another aspect, the subject matter described herein is directed to a functionalized cell comprising, a cell comprising a decorated cell surface, wherein the decorated cell surface comprises at least one covalently attached immune checkpoint molecule.

In another aspect, the subject matter described herein is directed to a functionalized cell having one of the following general structures:

wherein, X is an integer from 1 to 50, and y is an integer from 1 to 20.

In another aspect, the subject matter described herein is directed to an acellular pancreatic extracellular matrix comprising, a functionalized cell comprising, a cell comprising a decorated cell surface, wherein the decorated cell surface comprises at least one covalently attached immune checkpoint molecule; and decellularized pancreatic-derived proteins.

In another aspect, the subject matter described herein is directed to pharmaceutical compositions comprising: a functionalized cell comprising, a cell comprising a decorated cell surface, wherein the decorated cell surface comprises at least one covalently attached immune checkpoint molecule; or an acellular pancreatic extracellular matrix comprising, a functionalized cell comprising, a cell comprising a decorated cell surface, wherein the decorated cell surface comprises at least one covalently attached immune checkpoint molecule; and decellularized pancreatic-derived proteins; and a pharmaceutically acceptable excipient.

In another aspect, the subject matter described herein is directed to vaccines comprising: a functionalized cell comprising, a cell comprising a decorated cell surface, wherein the decorated cell surface comprises at least one covalently attached immune checkpoint molecule; or an acellular pancreatic extracellular matrix comprising, a functionalized cell comprising, a cell comprising a decorated cell surface, wherein the decorated cell surface comprises at least one covalently attached immune checkpoint molecule; and decellularized pancreatic-derived proteins; and a pharmaceutically acceptable liquid vehicle.

In another aspect, the subject matter described herein is directed to a method of treating or delaying onset of an autoimmune disease in a subject comprising, administering to the subject: a functionalized cell comprising, a cell comprising a decorated cell surface, wherein the decorated cell surface comprises at least one covalently attached immune checkpoint molecule; or an acellular pancreatic extracellular matrix comprising, a functionalized cell comprising, a cell comprising a decorated cell surface, wherein the decorated cell surface comprises at least one covalently attached immune checkpoint molecule, and decellularized pancreatic-derived proteins; or a pharmaceutical composition or vaccine comprising a functionalized cell or an acellular pancreatic extracellular matrix.

In another aspect, the subject matter described herein is directed to a method of reversing early-onset type 1 diabetes in a subject comprising, administering to the subject: a functionalized cell comprising, a cell comprising a decorated cell surface, wherein the decorated cell surface comprises at least one covalently attached immune checkpoint molecule; or an acellular pancreatic extracellular matrix comprising, a functionalized cell comprising, a cell comprising a decorated cell surface, wherein the decorated cell surface comprises at least one covalently attached immune checkpoint molecule, and decellularized pancreatic-derived proteins; or a pharmaceutical composition or vaccine comprising a functionalized cell or an acellular pancreatic extracellular matrix.

In another aspect, the subject matter described herein is directed to a method of modulating the T_{reg}:T_{eff} ratio in a subject comprising, administering to the subject: a functionalized cell comprising, a cell comprising a decorated cell surface, wherein the decorated cell surface comprises at least one covalently attached immune checkpoint molecule; or an acellular pancreatic extracellular matrix comprising, a functionalized cell comprising, a cell comprising a decorated cell surface, wherein the decorated cell surface comprises at least one covalently attached immune checkpoint molecule, and decellularized pancreatic-derived proteins; or a pharmaceutical composition or vaccine comprising a functionalized cell or an acellular pancreatic extracellular matrix.

In another aspect, the subject matter described herein is directed to a method of exhausting autoreactive effector T-cells in a subject comprising, administering to the subject: a functionalized cell comprising, a cell comprising a decorated cell surface, wherein the decorated cell surface comprises at least one covalently attached immune checkpoint molecule; or an acellular pancreatic extracellular matrix comprising, a functionalized cell comprising, a cell comprising a decorated cell surface, wherein the decorated cell surface comprises at least one covalently attached immune checkpoint molecule, and decellularized pancreatic-derived proteins; or a pharmaceutical composition or vaccine comprising a functionalized cell or an acellular pancreatic extracellular matrix.

In another aspect, the subject matter described herein is directed to a method of protecting pancreatic beta cells in a subject comprising, administering to the subject: a functionalized cell comprising, a cell comprising a decorated cell surface, wherein the decorated cell surface comprises at least one covalently attached immune checkpoint molecule; or an acellular pancreatic extracellular matrix comprising, a functionalized cell comprising, a cell comprising a decorated cell surface, wherein the decorated cell surface comprises at least one covalently attached immune checkpoint molecule, and decellularized pancreatic-derived proteins; or a pharmaceutical composition or vaccine comprising a functionalized cell or an acellular pancreatic extracellular matrix.

In another aspect, the subject matter described herein is directed to a method of preparing a functionalized cell, comprising: glycoengineering a cell to express a glycoengineered moiety comprising an azide moiety, a cyclooctyne moiety, or a tetrazine moiety; and covalently linking an immune checkpoint molecule through the azide moiety, cyclooctyne moiety, or tetrazine moiety, to prepare a functionalized cell.

In another aspect, the subject matter described herein is directed to a method of preparing a functionalized cell, comprising: covalently attaching an immune checkpoint molecule through a thiol-maleimide conjugation, to prepare a functionalized cell.

In another aspect, the subject matter described herein is directed to an in vivo method of preparing a functionalized cell, comprising: administering a cell labeling agent, such as azide containing sialic acid analogue either as free drug or in a nanoparticle formulation, followed by the administration of a single or multiple immune checkpoint ligands containing reactive group that can conjugate to the cell labeling agent, either as free checkpoint ligands or as a nanoparticle formulation.

In another aspect, the subject matter described herein is directed to an in vivo method for in vivo functionalization of a targeted cell through a two-step pretargeted method comprising: administering a targeted delivery vehicle that can deliver Ac₄ManNAz directly to the targeted cells (e.g., β cells), whereby the surface of the cell is azide modified; and administering a DBCO-functionalized effector component (e.g., DBCO-functionalized PD-L1-Ig) that binds to the azide modified surface, wherein the targeted cell is functionalized.

Additional aspects are also described herein.

### BRIEF DESCRIPTION OF THE SEVERAL VIEWS OF THE DRAWING(S)

Having thus described the invention in general terms, reference will now be made to the accompanying drawings, which are not necessarily drawn to scale.

FIG. 1 illustrates purposed mechanism of PD-L1/CD86/Gal-9-tri-functionalized 3 cells anergize autoreactive T cells and reverse early-onset hyperglycemia. (MHC denotes major histocompatibility complex; AG=antigen; TCR=T cell receptor.)

FIG. 2 illustrates functionalization of NIT-1/β cells via metabolic glycoengineering and biorthogonal click reaction.

FIG. 3a-b illustrates (a) Relative viabilities of NIT-1 cells after culture with different concentrations of Ac₄ManNAz in complete medium for 4 days. (b) Relative viabilities of different functionalized NIT-1 cells determined after culture for 4 days. The viabilities were related to viability of unmodified NIT-1 cells.

FIG. 4 illustrates FACS histograms of azide-functionalized NIT-1 cells after culture with different DBCO-functionalized A488 in Ham's F12 Nutrient Mixture medium at 37° C. for 1 h.

FIG. 5a-b illustrates functionalization of azide-modified NIT-1 cells with (a) DBCO-functionalized PD-L1 and (b) PD-L1-Dend.

FIG. 6a-b illustrates (a) functionalization of PD-L1 with DBCO. (b) azide via amine-NHS ester chemistry and SPACC.

FIG. 7a-c illustrates size-exclusion chromatographs of unfunctionalized and different functionalized (a) PD-L1, (b) CD86, and (c) Gal-9.

FIG. 8a-b illustrates preparation and characterization of DBCO-functionalized PAMAM G5. (a) Preparation of DBCO-PAMAM G5 via amine-NHS chemistry. Unreacted primary amines in the PAMAM G5 were reacted with an excess amount of acetic anhydride. (b)¹H NMR (400 MHz, D₂O) spectra of (i) unmodified PAMAM G5 and (ii) DBCO-functionalized PAMAM.

FIG. 9 illustrates fluorescence image of non-functionalized NIT-1 cells and different TR-PD-L1-functionalized NIT-1 cells.

FIG. 10 illustrates PD-L1 expressions of non-functionalized and different PD-L1-functionalized NIT-1 cells determined at different times after functionalization via FACS method.

FIG. 11 illustrates CLSM images of different PD-L1-functionalized NIT-1 cells recorded at different times after functionalization. The cells were stained with PE-labeled PD-L1 antibody.

FIG. 12 illustrates PD-L1, CD86 and Gal-9 expressions of non-functionalized NIT-1 cells and different mono-/tri-functionalized NIT-1 cells recorded at different times after functionalization quantified via FACS method.

FIG. 13 illustrates CLSM images of non-functionalized NIT-1 cells and different mono-/tri-functionalized NIT-1 cells recorded at different times after functionalization.

FIG. 14a-f illustrates intrapancreatic administration of different PD-L1-functionalized NIT-1 cells reverse early-onset hyperglycemia in NOD mice. (a) Treatment schedule. (b-d) (b) Blood glucose levels, (c) body condition index, and (d) bodyweight change of NOD mice recorded before and after intrapancreatic administration of different PD-L1-functionalized NIT-1 cells. (e) Blood glucose levels of hyperglycemic mice recorded 21 days post-onset of hyperglycemia. (f) Survival curves of mice after received different treatments. p<0.05 implies statistically significant, and p>0.05 implies statistically insignificant.

FIG. 15a-d illustrates intrapancreatic administration of different mono- and tri-functionalized NIT-1 cells reverse early-onset hyperglycemia in NOD mice. (a) Treatment schedule. (b) Blood glucose levels of NOD mice recorded before and after intrapancreatic administration of different mono- and tri-functionalized NIT-1 cells. (c) Blood glucose levels of hyperglycemic mice recorded 21 days post-onset of hyperglycemia. (d) Survival curves of mice after received different treatments. p<0.05 implies statistically significant, and p>0.05 implies statistically insignificant.

FIG. 16 illustrates body condition scores of NOD mice recorded before and after intrapancreatic administration of different mono- and tri-functionalized NIT-1 cells.

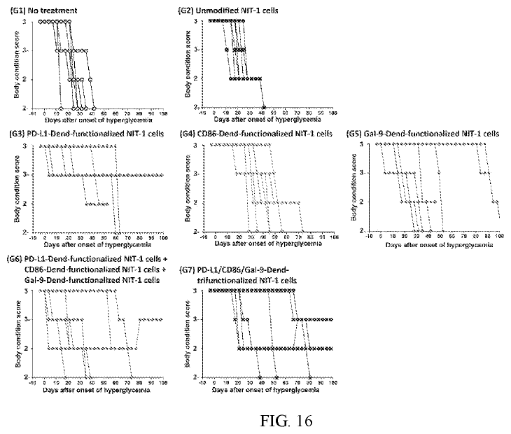

FIG. 17 illustrates bodyweight changes of NOD mice recorded before and after intrapancreatic administration of different mono- and tri-functionalized NIT-1 cells.

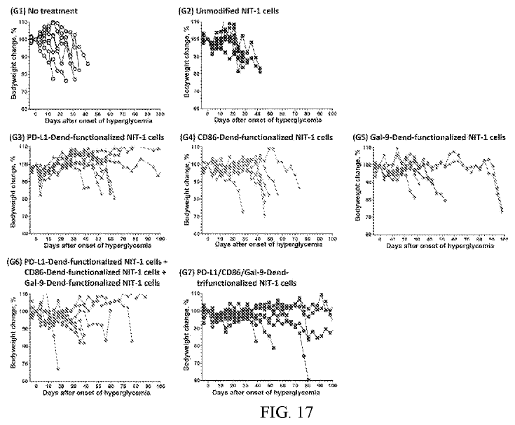

FIG. 18a-c illustrates characterization of decelled pancreatic ECM. (a) Representative H&E-stained images of native pancreas tissue and decelled pancreatic ECM. (b) Representative Oil Red O-stained images of native pancreas tissue and decelled pancreatic ECM. (c) Representative scanning electron microscope images of decelled pancreatic ECM.

FIG. 19a-b illustrates potential in vitro toxicity of pancreatic ECM. (a) Optical microscopy images of tri-functionalized NIT-1 cell cultured in the presence and absence of 40 μg per well of pancreatic ECM in serum-containing culture medium. (b) Relative viabilities of tri-functionalized NIT-1 cell after culture in the presence and absence of pancreatic ECM for 4 days, as determined by MTS assay.

FIG. 20a-b illustrates proliferation of tri-functionalized NIT-1 in serum-free medium contained different concentrations of pancreatic ECM. (a) Optical microscopy images of tri-functionalized NIT-1 cell cultured in the presence of pancreatic ECM in serum-free culture medium. (b) Relative viabilities of tri-functionalized NIT-1 cell after culture in the presence of pancreatic ECM for 4 days, as determined by MTS assay.

FIG. 21 illustrates representative SEM images of pancreatic ECM and tri-functionalized NIT-1 cells cultured in the presence of 10 μg/well of pancreatic ECM.

FIG. 22a-d illustrates (a) s.c. injection of CFSE-labeled NIT-1 cells in health NOD mouse at a site close to the pancreatic lymph nodes. (b) Ex vivo fluorescence images of NOD mice s.c. injected with CFSE-labeled NIT-1 cells in carrier-free and different pancreatic ECM formulations recorded one week post-injection of NIT-1 cells. (c) Average photon efficiencies of different NIT-1 cell grafts. (d) Representative H&E-stained images of different NIT-1 cell grafts.

FIG. 23a-e illustrates subcutaneous administration of PD-L1/CD86/Gal-9-tri-functionalized NIT-1 cells reverse early-onset hyperglycemia in NOD mice. (a) Treatment schedule. (b) Blood glucose levels of NOD mice recorded before and after s.c. administration of different tri-functionalized NIT-1 cells in different pan-ECM formulations. (c) Body condition scores of NOD mice recorded after s.c. administration of tri-functionalized NIT-1 cells in different pan-ECM formulations. (d) Bodyweight of NOD mice recorded after s.c. administration of tri-functionalized NIT-1 cells in different pan-ECM formulations. (e) Survival curves of NOD mice after received different treatments. p<0.05 implies statistically significant, and p>0.05 implies statistically insignificant.

FIG. 24 illustrates a volcano plot (left) showing a quantitative comparison between native and decelled murine pancreata. The green rectangle encompasses the proteins considered to be retained in the decelled samples (fold change>1). The table (right) summarizes the matrisome proteins retained in the PAN-ECM (n=3 biological replicates).

FIG. 25 illustrates PD-L1 Fc-Ig and CD86 Fc-Ig dual-functionalized MOG-expressing mouse Schwann cells (MSCs) or oligodendrocytes (MOL) exhaust MOG-specific T cells.

FIG. 26 illustrates functionalization of MSCs with PD-L1 Fc-Ig and CD86 Fc-Ig. MSCs were first treated with Ac₄ManNAz gave azide-modified MSCs. DBCO-functionalized PD-L1 Fc-Ig and CD86 Fc-Ig were then conjugated to the azide-modified MSCs via SPACC.

FIG. 27 illustrates functionalization of PD-L1 Fc-Ig and CD86 Fc-Ig with DBCO-EG13-NHS ester via amine-NHS ester chemistry. Characterization of PD-L1 Fc-Ig and CD86 Fc-Ig via UV-visible spectroscopy method.

FIG. 28 illustrates quantification of A488-labeled and DBCO-functionalized PD-L1 Fc-Ig and Texas Red-labeled DBCO-functionalized CD86 Fc-Ig retained on the azide-modified MSCs after conjugation via spectroscopic method.

FIG. 29 illustrates time-dependent PD-L1 and CD86 expressions of unmodified and different functionalized MSCs, as determined by FACS method.

FIG. 30 illustrates administration of PD-L1 Fc-Ig and CD86 Fc-Ig dual-functionalized MSCs in prevention treatment (1 days after immunization) delay the onset of EAE and relieve the maximum EAE clinical score.

FIG. 31 illustrates administration of PD-L1 Fc-Ig and CD86 Fc-Ig dual-functionalized MSCs in therapeutic treatment (17 days after immunization) partly reverse EAE and relieve the EAE score after onset.

FIG. 32 illustrates that PD-L1- and CD86-functionalized MSCs prevent and ameliorate active EAE in the mouse. The scheme illustrates the mechanism of actions of drug-free and LEF-encapsulated PD-L1-Ig/CD86-Ig NP-functionalized MSCs to prevent and treat EAE in the mouse. The myelin antigen-rich PD-L1-Ig/CD86-Ig NP-functionalized MSCs can simultaneously present the myelin antigen to the myelin-specific CD4⁺ T cells and inhibit PD-1/PD-L1 and CTLA-4/CD86 immune checkpoint pathways. In prophylactic treatment, the i.v. administered functionalized MSCs inhibit the activation of myelin-specific CD4⁺ T cells and the subsequent differentiation into pathogenic Tₕ1 and Tₕ17 cells, and promote the development of myelin-specific T_{reg} cells. In therapeutic treatment, the functionalized MSCs inhibit the activation of myelin-specific CD4⁺ T cells, reduce the pathogenic Tₕ1 and Tₕ17 cells, and promote the development of antigen-specific T_{reg} cells. In addition, the induced T_{reg} cells and i.v. administered MSCs can enter the CNS to inhibit the activation of pathogenic Tₕ1 and Tₕ17 cells and cytotoxic T cells. Furthermore, the encapsulated LEF release inside the CNS directly inhibits the proliferation of autoreactive CD4⁺ and CD8⁺ T cells and generates a less proinflammatory CNS microenvironment for the OL to repair the damaged myelin sheaths. The antigen-specific immunotherapy effectively prevents systemic immune suspension. (AG=antigen, TCR=T cell receptor, MCH II=major histocompatibility complex class II.)

FIG. 33a-d illustrates that bioengineering of PD-L1 and CD86 functionalized MSCs. a, (i) Bioengineering PD-L1 Fc-Ig and CD86 Fc-Ig directly functionalized MSCs through metabolic glycoengineering followed by SPAAC with DBCO-functionalized PD-L1 Fc-Ig and CD86 Fc-Ig. (ii, iii) Size distributions (ii), PD-L1, and CD86 expressions (iii) of unmodified and PD-L1 Fc-Ig and CD86 Fc-Ig directly functionalized MSCs. b, (i) Structures of drug-free DBCO and MTZ dual-functionalized PEG-PLGA NPs (DBCO/MTZ NPs) and LEF-encapsulated DBCO/MTZ NPs (DBCO/MTZ LEF NPs). (ii, iii) TEM images (ii), and intensity-average diameter (Dₕ) distributions (iii) of drug-free and LEF-encapsulated DBCO and MTZ dual-functionalized NPs. (iv) Drug-release profile of LEF-encapsulated DBCO and MTZ dual-functionalized NPs at physiological conditions in the presence of large excess of PBS. c, (i) Bioengineering of PD-L1 Fc-Ig and CD86 Fc-Ig NP-dual-functionalized MSCs. The dual-functionalized MSCs were engineered via 3 steps: first, metabolic labeling of Ac₄ManNAZ gave azide-modified MSCs; second, the conjugation of DBCO/MTZ NPs (or DBCO/MTZ LEF NPs) onto the azide-modified MSCs through SPAAC at the physiological conditions; and finally, the bioconjugation of TCO-functionalized PD-L1 Fc-Ig and CD86 Fc-Ig onto the DBCO/MTZ NP-functionalized MSCs via IEDDA at the physiological conditions. (ii-iv) Size-distributions (ii), scanning electron microscopy (SEM) images (iii), and PD-L1 and CD86 expressions (iv) of unmodified and PD-L1 Fc-Ig and CD86 Fc-Ig NP-functionalized MSCs. Pseudopodia can be identified from the SME images of both unmodified and functionalized MSCs. The red arrows in the SEM images highlighted the PD-L1-Ig/CD86-Ig LEF NPs grafted on the surface of the MSCs. d, Representative CLSM images of different as-functionalized MSCs.

FIG. 34a-e illustrates PD-L1- and CD86-functionalized MSCs upregulate PD-1 and CTLA-4 pathways in myelin-specific T cells, downregulate T cell activation and promote the development of induced regulatory T cells in vitro. a, b, PD-1 (a), and CTLA-4 (b) expressions of myelin-specific 2D2 T cells after incubated with different types of PD-L1-Ig- and/or CD86-Ig-functionalized MSCs for 48 h, as determined by FACS assay. Cells were initially gated at CD3⁺ cells. (n=4) c, d, ELISA analysis of INF-gamma (c) and TL-17A (d) secreted from 2D2 CD4⁺ T cells after incubated with different functionalized MSCs. Supernatants were collected 48 h post-incubation for the ELISA analysis. (n=4) e, Quantification of IL-10⁺ and FoxP3⁺ population in 2D2 CD4⁺ T cells after incubated with different functionalized MSCs for 48 h via FACS. Cells were initially gated at CD3⁺ cells. (n=3).

FIG. 35a-e illustrates that PD-L1-Ig and CD86-Ig directly functionalized MSCs prophylactically and therapeutically suppress MOG₃₅₋₅₅-induced EAE in vivo. a, Prophylactic and therapeutic treatment schedules after immunization with MOG₃₅₋₅₅ peptide. 2×10⁶ of unmodified or functionalized MSCs were i.v. administrated 1 day (prophylactic treatment) or 17 days (therapeutic treatment) post-immunization (p.i.). Body conditions were monitor daily until day 35 p.i. Mice were euthanized day 36 or 37 p.i. The spinal columns were preserved for further histopathological studies. b, Time-dependent mean clinical scores of EAE inflicted mice after received different prophylactic or therapeutic treatments. In the absence of treatment, EAE progress from partial tail paresis (score 0.5), complete tail paresis (score 1.0), limp tail and hind leg inhibition (score 1.5), limp tail and weakness of hind legs (score 2.0), limp tail and no movement in one leg (score 2.5), to complete hind limb paralysis (score 3.0). (n=9 mice per group.) c, Cumulative EAE scores of EAE inflicted mice after received different treatments. d, (i) Representative hematoxylin and eosin (H&E)-stained spinal cord sections preserved from healthy disease-free mouse and EAE-inflicted mice after received different prophylactic and therapeutic treatments with directly functionalized MSCs. (ii) Quantification of spinal inflammation from the H&E-stained images of spinal cords. (n=3 for the non-treatment group; n=8 for both prophylactic treatment groups; n=7 for therapeutic treatment group treated with the non-functionalized MSCs; n=6 for the therapeutic treatment group treated with the functionalized MSCs.) e, (i) Representative Luxol fast blue (LFB)-stained spinal cord sections preserved from healthy disease-free mouse and EAE-inflicted mice after received different prophylactic and therapeutic treatments with directly functionalized MSCs. Myelin fibers and phospholipids appear blue to green, neuropil appears pink, and nerve cells appear purple. (ii) Quantification of demyelination from the LFB-stained images of spinal cords. (n=3 for the non-treatment group; n=8 for both prophylactic treatment groups; n=7 for therapeutic treatment group treated with the non-functionalized MSCs; n=6 for the therapeutic treatment group treated with the functionalized MSCs.)

FIG. 36a-h illustrates that PD-L1- and CD86-conjugated NP-functionalized MSCs effectively suppress progressive chronic MOG₃₅₋₅₅-EAE model and relapsing-remitting PLP₁₇₈₋₁₉₁-EAE model in vivo, prophylactically, and therapeutically. a, Prophylactic and therapeutic treatment schedules with PD-L1-Ig/CD86-Ig NP-functionalized MSCs in C57BL/6 mice after immunization with MOG₃₅₋₅₅ peptide. Unmodified or functionalized MSCs were i.v. administrated 1 day (prophylactic treatment) or 17 days (therapeutic treatment) p.i. Body conditions were monitor daily until 35 days p.i. Mice were euthanized 36 or 37 days p.i., spinal columns were preserved for further histopathological studies. In control treatment groups 2, 3, 6, and 7, free or NP conjugated PD-L1 Fc-Ig, and CD86 Fc-Ig (plus unencapsulated LEF) were i.v. administrated 20 min before the non-functionalized MSCs. b, Time-dependent mean clinical scores of MOG₃₅₋₅₅-induced EAE inflicted mice after received different prophylactic and therapeutic treatments. (n=8 mice per group; one non-treatment group mouse was found dead 28 days p.i.) c, Cumulative EAE scores of MOG₃₅₋₅₅-EAE inflicted mice after received different treatments. d, (i) Representative H&E-stained spinal cord sections preserved from EAE-inflicted mice after received different prophylactic and therapeutic treatments with drug-free/LEF-encapsulated PD-L1-Ig/CD86-Ig NP-functionalized MSCs. (ii) Quantification of spinal inflammation from the H&E-stained images of spinal cords. (n=3 for the non-treatment group; n=6 for the prophylactic treatment group and therapeutic treatment group treated with drug-free PD-L1-Ig/CD86-Ig NP-functionalized MSCs; n=7 for the therapeutic treatment group treated with PD-L1-Ig/CD86-Ig LEF NP-functionalized MSCs.) e, (i) Representative LFB-stained spinal cord sections preserved from EAE-inflicted mice after received different prophylactic and therapeutic treatments with drug-free/LEF-encapsulated PD-L1-Ig/CD86-Ig NP-functionalized MSCs. (ii) Quantification of demyelination from the LFB-stained images of spinal cords. (n=3 for the non-treatment group; n=6 for the prophylactic treatment group and therapeutic treatment group treated with drug-free PD-L1-Ig/CD86-Ig NP-functionalized MSCs; n=7 for the therapeutic treatment group treated with PD-L1-Ig/CD86-Ig LEF NP-functionalized MSCs.) f, Prophylactic and therapeutic treatment schedules with PD-L1-Ig/CD86-Ig NP-functionalized MSCs in C57BL/6 mice after immunization with PLP₁₇₈₋₁₉₁ peptide. Unmodified or functionalized MSCs were i.v. administrated 1 day (prophylactic treatment) or 18 days (therapeutic treatment) p.i. Body conditions were monitor until 35 days p.i. g, Time-dependent mean clinical scores of MOG₃₅₋₅₅-induced EAE inflicted mice after received different prophylactic and therapeutic treatments. (n=8 mice per group, except n=7 for the therapeutic treatment group with unmodified MSCs.) h, Cumulative EAE scores of PLP₁₇₈₋₁₉₁-EAE inflicted mice after received different treatments.

FIG. 37a-e illustrates that PD-L1-Ig/CD86-Ig NP-functionalized MSCs effectively promote the development of MOG-specific T_{reg} cells in the MOG₃₅₋₅₅-EAE mouse model. a, Splenetic MOG-specific (i) Tₕ1, (ii) Tₕ17, and (iii) T_{reg} cells populations in EAE-inflicted mice 3 days after different prophylactic treatments (5 days p.i.). (n=5) b, Splenetic MOG-specific (i) Tₕ1, (ii) Tₕ17, and (iii) T_{reg} cells populations in EAE-inflicted mice 3 days after different therapeutic treatments (5 days p.i.). MOG-specific (iv) Tₕ1, (v) Tₕ17 and (vi) T_{reg} cells, and (vii) antigen non-specific INF-γ⁺ cytotoxic T cell populations in the spinal cord of EAE-inflicted mice 3 days after different therapeutic treatments (21 days p.i.). (n=5) c, Splenetic MOG-specific T_{reg} cells populations in EAE-inflicted mice 38 days p.i. after different prophylactic and therapeutic treatments. (n=6) d, Representative anti-CD4- and anti-FoxP3-stained immunofluorescence images of spinal cord preserved from non-treated EAE-inflicted mice and different treated EAE-inflicted mice 38 days p.i. e, Prophylactic and therapeutic treatments with PD-L1-Ig/CD86-Ig LEF NP-functionalized MSCs in MOG₃₅₋₅₅-immunized mice with and without T_{reg} cell depletion. Mice in T_{reg} cell depletion groups received 3 intraperitoneal (i.p.) injections of anti-CD25 (200 μg per injection) before and after the treatments with the MSCs to achieve T_{reg} cell depletion. (iii) Time-dependent mean clinical scores of MOG₃₅₋₅₅-induced EAE inflicted mice after received different (i) prophylactic and (ii) therapeutic treatments. (iii) Cumulative EAE scores of MOG₃₅₋₅₅-EAE inflicted mice after received different treatments with and without T_{reg} cell depletion. (n=6).

FIG. 38 illustrates that MSCs and MOLs express common myelin antigens. Representative FACS histograms of anti-MOG- and anti-PLP1-stained MSCs, MOLs, and MIN6 cells (insulinoma cells isolated C57BL/6 mice). Both anti-MOG and anti-PLP1 rabbit polyclonal antibodies were labeled via A488-labeled goat anti-rabbit IgG. The MIN6 cells were used for negative control.

FIG. 39a-d illustrates that MSCs and MOLs remain viable after incubated with small-molecule Ac₄ManNAz, small-molecule LEF, and after bioconjugation. a, In vitro viabilities of MSCs and MOLs after incubated with different concentrations of small-molecule Ac₄ManNAz, as quantified by MTS assay. b, In vitro viabilities of MSCs and MOLs after incubated with different concentrations of small-molecule LEF, as quantified by MTS assay. Small-molecule LEF showed moderate in vitro toxicity against MSCs and insignificant toxicity against MOLs. This suggests LEF would not affect OLs (that already in the CNS) to repair the damaged myelin. c, Relative viabilities of different directly functionalized MSCs, as determined by MTS assay. d, Relative viabilities of drug-free and LEF-loaded PD-L1-Ig/CD86-Ig NP-functionalized MSCs and MOLs, as determined by MTS assay. (n=8)

FIG. 40a-d illustrates that Characterization of DBCO-functionalized PD-L1-Ig and DBCO-functionalized CD86-Ig. a, The scheme illustrates covalent conjugation of DBCO-functionalized ethylene glycol (EG) to the PD-L1 and CD86-Ig fusion proteins through amine-N-hydroxysuccinimide (NHS) ester coupling reaction at different target degree of functionalization (D_{f, Target}). b, UV-visible absorption spectra of different DBCO-functionalized PD-L1-Ig and CD86-Ig fusion proteins (1 mg/mL). c, The plot of the actual degree of functionalization of PD-L1-Ig and CD86-Ig. DBCO-functionalized PD-L1-Ig (with 8 conjugated DBCO) and DBCO-functionalized CD86-Ig (with 9 conjugated DBCO) prepared at a D_{f, Target} of 45 were used for functionalization of MSCs and MOLs. d, Right spectra, UV-visible absorption spectra of TCO-functionalized PD-L1-Ig and CD86-Ig (1 mg/mL). Both TCO-functionalized fusion proteins were functionalized as with the DBCO-functionalized fusion proteins with a target degree of functionalization of 45; and left spectra, UV-visible absorption spectra of purified TCO-functionalized PD-L1-Ig and CD86-Ig after reacted with 5 molar equivalents of Cy5 tetrazine (probe) at 37° C. for 1 h (normalized to 1 mg/mL). The reactions were carried out at a protein concentration of 0.5 mg/mL in serum- and phenol red-free DMEM medium (the same fusion protein concentration that used in functionalization of MSCs). Unreactive dye and DMEM were removed via PD-10 desalting columns. Both functionalized fusion proteins contain less conjugated active TCO were removed via PD-10 desalting columns. Both functionalized fusion proteins contain less conjugated active TCO (an average of 2 active TCO molecule per fusion protein) than that functionalized with DBCO ligand because of trans-to-cis isomerization at the basic conjugation condition inactivated the TCO ligand and thiols in culture medium reacted with the conjugated TCO.

FIG. 41 illustrates that Characterization of A488-labeled DBCO-functionalized PD-L1-Ig and Texas Red-labeled DBCO-functionalized CD86-Ig. Representative UV-visible spectra of non-labeled DBCO-functionalized fusion PD-L1-Ig, CD86-Ig, A488-labeled DBCO-functionalized PD-L1-Ig fusion protein, and Texas Red-labeled DBCO-functionalized PD-L1-Ig fusion protein. It was calculated that the functionalized PD-L1-Ig fusion protein contains an average of one conjugated A488 molecule. The functionalized CD86-Ig fusion protein contains an average of two conjugated Texas Red molecules.

FIG. 42 illustrates that Covalently conjugated PD-L1-Ig and CD86-Ig gradually detached from mono- and dual-functionalized MSCs at the physiological conditions. Quantification of the detachment rate of A488-labeled PD-L1-Ig and Texas Red-labeled CD86-Ig from mono-and dual-functionalized MSCs at the physiological conditions via fluorescence spectroscopy. About half of the conjugated fusion proteins detached from the MSCs within 24 h after conjugation. (n=8, cell seeding density=10,000 cells per well.)

FIG. 43 illustrates that PD-L1-Ig/CD86-Ig Cy5-labeled NPs slowly detached from MSCs at the physiological conditions. Quantification of the detachment rate of PD-L1-Ig/CD86-Ig-conjugated Cy5-labeled NPs from MSCs at the physiological conditions via fluorescence spectroscopy. About half of the conjugated Cy5-labeled NPs retained on the MSCs 48 h after functionalization. (n=8, cell seeding density=10,000 cells per well.)

FIG. 44a-b illustrates that PD-L1 and CD86 expressions of PD-L1-Ig/CD86-Ig mono-/dual-directly functionalized MSCs gradually declined after functionalization. a, Representative FACS histograms show the PD-L1 and CD86 expressions of PD-L1-Ig and CD86-Ig mono- or dual-directly functionalized MSCs after stained with PE-labeled PD-L1 and A488-labeled CD86. The PD-L1 and CD86 expressions declined to the background level 3 days post-functionalization. (n=3) b, Representative FACS histograms show the PD-L1 and CD86 expressions unmodified (azido-free) MSCs after incubated with PD-L1-Ig and/or CD86-Ig at physiological conditions for 1 h. The incubated cells were washed before stained with anti-PD-L1 and anti-CD86 antibodies for the FACS study. The FACS study confirmed that the bioconjugation process does not induce significant non-specific binding of FcIg fusion proteins.

FIG. 45a-b illustrates that PD-L1-Ig/CD86-Ig NP slowly detached from the surface of azide-modified MSCs after functionalization. a, Representative FACS histograms show the Cy5 fluorescence intensities of PD-L1-Ig/CD86-Ig Cy5-labeled NP-functionalized MSCs recorded at different times after functionalization. b, Representative FACS histograms show the PD-L1 and CD86 expressions of PD-L1-Ig and CD86-Ig NP-functionalized MSCs after stained with PE-labeled PD-L1 and A488-labeled CD86. The PD-L1 and CD86 expressions slowly decline to the background level 3 days post-functionalization. (n=3).

FIG. 46 illustrates that PD-L1-Ig/CD86-Ig NP slowly detached from the surface of azide-modified MOLs after functionalization. Representative FACS histograms show the PD-L1 and CD86 expressions of PD-L1-Ig and CD86-Ig NP-functionalized MOLs after stained with PE-labeled PD-L1 and A488-labeled CD86. The PD-L1 and CD86 expressions slowly decline to the background level 3 days post-functionalization. (n=3).

FIG. 47 illustrates successful conjugation of PD-L1-Ig and/or CD86-Ig onto the surface of azide-modified MSCs. Representative CLSM images of unmodified and the as-functionalized MSCs after stained with PE-labeled anti-PD-L1 and A488-labeled anti-CD86 antibodies.

FIG. 48a-b illustrates that PD-L1- and CD86-bioengineered MSCs upregulate the PD1 and CTLA-4 expressions of the incubated 2D2 cells. a, Representative FACS histograms of A488-labeled anti-PD-1 stained 2D2 cells (MOG-specific CD4⁺ cells) after incubated with different functionalized MSCs at an effector:target ratio (E/T) of 10:1 for 48 h. b, Representative FACS histograms of PE-labeled anti-CTLA-4 stained 2D2 cells (MOG-specific CD4⁺ cells) after incubated with different functionalized MSCs at a E/T ratio of 10:1 for 48 h.

FIG. 49 illustrates that PD-L1-and CD86-bioengineered MSCs promote the development of antigen-specific IL10⁺ FoxP3⁺ T_{reg} cells. Representative two-dimensional FACS plots of A488-labeled anti-FoxP3- and PE-labeled anti-IL10-intracellular stained 2D2 cells after incubated with different functionalized MSCs at an E/T ratio of 10:1 for 3 days. The bioengineered MSCs promote the development of IL10⁺ and FoxP3⁺ T_{reg} cells. Cells were initially gated at CD3⁺ cells. (n=3).

FIG. 50a-b illustrates that PD-L1-and CD86-bioengineered MOLs upregulate the PD1 and CTLA-4 expressions of the incubated 2D2 cells. a, Representative FACS histograms of A488-labeled anti-PD-1 stained 2D2 cells (MOG-specific CD4⁺ cells) after incubated with PD-L1-Ig/CD86-Ig NP-functionalized MOLs at an E/T of 10:1 for 48 h. b, Representative FACS histograms of PE-labeled anti-CTLA-4 stained 2D2 cells (MOG-specific CD4⁺ cells) after incubated with PD-L1-Ig/CD86-Ig NP-functionalized MOLs at an E/T of 10:1 for 48 h. (n=4).

FIG. 51 illustrates that PD-L1-and CD86-bioengineered MOLs inhibit the proliferation of pathogenic CD4⁺ cells. IFN-γ and IL-17A released from 2D2 cells after incubated with PD-L1-Ig/CD86-Ig NP-functionalized MOLs at an E/T of 10:1 for 48 h, as quantified by the ELISA method. (n=4).

FIG. 52 illustrates that PD-L1-Ig/CD86-Ig NP-functionalized MOLs promote the development of antigen-specific IL10⁺ FoxP3⁺ T_{reg} cells. Representative two-dimensional FACS plots of A488-labeled anti-FoxP3- and PE-labeled anti-IL10-intracellular stained 2D2 cells were incubated with PD-L1-Ig/CD86-Ig NP-functionalized MOLs at an E/T of 10:1 for 3 days. The bioengineered MSCs promote the development of IL10⁺ and FoxP3⁺ T_{reg} cells. Cells were initially gated at CD3⁺ cells.

FIG. 53 illustrates that PD-L1-Ig/CD86-Ig NP-functionalized MSCs inhibit the proliferation of stimulated cytotoxic T cells in an antigen-non-specific behavior. CFSE-dilution assay of CFSE-labeled CD8⁺ T cells (isolated from wide-type C57BL/6 mice) after incubated with different functionalized MSCs at an E:T of 1:1 for 48 h. The cytotoxic T cells were cultured under stimulation conditions (i.e., in the presence of Dynabeads T Cell Activation beads at a 1:1 molar ratio). The proliferation of cytotoxic T cells was quantified via the FACS method. Cells were initially gated at CD8⁺ cells. (n=4).

FIG. 54a-c illustrates that Intravenous administration of unmodified MSCs and PD-L1-Ig/CD86-Ig NP-functionalized MSCs did not cause long-term side effects. a, Clinical chemistry of blood samples collected from healthy untreated C57BL/6 mice (female, about 15 weeks old) and healthy C57BL/6 mice after i.v. administration of unmodified MSCs or PD-L1-Ig/CD86-Ig NP-functionalized MSCs (2×10⁶ cells/mouse). The blood samples were collected 5 weeks post-administration of the MSCs. b, Bodyweight change of healthy C57BL/6 mice after i.v. administration of unmodified MSCs or PD-L1-Ig/CD86-Ig NP-functionalized MSCs (2×10⁶ cells/mouse). The red dotted line represents the normal range of each blood chemistry parameter. c, Representative H&E-stained heart, lung, spleen, kidney, and liver session preserved from C57BL/6 mice 5 weeks after i.v. administration of MSCs or PD-L1-Ig/CD86-Ig NP-functionalized MSCs. (n=6, excepted n=8 for the experimental group i.v. administered with the PD-L1-Ig/CD86-Ig NP-functionalized MSCs.)

FIG. 55a-c illustrates that PD-L1-Ig/CD86-Ig directly functionalized MSCs suppress active MOG₃₅₋₅₅-induced EAE, prophylactically and therapeutically. a, Maximum EAE scores in mice after received prophylactic treatment (at 1-day p.i.) with unmodified or different directly functionalized MSCs (2×10⁶ cells per mouse, via i.v. injection). b, EAE scores of MOG-induced EAE mice (at 35-days p.i.) after received prophylactic treatment (at 1-day p.i.) with unmodified or different directly functionalized MSCs. c, EAE scores of MOG-induced EAE mice (at 35-days p.i.) after received therapeutic treatment (at 17-days p.i.) with unmodified and directly functionalized MSCs. (n=9).

FIG. 56 illustrates that Drug-free and LEF-encapsulated PD-L1-Ig/CD86-Ig NP-functionalized MSCs effectively prevent and ameliorate active MOG₃₅₋₅₅-induced EAE by inhibiting spinal inflammation. Representative H&E-stained spinal cord cross-sections (and the corresponding inflammatory score) of healthy and EAE-inflicted mice after prophylactic and therapeutic treatment with drug-free and LEF-encapsulated PD-L1-Ig/CD86-Ig LEF NP-functionalized MSCs. Mice received prophylactic treatment on day 1 p.i., and therapeutic treatment on day 17 p.i. Spinal columns were preserved 36 or 37 days p.i.

FIG. 57 illustrates that Drug-free and LEF-encapsulated PD-L1-Ig/CD86-Ig NP-functionalized MSCs effectively prevent and ameliorate active MOG₃₅₋₅₅-induced EAE by preventing demyelination. Representative LEF-stained spinal cord cross-sections (and the corresponding inflammatory score) of healthy and EAE-inflicted mice after prophylactic and therapeutic treatment with drug-free and LEF-encapsulated PD-L1-Ig/CD86-Ig LEF NP-functionalized MSCs. Mice received prophylactic treatment on day 1 p.i., and therapeutic treatment on day 17 p.i. Spinal columns were preserved day 36 or 37 p.i.

FIG. 58a-c illustrates that PD-L1-Ig/CD86-Ig LEF NP-functionalized MSCs suppress active MOG₃₅₋₅₅-induced EAE, prophylactically and therapeutically. a, Maximum EAE scores in mice after received prophylactic treatment (at 1-day p.i.) with unmodified or different NP functionalized MSCs (2×10⁶ cells per mouse, via i.v. injection). b, EAE scores of MOG-induced EAE mice (at 35-days p.i.) after received prophylactic treatment (at 1-day p.i.) with unmodified or different NP functionalized MSCs. c, EAE scores of MOG-induced EAE mice (at 35 days p.i.) after received therapeutic treatment (at 17-days p.i.) with unmodified or different NP functionalized MSCs. (n=8 mice per group; one non-treatment group mouse was found dead 28 days p.i.)

FIG. 59a-e illustrates that drug-free and LEF-encapsulated PD-L1-Ig/CD86-Ig NP-functionalized MSCs are equally effective in preventing the development of severe EAE symptoms in the MOG₃₅₋₅₅-induced EAE model. a, Prophylactic treatment schedule. Drug-free and LEF-encapsulated PD-L1-Ig/CD86-Ig NP-functionalized MSCs (2×10⁶ cells per mouse) were i.v. administrated 24 h p.i. b, Time-dependent EAE scores after prophylactic treatment with drug-free and LEF-encapsulated PD-L1-Ig/CD86-Ig NP-functionalized MSCs. c, Maximum EAE after different prophylactic treatments. d, EAE score recorded at day 35 p.i. after different prophylactic treatments. e, Cumulative EAE score (up to 35-days p.i.) after different prophylactic treatments. (n=6)

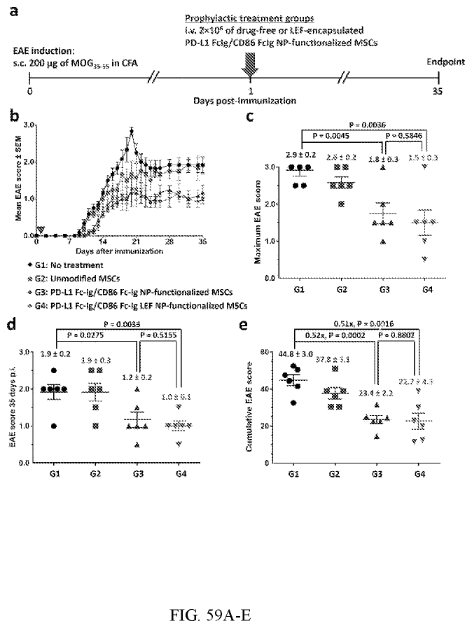

FIG. 60 illustrates that Drug-free and LEF-encapsulated PD-L1-Ig/CD86-Ig NP-functionalized MSCs effectively prevent and ameliorate active MOG₃₅₋₅₅-induced EAE by inhibiting spinal inflammation. Representative H&E-stained spinal cord cross-sections (and the corresponding inflammatory score) of healthy and EAE-inflicted mice after prophylactic and therapeutic treatment with drug-free and LEF-encapsulated PD-L1-Ig/CD86-Ig LEF NP-functionalized MSCs. Mice received prophylactic treatment on day 1 p.i., and therapeutic treatment on day 17 p.i. Spinal columns were preserved 36 or 37 days p.i.

FIG. 61 illustrates that Drug-free and LEF-encapsulated PD-L1-Ig/CD86-Ig NP-functionalized MSCs effectively prevent and ameliorate active MOG₃₅₋₅₅-induced EAE by preventing demyelination. Representative LEF-stained spinal cord cross-sections (and the corresponding inflammatory score) of healthy and EAE-inflicted mice after prophylactic and therapeutic treatment with drug-free and LEF-encapsulated PD-L1-Ig/CD86-Ig LEF NP-functionalized MSCs. Mice received prophylactic treatment on day 1 p.i., and therapeutic treatment on day 17 p.i. Spinal columns were preserved day 36 or 37 p.i.

FIG. 62a-c illustrates that a booster dose of therapeutic treatment with PD-L1-Ig/CD86-Ig LEF NP-functionalized MSCs is more effective in suppressing active MOG₃₅₋₅₅-induced EAE. a, Time-dependent EAE score after therapeutic treatments (at day 18 and 36 p.i.) with PD-L1-Ig/CD86-Ig LEF NP-functionalized MSCs (2×10⁶ cells per mouse). b, EAE scores were recorded at day 35 (before second treatment) and day 50 p.i. (study endpoint). c, right: cumulative EAE score of non-treatment and therapeutic treatment groups recorded between day 18 and 36 p.i. after the first treatment; left: Cumulative EAE score of non-treatment and therapeutic treatment groups recorded between day 37 and 50 p.i. after the second treatment. (n=6. The mice reported in this study were identical to the non-treatment group and therapeutic treatment group (without T_{reg} cell depletion) mice reported in the mechanistic study (statistical analysis ended on day 28 p.i.).)

FIG. 63 illustrates that PD-L1-Ig/CD86-Ig LEF NP-functionalized MSCs suppress active PLP₁₇₈₋₁₉₁-induced EAE, prophylactically and therapeutically. EAE scores of PLP₁₇₈₋₁₉₁-induced EAE mice (recorded at 35-days p.i.) after received prophylactic treatment (at 1-day p.i.) or therapeutic treatment (at 18-days p.i.) with unmodified or different NP functionalized MSCs. (n=8 mice per group, except n=7 for the therapeutic treatment group with unmodified MSCs.)

FIG. 64a-c illustrates that a second dose of therapeutic treatment with PD-L1-Ig/CD86-Ig LEF NP-functionalized MSCs effectively suppresses active PLP₁₇₈₋₁₉₁-induced EAE. a, Time-dependent EAE score after therapeutic treatments (at day 18 and 35 p.i.) with PD-L1-Ig/CD86-Ig LEF NP-functionalized MSCs (2×10⁶ cells per mouse). The gradient of the plot represents the progression of the disease. Without any treatment, the progression rate was 0.0038 day⁻¹. The disease proregression rate was 0.0402 day⁻¹ after the first therapeutic treatment. The disease progression rate dropped to 0.0044 day⁻¹ after the second therapeutic treatment. b, Cumulative EAE score of non-treatment and therapeutic treatment group EAE-inflicted mice recorded between day 18 and 35 p.i. after the first treatment. c, Cumulative EAE score of non-treatment and therapeutic treatment group EAE-inflicted mice recorded between day 35 and 70 p.i. after the second treatment. (n=8; one mouse in the therapeutic treatment Group 6 was found dead at day 37 p.i. (1 day after the second treatment).)

FIG. 65a-c illustrates that 50 Gy X-ray irradiation kills PD-L1-Ig/CD86-Ig LEF NP-functionalized MSCs. a, Time-dependent optical microscopy images of non-irradiated and 50 Gy X-ray-irradiated PD-L1-Ig/CD86-Ig LEF NP-functionalized MSCs. b, Relative viabilities of 50 Gy X-ray-irradiated PD-L1-Ig/CD86-Ig LEF NP-functionalized MSCs at different times after irradiation. (n=8) c, Digital photograph of non-irradiated and 50 Gy X-ray-irradiated PD-L1-Ig/CD86-Ig LEF NP-functionalized MSCs after cultured at physiological conditions for 10 days. Colonies were stained by 1% crystal violet.

FIG. 66a-d illustrates that LEF-encapsulated PD-L1-Ig/CD86-Ig NP-functionalized MOLs effectively ameliorate in the MOG₃₅₋₅₅-immunized EAE mice. a, Therapeutic treatment schedule. Unmodified MOLs and LEF-encapsulated PD-L1-Ig/CD86-Ig NP-functionalized MOLs (2×10⁶ cells per mouse) were i.v. administrated 17 h p.i. b, Time-dependent EAE scores after therapeutic treatment with LEF-encapsulated PD-L1-Ig/CD86-Ig NP-functionalized MOLs. c, EAE score recorded at day 35 p.i. after different therapeutic treatments. d, Cumulative EAE score (up to 35-days p.i.) after therapeutic treatments. (n=7, except n=8 for the therapeutic treatment group with PD-L1-Ig/CD86-Ig LEF NP-functionalized MOLs.)

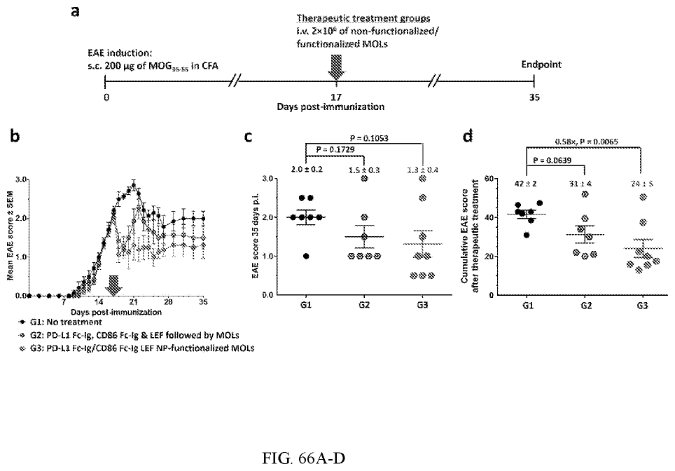

FIG. 67a-c illustrates that intramuscular administration of drug-free/LEF-encapsulated PD-L1-Ig/CD86-Ig LEF NP-functionalized MSCs and MOLs effectively ameliorate MOG₃₅₋₅₅-induced-induced EAE. a, Time-dependent EAE score after different therapeutic treatments with two i.m. administration of drug-free/LEF-encapsulated PD-L1-Ig/CD86-Ig NP-functionalized MSCs and MOLs at day 18 and day 28 p.i. b, Cumulative EAE score after the first therapeutic treatment. c, Cumulative EAE score after the second therapeutic treatment.

FIG. 68a-c illustrates that Biodistribution of i.v. administered non-functionalized and PD-L1-Ig/CD86-Ig NP-functionalized VT680-labeled MSCs in MOG₃₅₋₅₅-induced EAE mice. a, Ex vivo imaging schedules. EAE-inflicted mice were euthanized 48 h after i.v. administration of different VT680-labeled MSCs, either in a prophylactic study (at day 3) or therapeutic study (at day 19). b, Ex vivo fluorescent images of the brain (BR), lung (LU), heart (HE), liver (LI), spleen (SP), kidney (KI), and spinal cord (SC) preserved from non-treatment and different treatment group mice. c, Biodistribution of i.v. administered VT680-labeled MSCs. (n=5).

FIG. 69a-c illustrates that biodistribution of i.v. administered non-functionalized and PD-L1-Ig/CD86-Ig NP-functionalized VT680-labeled MSCs in MOG₃₅₋₅₅-induced EAE mice. a, Ex vivo imaging schedules. EAE-inflicted mice were euthanized 48 h after i.v. administration of different VT680-labeled MSCs, either in a prophylactic study (at day 3) or therapeutic study (at day 19). b, Ex vivo fluorescent images of the brain (BR) and spinal cord (SC) preserved from non-treatment and different treatment group mice. c, Biodistribution of i.v. administered VT680-labeled MSCs. (n=5)

FIG. 70 illustrates representative FACS gating strategy for analyzing autoreactive CD8⁺ T cell and different MOG-specific CD4⁺ T cell populations in the spinal cord and spleen. Diagram summarizes the gating strategy for analysis the IFN-γ⁺ CD8⁺ T cells (autoreactive cytotoxic T cells), MOG-specific pathogenic Tₕ1 (MOG⁺ IFN-γ⁺ CD4⁺) and Tₕ17 (MOG⁺ IL17A⁺ CD4⁺) cells, and suppressive T_{reg} cells (MOG⁺ FoxP3⁺ CD4⁺) in the isolated spinal lymphocytes. An identical gating strategy was used to analyze MOG-specific Tₕ1 (MOG⁺ T-bet⁺ CD4⁺), Tₕ17 (MOG⁺ RORγt⁺ CD4⁺), and T_{reg} cells (MOG⁺ FoxP3⁺ CD4⁺) in the isolated splenic lymphocytes.

FIG. 71a-c illustrates that drug-free and LEF-encapsulated PD-L1-Ig/CD86-Ig NP-functionalized MSCs are equally effective to induce the development of splenic MOG-specific T_{reg} cells to prevent the development of severe EAE symptoms. a, Two-dimensional FACS density plots showing the population of pathogenic MOG⁺ T-bet⁺ helper T cells (Tₕ1 cells) in the spleen 3 days after different therapeutic treatments. b, Two-dimensional FACS density plots showing the population of pathogenic MOG⁺ RORγt⁺ helper T cells (Tₕ17 cells) in the spleen 3 days after different therapeutic treatments. c, Two-dimensional FACS density plots showing the population of suppressive MOG⁺ FoxP3⁺ helper T cells (T_{reg} cells) in the spleen 3 days after different therapeutic treatments.

FIG. 72a-c illustrates that drug-free and LEF-encapsulated PD-L1-Ig/CD86-Ig NP-functionalized MSCs are equally effective to induce the development of splenic MOG-specific T_{reg} cells to ameliorate severe EAE symptoms. a, Two-dimensional FACS density plots showing the population of pathogenic MOG⁺ T-bet⁺ helper T cells (T^{h}1 cells) in the spleen 3 days after different therapeutic treatments. b, Two-dimensional FACS density plots showing the population of pathogenic MOG⁺ RORγt⁺ helper T cells (Tₕ17 cells) in the spleen 3 days after different therapeutic treatments. c, Two-dimensional FACS density plots showing the population of suppressive MOG⁺ FoxP3⁺ helper T cells (T_{reg} s) in the spleen 3 days after different therapeutic treatments.

FIG. 73a-d illustrates that LEF-encapsulated PD-L1-Ig/CD86-Ig LEF NP-functionalized MSCs are more effective than drug-free PD-L1-Ig/CD86-Ig NP-functionalized MSCs to inhibit autoreactive cytotoxic T cells in the spinal cord and induce the development of spinal MOG-specific T_{reg} cells to ameliorate EAE symptoms. a, Two-dimensional FACS density plots showing the population of pathogenic MOG⁺ INF-γ⁺ helper T cells (Tₕ1 cells) in the spinal cord 3 days after different therapeutic treatments. b, Two-dimensional FACS density plots showing the population of pathogenic MOG⁺ IL17A⁺ helper T cells (Tₕ17 cells) in the spinal cord 3 days after different therapeutic treatments. c, Two-dimensional FACS density plots showing the population of suppressive MOG⁺ FoxP3⁺ helper T cells (T_{reg} cells) in the spinal cord 3 days after different therapeutic treatments. d, Two-dimensional FACS density plots showing the population of autoreactive INF-γ⁺ cytotoxic T cells in the spinal cord 3 days after different therapeutic treatments.

FIG. 74 illustrates that drug-free and LEF-encapsulated PD-L1-Ig/CD86-Ig NP-functionalized MSCs induced the development of suppressive T_{reg} cells long after the prophylactic and therapeutic treatments. Two-dimensional FACS density plots showing the population of suppressive MOG⁺ FoxP3⁺ T_{reg} cells in the spleen 38 days p.i. after different prophylactic and therapeutic treatments.

FIG. 75 depicts in vivo functionalization of 3 cells with PD-L1-Ig through a 2-step, 2-component pretargeted strategy for in vivo bioengineering of β cells to reverse early onset T1DM. Intravenous administration of β cell-targeted Ac₄ManNAz NPs targeted delivery of Ac₄ManNAz to the β cells in the pancreas. Metabolic glycoengineering converts the intracellular ManNAz to azide sialic acid derivatives on the cells' surface proteins. The azide-modified β cells provide sties for SPAAC with the subsequently i.v. administered DBCO-functionalized PD-L1-Ig. The PD-L1-Ig-functionalized β cells simultaneously present a broad range of antigens (AG) to the CD8⁺ cytotoxic T cell and upregulate the PD-1/PD-L1 pathway, which anergizes the T cells and induces antigen-specific immunotolerance [MHIC I=major histocompatibility complex class I; TCR=T cell receptor].

FIG. 76a-i depict fabrication of a 2-component pretargeted system for in vivo functionalization of β cells. a, Fabrication of β cell-targeted Ac₄ManNAz-encapsulated NPs. b, Intensity-average diameter distribution curves recorded for biotin-functionalized Ac₄ManNAz-encapsulated NPs, avidin-functionalized Ac₄ManNAz-encapsulated NPs, β cell-targeted Ac₄ManNAz-encapsulated NPs, avidin, and exendin-4, as determined by the dynamic light scattering method. c, TEM images recorded for non-targeted Ac₄ManNAz-encapsulated NPs, biotin-functionalized Ac₄ManNAz-encapsulated NPs, and β cell-targeted Ac₄ManNAz-encapsulated NPs. d, In vitro Ac₄ManNAz release study was performed under physiological conditions. Unreleased Ac₄ManNAz was quantified by LC-MS. e, An in vitro NP-binding assay was performed in NIT-1 and MIN-6 cells. Different concentrations of β cell-targeted and non-targeted Cy5-labeled NPs were incubated with different β cells in complete cell culture media at physiological conditions for 1 h and washed 3 times before the fluorescence imaging study (n=4; 2×10⁴ cells per well, cells were seeded 24 h before the in vitro binding study). f, (i-ii) Ex vivo biodistribution study of β cell-targeted and non-targeted Cy5-labeled NPs (5 mg/mouse) in diabetic NOD mice (blood glucose=300-450 mg/dL) performed 3 h after i.v. administration of the Cy5-labeled NPs. Pancreas and selected organs were preserved for ex vivo imaging study 3 h after i.v. administration of the Cy5-labeled NPs. (iii) Histopathological images of the pancreas preserved from mice i.v. administered with different Cy5-labeled NPs. β cell-rich islets were stained with anti-insulin (green). g, Functionalization of PD-L1-Ig with DBCO-EG13 ligand through amine-NHS ester chemistry. The target degree of functionalization was 60. h, UV-visible absorption spectra of 1 mg/mL of PD-L1-Ig, DBCO-functionalized PD-L1-Ig, and DBCO-functionalized TexRed-labeled PD-L1-Ig. Each DBCO-functionalized PD-L1-Ig was calculated to conjugate with an average of 9 DBCO ligands. The TexRed-labeled PD-L1-Ig was functionalized with an average of 9 DBCO ligands and 2 TexRed ligands. i, Number-average distribution curves of unfunctionalized PD-L1-Ig and DBCO-functionalized PD-L1-Ig, as determined by SEC-MALS.

FIG. 77a-e depict PD-L1-Ig-functionalized β cells bioengineered through different pre-targeted strategies effectively anergize cytotoxic T cells in vitro. a, Scheme summarizes in vitro functionalization of NIT-1 cells through 2-step pre-targeted strategy. NIT-1 cells were cultured with different formulations of Ac₄ManNAz (50 μM) for 1 h and washed before culturing in a complete cell culture medium for 4 days. The azide-modified NIT-1 cells were functionalized with DBCO-functionalized PD-L1-Ig at a target degree of functionalization of 5 μg DBCO-functionalized PD-L1-Ig/10⁶ cells. b, PD-L1 expressions of different PD-L1-Ig-functionalized NIT-1 cells functionalized through a different pre-targeted method, as determined by the FACS method after being stained with an anti-PD-L1 antibody. c, CLSM images of different PE-labeled anti-mouse PD-L1 antibody-stained PD-L1-Ig-functionalized NIT-1 cells biofunctionalized using different Ac₄ManNAz formulations. d, PD-1 expressions of 8.3 T cells after being cultured with different non-functionalized and PD-L1-Ig-functionalized NIT-1 cells in the presence of IGRP₂₀₆₋₂₁₄ peptide at an effector:target ratio of 10:1 for 72 h, as determined by the FACS method. e, Intracellular IFN-gamma expressions of 8.3 T cells after being cultured with different non-functionalized and PD-L1-Ig-functionalized NIT-1 cells in the presence of IGRP₂₀₆₋₂₁₄ peptide at an effector:target ratio of 10:1 for 72 h, as determined by the FACS method.

FIG. 78a-e depict Pre-targeted functionalization through β cell-targeted Ac₄ManNAz NPs effectively in vivo bioengineered PD-L1-Ig-functionalized pancreatic β cells in vivo. a, Ex vivo fluorescence images of the pancreas and other key organs were recorded 48 h after the i.v. administration of DBCO-functionalized TexRed-labeled PD-L1-Ig (80 μg/mouse) to healthy non-diabetic NOD mice. The DBCO-functionalized TexRed-labeled PD-L1-Ig was administered 3 days after i.v. administration of different Ac₄ManNAz formulations (180 μg/mouse) (n=4, except n=5 for group 5). b, Biodistributions of DBCO-functionalized TexRed-labeled PD-L1-Ig determined for different pretargeted functionalization strategies in non-diabetic NOD mice (n=4, except n=5 for group 5). c, Representative immunofluorescence images of pancreas sections preserved after pretargeted functionalization with DBCO-functionalized TexRed-labeled PD-L1-Ig. The pancreas sections were stained with anti-insulin to label the β cell-rich islet. d, Biodistributions of DBCO-functionalized TexRed-labeled PD-L1-Ig (80 μg/mouse) after pretargeted administration of β cell-targeted Ac₄ManNAz NPs (5 mg NPs or 180 μg encapsulated Ac₄ManNAz per mouse) in diabetic NOD mice (blood sugar level=300-450 mg/dL). The DBCO-functionalized TexRed-labeled PD-L1-Ig was i.v. administered 3 days after the i.v. administration of β cell-targeted Ac₄ManNAz NPs. Insert shows the ex vivo fluorescence images of pancreata preserved from the untreated group and pretargeted group of diabetic NOD mice (n=4). e, Representative pancreas sections preserved from untreated diabetes NOD mouse and NOD mouse after pretargeted treatment with β cell-targeted Ac₄ManNAz NPs followed by DBCO-functionalized TexRed-labeled PD-L1-Ig. Pancreata were preserved at day 12 after the onset of T1DM (5 days after the administration of DBCO-functionalized TexRed-labeled PD-L1-Ig).

FIG. 79a-d depict in vivo PD-L1-Ig-functionalized pancreatic β cells effectively reverse early onset T1DM. a, Treatment schedule. Mice in the treatment groups were i.v. tail-vein injected with 150 μg of encapsulated Ac₄ManNAz (at day 4 after onset) and/or 80 μg of DBCO-functionalized PD-L1-Ig (at day 7 after onset). Mice in the two pretargeted treatment groups (group 5) received the second i.v. administration of β cell-targeted Ac₄ManNAz NPs at day 11 post-onset and DBCO-functionalized PD-L1-Ig at day 14 post-onset. b, Time-dependent blood glucose levels of NOD mice after different treatments (n=7 for groups 1 to 3, n=8 for group 4, and n=9 for group 5). c, Blood glucose levels of NOD mice recorded at day 14 after onset. d, Progression-free survival curves of non-treatment and treatment group mice after receiving different treatments.

FIG. 80a-e depict in vivo PD-L1-Ig-functionalized pancreatic β cells reverse early onset T1DM by anergizing cytotoxic T cells and inducing antigen-specific immunotolerance. a, Quantification of pancreas-infiltrated CD4⁺ helper T cells and CD8⁺ cytotoxic T cells 12 days after onset of T1DM through the FACS method. Mice in the treatment groups received pretargeted treatment with Ac₄ManNAz NPs at day 4 after the onset of T1DM, followed by DBCO-functionalized PD-L1-Ig at day 7 after the onset of T1DM. b, Quantification of pancreas-infiltrated IFN-gamma-expressing CD8⁺ T cells 12 days after the onset by the FACS method. c, Quantification of pancreatic-infiltrated FoxP3-expressing CD4⁺ Treg cells after different treatments (n=5, except n=6 for pretargeted treatment group 4). d, Representative H&E-stained pancreas sections preserved from untreated diabetic NOD mouse and diabetic NOD mouse that received pretargeted treatment with β cell-targeted Ac₄ManNAz NPs followed by DBCO-functionalized PD-L1-Ig. e, Representative immunofluorescence images of anti-insulin/anti-CD3 dual-stained pancreas sections preserved from untreated diabetic NOD mice and diabetic NOD mice received pretargeted treatment with β cell-targeted Ac₄ManNAz NPs followed by DBCO-functionalized PD-L1-Ig. The pancreas sections were preserved from diabetic NOD mice 12 days after the onset of T1DM (5 days after the i.v. administration of DBCO-functionalized PD-L1-Ig).

FIG. 81 depicts characterization of non-targeted Ac₄ManNAz NPs (suspended in 0.1 M PBS) by DLS method.

FIG. 82 shows representative immunofluorescence images of mouse pancreas sections preserved after the ex vivo fluorescence imaging study. β cell-rich insulin-producing islets were stained with anti-insulin (green).

FIG. 83a-c. a, In vitro toxicity of small-molecule (“free”) Ac₄ManNAz in NIT-1 cells, as determined by MTS assay. NIT-1 cells were cultured with small-molecule Ac₄ManNAz for 4 days (without removal of unbound Ac₄ManNAz). b, Relative viabilities of NIT-1 cells after culture with different formulations of Ac₄ManNAz. Cells were cultured with 50 μM of small-molecule or NP-encapsulated Ac₄ManNAz for 1 h, washed (with complete cell culture medium to remove unbound Ac₄ManNAz or NPs) before incubated at the physiological conditions for 4 days. Viabilities were determined by MTS assay, and calculated by compare the viability of untreated cells. c, Relative viabilities of PD-L1-Ig-functionalized NIT-1 cells. NIT-1 cells were cultured with after culture with different formulations of Ac₄ManNAz for 4 days (washed once 1 h after initial incubation), functionalized with DBCO-functionalized PD-L1-Ig, before incubated at complete cell culture medium for 4 days. Viabilities were determined by MTS assay, and calculated by compare the viability of untreated cells.

FIG. 84 shows immunofluorescence images of pancreas section preserved from mouse pretargeted with β cell-targeted Ac₄ManNAz NPs followed by DBCO-functionalized PD-L1-Ig.

FIG. 85a-b depict in vivo treatment with β cell-targeted Ac₄ManNAz NPs followed by DBCO-functionalized PD-L1-Ig did not induce significant a, hepatotoxicities, and b, nephrotoxicities in healthy BALB/c mice. (n=5)

FIG. 86 shows ex vivo florescent images of liver (LI), kidney (K), spleen (S), heart (H) and lung (LU) preserved from diabetic mice 5 days after i.v. administration of TexRed-labeled DBCO-functionalized PD-L1-Ig (12 days after onset of T1DM).

FIG. 87 depicts survival curves of diabetic NOD mice after different treatments. (n=7, except n=8 for group 4 (G4), and n=9 for group 5 (G5).)

FIG. 88a-c. a, Two-dimension FACS density plots showing the populations of pancreas-infiltrated CD4⁺ CD8⁻ helper T cells and CD4⁻ CD8⁺ cytotoxic T cells. b, Two-dimension FACS density plots showing the populations of IFN-gamma-expressing pancreas-infiltrated CD4⁻ CD8⁺ cytotoxic T cells. c, Two-dimension FACS density plots showing the populations of FoxP3-expressing pancreas-infiltrated CD4⁺ CD8⁻ regulatory T cells.

### DETAILED DESCRIPTION

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Overview

As mentioned above, the immune system evolved to elicit robust immune responses against foreign antigens while tolerating self-antigens to avoid autoimmunity^{14, 15} Failure to establish peripheral immune tolerance leads to the development of autoimmune diseases, ranging from type 1 diabetes to MS^{14, 15} T_{reg} cells are required to maintain immune tolerance and homeostasis²⁸. Numerous in vivo studies and clinical trials employed stimulated bulk T_{reg} cells for the treatment of autoimmune diseases^{36, 66}; however, the absence of antigen specificity increases the risk of systemic immunesuspension^{36, 66}.

Insulin-dependent diabetes mellitus (also known as type 1 diabetes, T1D) is a chronic autoimmune disease characterized by insulin deficiency that occurs when autoreactive T cells destroy the insulin-producing pancreatic beta (β) cells.¹⁻³ Each year, there are more than a million new T1D cases worldwide, with approximately half of them diagnosed in individuals' adulthood.⁴ T1D has complicated pathogenesis that can be generally divided into pre-symptomatic and symptomatic stages.^{1, 2, 5, 6} Once it progresses to the symptomatic stage, there is often rapid progression to total β cell loss within a year.^{1, 2, 5, 6} Most T1D patients maintain their blood glucose levels using multiple insulin injections per day or through insulin-pump therapy.¹⁻³ Still, less than a third of the T1D patients consistently achieve their target blood glucose levels. Despite major advances in disease management and care, T1D remains associated with a considerably higher probability that patients will develop acute diseases like neuropathy, nephropathy, retinopathy, and cardiovascular disease, along with a higher rate of premature death than in the general population.¹⁻⁴ There is considerable interest in the development of new immunotherapy strategies for delaying and even reversing early-onset T1D because a substantial mass of β cells is still present at the early-symptomatic stages. This can allow the patient to regain metabolic control. In recent years, several clinical trials have investigated the use of proinsulin peptide-based vaccines to reverse early-onset hyperglycemia, but the results have been disappointing.⁷⁻¹⁰

Autoantigen-specific chimeric antigen receptor T_{reg} cells are available to suppress MS³⁵, although the clinical outcomes are disappointing because of the rapid mutation of autoantigens and insufficient long-term potency of the infused T_{reg} cells³⁶. Recent studies have focused on the administration of encephalitogenic peptide-conjugated microparticles⁶⁷ and encephalitogenic peptide-conjugated isologues leukocytes^{68, 69} to induce antigen-specific immune tolerance through the reduction the population of pathogenic helper T cells and induction of antigen-specific T_{reg} cells. However, clinical trials showed that only a small group of MS patients with human leukocyte antigen haplotypes DR2 or DR4 benefit from these treatments⁶⁹. Further, the long-term treatment response of these highly antigen-specific treatments is often compromised by the epitope shift and autoantigen mutation⁷⁰.

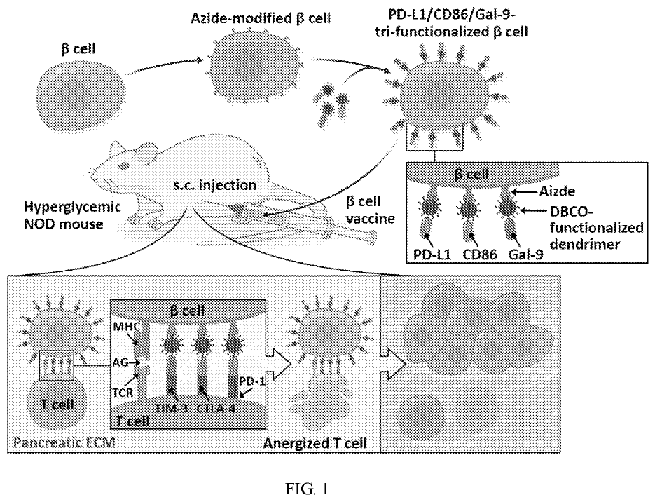

Metabolic glycoengineering^{20, 21} and biorthogonal click chemistry²²⁻²⁴ are available tools. As described herein, these can be used to facilitate unique chemical decoration of immune checkpoint molecules onto the targeted cells. As described herein, immune checkpoint molecules (PD-L1, CD86, and Gal-9) can be decorated onto β cells through metabolic glycoengineering and biorthogonal click reactions. These β cells can be used as live-cell vaccines to induce immune tolerance in autoreactive T cells and reverse the effects of early-onset hyperglycemia. The immune checkpoint molecule-decorated β cells effectively exhausted T cells in vitro. Intrapancreatic administration of PD-L1/CD86/Gal-9-tri-functionalized NIT-1 cells can reverse early-onset hyperglycemia in NOD mice. A novel s.c.-injectable vaccine based on PD-L1/CD86/Gal-9-tri-functionalized NIT-1 cell-embedded pan-ECM was developed to reverse early-onset hyperglycemia. The acellular pan-ECM not only functions as a scaffold for the localization of the functionalized β cells but it also regenerates an immunogenic pancreas microenvironment for the β cells to interface with autoreactive T cells and evoke strong antigen-specific T_{eff} inhibition (FIG. 1). In one embodiment, described herein is a live-cell vaccine platform for autoimmune diseases that generating a broad range of T_{eff} responses, from immunity to tolerance.

Also disclosed herein, is the use of metabolic glycoengineering and bioorthogonal click chemistry to bioengineer PD-L1- and CD86-functionalized SCs to prevent and treat MS. In MS, autoreactive T cells attack the myelin in the central nervous system (CNS), which disrupts communication between the brain and peripheral system. Most patients initially experience episodes of reversible neurological deficits, followed by remission, before chronic neurological deterioration leads to severe, irreversible disabilities. Unfortunately, MS cannot be completely cured, although available immunomodulatory therapies reduce the frequency and severity of MS relapses by inducing antigen-specific immunotolerance, thus delaying the accumulation of disabilities. New treatment strategies involve the induction of antigen-specific T_{reg} cells that suppress inflammatory pathogens and restore peripheral immunotolerance without causing systemic immunosuppression. As mentioned above, in the case of multiple sclerosis (MS), autoreactive T cells attack the myelin in the central nervous system (CNS), which disrupts communication between the brain and peripheral system^{29, 31}. Some newer treatment strategies involve the induction of antigen-specific T_{reg} cells^{35, 36} that suppress inflammatory pathogens and restore peripheral immunotolerance without causing systemic immunosuppression. However, in contrast to other antigen-specific MS treatment strategies, the functionalized SCs described herein were designed to present a broad range of myelin antigens to engaged pathogenic helper T cells, to inhibit their activation, and to induce the development of myelin antigen-specific T_{reg} cells to suppress the autoreactive immune cells. Comprehensive in vitro and in vivo studies show that immune checkpoint ligand-functionalized SCs effectively inhibited the differentiation of myelin-specific helper T cells into pathogenic Tₕ1 and Tₕ17 cells, promoted the development of antigen-specific T_{reg} cells and resolved the inflammatory CNS microenvironment in established mouse EAE models. The less proinflammatory microenvironment allows the OLs to repair myelin damage and ameliorate EAE clinical signs. The facile bioorthogonal conjugation strategy reported here allows on-demand modular-based functionalization of SCs. This reversible bioconjugation strategy was associated with low toxicity and prevented potential irreversible adverse effects associated with inhibitory immune checkpoint pathways. The present study provides a new framework for treating MS and supports its further evaluation in other models of autoimmune disease.

Described herein, in embodiments, are methods for bioengineering programmed death-ligand 1 and cluster of differentiation 86-functionalized mouse Schwann cells to prevent and ameliorate multiple sclerosis in established mouse models of chronic and relapsing-remitting experimental autoimmune encephalomyelitis (EAE). The data herein show that the intravenous administration of immune checkpoint ligand-functionalized mouse Schwann cells modifies the course of disease and ameliorates EAE. Further, such bioengineered mouse Schwann cells inhibit the differentiation of myelin-specific helper T cells into pathogenic T helper type 1 and type 17 cells, promote the development of tolerogenic myelin-specific regulatory T cells and resolve inflammatory CNS microenvironments without inducing systemic immunosuppression.

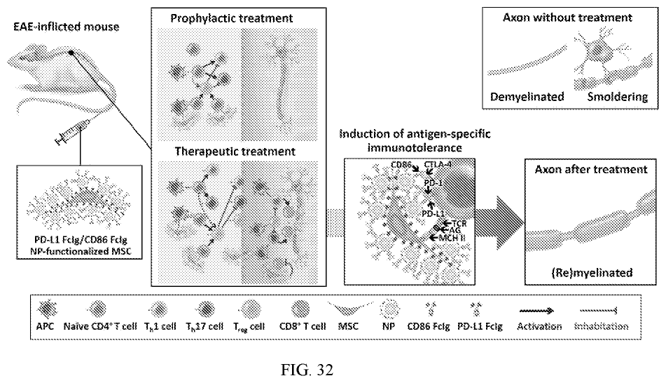

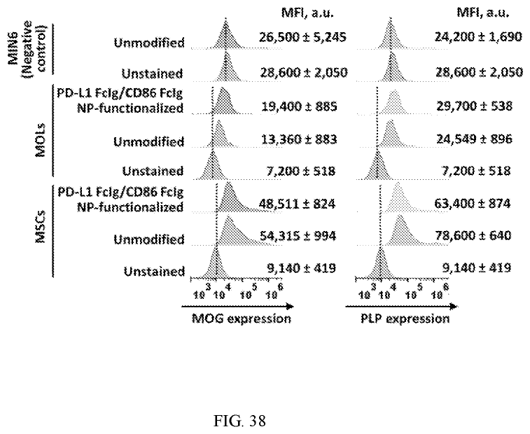

The data provided herein report on the intravenous (i.v.) or intramuscular (i.m.) administration of coinhibitory immune checkpoint ligand-bioengineered glia for preventing the development of early-onset MS or reversed its course through inhibiting the activation of pathogenic CD4⁺ lymphocyte T helper type 1 (Tₕ1) and type 17 (Tₕ17) cells as well promoting the development of myelin-specific T_{reg} cells (FIG. 32). Further, creating a less proinflammatory CNS microenvironment through local cotreatment with an immunomodulatory drug (e.g., leflunomide (LEF)^{42, 43}) can confer the ability of oligodendrocytes (OLs) to repair myelin damage¹⁹ and ameliorate MS symptoms (FIG. 32). To achieve this, bioengineered Schwann cells (SCs) (glial cells of the peripheral nervous system) or oligodendrocytes (OLs) with LEF-encapsulated nanoparticles (NPs) functionalized with PD-L1 and CD86 to upregulate the PD-1 and CTLA-4 signaling pathways in the engaged myelin-specific CD4⁺ T cells were developed (FIG. 32). In embodiments, SCs show particular utility because they express diverse myelin-specific antigens such as myelin oligodendrocyte glycoprotein (MOG) and proteolipid protein (PLP) (FIG. 38). Furthermore, protocols have been established for autologous SC transplant⁴⁵⁻⁴⁷.

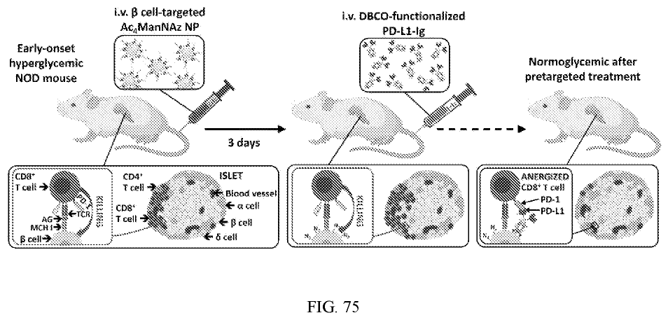

Additionally, described herein is a two-step translatable in vivo bioconjugation strategy to decorate PD-L1 onto pancreatic β cells to reverse early onset T1DM. The two-step, two-component pretargeted bioconjugation strategy comprises β cell-targeted, Ac₄ManNAz-encapsulated nanoparticles (Ac₄ManNAz NPs) (pretargeting component) and a dibenzylcyclooctyne (DBCO)-functionalized PD-L1 immunoglobin Fc-fusion protein (effector) (see FIG. 75). The β cell-targeted exendin-4-functionalized NPs selectively deliver Ac₄ManNAz to the glucagon-like peptide 1 receptor (GLP-1R)-overexpressed β cells⁷⁴ after i.v. administration. Upon binding to the GLP-1R, the exendin-4-functionalized Ac₄ManNAz NPs can rapidly internalize the β cells,⁷⁵ and enable the controlled release of the encapsulated Ac₄ManNAz, which converts to azido sialic acid derivatives for N-linked glycosylation of cell surface proteins.^{20, 21, 23} The azide-modified β cells provide sites for strain-promoted azide-alkyne cycloaddition (SPAAC)^{23, 24} with the i.v.-administrated DBCO-functionalized PD-L1-Ig. Comprehensive in vitro and in vivo studies performed in early onset NOD mice confirmed that the in vivo PD-L1-bioengineered β cell can simultaneously present islet-specific antigen and PD-L1 to the engaging T cells, anergize the autoreactive T cells, induce antigen-specific immunotolerance, and reverse early onset T1DM without inducing long-term systemic immunosuppression (see FIG. 75). Disclosed herein is a translatable two-step, two-component pretargeted method for the in vivo bioengineering of PD-L1-functionalized pancreatic β cells to reverse early onset T1DM. A comprehensive mechanistic study confirmed that the in vivo functionalized β cells can reverse early onset T1DM by anergizing pancreas-infiltrated IFN-γ-expressing cytotoxic T cells¹³ and inducing antigen-specific immunotolerance through the maintenance of immunosuppressive Treg cells.²⁸ In contrast to other immune checkpoint therapies for T1DM,^{76, 5} this in vivo bioengineering method does not induce long-term irreversible immunosuppression. In addition, this strategy can be easily adapted to other autoimmune diseases by changing the targeting moiety in the pretargeting component.

### II. Compositions

### Functionalized Cells

In one embodiment, a functionalized cell comprising, a cell comprising a decorated cell surface, wherein the decorated cell surface comprises at least one covalently attached immune checkpoint molecule. As used herein, the term “decorated cell surface” refers to a cell that comprises at least one covalent modification whereby an immune checkpoint molecule is covalently attached to the cell surface through a chemical linking strategy, such as those described herein. The covalent modification results in a functionalized cell.

In another aspect, the subject matter described herein is directed to a functionalized cell having one of the following general structures:

wherein, X is an integer from 1 to 100, and y is an integer from 1 to 100. In embodiments, X is an integer from 1 to 80, from 1 to 60, from 1 to 50, from 1 to 40, from 1 to 30, from 1 to 20 or from 1 to 10; or from 10 to 90, 10 to 70, or 10 to 50, such as any integer from 1 to 100. In embodiments, Y is an integer from 1 to 80, from 1 to 60, from 1 to 50, from 1 to 40, from 1 to 30, from 1 to 20 or from 1 to 10; or from 10 to 90, 10 to 70, or 10 to 50, such as any integer from 1 to 100.

In embodiments, the cell is a beta cell, a cell associated with myelin sheath (e.g., Schwann cells, oligodendrocytes), or any target cells of autoimmune disease, such as pneumocytes, platelets, epithelial cells, hepatocytes, or synovial cells.

In embodiments, the functionalized cell is a living cell. In embodiments, the functionalized cell is viable for about 1 day to about 7 days, about 2 days to about 6 days, about 3 days to about 4 days, about 5 days to about 21 days, or about 7 days to about 14 days under physiological conditions. In embodiments, the functionalized cell is viable for about 1 day, about 2 days, about 3 days, about 4 days, about 5 days, about 6 days, about 7 days, about 10 days, about 12 days, about 14 days, about 16 days, about 18 days, or about 21 days under physiological conditions.

In embodiments, the immune checkpoint molecule is PD-L1, CD86, Gal-9, PD-L2, TIGIT, TIM-1, TIM-3, TNFR1, VISTA, BTLA, NKG2A, CTLA-4, B7-H3, B7-H4, B7-H5, B7-H6, B7-H7, ICOS, NKp30, LAG3, CD137, or CD96. In one embodiment, the immune checkpoint molecule is PD-L1, CD86, or Gal-9. In one embodiment, the functionalized cell comprises at least one PD-L1, at least one CD86, and at least one Gal-9. In embodiments, the immune checkpoint molecule can be a fusion protein, fro example, PD-L1 can be a PD-L1-Ig.

PD-L1, Programmed death-ligand 1 (Uniprot: Q9NZQ7), is a 40 kDa type 1 transmembrane protein. PD-L1 is a ligand for PD-1. PD-L1 is also known as B7-H1 (B7 homolog 1).

CD86, T-lymphocyte activation antigen CD86 (Uniprot: P42081), is a type I membrane protein. CD86 is a ligand for CTLA-4 in activated T cells. CD86 (along with CD80) provides costimulatory signals necessary for T-cell activation and survival.

Gal-9, Galectin 9 (Uniprot: 000182) is a 36 kDa beta-galactoside lectin protein. Gal-9 is a ligand for TIM-3.

In embodiments, the subject matter described herein is directed to a functionalized cell comprising a glycoengineered moiety having the structure: (a transmembrane glycoprotein)-(a residue of an azide-containing molecule)-(a residue of a cyclooctyne)-(a linker 1)-(a residue of a functionalized dendrimer)_{q}-(a residue of an immune checkpoint molecule), wherein, q is one or zero; and, the dash represents a covalent bond. In one embodiment, a functionalized cell comprises a glycoengineered moiety having the structure: (a transmembrane glycoprotein)-(a residue of an cycoloctyne-containing molecule)-(a residue of a azide)-(a linker 1)-(a residue of a functionalized dendrimer)_{q}-(a residue of an immune checkpoint molecule), wherein, q is one or zero; and, the dash represents a covalent bond. When q is one, then the dendrimer is present. When q is zero, the dendrimer is absent which results in the DBCO direct conjugation strategy. As used herein, the term “residue” or “residue of” a chemical moiety refers to a chemical moiety that is bound to a molecule, whereby through the binding, at least one covalent bond has replaced at least one atom of the original chemical moiety, resulting in a residue of the chemical moiety in the molecule.

In another embodiment, the subject matter described herein is directed to a functionalized cell comprising a glycoengineered moiety having the structure: (a transmembrane glycoprotein)-(a residue of an azide-containing molecule)-(a residue of a cycoloctyne)-(a linker 1)-(immune checkpoint molecule FcIg fusion protein), wherein, the dash represents a covalent bond. In another embodiment, the subject matter described herein is directed to a functionalized cell comprising a glycoengineered moiety having the structure: (a transmembrane glycoprotein)-(a residue of an cycoloctyne-containing molecule)-(a residue of a azide)-(a linker 1)-(immune checkpoint molecule FcIg fusion protein), wherein, the dash represents a covalent bond. In embodiments, the immune checkpoint molecule/immune checkpoint molecule FcIg fusion protein can be conjugate via amine-NHS ester chemistry, or thiol-maleimide chemistry. In another embodiment, the subject matter described herein is directed to a functionalized cell comprising a glycoengineered moiety having the structure: ((a transmembrane glycoprotein)-(a residue of an azide-containing molecule)-(a residue of a cycoloctyne)-(a nanoparticle)-((a linker, such as linker 1)-(immune checkpoint molecule))_{y})ₓ, wherein, the dash represents a covalent bond and x and y are as described herein.

In embodiments, thiol-maleimide click chemistry can be used to modify the surface of a cell. Generally, free thiol groups on the surface can be made to react with maleimide-functionalized biomolecule through stable thioester bond to form stable functionalized cells. Maleimide-functionalized biomolecules can be prepared by amine-NHS reaction between desired biomolecule and NHS-maleimide crosslinker (e.g., sulfosuccinimidyl 4-(N-maleimidomethyl)cyclohexane-1-carboxylate (sulfo-SMCC)).

In embodiments, the subject matter described herein is directed to a functionalized cell, wherein the residue of a functionalized dendrimer has the structure: -(dendrimer)-(a linker 2)-(a residue of a cyclooctyne)-(a residue of an azide-containing molecule)-. In one embodiment, the linker 2 has the structure:

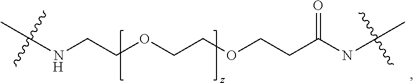

wherein, z is an integer from 0 to 10.

In embodiments, z is 1, 2, 3, 4, 5, 6, 7, 8, 9, or 10. In embodiments, z is 3. In one embodiment, z is an integer from 0 to 100,000. In one embodiment, z is an integer from 0 to 10, 0 to 100, 0 to 1,000, 0 to 5,000, or 0 to 10,000. In one embodiment, z is an integer from 10 to 100,000, 100 to 100,000, 1,000 to 100,000, 5,000 to 100,000, or 10,000 to 100,000.

In one embodiment, the functionalized cell comprises from about 0.5 μg to about 100 μg of the at least one covalently attached immune checkpoint molecule per about 1 million functionalized cells. In one embodiment, the functionalized cell comprises from about 0.5 μg to about 100.0 μg, about 0.5 μg to about 75.0 μg, about 1 μg to about 60.0 μg, about 1 μg to about 50.0 μg, about 10 μg to about 50.0 μg, about 20 μg to about 50.0 μg, about 30 μg to about 50.0 μg, about 40 μg to about 50.0 μg, about 0.5 μg to about 40.0 μg, about 0.5 μg to about 30.0 μg, about 0.5 μg to about 20.0 μg, or about 0.5 μg to about 10.0 μg of the at least one covalently attached immune checkpoint molecule per about 1 million functionalized cells. In one embodiment, the functionalized cell comprises from about 0.5 μg, about 1 μg, about 10.0 μg, about 20.0 μg, about 30.0 μg, about 40.0 μg, about 50.0 μg, about 60.0 μg, or about 75.0 μg of at least one covalently attached immune checkpoint molecule per about 1 million functionalized cells. The total amount of immune checkpoint molecule can be quantified, for example, by fluorescence spectroscopy (via fluorescence labeled protein) or quantitative Western blot (e.g., AutoWest).

In embodiments, the subject matter described herein is directed to a glycoengineered moiety comprising an azide moiety, a cyclooctyne moiety, or a tetrazine moiety.

In embodiments, the at least one covalently attached immune checkpoint molecule is attached through a glycoengineered moiety. In embodiments, the at least one covalently attached immune checkpoint molecule is an immune checkpoint molecule-functionalized nanoparticle or polymer. In embodiments, the covalent attachment is via conjugating to thiol groups on cells.

In embodiments, the glycoengineered moiety comprises a residue of an amide of mannosamine or galactosamine. In embodiments, the glycoengineered moiety further comprises a residue of an azide, a dibenzocyclooctyne, or a tetrazine covalently attached to the residue of an amide of mannosamine or galactosamine. In embodiments, the dibenzocyclooctyne is DBCO.

In another embodiment, the glycoengineered moiety further comprises a residue of a dendrimer, a linear polymer, a nanoparticle, or a Fc fusion protein. In one embodiment, the nanoparticle is a dendrimer, a liposome, an inorganic nanoparticle, or a polymeric nanoparticle. In one embodiment, the nanoparticle is about 2 nm to about 10 nm, about 10 nm to about 100 nm, or about 100 nm to about 1000 nm. In embodiments, the nanoparticle is about 2 nm to about 1000 nm, about 2 nm to about 750 nm, about 2 nm to about 500 nm, about 2 nm to about 250 nm, about 2 nm to about 200 nm, about 2 nm to about 100 nm, or 2 nm to about 50 nm. In embodiments, the nanoparticle is about 10 nm to about 1000 nm, about 25 nm to about 1000 nm, about 50 nm to about 1000 nm, about 100 nm to about 1000 nm, about 200 to about 1000 nm, about 500 nm to about 1000 nm, or 750 nm to about 1000 nm. In embodiments, the nanoparticle is about 2 nm, about 5 nm, about 10 nm, about 50 nm, about 100 nm, about 200 nm, about 300 nm, about 400 nm, about 500 nm, about 600 nm, about 700 nm, about 800 nm, about 900 nm, or about 1000 nm. In embodiments, the nanoparticle is further covalently attached through a linker to one or more immune checkpoint molecules as described herein. In one embodiment, the dendrimer is a multivalent dendrimer. In one embodiment, the multivalent dendrimer is a polyamidoamine dendrimer. In embodiments, the nanoparticle is a pegylated nanoparticle (e.g., DBCO-functionalized PEG-PLGA nanoparticle). In embodiments, the pegylated nanoparticle is less than 200 nm in diameter.

In one embodiment, the polyamidoamine dendrimer has a MW of from about 500 to about 1,000,000. In one embodiment, the polyamidoamine dendrimer has a MW of from about 1000 to about 1,000,000, about 5000 to about 1,000,000, about 10,000 to about 1,000,000, about 15,000 to about 1,000,000, about 20,000 to about 1,000,000, about 500 to about 100,000, about 500 to about 50,000, or about 500 to about 35,000.

In one embodiment, the polyamidoamine dendrimer has a MW of from about 20,000 to about 35,000. In one embodiment, the polyamidoamine dendrimer has a MW of from about 20,000 to about 30,000. In one embodiment, the polyamidoamine dendrimer has a MW of from about 25,000 to about 30,000.

In one embodiment, the polyamidoamine dendrimer has a MW of about 20,000, about 21,000, about 22,000, about 23,000, about 24,000, about 25,000, about 26,000, about 27,000, about 28,000, about 29,000, about 30,000, about 31,000, about 32,000, about 33,000, about 34,000, or about 35,000. In one embodiment, the polyamidoamine dendrimer has a MW of about 28,000.

In certain aspects of this embodiment, the subject matter described herein is directed to a functionalized cell that has been prepared by an in vivo method of preparing a functionalized cell in an organism, comprising: administering to the organism in any order: a cell labeling agent comprising a ligand reactive group, and one or more active agents comprising a covalently bound ligand that reacts with the ligand reactive group, wherein the functionalized cell is prepared in vivo.

As used herein, the term “systemic immunosuppression” refers to a reduction of the activation or efficacy of the immune system. As used herein the phrase “no long-term broad systemic immunosuppression” and the like refer to the lack of a clinically relevant systemic immunosuppression, which can be associated with continuous administration of immunosuppressive therapy.

As used herein, the term “autoreactive T cell” refers to a T cell that recognize antigenic peptides presented to them in the context of a host's antigen presenting HLA molecule and become activated if the appropriate signals are provided, whereby the autoreactive T cell are specific for peptides representing “self,” as opposed to “foreign” proteins, pathogens, etc.

As used herein, the term “anergy” and “anergized” and the like refer to a process or result of a lack of reaction by the body's defense mechanisms to foreign substances, and consists of a direct induction of peripheral lymphocyte tolerance. An cell in a state of anergy is unable to mount a normal immune response against a specific antigen, usually a self-antigen.

As used herein, the term “physiological conditions” refers to the range of conditions of temperature, pH, and tonicity (or osmolality) normally encountered within tissues in the body of a living human.

The term “in vitro” refers to artificial environments and to processes or reactions that occur within an artificial environment (e.g., a test tube).

The term “in vivo” refers to natural environments (e.g., a cell or organism or body) and to processes or reactions that occur within a natural environment.

Designation of a range of values includes all integers within or defining the range, and all subranges defined by integers within the range.

Unless otherwise apparent from the context, the term “about” encompasses values within a standard margin of error of measurement (e.g., SEM) of a stated value or variations±0.5%, 1%, 5%, or 10% from a specified value.

Compositions or methods “comprising” or “including” one or more recited elements may include other elements not specifically recited. For example, a composition that “comprises” or “includes” a protein may contain the protein alone or in combination with other ingredients.

The singular forms of the articles “a,” “an,” and “the” include plural references unless the context clearly dictates otherwise. For example, the term “an antigen” or “at least one antigen” can include a plurality of antigens, including mixtures thereof.

Statistically significant means p≤0.05.

### Acellular Extracellular Matrices

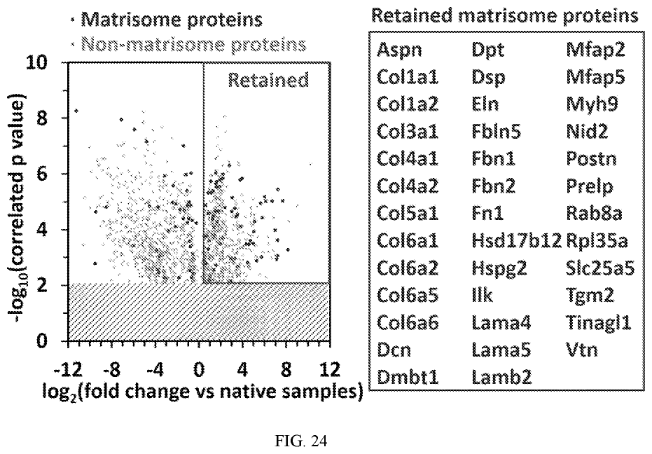

In one embodiment, described herein is an acellular pancreatic extracellular matrix comprising, a functionalized cell as described herein; and decellularized pancreatic-derived proteins. Examples of decellularized pancreatic-derived proteins are listed in FIG. 24. In another embodiment, the functionalized cells form three-dimensional spheroid colonies.

In another embodiment, the acellular pancreatic extracellular matrix is in the form of an injectable. In one embodiment, the acellular pancreatic extracellular matrix is in the form of an injectable that is not a gel. In one embodiment, the acellular pancreatic extracellular matrix is in the form of an injectable that is a gel. In one embodiment, the acellular pancreatic extracellular matrix is in the form of an injectable that is a gel that is not a thermal responsive hydrogel.

### Pharmaceutical Compositions

In one embodiment, described herein is a pharmaceutical composition comprising a functionalized cell as described herein or an acellular pancreatic extracellular matrix as described herein, and a pharmaceutically acceptable excipient.

In one embodiment, described herein is a vaccine comprising a functionalized cell as described herein or an acellular pancreatic extracellular matrix as described herein, and a pharmaceutically acceptable liquid vehicle.

The term “vaccine” refers to a composition that elicits an immune response and that may prevent a subject from contracting or developing a disease or condition and/or a vaccine may be therapeutic to a subject having a disease or condition.

A “pharmaceutically acceptable excipient” refers to a vehicle for containing a functionalized cell or an acellular extracellular matrix that can be introduced into a subject without significant adverse effects and without having deleterious effects on the functionalized cell or acellular extracellular matrix. That is, “pharmaceutically acceptable” refers to any formulation which is safe, and provides the appropriate delivery for the desired route of administration of an effective amount of at least one functionalized cell or acellular extracellular matrix for use in the methods disclosed herein. Pharmaceutically acceptable carriers or vehicles or excipients are well known. Descriptions of suitable pharmaceutically acceptable carriers, and factors involved in their selection, are found in a variety of readily available sources such as, for example, Remington's Pharmaceutical Sciences, 18th ed., 1990, herein incorporated by reference in its entirety for all purposes. Such carriers can be suitable for any route of administration (e.g., parenteral, enteral (e.g., oral), or topical application). Such pharmaceutical compositions can be buffered, for example, wherein the pH is maintained at a particular desired value, ranging from pH 4.0 to pH 9.0, in accordance with the stability of the functionalized cell or acellular extracellular matrix and route of administration.

Suitable pharmaceutically acceptable carriers include, for example, sterile water, salt solutions such as saline, glucose, buffered solutions such as phosphate buffered solutions or bicarbonate buffered solutions, alcohols, gum arabic, vegetable oils, benzyl alcohols, polyethylene glycols, gelatine, carbohydrates (e.g., lactose, amylose or starch), magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, and the like. Pharmaceutical compositions or vaccines may also include auxiliary agents including, for example, diluents, stabilizers (e.g., sugars and amino acids), preservatives, wetting agents, emulsifiers, pH buffering agents, viscosity enhancing additives, lubricants, salts for influencing osmotic pressure, buffers, vitamins, coloring, flavoring, aromatic substances, and the like which do not deleteriously react with the a functionalized cell or a acellular extracellular matrix.

For liquid formulations, for example, pharmaceutically acceptable carriers may be aqueous or non-aqueous solutions, suspensions, emulsions, or oils. Non-aqueous solvents include, for example, propylene glycol, polyethylene glycol, and injectable organic esters such as ethyl oleate. Aqueous carriers include, for example, water, alcoholic/aqueous solutions, emulsions or suspensions, including saline and buffered media. Examples of oils include those of petroleum, animal, vegetable, or synthetic origin, such as peanut oil, soybean oil, mineral oil, olive oil, sunflower oil, and fish-liver oil. Solid carriers/diluents include, for example, a gum, a starch (e.g., corn starch, pregeletanized starch), a sugar (e.g., lactose, mannitol, sucrose, or dextrose), a cellulosic material (e.g., microcrystalline cellulose), an acrylate (e.g., polymethylacrylate), calcium carbonate, magnesium oxide, talc, or mixtures thereof.

Optionally, sustained or directed release pharmaceutical compositions or vaccines can be formulated. This can be accomplished, for example, through use of liposomes or compositions wherein the active compound is protected with differentially degradable coatings (e.g., by microencapsulation, multiple coatings, and so forth). Such compositions may be formulated for immediate or slow release. It is also possible to freeze-dry the compositions and use the lyophilisates obtained (e.g., for the preparation of products for injection).

### III. Therapeutic Methods

In another embodiment, the subject matter described herein is directed to a method of treating or delaying onset of an autoimmune disease in a subject, comprising administering to the subject, a functionalized cell as described herein or an acellular extracellular matrix as described herein. In one embodiment, the subject is administered a pharmaceutical composition or a vaccine comprising the functionalized cell or acellular extracellular matrix.

In embodiments, the subject matter described herein is directed to a method of treating or delaying onset of type 1 diabetes, multiple sclerosis, autoimmune colitis, arthritis, lupus, or psoriasis comprising administering to the subject, a functionalized cell or an acellular extracellular matrix described herein. In embodiments, the autoimmune colitis is ulcerative colitis or crohn's disease. In embodiments, the arthritis is rheumatoid arthritis.

In embodiments, the type 1 diabetes is early-onset type 1 diabetes or early-onset hyperglycemia. In another embodiment, the subject matter described herein is directed to a method of reversing early-onset type 1 diabetes in a subject comprising administering to the subject, a functionalized beta cell or an acellular pancreatic extracellular matrix or a pharmaceutical composition or a vaccine comprising the same. In embodiments, the subject matter described herein is directed to a method of protecting pancreatic beta cells in a subject comprising administering to the subject, a functionalized beta cell or an acellular pancreatic extracellular matrix or a pharmaceutical composition or a vaccine comprising the same.

In embodiments, the subject matter described herein is directed to a method of treating an autoimmune disease in a subject, comprising: administering to the subject in any order: a cell labeling agent comprising a ligand reactive group, and one or more active agents comprising a covalently bound ligand that reacts with the ligand reactive group, wherein a functionalized cell is prepared in vivo, and wherein the autoimmune disease is treated. In embodiments, the autoimmune disease is Type 1 diabetes mellitus.

In embodiments, the subject matter described herein is directed to a method of anergizing an autoreactive immune cell in a subject, comprising: contacting the autoreactive immune cell with a functionalized cell, wherein the functionalized cell is prepared by administering to the subject in any order: a cell labeling agent comprising a ligand reactive group, and one or more active agents comprising a covalently bound ligand that reacts with the ligand reactive group, wherein the functionalized cell is prepared in vivo, and wherein the functionalized cell contacts the autoreactive immune cell, and wherein the autoreactive immune cell is anergized. In embodiments, the autoreactive immune cell is anergized and systemic immunosuppression is not induced. In embodiments, the systemic immunosuppression that does not occur is long-term broad systemic immunosuppression. In embodiments, the systemic immunosuppression that does not occur is long-term broad systemic immunosuppression and is irreversible. In embodiments, the autoreactive immune cell is an autoreactive T-cell.

In embodiments, the subject is at risk of developing diabetes or has diabetes or wherein the subject is at risk of developing multiple sclerosis or has multiple sclerosis.

In embodiments, treating an autoimmune disease is reducing the severity of symptoms of the autoimmune disease. In one embodiment, treating the subject with multiple sclerosis is reducing the severity of multiple sclerosis symptoms.

In embodiments, a method of modulating the T_{reg}:T_{eff} ratio in a subject or a method of exhausting autoreactive effector T-cells in a subject comprising administering to the subject, a functionalized beta cell or an acellular pancreatic extracellular matrix or a pharmaceutical composition or a vaccine comprising the same.

Thus, treatment includes ameliorating or preventing the worsening of existing disease symptoms, preventing additional symptoms from occurring, ameliorating or preventing the underlying metabolic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “treat” or “treating” refers to both therapeutic treatment and prophylactic or preventative measures, wherein the object is to prevent or lessen or reducing the severity of the symptoms of the autoimmune disease. Treating may include one or more of directly affecting or curing, suppressing, inhibiting, preventing, reducing the severity of, delaying the onset of, slowing the progression of, stabilizing the progression of, reducing/ameliorating symptoms associated with the autoimmune disease, or a combination thereof. The term “reducing the severity” refers to clinical or subjective determination of a lessening of an indication or symptom after treatment.

The term “subject” refers to a mammal (e.g., a human) in need of therapy for, or susceptible to developing, an autoimmune disease. The term subject also refers to a mammal (e.g., a human) that receives either prophylactic or therapeutic treatment. The subject may include dogs, cats, pigs, cows, sheep, goats, horses, rats, mice, non-human mammals, and humans. The term “subject” does not necessarily exclude an individual that is healthy in all respects and does not have or show signs of an autoimmune disease.

As used herein, the term “organism” includes, but is not limited to, a human, a non-human primate, such as those mentioned above, and any transgenic species thereof, and further includes any living eukaryote.

The terms “effective amount” or “therapeutically effective amount” refer to a sufficient amount of the composition to provide the desired biological result. That result can be reduction and/or alleviation of the signs, symptoms, or causes of a disease or medical condition, or any other desired alteration of a biological system. For example, an “effective amount” for therapeutic use is the amount of a composition that is required to provide a clinically relevant change in a disease state, symptom, or medical condition. An appropriate “effective” amount in any individual case may be determined by one of ordinary skill in the art using routine experimentation. Thus, the expression “effective amount” generally refers to the quantity for which the active substance has a therapeutically desired effect. Effective amounts or doses of the compositions of the embodiments may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about 1 μg to 10 mg of active agent per kilogram of subject's body weight per day. The total dosage may be given in single or divided dosage units (e.g., BID, TID, QID). Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### IV. Methods of Making

In an embodiment, described herein is a method of preparing a functionalized cell comprising glycoengineering a cell to express a glycoengineered moiety, which can comprise a residue of an amide of mannosamine or galactosamine, and can further comprise an azide moiety, a cyclooctyne moiety, or tetrazine moiety; and covalently linking an immune checkpoint molecule through the glycoengineered moiety, to prepare a functionalized cell. In one embodiment, the method further comprises harvesting the cell from a subject prior to the glycoengineering. In one embodiment, the method further comprises preserving the functionalized cell after the linking.

In an embodiment, the functionalized cells are prepared in situ. A non-limiting example of in vivo preparation is described in Example 18. In certain aspects of this embodiment, the subject matter described herein is directed to an in vivo method of preparing a functionalized cell in an organism, comprising: administering to the organism in any order: a cell labeling agent comprising a ligand reactive group, and one or more active agents comprising a covalently bound ligand that reacts with the ligand reactive group, wherein the functionalized cell is prepared in vivo. In certain aspects, the ligand reactive group comprises an azide moiety. In certain aspects, the cell is a beta cell, a Schwann cell, oligodendrocytes, a pneumocyte, a platelet, a epithelial cell, a hepatocyte, or a synovial cell. In aspects of this embodiment, the in vivo method utilizes a two-step, two-component pretargeted bioconjugation strategy, comprising: administering a cell labeling agent, such as azide containing sialic acid analogue either as free drug or in a nanoparticle formulation, followed by the administration of a single or multiple immune checkpoint ligands containing reactive group that can conjugate to the cell labeling agent, either as free checkpoint ligands or as a nanoparticle formulation. Preferably, the administration is i.v. administration. In aspects of this embodiment, β cell-targeted exendin-4-functionalized NPs selectively deliver Ac₄ManNAz to the glucagon-like peptide 1 receptor (GLP-1R)-overexpressed β cells after i.v. administration. Upon binding to the GLP-1R, the exendin-4-functionalized Ac₄ManNAz NPs can rapidly internalize the β cells, enable the controlled release of the encapsulated Ac₄ManNAz, which convert to azido sialic acid derivatives for N-linked glycosylation of cell surface proteins. The azide-modified β cells provide sites for strain-promoted azide-alkyne cycloaddition (SPAAC) with the i.v.-administrated DBCO-functionalized PD-L1-Ig.

In all of the preparation methods, glycoengineering a cell comprises contacting the cell with a compound, such as N-azidoacetylmannosaminetetraacelate, N-azidoacetylmannosamine, acetylated, N-azidoacetylgalactosamine-tetraacylated, or N-azidoacetylglucosamine, acetylated, to prepare a cell having an azide moiety, a cyclooctyne moiety, or tetrazine moiety, or mixtures thereof (referred to in each instance as a glycoengineered moiety) on the cell surface.

Covalently linking the moiety on the cell to an immune checkpoint molecule comprises attaching the immune checkpoint molecule through the glycoengineered moiety on the cell surface by one of the strategies described herein.

Harvesting and preserving cells are known in the field. Any known method for obtaining harvested cells and preserving cells can be employed.

The disclosed subject matter is further described in the following non-limiting Examples. It should be understood that these Examples, while indicating preferred embodiments of the invention, are given by way of illustration only.

### EXAMPLES

### Materials

N-azidoacetylmannosamine tetraacylated (Ac₄ManNAz), dibenzocyclooctyne-functionalized oligoethylene glycol N-hydroxysuccinimide ester (DBCO-PEG13-NHS ester; 95%), and trans-cyclooctene-functionalized oligoethylene glycol N-hydroxysuccinimide ester (TCO-PEG4-NHS ester, ≥95%) were purchased from Click Chemistry Tools (Scottsdale, AZ). Leflunomide (Pharmaceutical Secondary Standard), water (BioReagent), acetonitrile (HPLC grade, ≥99%), dimethyl sulfoxide (anhydrous, ≥99.9%), poly(lactide-co-glycolide) (PLGA, ester terminated; M_{w}=50 kDa-70 kDa), and formaldehyde solution (4%, buffered, pH 6.9) were purchased from Sigma (St Louis, MO).

Poly(lactide-co-glycolide)-block-poly(ethylene glycol)-dibenzocyclooctyne endcap (DBCO-PEG-PLGA; M_{w}=(5+10) kDa=15 kDa) was purchased from Nanosoft Polymers (Winston-Salem, NC). Poly(lactide)-block-poly(ethylene glycol)-methyltetrazine endcap (MTZ-PEG-PLA; AI150; M_{w}=(16+5) kDa=21 kDa), methoxy poly(ethylene glycol)-b-poly(D,L-lactic-co-glycolic) acid copolymer (mPEG-PLGA; AK10; M_{w}=(3+20) kDa=23 kDa), and poly(lactide-co-glycolide)-Cyanine 5 (Cy5-PLGA; AV034, M_{w}=30-55 kDa) were purchased from Akina, Inc (West Lafayette, IN).

Alexa Fluor 488 NHS ester, Texas Red-X NHS ester (mixture of isomers), Zeba Spin 7K MWCO Desalting Columns (Thermo Fisher), VivoTack 680 NIR fluorescent Imaging Agent (Perkin Elmer LLC), sulfo-cyanine 5 tetrazines (Lumiprobe), Dynabeads™ Mouse T-Activator CD3/CD28 T cells Activation Beads (Gibco), EasySep™ Mouse CD4⁺ T Cell Isolation Kit (STEMCELL Technologies), EasySep™ Mouse CD8⁺ T Cell Isolation Kit (STEMCELL Technologies), recombinant mouse IL-2 (R&D Systems) and CellTiter96© AQueous MTS Powder (Promega) were purchased from Fisher Scientific (Hampton, NH). Unless specified, all antibodies for flow cytometry studies were purchased from Fisher Scientific (Hampton, NH).

Recombinant mouse PD-L1-Ig fusion protein (PD-L1-Ig; molecular weight=102 kDa; PR00112-1.9), and recombinant mouse CD86-Ig fusion protein (CD86-Ig; molecular weight=103 kDa; PR00226-1.9) were purchased from Absolute Antibody NA (Boston, MA). Both fusion proteins were supplied in sterilized 1×PBS. The Mouse Interferon gamma ELISA Kit (ab100689) and mouse IL-17A ELISA Kit (ab199081) were purchased from Abcam PLC (Cambridge, MA).

Anti-CD25 antibody (InVivoMAb, clone: PC-61.5.3, catalog number: BE0012) Was purchased from BioXCell (Lebanon, NH).

EAE induction kits (MOG₃₅₋₅₅/CFA emulsion (contain 1 mg/mL of MOG₃₅₋₅₅) and a tailor-made PLP₁₇₈₋₁₉₁/CFA emulsion (contain 0.25 mg/mL of PLP₁₇₈₋₁₉₁)⁶⁴) were purchased from Hooke Laboratories, Inc (Lawrence, MA).

### Methods

Functionalization of PD-L1-Ig and CD86-Ig fusion proteins: PD-L1-Ig and CD86-Ig fusion proteins were functionalized via amine-NHS ester coupling chemistry^{51, 71}. DBCO-functionalized fusion proteins were functionalized via amine-NHS ester coupling reaction between the fusion protein and DBCO-PEG13-NHS ester at pH 8.0 (20° C.) for 2 h. The target degrees of functionalization were 15, 30, and 45 for the pilot functionalization study, and a target degree of 45 (leading to an actual degree of function of approximately 9) was used for the subsequent functionalization study. The functionalized fusion proteins were purified by Zeba Spin 7K MWCO desalting column according to the manufacturer's protocol. The concentrations and degrees of the DBCO incorporation of different purified DBCO-conjugated fusion proteins were determined spectroscopically using an absorption coefficient of DBCO at 310 nm (ε_{DBCO, 310 nm})=12,000 M⁻¹ L cm⁻¹, an absorption coefficient of mouse immunoglobulin at 280 nm (ε_{280 nm})=1.26 mg⁻¹ mL cm⁻¹ (for PD-L1-Ig)/1.34 mg⁻¹ mL cm⁻¹ (for CD86-Ig), and a DBCO correction factor at 280 nm (CF_{DBCO, 280 nm})=1.089 according to the manufacturer's instructions. The TCO-functionalized fusion proteins were prepared via the same method with a target degree of functionalization of 45. A488-labeled DBCO-functionalized PD-L1-Ig and Texas Red (TexRed)-labeled DBCO-functionalized CD86-Ig were prepared via the same method with a target degree of functionalization of 45 and 5 respectively. The concentrations of the purified dye-labeled fusion proteins were quantified via the Pierce BCA Protein assay kit (Thermo Fisher). The number of conjugated dye molecules belonging to the known concentration of fusion protein was calculated from the corresponding UV-visible absorption spectrum that used an absorption coefficient of 71,000 M⁻¹ L cm⁻¹ (at 495 nm) for the conjugated A488 dye or 80,000 M⁻¹ L cm⁻¹ (at 595 nm) for the conjugated Texas Red.

Preparation of drug-free/LEF-encapsulated DBCO/MTZ-functionalized PEG-PLGA NPs: Drug-free DBCO/MTZ-functionalized PEG-PLGA NPs (DBCO/MTZ NPs) were prepared via the nanoprecipitation method⁷¹. For the preparation of 30 mg of DBCO/MTZ NPs, 9 mg of DBCO-PEG-PLGA, 9 mg of MTZ-PEG-PLA, 12 mg of mPEG-PLGA, and 6 mg PLGA (consider as payload) were first dissolved into 3 mL of acetonitrile. The polymer blend was then added slowly (1 mL/min) to 12 mL of deionized water under constant stirring (1,000 rpm). The mixture was stirred under reduced pressure for 2 h before purifying it 3 times via Amicron Ultra ultrafiltration membrane filter (MWCO 100,000) as per the manufacturer's protocol. Cy5-labeled DBCO/MTZ NPs were prepared via the same method, except using Cy5-labeled PLGA instead of non-functionalized PLGA.

LEF-encapsulated DBCO/MTZ-functionalized PEG-PLGA NPs (DBCO/MTZ LEF NPs) were prepared via the same nanoprecipitation method with the addition of 7.25 wt/wt % of LEF in the polymer blend for preparing the NPs. The LEF loading in the purified NPs was quantified via fluorescence spectroscopy (excitation wavelength=280±20 nm; emission wavelength=410±20 nm), as previously reported. An in vitro drug release study was performed via Slide-A-Lyzer MINI Dialysis Devices (20K MWCO, Thermo Fisher) in the presence of a large excess of 1×PBS at 37° C. (in the dark). Unreleased LEF in the NPs was quantified via fluorescence spectroscopy⁵³.

Drug-free and LEF-encapsulated NPs suspended in IX PBS were characterized by transmission electron microscopy (TEM) and the dynamic light scattering method. TEM images were recorded in a JEOL 1230 transmission electron microscope in Microscopy Services Laboratory (MSL) at the UNC School of Medicine. Before the imaging study, carbon-coated copper grids were glow discharged, and the samples were negatively stained with tungsten acetate (pH 7). The intensity-average diameter of both purified NPs (suspended in 1×PBS) was determined by a Zetasizer Nano ZSP Dynamic Light Scattering Instrument (Malvern).

### In Vitro Studies

Cell lines. Mouse Schwann cells (MSCs, catalog number: T0295), isolated from the C57BL/6 mice, were purchased from Applied Biological Materials Inc. (ABM Inc.; Richmond, BC). MSCs were cultured in G422 Applied Cell Extracellular Matrix-coated cell culture flashes (catalog number: G422; ABM Inc.) in Prigow III Medium (catalog number TM003; ABM Inc.). This was supplemented with 10% FBS (Sigma) according to the manufacturer's protocol.

Mouse oligodendrocytes (MOLs, catalog number: 11004-02), isolated from the C57BL/6 mice, were purchased from Celprogen, Inc. (San Pedro, CA). MOLs were cultured in G422 Applied Cell Extracellular Matrix-coated cell culture flashes (catalog number: G422; ABM Inc.) in mouse oligodendrocytes primary cell culture complete medium with serum (catalog number: M11004-25; Celprogen, Inc) according to manufacturer's protocol.

The MOG and PLP expressions of MSCs and MOLs were separately quantified via the FACS method after stained with anti-myelin oligodendrocyte glycoprotein antibody (catalog number: A3992, ABclonal) and anti-PLP1 polyclonal antibody (catalog number: A20009, Abclonal). Both non-labeled rabbit antibodies were visualized by A488-labeled anti-rabbit IgG (H+L) Cross-Adsorbed Antibody (catalog number: A-11008, Invitrogen). MIN-6 cells (ATCC), established by the insulinoma cell line and isolated from C57BL/6 mice, were used as a negative control for both antibodies.

MOG-specific CD4⁺ T cells (2D2 cells) were isolated from 2D2 mice as previously reported⁵⁶. Briefly, CD4⁺ T cells were isolated from the splenocytes of 2D2 mice (C57BL/6-Tg (Tcra2D2, Tcrb2D2) 1Kuch/J; female, 7-8 weeks old, stock number: 006912, The Jackson Laboratory) using the immunomagnetic negative selection method via an EasySep™ Mouse CD4⁺ T Cell Isolation Kit (STEMCELL Technologies), as per the given manufacturer's rules.

CD8⁺ T cells were isolated from the splenocytes of wild-type C57BL/6 mice (female, about 8 weeks old; Charles River Laboratories) using the immunomagnetic negative selection method via an EasySep™ Mouse CD8⁺ T Cell Isolation Kit (STEMCELL Technologies). After isolation, CD8⁺ T cells were seeded into a 24-well plate at a density of 2×10⁶ cells per well with a 2 mL medium. T cells were expanded with anti-CD3/antiCD28 antibody-conjugated beads (Life Technologies, Grand Island, NY) at a bead-to-cells ratio of 2:1 in the presence of 2,000 IU/mL of recombinant mouse IL-2 (R&D Systems, Minneapolis, MN) in complete RPMI 1640 (Gibco) medium supplemented with 10% v/v fetal bovine serum (FBS, Seradigm), 2 mM GlutaMAX Supplement (Gibco), and antibiotic-antimycotic (Anti-Anti; 100 units of penicillin, 100 μg/mL of streptomycin, and 0.25 μg/mL of amphotericin B; Gibco) for 48 h before further studies.

In vitro toxicity of Ac₄ManNAz and LEF, and viabilities of functionalized MSCs and MOLs: In vitro toxicities of Ac₄ManNAz and LEF against MSCs and MOLs, and the viabilities of functionalized MSCs and MOLs were quantified by MTS assay. Briefly, treated/functionalized cells were cultured in complete media for 4 days. The phenol-red media was replaced by phenol red-free DMEM (supplemented with 10% FBS) before quantifying the viabilities via MTS assay according to the manufacturer's protocol. The MSCs were seeded at a density of 2×10⁴ cells per well and the MOLs were seeded at a density of 1×10⁴ cells per well in a 96-well plate.

Preparation of azide-modified MSCs and MOLs: Azide-modified MSCs and MOLs were generated by the culture in a complete growth medium containing 50 μM of Ac₄ManNAz for 4 days. The Ac₄ManNAz-containing culture medium was refreshed every 48 h. Azide-modified cells were detached via TrypLE™ Express Enzyme (Gibco) according to the manufacturer's protocol for subsequent studies. The Ac₄ManNAz-containing culture medium was refreshed every 48 h.

Functionalization of azide-modified MSCs and MOLs with PD-L1-Ig and CD86-Ig: Two bioconjugation methods were investigated to functionalize MSCs and MOLs.

In the direct bioconjugation method, DBCO-functionalized PD-L1-Ig and/or CD86-Ig were conjugated to azide-modified MSCs or MOLs via SPAAC at 37° C. for 1 h. The target degree of functionalization was 5 μg fusion protein per one million cells. The bioconjugation was carried out at 20 million cells per mL. Functionalized MSCs or MOLs were purified via centrifugation (300 g, 3-4 min, 3 times) and resuspended in complete media for subsequent in vitro studies or 1×PBS for subsequent in vivo studies.

In the NP-pre-anchoring method, DBCO/MTZ NPs were first conjugated to the azide-modified MSCs or MOLs via SPAAC at 37° C. for 1 h. The target degree of functionalization was 500 μg of DBCO/MTZ NPs per one million cells (cell concentration: 20 million cells per mL). NP-functionalized MSCs or MOLs were purified via centrifugation (300 g, 3-4 min, 3 times). TCO-functionalized PD-L1-Ig and/or CD86-Ig were added to the NP-functionalized MSCs/MOLs via IEDDA at 37° C. for 1 h. As with the first bioconjugation method, the target degree of functionalization was 5 μg fusion protein(s) per million cells. Functionalized MSCs or MOLs were purified via centrifugation (300 g, 3-4 min, 3 times) and resuspended in complete media for subsequent in vitro studies or 1×PBS for subsequent in vivo studies. For selected in vivo experimental groups, functionalized MSCs were subjected to 100 Gy X-ray irradiation (via a RS2000 Biological Irradiator, operated at 160 kV and 24 mA) before administrated to the EAE mice.

The amount(s) of conjugated fusion protein(s) were quantified via fluorescence spectroscopy using A488-labeled PD-L1-Ig (excitation wavelength=480±20 nm; emission wavelength=525±20 nm) or Texas Red-labeled CD86-Ig (excitation wavelength=550±20 nm; emission wavelength=640±20 nm) for the bioconjugation. The amounts of MSC- and MOL-conjugated NPs were quantified via fluorescence spectroscopic method using Cy5-labeled DBCO/MTZ NPs (excitation wavelength=640±20 nm; emission wavelength=780±20 nm) for the bioconjugation. Functionalized dye-labeled cells were exchanged into PBS before fluorescence spectroscopic measurements. The detachment of the dye-labeled fusion proteins and NPs were monitored via fluorescence spectroscopy.

A time-dependent FACS study was used to quantify the PD-L1 and CD86 expressions of unmodified and functionalized MSCs and MOLs. At a desired point of time, cells were detached and blocked with rat anti-mouse CD16/CD32 (mouse BD Fc Block; BD Bioscience) before being stained with PE-labeled anti-mouse PD-L1 antibody (clone: MIH5, catalog number: 12-5982-82; Invitrogen) and FITC-labeled anti-mouse CD86 antibody (clone: GL1; catalog number: 11-0862-82; Invitrogen). Stained cells were then fixed with 4% paraformaldehyde (4% PFA; Sigma) and kept in dark at 4° C. before further FACS study. The PD-L1 and CD86 expressions of different functionalized MSCs were further evaluated by CLSM method after stained with PE-labeled anti-mouse PD-L1 antibody (clone: MIH5, catalog number: 12-5982-82; Invitrogen) and FITC-labeled anti-mouse CD86 antibody (clone: GL1; catalog number: 11-0862-82; Invitrogen).

For the CLSM study, MSCs were seeded in G422 Applied Cell Extracellular Matrix-coated microscope coverslips (1 cm diameter) in a 12-well plate. Cells were cultured with 50 μM of Ac₄ManNAz for 4 days, before functionalized with DBCO-functionalized PD-L1-Ig and/or CD86-Ig, or DBCO/MTZ NPs followed by TCO-functionalized PD-L1-Ig and CD86-Ig. Next, the MSCs were stained with PE-labeled anti-PD-L1, and FITC-labeled anti-CD86 were recorded in a Zeiss LSM 710 Spectral Confocal Laser Scanning Microscope.

For field-emission scanning electron microscopy study, MSCs were seeded in G422 Applied Cell Extracellular Matrix-coated microscope coverslips (1 cm diameter) in a 12-well plate. Cells were cultured with 50 μM of Ac₄ManNAz for 4 days, before functionalized with DBCO/MTZ NPs, followed by TCO-functionalized PD-L1-Ig and CD86-Ig. After functionalization, MSCs were then washed with 1×PBS containing 10 mM magnesium chloride three times before fixing with 10% neutral-buffered formalin. The FESEM images were recorded using a Zeiss Supra 25 FESEM microscope in the MSL at the UNC School of Medicine.

Myelin-specific CD4 T cell in vitro activation: Mouse IFN-γ and mouse IL-17A secreted from the activated myelin-specific 2D2 cells were quantified by ELISA assays as previously reported⁵⁶. The PD-1 and CTLA-4 expressions of myelin-specific 2D2 cells were quantified via the FACS method. Briefly, 2D2 cells (effector cells (E)) were cultured with different non-functionalized and functionalized MSCs and MOLs (target cells (T): 5×10⁴ cells per well in a 6-well plate that were seeded for 4 h before co-cultured with the 2D2 cells) at an E:T ratio of 10:1 for 48 h. The cell culture media (contain mainly the 2D2 cells) were preserved. 2D2 cells were collected from the cultured media via centrifugation at 1,000 g for 10 min. The moue IFN-γ and mouse IL-17A concentrations in the supernatants were quantified via mouse IFN-γ ELISA kit (ab100689; Abcam, Cambridge, MA) and mouse IL-17A ELISA kit (ab199081; Abcam, Cambridge, MA), according to manufacturer's instructions. The PD-1 and CTLA-4 expressions of the isolated 2D2 cells were quantified via FACS method after stained with A488-labeled anti-mouse PD-1 antibody (clone: MIH4, catalog number: 53-9969-42, Invitrogen), PE-labeled anti-mouse CTLA-4 antibody (clone: UC10-4B9, catalog number: 50-106-52, Invitrogen), and eFluor 660-labeled anti-mouse CD3 antibody (clone: 17A2, catalog number: 50-0032-82, Invitrogen)⁵⁶. Stained cells were fixed with 4% paraformaldehyde (4% PFA; Sigma) and kept in dark at 4° C. before further FACS study.

The differentiation of naïve 2D2 cells into IL10⁺ FoxP3⁺ T_{reg} cells was quantified by FACS as previously reported⁵⁶. The 2D2 cells were briefly cultured with different non-functionalized and functionalized MSCs and MOLs (5×10⁴ cells per well in a 6-well plate that seeded for 4 h before co-cultured with the 2D2 cells) at an E:T ratio of 10:1 for 72 h. 2D2 cells were collected from the cultured media via centrifugation at 1,000 g for 10 min. The isolated cells were first stained with eFluor 660-labeled anti-mouse CD3 antibody (clone: 17A2, catalog number: 50-0032-82, Invitrogen). They were then fixed with 4% PFA before permeabilization using the intracellular staining permeabilization wash buffer (Biolegend). Further, they were stained with A488-labeled anti-mouse FoxP3 antibody (clone: MF23, catalog number: 560403, BD Bioscience) and PE-labeled anti-mouse IL10 antibody (clone: JES5-16E3, catalog number: 561060, BD Bioscience) for FACS study.

Quantification of antigen-non-specific cytotoxic T cell inhabitation: The abilities for the functionalized MSCs to inhibit cytotoxic T cell activation were quantified by CellTrace CFSE Cell Proliferation assay (Thermo Fisher). Briefly, the CFSE-labeled expanded CD8⁺ T cells (isolated from wide-type C57BL/6 mice) were cultured with seeded unmodified/functionalized MSCs at an E:T ratio of 10:1 for 48 h in the presence of 1 molar equivalent (vs CD8⁺ T cells) of Dynabeads™ Mouse T-Activator CD3/CD28 T cells Activation Beads (Gibco)⁷². The proliferation of CFSE-labeled CD8⁺ T cells was quantified via FACS.

### In Vivo Studies

Animals were maintained in the Division of Comparative Medicine (an AAALAC-accredited experimental animal facility) under sterile environments at the University of North Carolina. All procedures involving the experimental animals were performed following the protocols that the University of North Carolina Institutional Animal Care and Use Committee has approved, and they conformed with the Guide for the Care and Use of Laboratory Animals (NIH publication no. 86-23, revised 1985).

In vivo toxicity of i.v. administered unmodified and PD-L1-Ig/CD96 FcIg NP-functionalized MSCs: The long-term in vivo toxicities of the i.v. administered MSCs and PD-L1-Ig/CD86-Ig NP-functionalized MSCs (2 million cells/mouse) were evaluated in healthy C56BL\6 mice (15 weeks old, female, Charles River Laboratories). The mice's body weight was monitored weekly after the administration. 5 weeks later, the mice were euthanized via an overdose of ketamine. Full blood and key organs were preserved for clinical chemistry and histopathological studies.

EAE induction and clinical evaluation: EAE was induced in wide-type C57BL/6 mice (female, 15-16 weeks old) through an active immunization method. For the induction of MOG₃₅₋₅₅ EAE in C56BL/6 mice, 200 μl of MOG₃₅₋₅₅/CFA emulsion (containing 200 μg of MOG₃₅₋₅₅ and about 0.8 mg of heat-killed Mycobacterium tuberculosis; Hooke Laboratories, Lawrence, MA) was subcutaneously administrated to each C56BL\6 mouse. For the induction of PLP₁₇₈₋₁₉₁ EAE in C56BL/6 mice, 200 μl of PLP₁₇₈₋₁₉₁/CFA emulsion (containing 50 μg of PLP₁₇₈₋₁₉₁ and about 0.8 mg of heat-killed Mycobacterium tuberculosis; Hooke Laboratories, Lawrence, MA) was subcutaneously given to each C56BL/6 mouse. No pertussis toxin was administered for the EAE induction. The body weight and clinical signs were monitor daily post-immunization. The EAE clinical signs were scored on 0 to 5.0 scale as follows: score 0: normal mouse; score 0.5: partial tail paresis; score 1.0: complete tail paresis; score 1.5: limp tail and hind leg inhibition; score 2.0: limp tail and weakness of hind legs; score 2.5: limp tail and no movement in one leg; score 3.0: complete hind limb paralysis; score 4.0: hind limb paralysis and forelimb weakness; score 5.0: moribund. The paralyzed mice were afforded easier access to food and water. Unless specified, MSCs and MOLs were administrated via tail vein i.v. injection. For the prophylactic study, unmodified MSCs or functionalized MSCs (2 million cells per mouse) were administered 1-day post-immunization. For the therapeutic treatment study, unmodified MSCs or functionalized MSC (2 million cells per mouse) were administered on day 17 or 18 p.i., when the EAE-inflicted mice showed severe EAE symptoms (EAE score≈2.0). For the selected studies, a booster dose of functionalized MSCs was i.v. administered on day 28 or 35 p.i. In a selected in vivo study, functionalized MSCs and MOLs were intramuscularly administrated to the tight muscles at the hind limb. Unless specified, mice were euthanized, and spinal columns were preserved on day 36 or 37 p.i. via full-body perfusion method for further histopathological studies. Preserved spinal columns were processed by the Animal Histopathology and Lab Medicine Core at the UNC School of Medicine for hematoxylin and eosin (H&E), Luxol fast blue (LFB), and anti-CD4 and anti-FoxP3 immunohistochemistry stains. H&E- and LFB-stained slides were imaged via a ScanScope AT2 (Leica Biosystems) pathology slide scanner. Spinal inflammation was quantified from representative H&E-stained sections⁷³. Anti-CD4 and anti-FoxP3 immunofluorescence-stained slides were imaged via a ScanScope FL (Leica Biosystems) pathology slide scanner.

T_{reg} cell depletion study: T_{reg} cell depletion study was performed in MOG₃₅₋₅₅ EAE-inflicted mice to demonstrate that the T_{reg} cells induced by the bioengineered MSCs play a key role in maintaining immunotolerance. The T_{reg} cells were depleted by an i.p. administration of 750 μg of anti-CD25 antibody (InVivoMAb, clone: PC-61.5.3, catalog number: BE0012; BioXCell), as previously reported. For the prophylactic study, the anti-CD25 antibody was administered on days 1, 3, and 5 p.i. (3×250 μg of anti-CD25)⁶⁵. PD-L1-Ig/CD86-Ig NP-functionalized MSCs were i.v. administrated on day 2 p.i. For the therapeutic study, the anti-CD25 antibody was administered on days 17, 19, and 21 p.i. (3×250 μg of anti-CD25). PD-L1-Ig/CD86-Ig NP-functionalized MSCs were i.v. administrated on day 18 p.i., when the mice had an average clinical score of 2.0. Bodyweight and clinical signs were monitored daily after immunization. Control groups EAE-inflicted mice did not receive i.p. injections of anti-CD25 before and after the treatment with the functionalized MSCs.

In vivo biodistribution study of i.v. administered MSCs in MOG₃₅₋₅₅ EAE-inflicted mice: The biodistribution of i.v. administered MSCs was determined by the ex vivo NIR fluorescence imaging method. For the biodistribution study, non-functionalized or azide-functionalized MSCs were first labeled with VivoTag 680 (VT680) Fluorescent Dye (Perkin Elmer), according to the manufacturer's protocol. VT680-labeled azide-modified MSCs were functionalized via the same method as the non-labeled MSCs. For the prophylactic imaging groups, different VT680-labeled MSCs were i.v. administrated 1 day p.i. The mice were euthanized 48 h after the administration of MSCs, and the key organs were preserved for ex vivo imaging study in an AMI HT Optical Imaging System (excitation wavelength=675±25 nm, emission wavelength=730±25 nm, exposure time=30 s, excitation power=40%) in the Biomedical Research Imaging Center at the UNC School of Medicine. For the therapeutic imaging groups, different VT680-labeled MSCs were i.v. administrated 17 days p.i. The mice were euthanized 48 h after the administration of the labeled MSCs. Key organs were preserved for ex vivo imaging study in an AMI HT Optical Imaging System (excitation wavelength=675±25 nm, emission wavelength=730±25 nm, exposure time=30 s, excitation power=40%) in the Biomedical Research Imaging Center at the UNC School of Medicine. The percentage of injected dose (% ID) of VT-680-labeled MSCs that was accumulated in each organ was calculated by comparing the fluorescence intensities of different standard VT680-labeled MSC samples.

In vivo mechanistic study: A mechanistic study was performed on the MOG₃₅₋₅₅ EAE-inflicted mice. For the prophylactic treatment groups, the mice received i.v. administration of unmodified/functionalized MSCs on day 2 p.i. The mice were euthanized on day 5 or 38 p.i., and spleens were preserved for further mechanistic study. On the other hand, the mice from the therapeutic treatment groups received i.v. administration of unmodified/functionalized MSCs on day 18 p.i. The treated mice were then euthanized on day 21 or 38 p.i., and spleens and spinal cords were preserved for further mechanistic study.

All the cell-based analyses were performed on single-cell suspensions of the spleen and spinal cord. For the isolation of splenocytes, the freshly preserved spleen was mashed through a cell strainer (70 μm; Fisher) in HBBS buffer. Erythrocytes were removed by ACK Lysis Buffer (Gibco) according to the manufacturer's protocol. The isolated splenocytes were first stained with T-Select I-Ab MOG35-55 Tetramer PB (Catalog number: TS0M704-1; MBL International, Woburn, MA). After the removal of the unbound tetramer, the cells were stained with Fixable Viability Stain 510 (catalog number: 564406; BD Bioscience), followed by A488-labeled anti-mouse CD4 antibody (clone: GK1.5; Invitrogen). The cells were then fixed with 4% PFA (Sigma) before permeabilization using the intracellular staining permeabilization wash buffer (Biolegend). They were then stained with DyLight 650 anti-mouse FoxP3 polyclonal antibody (catalog number: PA5-22773, Invitrogen), PE-Cyanine 7-labeled anti-mouse ROR-γ antibody (clone: B2D; catalog number: 25-6981-82, Invitrogen), and PE-Cyanine 5-labeled anti-mouse T-bet antibody (clone: 4B10; catalog: 15-5825-82) for FACS study.

The CNS-infiltrated lymphocytes were isolated from the freshly preserved spinal cord as previously reported. The isolated spinal cord was cut into small pieces and digested in a buffer solution that contained collagenase D (1 mg/mL; Roche) and DNase I (0.1 mg/mL, Roche) at 37° C. for 20 min. The tissues were mashed through a cell strainer (70 μm; Fisher) to collect single cells. Lymphocytes (at the interface of between 37% and 70% Percoll gradient) were isolated using Percoll gradients (GE Healthcare) via the centrifugation method as previously reported. The isolated lymphocytes were divided into two halves. One half of the lymphocytes were first stained with Fixable Viability Stain 510 (catalog number: 564406; BD Bioscience), followed by A488-labeled anti-mouse CD8a antibody (clone: 53-6.7; catalog: 53-0081-82, Invitrogen). Cells were then fixed with 4% PFA (Sigma) before permeabilization using the intracellular staining permeabilization wash buffer (Biolegend), and this was followed by staining with PE-Cyanine 7 anti-mouse IFN-gamma antibody (clone: XMG1.2; catalog number: 25-7311-41, Invitrogen) for FACS study. For the other half of the isolated lymphocytes, cells were first stained with T-Select I-Ab MOG35-55 Tetramer PB (Catalog number: TSOM704-1; MBL International, Woburn, MA) according to the manufacturer's protocol. After the removal of the unbound tetramer, cells were stained with Fixable Viability Stain 510 (catalog number: 564406; BD Bioscience) and A488-labeled anti-mouse CD4 antibody (clone: GK1.5; Invitrogen). Similar to the previous steps, the cells were fixed with 4% PFA (Sigma) before permeabilization using an intracellular staining permeabilization wash buffer (Biolegend). Finally, they were stained with DyLight 650 anti-mouse FoxP3 polyclonal antibody (catalog number: PA5-22773, Invitrogen), PE-Cyanine 7 anti-mouse IFN-gamma antibody (clone: XMG1.2; catalog number: 25-7311-41, Invitrogen), and PE-eFluor 610-labeled anti-mouse IL-17A antibody (clone: 17B7; catalog number: 61-7177-82, Invitrogen) for FACS study.

Statistical Analysis: No statistical methods were used to pre-determine the sample size of the experiment. Quantitative data were expressed as mean±standard error of the mean (SEM). The analysis of variance was completed using two-tailed t-tests in the Graph Pad Prism 6 software pack. *P<0.05 was considered statistically significant.

### Example 1: Functionalization of NIT-1 Cells

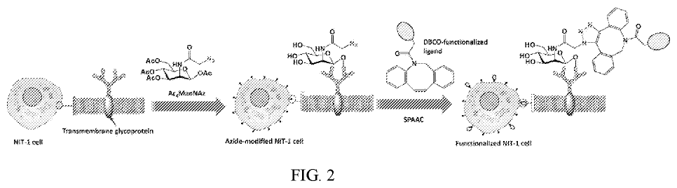

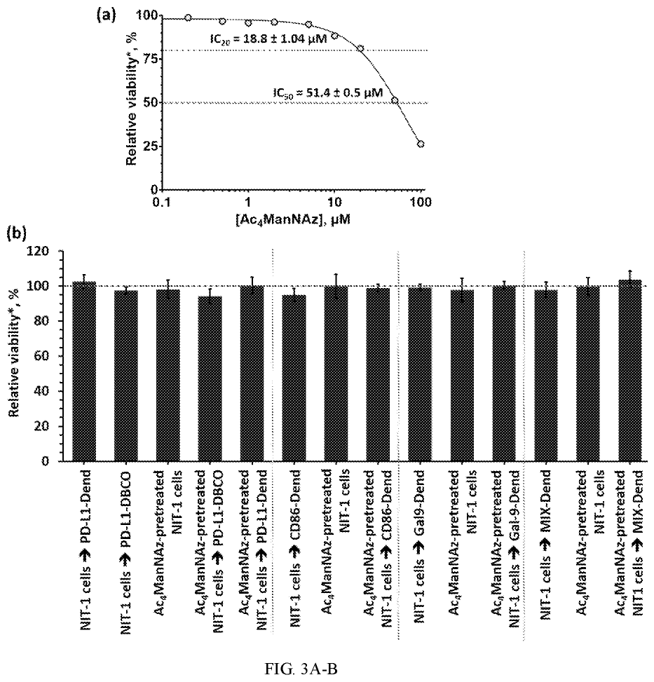

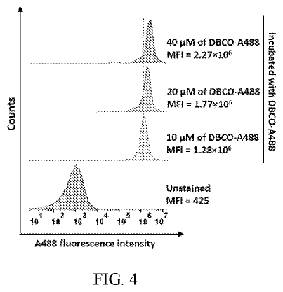

To demonstrate that metabolic glycoengineering and biorthogonal click chemistry facilitate the functionalization of NIT-1 cells (pancreatic β cells isolated from pre-diabetic NOD mice) with immune checkpoint molecules, PD-L1 was used as a model ligand to test two strain-promoted alkyne-azide cycloaddition (SPACC) functionalization strategies on azide-modified NIT-1 cells (FIG. 2). Azide-modified NIT-1 cells were obtained by in vitro culturing with 20 μM of N-azidoacetylmannosamine-tetraacylated (Ac₄ManNAz) for four days (FIG. 3(a)). The metabolism of Ac₄ManNAz incorporates ManNAz into mucin-type O-linked glycoproteins on the cell membrane of NIT-1 cells. The presence of azide groups on the modified NIT-1 cells was confirmed using labeling by Alexa Fluor 488 (A488)-functionalized dibenzocyclooctyne (DBCO) (FIG. 4).

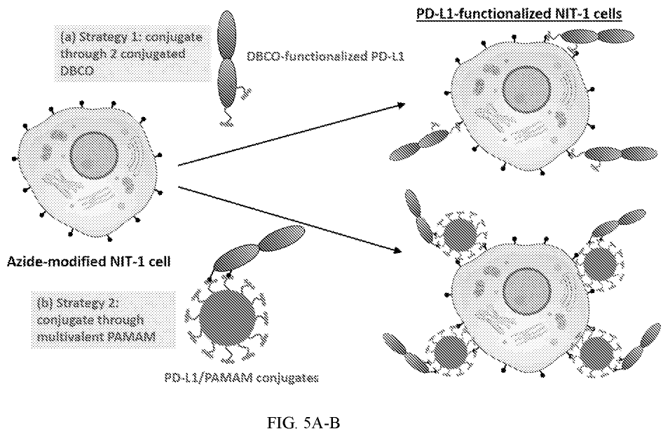

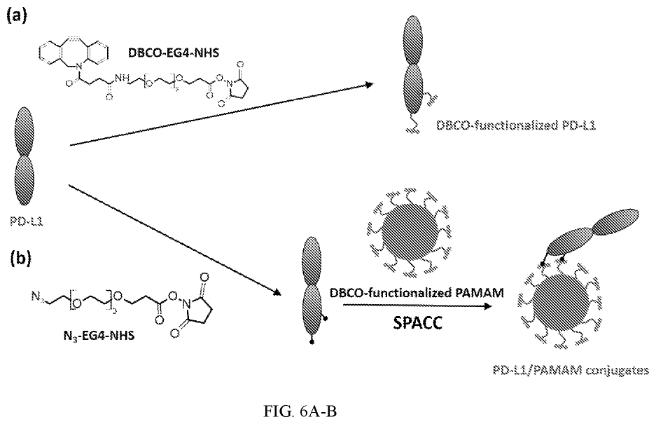

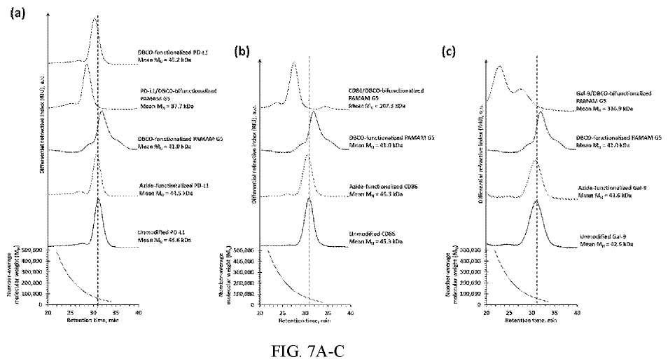

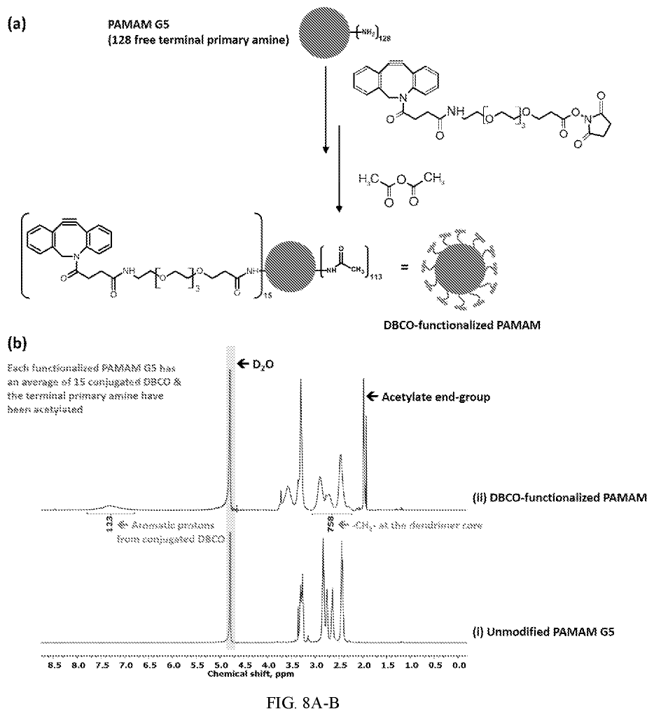

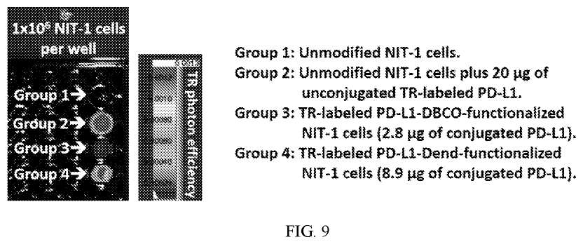

The conjugation efficiencies of two different functionalization strategies were examined (FIG. 5). One strategy used bivalent DBCO-functionalized PD-L1 (PD-L1-DBCO) (FIG. 5(a)). The other strategy used multivalent DBCO-functionalized dendrimer-conjugated PD-L1 (PD-L1-Dend) (FIG. 5(b)). The PD-L1-DBCO ligand was functionalized with an average of two DBCO ligands conjugated via the amine-N-hydroxysuccinimide (NHS) ester coupling reaction (FIGS. 6(a) and 7(a)). PD-L1-Dend was prepared using SPACC between DBCO-functionalized polyamidoamine dendrimer G5 (functionalized with an average of 15 DBCO molecules; FIG. 8) and a molar equivalent amount of azide-functionalized PD-L1 (FIGS. 6(b) and 7(a)). Both functionalized PD-L1 ligands were conjugated to the azide-modified NIT-1 cells via biorthogonal SPACC at a target loading of 10 μg of functionalized PD-L1 per million cells (FIG. 5). By using Texas Red-labeled PD-L1 (TR-PD-L1) in the labeling study, it was determined that each batch of one million NIT-1 cells was functionalized with 1.4 μg of TR-PD-L1-DBCO or 4.4 μg of TR-PD-L1-Dend (FIG. 9). The higher conjugation efficiency recorded for the TR-PD-L1-Dend can be explained by the multivalent effect caused by the dendrimer.

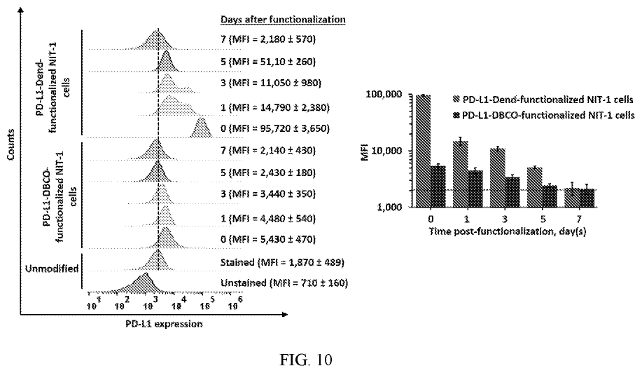

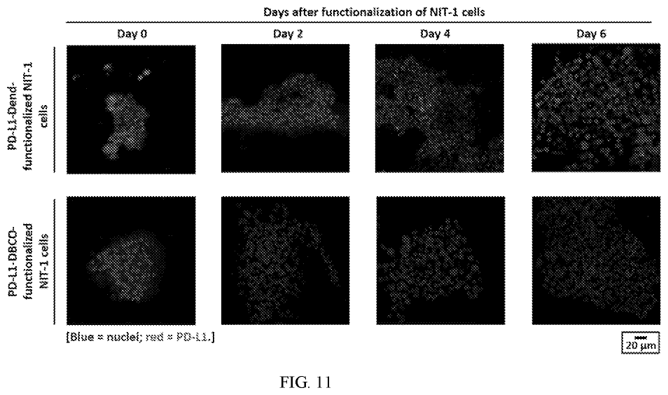

In addition, flow cytometry (FACS; FIG. 10) and confocal fluorescence microscopy (CLSM; FIG. 11) studies revealed that the as-prepared PD-L1-Dend-functionalized NIT-1 cells contained approximately 26 times more active PD-L1 on the surface of NIT-1 cells than those functionalized through DBCO. This suggests that a significant number of conjugated PD-L1-DBCO molecules were incorrectly orientated after conjugation onto the NIT-1 cells.

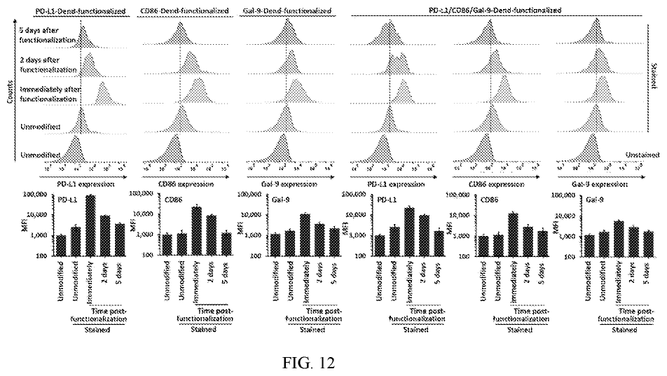

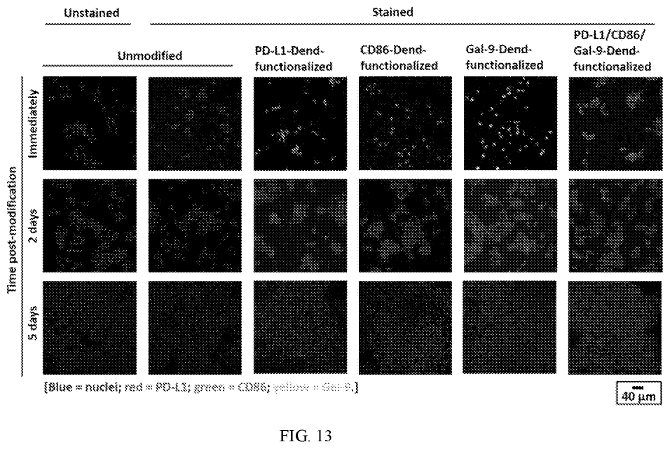

A further time-dependent study revealed that the PD-L1 expressions of PD-L1-functionalized NIT-1 cells gradually declined after conjugation owing to mitotic division and glycan/membrane recycling. The PD-L1 expressions of PD-L1-DBCO-functionalized NIT-1 cells dropped to a background level within three days after conjugation, while the PD-L1-Dend-functionalized NIT-1 cells maintained a constant level of PD-L1 for at least five days (FIG. 10). This indicates that the multivalent dendrimer-based functionalization approach facilitates more effective conjugation and can retain the functionality of the conjugated biomolecule for a longer period. Further in vitro toxicity studies confirmed that neither metabolic labeling method affected the proliferation of the engineered NIT-1 cells (FIG. 3(b)). Therefore, the dendrimer-based conjugation strategy was used to engineer CD86- and Gal-9-mono-functionalized NIT-1 cells, along with PD-L1/CD86/Gal-9-tri-functionalized NIT-1 cells. FACS and CLSM studies confirmed the successful decoration of multiple immune checkpoint molecule(s) onto the NIT-1 cells (FIGS. 3(b), 12 and 13).

### Example 2: PD-L1-Functionalized NIT-1 Cells Induces Immunological Tolerance in Autoreactive T Cells and Reverses Early-Onset Hyperglycemia

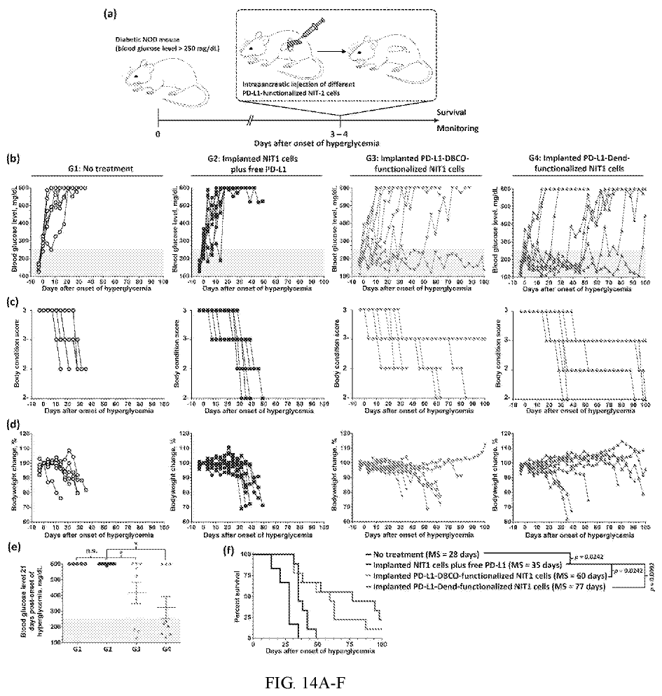

To demonstrate that PD-L1-functionalized NIT-1 cells can induce immunological tolerance in autoreactive T cells and reverse early-onset hyperglycemia (glycemia>250 mg/dl) in NOD mice, PD-L1-functionalized NIT-1 cells were intrapancreatically administered to early-onset hyperglycemic mice to allow the functionalized β cells to directly interface with the autoreactive T cells (FIG. 14). Two-thirds of the mice treated with the PD-L1-Dend-functionalized NIT-1 cells showed an initial response to the treatment (i.e., became normoglycemic for at least three weeks after the treatment; FIG. 14(b-e)), and they had significantly prolonged survival (median survival, MS=77 days; FIG. 14(f)). The mice treated with azide-modified NIT-1 cells and unconjugated (“free”) PD-L1 had an MS=35 days (p=0.0242 versus the non-treatment group; FIG. 14(f)). On the other hand, only one-third of the mice treated with PD-L1-DBCO-functionalized NIT-1 cells showed an initial response to the treatment (FIG. 14(b-e)), and the treatment only slightly prolonged the MS (MS=25 days; FIG. 14(f)) compared to that treated with non-functionalized NIT-1 cells. The weaker immune response observed for the DBCO direct conjugation strategy was due to the rapid dissociation of conjugated PD-L1. Thus, further investigation was conducted on the therapeutic responses of β cells indirectly functionalized through the multivalent dendrimer.

### Example 3: NIT-1 Cells Co-Functionalized with PD-L1, CD86, and Gal-9

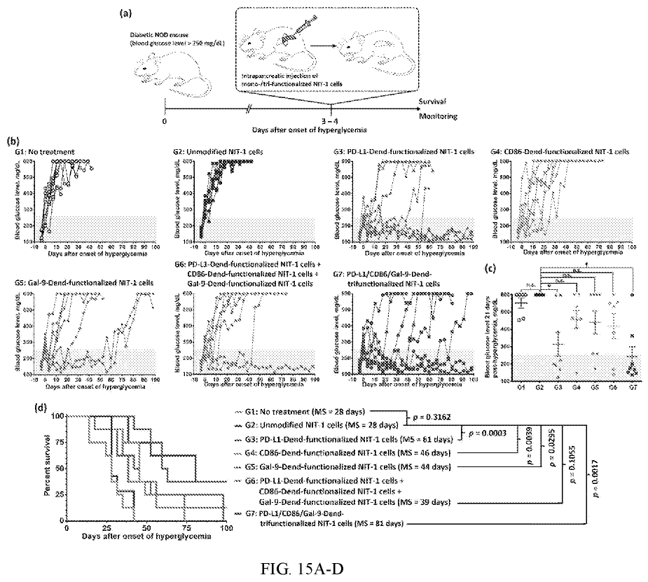

Further correlative study to compare the efficiencies of different immune checkpoint molecule-functionalized NIT-1 cells in reversing newly onset hyperglycemia was conducted. The functionalized β cells were intrapancreatically administrated to allow them to directly interface between the functionalized β cells and autoreactive T cells (FIG. 15(a)). Further, unmodified NIT-1 cells alone did not reverse hyperglycemia or prolong survival (MS=28 days versus 28 days recorded for non-treatment group; p=0.3162;

FIGS. 15(b)-(d), 16 and 17). Three-quarters of the mice treated with the PD-L1-Dend-functionalized NIT-1 cells responded partially to the treatment, and more than half of them remained diabetes-free for at least 50 days after the treatment (MS=61 days; p=0.0003 versus treatment with unmodified NIT-1 cells; FIGS. 15(b)-(d), 16 and 17). Although CD86 and Gal-9 play critical roles in inducing immuno-tolerance, most early-onset hyperglycemic mice did not respond very well to the treatment using CD86- and Gal-9-functionalized NIT-1 cells. Treatment with CD86-functionalized cells only slowed down the progression of the disease and it slightly prolonged their survival (MS=46 days; p=0.0039 versus treatment with non-functionalized NIT-1 cells; FIGS. 15(b)-(d), 16 and 17). A quarter of the mice treated with the Gal-9-functionalized cells partially reversed hyperglycemia for about 60 days, but the treatment increased the MS only slightly to about 44 days (p=0.00295 versus treatment with non-functionalized NIT-1 cells; FIGS. 15(b)-(d), 16 and 17). The different treatment responses can be explained by the different inhabitation mechanisms of different checkpoint molecules. Moreover, the heterogeneity of T1D. PD-L1-functionalized NIT-1 cells are the most effective at reverting hyperglycemia because they directly exhaust the autoreactive effector T cells.

### Example 4: NIT-1 Cells Co-Functionalized with PD-L1, CD86, and Gal-9

Further studies were conducted on whether the combination of PD-L1, CD86, and Gal-9 could reverse newly onset hyperglycemia. NIT-1 cells co-functionalized with PD-L1, CD86, and Gal-9, or a combination of three different mono-functionalized NIT-1 cells was intrapancreatically administered in the same amount (FIG. 15(a)). Only a quarter of the diabetic mice implanted with the three different mono-functionalized NIT-1 cells showed initial responses to the treatment and achieved long-term survival (MS=39 days; p=0.0039 versus treatment with non-functionalized NIT-1 cells; FIGS. 15(b)-(d), 16 and 17). On the other hand, more than 85% of the diabetic mice treated with the tri-functionalized NIT-1 cells showed an initial response to the treatment. Half of the treated mice reverted hyperglycemia for at least 40 days and achieved long-term survival (MS=90 days; p=0.0017 versus treatment with non-functionalized NIT-1 cells, and p=0.0375 versus treatment with the combination of 3 different mono-functionalized NIT-1 cells; FIGS. 15(b)-(d), 16 and 17). The implantation of tri-functionalized NIT-1 cells showed survival benefits comparable to the implantation of PD-L1-functionalized NIT-1 cells (p=0.9648). However, the tri-functionalized cells contained only one-third of the conjugated PD-L1. They showed higher initial response rates than those for PD-L1-functionalized NIT-1 cells. In addition, the exhaustion or inhibition of more than one immune checkpoint pathway prevented the deficiency or mutation of one pathway affecting the therapeutic outcomes.

### Example 5: Tri-Functionalized NIT-1 Cell-Embedded Pan-ECM

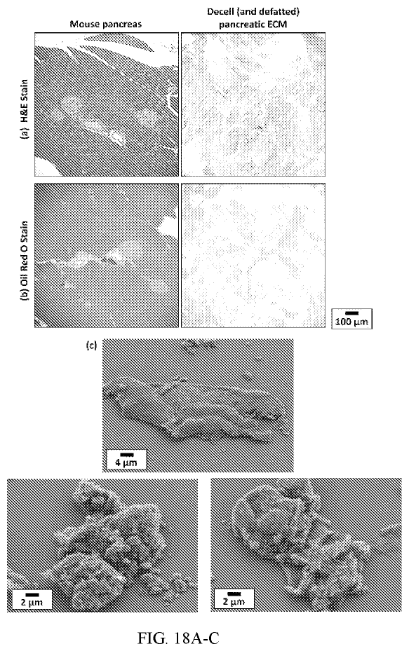

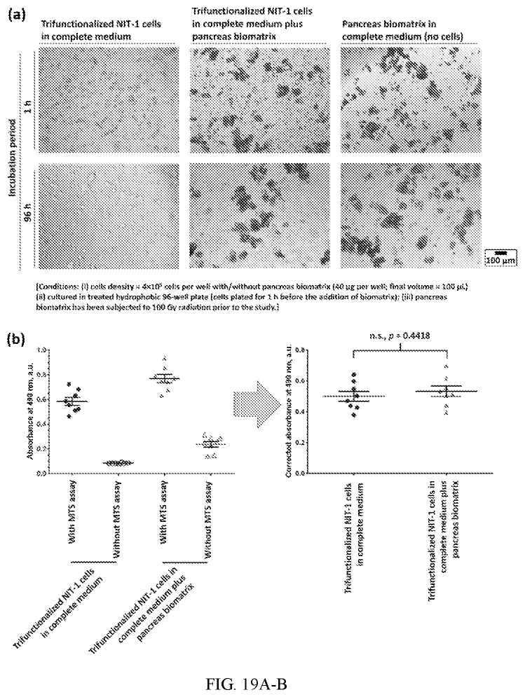

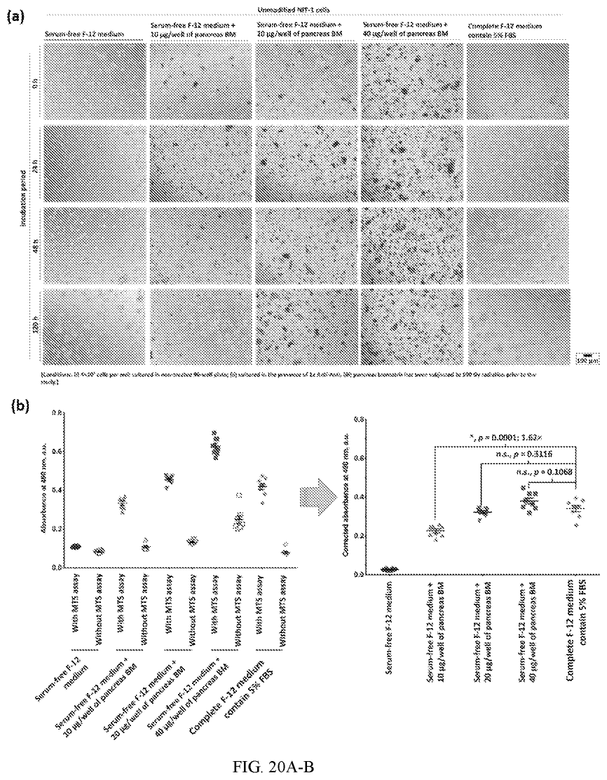

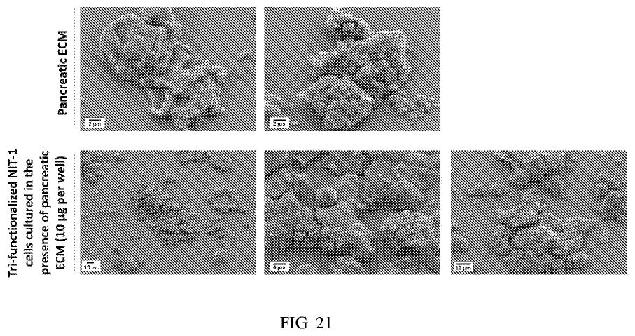

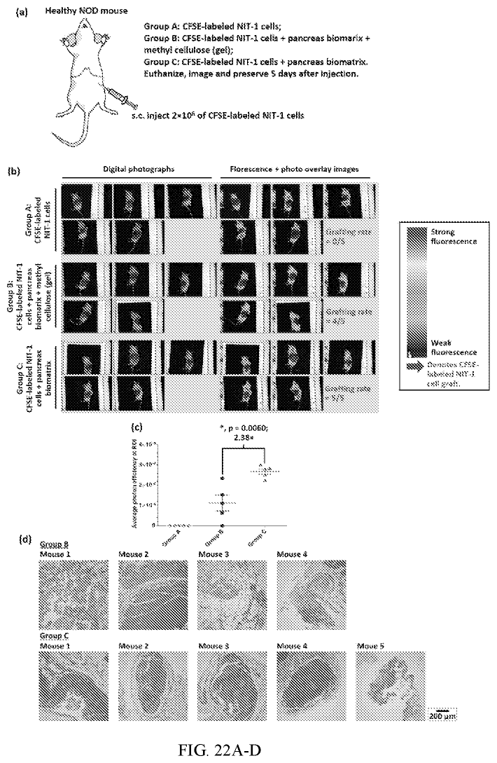

Although the intrapancreatic administration of PD-L1/CD86/Gal-9-tri-functionalized NIT-1 cells can partially revert early-onset hyperglycemia, this treatment strategy is difficult to translate to human subjects. Moreover, repeated intrapancreatic injections may cause surgery-related complications. To address this, an s.c. injectable pan-ECM scaffold was engineered to provide a tissue-specific microenvironment for the β cell vaccine. The acellular pan-ECM scaffold was prepared from healthy murine pancreata through a spin-decell method. The isolated pancreas ECM was lyophilized and ball-milled before further use (FIG. 18). Proteomic analysis revealed that the spin-decell protocol preserved the physiological levels of pancreatic ECM-associated and cellular proteins (Table 1). These are important for guiding cell migration, stimulating cell proliferation, and modulating cellular response.²⁵ The tri-functionalized NIT-1 cells proliferated and spontaneously formed three-dimensional spheroid colonies with pan-ECM in a serum-free culture medium in vitro (FIGS. 19, 20 and 21). In contrast, the NIT-1 cells did not survive in the serum-free culture medium under the same in vitro culture conditions (FIG. 20). To demonstrate that pan-ECM improved the retention of s.c.-injected β cells, we s.c. inoculated carrier-free CFSE-labeled NIT-1 cells and CFSE-labeled NIT-1 cell-embedded pan-ECM into a site close to the pancreatic lymph nodes in healthy NOD mice (FIG. 22(a)). An ex vivo fluorescence imaging study performed one week after the injection confirmed that the β cell-embedded pan-ECM had been retained at the injection site (FIGS. 22(b) and (c)). In contrast, no carrier-free CFSE-labeled NIT-1 cells could be identified at the injection site (FIGS. 22(b) and (c)). The addition of 6 wt/wt % of methylcellulose (MC) to the pan-ECM scaffold to form a β cell-embedded thermal-responsive hydrogel did not improve the survival of CFSE-labeled NIT-1 cells, and it reduced the grafting rate (60%) (FIGS. 22(b) and (c)). The lower grafting rate presumably occurred because of the rapid gelation of the MC-pan-ECM formulation reducing the number of viable cells injected into the inoculation site.

### Example 6: Tri-Functionalized NIT-1 Cell-Embedded Pan-ECM as a Vaccine Reverse Early-Onset Hyperglycemia

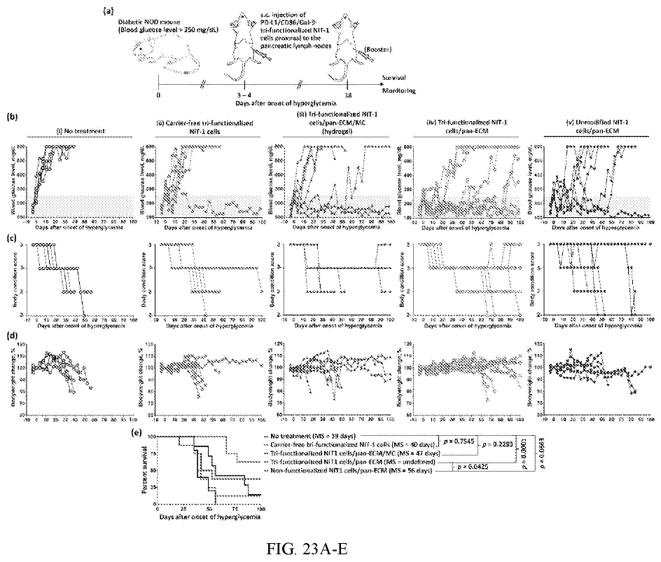

To demonstrate that the tri-functionalized NIT-1 cell-embedded pan-ECM can be used as a vaccine to reverse early-onset hyperglycemia, the β cell-embedded pan-ECM was administered s.c. to hyperglycemic NOD mice within three days of onset. A booster was administered two weeks after the initial treatment (FIG. 23(a)). All hyperglycemic mice treated with the tri-functionalized NIT-1 cell-embedded pan-ECM showed a complete initial response, with about 60% of them being diabetes-free for more than 50 days after the initial treatment (FIGS. 23(b), (c) and (d)). In addition, more than 60% of the treated mice achieved long-term survival (FIG. 23(e)), whereas the medium survival of non-treated mice was only 39 days (FIG. 23(e)). Control studies indicated that the s.c. administration of carrier-free tri-functionalized NIT-1 cells and non-functionalized NIT-1 cell-embedded pan-ECM did not provide a significant immune response to revert hyperglycemia (FIGS. 23(b), (c), (d) and (e)).
Tian, X., et al., Organ-specific metastases obtained by culturing colorectal cancer cells on tissue-specific decellularized scaffolds. Nat Biomed Eng, 2018. 2: p. 443-452.

### Example 7: PD-L1 and CD86 Dual-Functionalized Schwann Cells Delay and Reverse Experimental Autoimmune Encephalomyelitis

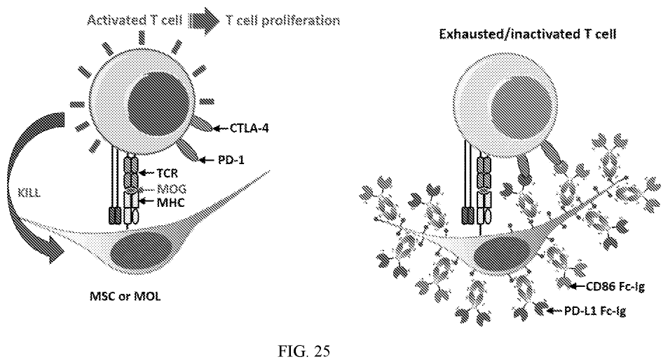

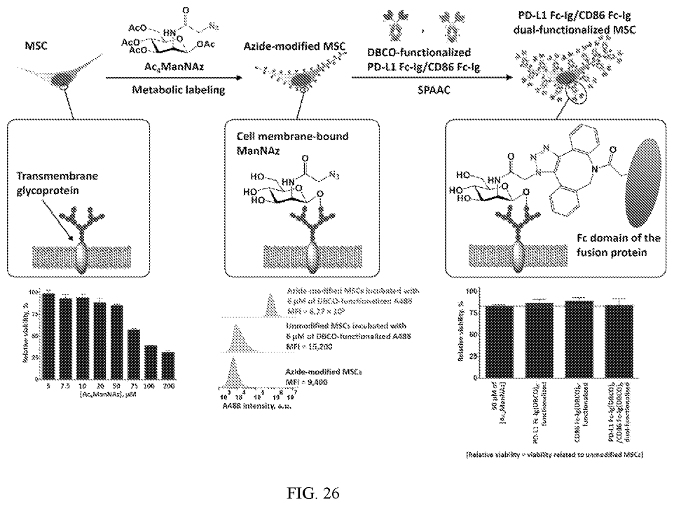

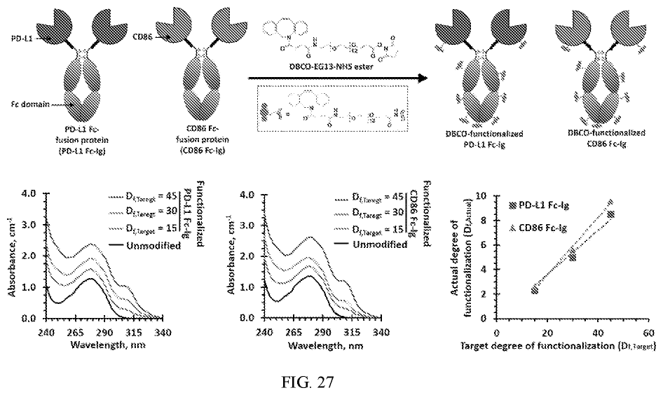

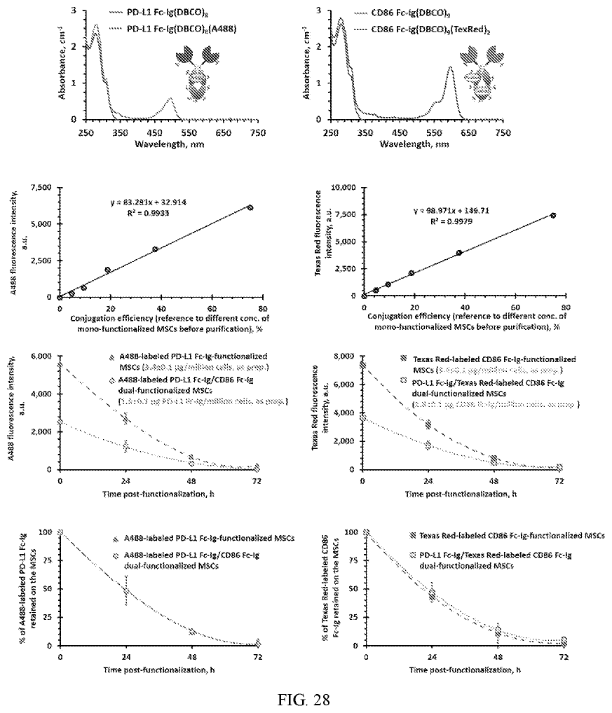

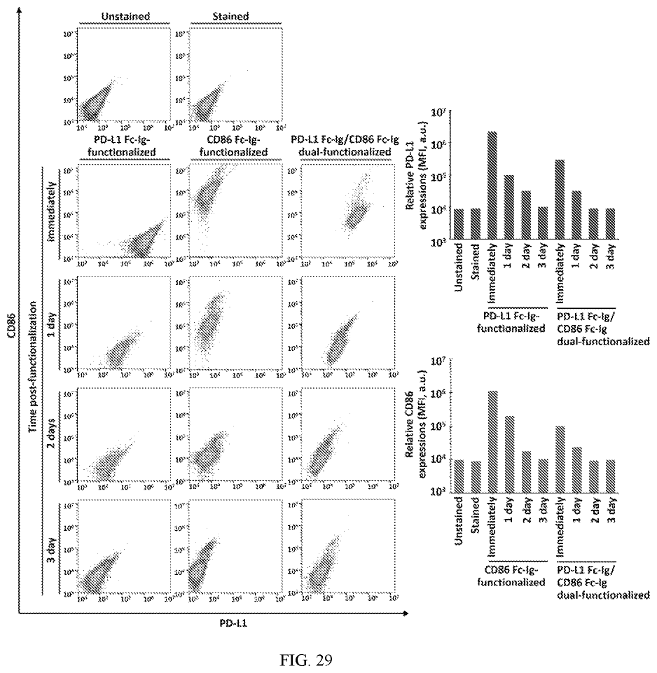

PD-L1 Fc fusion protein (PD-L1 Fc-Ig) and CD86 Fc fusion protein (CD86 Fc-Ig)-functionalized mouse Schwann cells (MSCs) were engineered to prevent or relieve the symptoms of experimental autoimmune encephalomyelitis (EAE, an experimental model for multiple sclerosis; Mendel, I. et al., A myelin oligodendrocyte glycoprotein peptide induces typical chronic experimental autoimmune encephalomyelitis in H-2b mice: Fine specificity and T cell receptor Vβ expression of encephalitogenic T cells. European Journal of Immunology 1995. 25(7):1951-1959) in the mouse (FIG. 25). Azide-modified MSCs were obtained by in vitro culturing in Prigrow III Medium contained 50 μM of Ac₄ManNAz for 5 days in Applied Cell Extracellular Biomatrix-coated tissue culture flasks (FIG. 26). DBCO-functionalized PD-L1 Fc-Ig and CD86 Fc-Ig were prepared via amine-NHS ester chemistry between DBCO-EG13-NHS ester and PD-L1 Fc-Ig or CD86 Fc-Ig. The target degree of functionalization was 45, and the actual degree of functionalization was about 9 (FIG. 27). PD-L1 Fc-Ig and CD86 Fc-Ig mono-/dual-functionalized MSCs were prepared via SPACC between azide-modified MSCs and DBCO-functionalized PD-L1 Fc-Ig and/or CD86 Fc-Ig (FIG. 26) at physiological conditions for 1 h. The conjugation of PD-L1 Fc-Ig and/or CD86 Fc-Ig were confirmed by fluorescence spectroscopy (FIG. 28) and FACS methods (FIG. 29).

EAE is induced in C57BL/6 mice by active immunization with emulsion of MOG₃₅₋₅₅ peptide (200 μg per mouse) in complete Freund's adjuvant. The clinical signs of EAE were monitored daily after immunization and graded using the following scale: 0.0 no changes in motor function, 0.5 half-tail paralyzed, 1.0 full-tail paralyzed, 1.5 hind limb weakness, 2.0 tail and hind limb weakness, 2.5 partial hind limb paralyzed, 3.0 complete hind limb paralyzed. Typical EAE onset is 10-12 days after immunization, with peak of disease 6-8 days after onset of each mouse.

Prophylactic studies assess if treatment will affect the course of disease both before and after the first clinical signs of EAE. In a prophylactic study, median time to disease onset is sensitive and maximum EAE score measure of treatment efficacy. In prophylactic treatment, unmodified or functionalized MSCs (2×10⁶ cells per mouse) were intravenously administered to the EAE-induced mice 1 day after immunization with MOG₃₅₋₅₅ peptide.

Therapeutic study assesses if treatment will reverse the course of disease or improve the recovery from EAE. In therapeutic treatment, unmodified or functionalized MSCs (2×10⁶ cells per mouse) were intravenously administered to the EAE-induced mice 17 day after immunization with MOG₃₅₋₅₅ peptide, when the mice showed maximum EAE score.

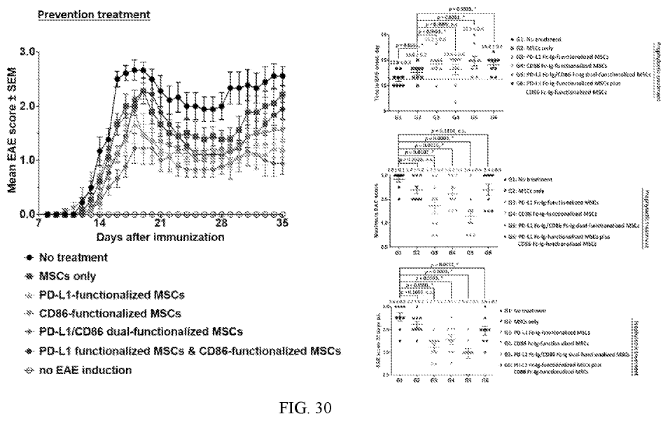

In the prevention study (FIG. 30), the administration of PD-L1 Fc-Ig and CD86 Fc-Ig dual-functionalized MSCs delayed the onset of EAE by 2 days (compared with non-treatment group), and significantly reduced the maximum EAE score from 2.8±0.1 (for the non-treatment group) to 1.3±0.3. PD-L1 Fc-Ig mono-functionalized MSCs also effectively delayed the onset of EAE but it was slightly less effectively in reducing the maximum EAE symptom (1.7±0.1). The CD86 Fc-Ig mono-functionalized MSCs were less effective at delaying the onset of EAE and reduce the maximum EAE score than PD-L1 Fc-Ig mono-functionalized MSCs. The 1:1 combination of PD-L1 Fc-Ig mono-functionalized MSCs and CD86 Fc-Ig mono-functionalized MSCs were not as effective as the dual-functionalized MSCs to reduce the maximum EAE score (2.4±0.3 vs 1.3±0.3 recorded for the dual-functionalized MSCs).

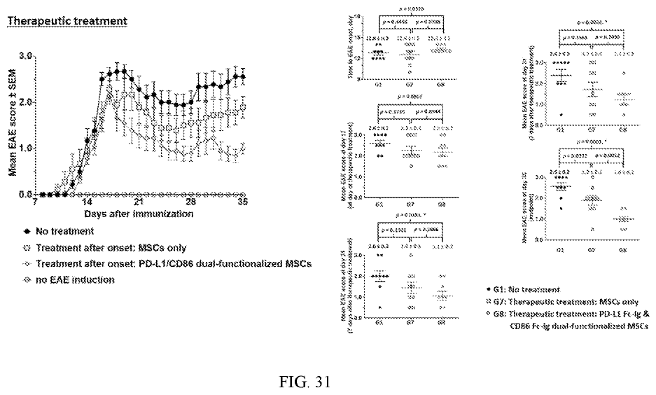

In the therapeutic treatment (FIG. 31), the administration PD-L1 Fc-Ig and CD86 Fc-Ig of dual-functionalized MSCs significantly reduced the average EAE score by 0.9 compared with the non-treatment group 1 week after treatment (P=0.0131). Conversely, the administration of non-functionalized MSCs non-significantly reduced the average EAE score by 0.7 (P=0.1501). At the study endpoint (35 days post-immunization), the dual-functionalized MSC reduced the average EAE score by 1.6 compared with the non-treatment group (1.0±0.1 vs 2.6±0.2), whereas the non-functionalized MSCs reduced the average EAE score by 0.7 compared with the non-treatment group (1.9±0.2 vs 2.6±0.2).

The PD-L1 Fc-Ig and CD86 Fc-Ig of dual-functionalized MSCs can effectively delay and relieve the clinical symptoms of EAE.

TABLE 1
Extracellular and cellular protein compositions of native murine pancreata and pancreatic ECM.

| | | | | Native pancreas | | | |
|---|---|---|---|---|---|---|---|
| | | | | tissue | | Pancreatic ECM | |
| | | | | Mean of | SEM of | Mean of | SEM of |
| Majority | | | | log₂(inte | log₂(inte | log₂(inten- | log₂(inten- |
| protein | | Gene | ECM | nsities), | nsities), | sities), | sities), |
| IDs | Protein names | names | ? | a.u. | a.u. | a.u. | a.u. |
| Q8C6K9 | Collagen alpha-6(VI) chain | Col6a6 | √ | 23.42 | 0.38 | 31.53 | 0.70 |
| P08121 | Collagen alpha-1(III) chain | Col3a1 | √ | 22.28 | 0.24 | 29.90 | 0.12 |
| Q61555 | Fibrillin-2 | Fbn2 | √ | 21.84 | 0.16 | 29.02 | 0.12 |
| Q9QZJ6 | Microfibrillar-associated | Mfap5 | √ | 22.53 | 0.80 | 29.69 | 0.03 |
| | protein 5 | | | | | | |
| P55002 | Microfibrillar-associated | Mfap2 | √ | 21.66 | 0.57 | 28.80 | 0.08 |
| | protein 2 | | | | | | |
| P54320 | Elastin | Eln | √ | 21.75 | 0.83 | 28.82 | 0.19 |
| Q61001 | Laminin subunit alpha-5 | Lama5 | √ | 21.94 | 0.44 | 28.87 | 0.09 |
| A6H584 | Collagen alpha-5(VI) chain | Col6a5 | √ | 21.59 | 0.21 | 28.23 | 0.11 |
| Q9QZZ6 | Dermatopontin | Dpt | √ | 22.79 | 0.21 | 28.88 | 0.05 |
| Q61292 | Laminin subunit beta-2 | Lamb2 | √ | 23.35 | 0.16 | 29.30 | 0.10 |
| O88322 | Nidogen-2 | Nid2 | √ | 23.55 | 0.29 | 29.32 | 0.04 |
| P11276 | Fibronectin; Anastellin | Fn1 | √ | 22.67 | 0.44 | 28.33 | 0.48 |
| Q02788 | Collagen alpha-2(VI) chain | Col6a2 | √ | 26.68 | 0.19 | 32.30 | 0.08 |
| O88207 | Collagen alpha-1(V) chain | Col5a1 | √ | 22.61 | 0.64 | 27.84 | 0.12 |
| Q61554 | Fibrillin-1 | Fbn1 | √ | 29.71 | 0.25 | 34.79 | 0.12 |
| Q99JR5 | Tubulointerstitial nephritis | Tinagl1 | √ | 22.50 | 0.33 | 27.48 | 0.06 |
| | antigen-like | | | | | | |
| Q9WVH9 | Fibulin-5 | FbIn5 | √ | 21.21 | 0.41 | 26.11 | 0.10 |
| Q04857 | Collagen alpha-1(VI) chain | Col6a1 | √ | 27.49 | 0.36 | 32.14 | 0.11 |
| Q01149 | Collagen alpha-2(1) chain | Col1a2 | √ | 29.57 | 0.38 | 34.11 | 0.10 |
| P29788 | Vitronectin | Vtn | √ | 21.35 | 0.56 | 25.77 | 0.08 |
| P11087 | Collagen alpha-1(I) chain | Col1a1 | √ | 30.10 | 0.36 | 34.40 | 0.09 |
| P97927 | Laminin subunit alpha-4 | Lama4 | √ | 22.91 | 0.08 | 26.84 | 0.04 |
| P08122 | Collagen alpha-2(IV) | Col4a2 | √ | 27.72 | 0.22 | 31.52 | 0.07 |
| | chain; Canstatin | | | | | | |
| P28654 | Decorin | Dcn | √ | 27.13 | 0.06 | 30.85 | 0.06 |
| O55222 | Integrin-linked protein | Ilk | √ | 23.20 | 0.46 | 26.67 | 0.16 |
| | kinase | | | | | | |
| P02463 | Collagen alpha-1(IV) | Col4a1 | √ | 28.24 | 0.16 | 31.70 | 0.04 |
| | chain; Arresten | | | | | | |
| E9Q557 | Desmoplakin | Dsp | √ | 24.49 | 0.15 | 27.85 | 0.07 |
| Q99MQ4 | Asporin | Aspn | √ | 24.36 | 0.37 | 27.61 | 0.09 |
| P21981 | Protein-glutamine gamma- | Tgm2 | √ | 26.99 | 0.12 | 30.23 | 0.06 |
| | glutamyltransferase 2 | | | | | | |
| Q05793 | Basement membrane- | Hspg2 | √ | 29.19 | 0.08 | 32.20 | 0.06 |
| | specific heparan sulfate | | | | | | |
| | proteoglycan core | | | | | | |
| | protein; Endorepellin;LG3 | | | | | | |
| | peptide | | | | | | |
| Q8VDD5 | Myosin-9 | Myh9 | √ | 30.17 | 0.13 | 33.14 | 0.03 |
| Q9JK53 | Prolargin | Prelp | √ | 23.08 | 0.32 | 25.95 | 0.12 |
| Q62009 | Periostin | Postn | √ | 22.23 | 0.55 | 25.09 | 0.10 |
| Q60997 | Deleted in malignant brain | Dmbt1 | √ | 30.73 | 0.04 | 33.47 | 0.03 |
| | tumors 1 protein | | | | | | |
| O70503 | Very-long-chain 3-oxoacyl- | Hsd17b12 | √ | 26.36 | 0.05 | 28.70 | 0.08 |
| | CoA reductase | | | | | | |
| P55258 | Ras-related protein Rab-8A | Rab8a | √ | 24.60 | 0.11 | 26.76 | 0.13 |
| P51881 | ADP/ATP translocase | Slc25a5 | √ | 32.34 | 0.04 | 34.48 | 0.02 |
| | 2; ADP/ATP translocase 2, | | | | | | |
| | N-terminally processed | | | | | | |
| O55142 | 60S ribosomal protein | Rpl35a | √ | 29.44 | 0.03 | 31.58 | 0.03 |
| | L35a | | | | | | |
| O35206 | Collagen alpha-1(XV) | Col15a1 | √ | 26.38 | 0.14 | 28.29 | 0.09 |
| | chain; Restin | | | | | | |
| P67984 | 60S ribosomal protein L22 | Rpl22 | √ | 29.34 | 0.03 | 31.21 | 0.04 |
| P62830 | 60S ribosomal protein L23 | Rpl23 | √ | 31.28 | 0.02 | 33.15 | 0.04 |
| P62245 | 40S ribosomal protein | Rps15a | √ | 30.95 | 0.07 | 32.68 | 0.03 |
| | S15a | | | | | | |
| Q8VEK3 | Heterogeneous nuclear | Hnrnpu | √ | 28.28 | 0.07 | 29.90 | 0.06 |
| | ribonucleoprotein U | | | | | | |
| P62301 | 40S ribosomal protein S13 | Rps13 | √ | 31.87 | 0.02 | 33.47 | 0.03 |
| P14131 | 40S ribosomal protein S16 | Rps16 | √ | 31.19 | 0.03 | 32.74 | 0.02 |
| P62281 | 40S ribosomal protein S11 | Rps11 | √ | 31.51 | 0.03 | 33.02 | 0.01 |
| Q9CXW4 | 60S ribosomal protein L11 | Rpl11 | √ | 31.95 | 0.02 | 33.46 | 0.03 |
| Q8BTM8 | Filamin-A | Flna | √ | 26.15 | 0.20 | 27.65 | 0.05 |
| P10493 | Nidogen-1 | Nid1 | √ | 27.75 | 0.05 | 29.20 | 0.04 |
| P02468 | Laminin subunit gamma-1 | Lamc1 | √ | 28.48 | 0.07 | 29.87 | 0.07 |
| Q9DOE1 | Heterogeneous nuclear | Hnrnpm | v | 26.17 | 0.19 | 27.51 | 0.07 |
| | ribonucleoprotein M | | | | | | |
| P51410 | 60S ribosomal protein L9 | Rpl9 | √ | 30.67 | 0.07 | 31.98 | 0.05 |
| P62889 | 60S ribosomal protein L30 | Rpl30 | √ | 30.97 | 0.03 | 32.28 | 0.06 |
| P62082 | 40S ribosomal protein S7 | Rps7 | √ | 31.86 | 0.03 | 33.12 | 0.02 |
| P62908 | 40S ribosomal protein S3 | Rps3 | √ | 32.47 | 0.05 | 33.70 | 0.03 |
| P62806 | Histone H4 | Hist1h4a | √ | 33.82 | 0.02 | 35.02 | 0.06 |
| Q91YQ5 | Dolichyl- | Rpn1 | √ | 32.38 | 0.03 | 33.48 | 0.01 |
| | diphosphooligosaccharide— | | | | | | |
| | protein | | | | | | |
| | glycosyltransferase subunit | | | | | | |
| | 1 | | | | | | |
| P60867 | 40S ribosomal protein S20 | Rps20 | √ | 31.21 | 0.02 | 32.26 | 0.02 |
| Q61656 | Probable ATP-dependent | Ddx5 | √ | 26.93 | 0.17 | 27.93 | 0.04 |
| | RNA helicase DDX5 | | | | | | |
| Q9ERE2 | Keratin, type II cuticular | Krt81 | | 21.31 | 0.15 | 31.52 | 0.07 |
| | Hb1 | | | | | | |
| CON__ | Keratin, type I cuticular | Krt34 | | 21.97 | 0.35 | 30.92 | 0.05 |
| Q9D646; | Ha4 | | | | | | |
| Q9D646 | | | | | | | |
| O08638 | Myosin-11 | Myh11 | | 23.34 | 0.44 | 31.52 | 0.05 |
| Q8BFZ3 | Beta-actin-like protein 2 | Actbl2 | | 21.64 | 0.20 | 28.90 | 0.04 |
| Q8K0Y2 | Keratin, type I cuticular | Krt33a | | 21.08 | 0.76 | 28.01 | 0.04 |
| | Ha3-1 | | | | | | |
| P56695 | Wolframin | Wfs1 | | 20.88 | 0.32 | 27.64 | 0.11 |
| P49817 | Caveolin-1 | Cav1 | | 22.02 | 0.67 | 28.77 | 0.10 |
| Q8K0E8 | Fibrinogen beta | Fgb | | 22.94 | 0.78 | 29.54 | 0.05 |
| | chain; Fibrinopeptide | | | | | | |
| | B;Fibrinogen beta chain | | | | | | |
| Q61897; | Keratin, type I cuticular | Krt33b | | 21.08 | 0.54 | 27.41 | 0.11 |
| CON:XP_ | Ha3-11 | | | | | | |
| 986630 | | | | | | | |
| P48962 | ADP/ATP translocase 1 | Slc25a4 | | 22.11 | 0.65 | 28.43 | 0.01 |
| Q91YH5 | Atlastin-3 | Atl3 | | 21.23 | 0.47 | 27.53 | 0.07 |
| Q6URW6 | Myosin-14 | Myh14 | | 20.91 | 0.21 | 27.07 | 0.04 |
| Q6IMF0 | Keratin, type II cuticular | Krt83 | | 21.61 | 1.10 | 27.70 | 0.07 |
| | Hb3 | | | | | | |
| P46735 | Unconventional myosin-lb | Myo1b | | 23.02 | 0.15 | 29.01 | 0.13 |
| O88697 | Serine/threonine-protein | Stk16 | | 21.01 | 0.56 | 26.83 | 0.23 |
| | kinase 16 | | | | | | |
| Q91VS7 | Microsomal glutathione S- | Mgst1 | | 21.13 | 0.64 | 26.73 | 0.09 |
| | transferase 1 | | | | | | |
| Q9D023 | Mitochondrial pyruvate | Mpc2 | | 22.02 | 0.92 | 27.53 | 0.16 |
| | carrier 2 | | | | | | |
| Q92511 | ATPase family AAA | Atad3 | | 21.76 | 1.14 | 27.13 | 0.20 |
| | domain-containing protein | | | | | | |
| | 3 | | | | | | |
| P04919 | Band 3 anion transport | Slc4a1 | | 22.07 | 0.37 | 27.43 | 0.14 |
| | protein | | | | | | |
| P97858 | Solute carrier family 35 | Slc35b1 | | 21.66 | 0.54 | 26.98 | 0.30 |
| | member B1 | | | | | | |
| Q9WTI7 | Unconventional myosin-Ic | Myo1c | | 23.79 | 0.20 | 29.07 | 0.06 |
| Q9CR64 | Protein kish-A | Tmem167a | | 21.06 | 0.44 | 26.22 | 0.13 |
| Q9D6M3 | Mitochondrial glutamate | Slc25a22 | | 21.79 | 0.46 | 26.91 | 0.01 |
| | carrier 1 | | | | | | |
| Q6NS46 | Protein RRP5 homolog | Pdcd11 | | 21.11 | 0.57 | 26.22 | 0.01 |
| CON__ | Keratin, type I cuticular | Krt31 | | 21.54 | 0.20 | 26.58 | 0.12 |
| A2A5Y0; | Ha1 | | | | | | |
| Q61765 | | | | | | | |
| Q5SYD0 | Unconventional myosin-Id | Myo1d | | 22.08 | 0.25 | 27.11 | 0.07 |
| Q91ZW3 | SWI/SNF-related matrix- | Smarca5 | | 20.98 | 0.22 | 25.96 | 0.07 |
| | associated actin- | | | | | | |
| | dependent regulator of | | | | | | |
| | chromatin subfamily A | | | | | | |
| | member 5 | | | | | | |
| Q9Z329 | Inositol 1,4,5- | Itpr2 | | 21.31 | 0.28 | 26.22 | 0.01 |
| | trisphosphate receptor | | | | | | |
| | type 2 | | | | | | |
| Q64331 | Unconventional myosin-VI | Myo6 | | 22.03 | 0.15 | 26.92 | 0.08 |
| Q6P5B0 | RRP12-like protein | Rrp12 | | 21.32 | 0.28 | 26.17 | 0.07 |
| Q9D0M5 | Dynein light chain 2, | Dynll2 | | 21.94 | 0.35 | 26.75 | 0.03 |
| | cytoplasmic | | | | | | |
| Q60634 | Flotillin-2 | Flot2 | | 21.55 | 0.47 | 26.33 | 0.09 |
| Q91VW5 | Golgin subfamily A | Golga4 | | 21.95 | 0.20 | 26.70 | 0.01 |
| | member 4 | | | | | | |
| Q6A009 | E3 ubiquitin-protein ligase | Ltn1 | | 21.11 | 0.20 | 25.83 | 0.07 |
| | listerin | | | | | | |
| Q8BVY0 | Ribosomal L1 domain- | Rsl1d1 | | 21.42 | 0.22 | 26.14 | 0.05 |
| | containing protein 1 | | | | | | |
| Q8BKE6 | Cytochrome P450 20A1 | Cyp20a1 | | 25.13 | 0.21 | 29.83 | 0.34 |
| Q9DBS1 | Transmembrane protein | Tmem43 | | 22.45 | 0.19 | 27.14 | 0.03 |
| | 43 | | | | | | |
| Q99ME9 | Nucleolar GTP-binding | Gtpbp4 | | 22.19 | 0.26 | 26.86 | 0.03 |
| | protein 1 | | | | | | |
| Q61879 | Myosin-10 | Myh10 | | 21.62 | 0.30 | 26.24 | 0.16 |
| Q9Z0R9 | Fatty acid desaturase 2 | Fads2 | | 21.09 | 0.68 | 25.70 | 0.06 |
| Q8VCM7 | Fibrinogen gamma chain | Fgg | | 24.80 | 0.44 | 29.37 | 0.05 |
| Q8K224 | N-acetyltransferase 10 | Nat10 | | 21.07 | 0.44 | 25.63 | 0.41 |
| Q9WVC3 | Caveolin-2 | Cav2 | | 21.09 | 0.64 | 25.62 | 0.06 |
| Q922J3 | CAP-Gly domain- | Clip1 | | 21.90 | 0.62 | 26.41 | 0.09 |
| | containing linker protein 1 | | | | | | |
| P0DN34 | | | | 22.22 | 0.40 | 26.72 | 0.51 |
| Q9JJ80 | Ribosome production | Rpf2 | | 22.28 | 0.63 | 26.78 | 0.08 |
| | factor 2 homolog | | | | | | |
| P20918 | Plasminogen; Plasmin | Plg | | 21.76 | 0.31 | 26.25 | 0.05 |
| | heavy chain A; Activation | | | | | | |
| | peptide; Angiostatin; Plasmin | | | | | | |
| | heavy chain A, short | | | | | | |
| | form; Plasmin light chain B | | | | | | |
| Q6PHZ2 | Calcium/calmodulin- | Camk2d | | 21.45 | 0.74 | 25.75 | 0.09 |
| | dependent protein kinase | | | | | | |
| | type II subunit delta | | | | | | |
| Q8JZU2 | Tricarboxylate transport | Slc25a1 | | 24.84 | 0.40 | 29.12 | 0.09 |
| | protein, mitochondrial | | | | | | |
| Q8K268 | ATP-binding cassette sub- | Abcf3 | | 22.12 | 0.30 | 26.38 | 0.15 |
| | family F member 3 | | | | | | |
| Q8BL66 | Early endosome antigen 1 | Eea1 | | 22.34 | 0.20 | 26.53 | 0.01 |
| Q3UN02 | Lysocardiolipin | Lclat1 | | 21.97 | 0.58 | 26.12 | 0.03 |
| | acyltransferase 1 | | | | | | |
| Q64511 | DNA topoisomerase 2-beta | Top2b | | 21.83 | 0.37 | 25.95 | 0.04 |
| Q9QZD8 | Mitochondrial | Slc25a10 | | 24.19 | 0.76 | 28.30 | 0.16 |
| | dicarboxylate carrier | | | | | | |
| Q3UUQ7 | GPI inositol-deacylase | Pgap1 | | 22.01 | 0.62 | 26.09 | 0.26 |
| Q9EQP2 | EH domain-containing | Ehd4 | | 22.52 | 0.30 | 26.56 | 0.20 |
| | protein 4 | | | | | | |
| Q91W34 | RUS1 family protein C16orf58 homolog | | | 21.99 | 0.14 | 26.01 | 0.04 |
| Q8BPS4 | Integral membrane protein | Gpr180 | | 22.09 | 0.27 | 26.07 | 0.04 |
| | GPR180 | | | | | | |
| P19324 | Serpin H1 | Serpinh1 | | 24.46 | 0.42 | 28.43 | 0.26 |
| E9PV24 | Fibrinogen alpha | Fga | | 24.92 | 0.43 | 28.88 | 0.04 |
| | chain; Fibrinopeptide | | | | | | |
| | A; Fibrinogen alpha chain | | | | | | |
| Q5U458 | DnaJ homolog subfamily C | Dnajc11 | | 22.08 | 0.37 | 26.03 | 0.05 |
| | member 11 | | | | | | |
| P55096 | ATP-binding cassette sub- | Abcd3 | | 21.01 | 0.37 | 24.94 | 0.02 |
| | family D member 3 | | | | | | |
| Q3TEA8 | Heterochromatin protein | Hp1bp3 | | 23.12 | 0.76 | 27.03 | 0.23 |
| | 1-binding protein 3 | | | | | | |
| Q91VE0 | Long-chain fatty acid | Slc27a4 | | 21.42 | 0.14 | 25.33 | 0.18 |
| | transport protein 4 | | | | | | |
| P70227 | Inositol 1,4,5- | Itpr3 | | 21.85 | 0.11 | 25.73 | 0.04 |
| | trisphosphate receptor | | | | | | |
| | type 3 | | | | | | |
| P42867 | UDP-N-acetylglucosamine— | Dpagt1 | | 22.62 | 0.69 | 26.44 | 0.37 |
| | dolichyl-phosphate N- | | | | | | |
| | acetylglucosaminephospho- | | | | | | |
| | transferase | | | | | | |
| P03888 | NADH-ubiquinone | Mtnd1 | | 22.15 | 0.35 | 25.93 | 0.06 |
| | oxidoreductase chain 1 | | | | | | |
| Q91VE6 | MKI67 FHA domain- | Nifk | | 21.74 | 0.49 | 25.52 | 0.10 |
| | interacting nucleolar | | | | | | |
| | phosphoprotein | | | | | | |
| Q922K7 | Probable 28S rRNA | Nop2 | | 21.59 | 0.58 | 25.34 | 0.15 |
| | (cytosine-C(5))- | | | | | | |
| | methyltransferase | | | | | | |
| Q9DBU0 | Transmembrane 9 | Tm9sf1 | | 21.68 | 0.72 | 25.42 | 0.11 |
| | superfamily member 1 | | | | | | |
| Q80WV3 | Carbohydrate | Chst2 | | 21.76 | 0.59 | 25.47 | 0.19 |
| | sulfotransferase 2 | | | | | | |
| O35682 | Myeloid-associated | Myadm | | 21.74 | 0.29 | 25.42 | 0.06 |
| | differentiation marker | | | | | | |
| O54724 | Polymerase I and | Ptrf | | 23.92 | 0.20 | 27.59 | 0.09 |
| | transcript release factor | | | | | | |
| Q9WVD5 | Mitochondrial ornithine | Slc25a15 | | 21.86 | 0.49 | 25.53 | 0.02 |
| | transporter 1 | | | | | | |
| Q925H3 | Keratin-associated protein | Krtap6-5 | | 22.33 | 0.31 | 25.99 | 0.04 |
| | 6-5 | | | | | | |
| Q8BS95 | Golgi pH regulator | Gpr89a | | 21.18 | 0.43 | 24.82 | 0.07 |
| B2RY56 | RNA-binding protein 25 | Rbm25 | | 21.21 | 0.52 | 24.86 | 0.07 |
| Q8BXQ2 | GPI transamidase | Pigt | | 21.02 | 0.33 | 24.67 | 0.10 |
| | component PIG-T | | | | | | |
| Q9QZU5 | Keratin-associated protein | Krtap15-1 | | 21.55 | 0.63 | 25.18 | 0.08 |
| | 15-1 | | | | | | |
| Q9EQ06 | Estradiol 17-beta- | Hsd17b11 | | 21.32 | 0.60 | 24.95 | 0.13 |
| | dehydrogenase 11 | | | | | | |
| Q5SWT3 | Solute carrier family 25 | Slc25a35 | | 26.48 | 0.05 | 30.08 | 0.03 |
| | member 35 | | | | | | |
| Q925N2 | Sideroflexin-2 | Sfxn2 | | 23.56 | 0.15 | 27.14 | 0.14 |
| Q8K2A8 | Dol-P- | Alg3 | | 22.01 | 0.52 | 25.59 | 0.08 |
| | Man: Man(5)GlcNAc(2)-PP- | | | | | | |
| | Dol alpha-1,3- | | | | | | |
| | mannosyltransferase | | | | | | |
| Q8CII2 | Cell division cycle protein | Cdc123 | | 20.07 | 0.20 | 23.64 | 0.08 |
| | 123 homolog | | | | | | |
| Q3UIU2 | NADH dehydrogenase | Ndufb6 | | 24.20 | 0.28 | 27.76 | 0.03 |
| | [ubiquinone] 1 beta | | | | | | |
| | subcomplex subunit 6 | | | | | | |
| Q91YR7 | Pre-mRNA-processing | Prpf6 | | 21.61 | 0.51 | 25.17 | 0.03 |
| | factor 6 | | | | | | |
| Q6TEK5 | Vitamin K epoxide | Vkorc1l1 | | 23.81 | 0.06 | 27.36 | 0.01 |
| | reductase complex subunit | | | | | | |
| | 1-like protein 1 | | | | | | |
| Q9D8Y1 | Transmembrane protein | Tmem126a | | 21.27 | 0.14 | 24.82 | 0.04 |
| | 126A | | | | | | |
| Q8BGS7 | Choline/ethanolaminephos- | Cept1 | | 21.85 | 0.22 | 25.39 | 0.11 |
| | photransferase 1 | | | | | | |
| Q8R570 | Synaptosomal-associated | Snap47 | | 21.23 | 0.17 | 24.74 | 0.15 |
| | protein 47 | | | | | | |
| Q60766 | Immunity-related GTPase | Irgm1 | | 21.46 | 0.32 | 24.96 | 0.09 |
| | family M protein 1 | | | | | | |
| Q8BKS9 | Pumilio domain-containing | Kiaa0020 | | 21.62 | 0.51 | 25.11 | 0.31 |
| | protein KIAA0020 | | | | | | |
| Q9JKN1 | Zinc transporter 7 | Slc30a7 | | 22.62 | 0.74 | 26.10 | 0.13 |
| O70572 | Sphingomyelin | Smpd2 | | 23.28 | 0.21 | 26.76 | 0.07 |
| | phosphodiesterase 2 | | | | | | |
| Q8CFJ7 | Solute carrier family 25 | Slc25a45 | | 23.29 | 0.30 | 26.77 | 0.19 |
| | member 45 | | | | | | |
| Q8BK08 | Transmembrane protein | Tmem11 | | 22.68 | 0.60 | 26.15 | 0.01 |
| | 11, mitochondrial | | | | | | |
| P35821 | Tyrosine-protein | Ptpn1 | | 21.10 | 0.16 | 24.58 | 0.01 |
| | phosphatase non-receptor | | | | | | |
| | type 1 | | | | | | |
| Q8BXA5 | Cleft lip and palate | Clptm1l | | 24.61 | 0.16 | 28.07 | 0.11 |
| | transmembrane protein 1- | | | | | | |
| | like protein | | | | | | |
| Q9Z2Z6 | Mitochondrial | Slc25a20 | | 21.70 | 0.48 | 25.15 | 0.08 |
| | carnitine/acylcarnitine | | | | | | |
| | carrier protein | | | | | | |
| Q9CQW1 | Synaptobrevin homolog | Ykt6 | | 21.85 | 0.61 | 25.29 | 0.04 |
| | YKT6 | | | | | | |
| Q9DCA5 | Ribosome biogenesis | Brix1 | | 22.17 | 0.32 | 25.61 | 0.15 |
| | protein BRX1 homolog | | | | | | |
| Q3UGP8 | Putative Dol-P- | Alg10b | | 21.36 | 0.61 | 24.79 | 0.03 |
| | Glc:Glc(2)Man(9)GlcNAc(2)- | | | | | | |
| | PP-Dol alpha-1,2- | | | | | | |
| | glucosyltransferase | | | | | | |
| Q8BWW4 | La-related protein 4 | Larp4 | | 21.60 | 0.29 | 25.03 | 0.08 |
| Q8BZ36 | RAD50-interacting protein | Rint1 | | 22.50 | 0.35 | 25.92 | 0.12 |
| | 1 | | | | | | |
| Q8BXL7 | ADP-ribosylation factor- | Arfrp1 | | 20.83 | 0.50 | 24.25 | 0.08 |
| | related protein 1 | | | | | | |
| Q4VA53 | Sister chromatid cohesion | Pds5b | | 21.56 | 0.53 | 24.96 | 0.02 |
| | protein PDS5 homolog B | | | | | | |
| Q8BYL4 | Tyrosine—tRNA ligase, | Yars2 | | 21.60 | 0.20 | 25.00 | 0.04 |
| | mitochondrial | | | | | | |
| Q8BFZ9 | Erlin-2 | Erlin2 | | 22.66 | 0.56 | 26.06 | 0.02 |
| Q9JHW4 | Selenocysteine-specific | Eefsec | | 22.25 | 0.10 | 25.63 | 0.08 |
| | elongation factor | | | | | | |
| E9Q4Z2 | Acetyl-CoA carboxylase | Acacb | | 22.11 | 0.71 | 25.49 | 0.14 |
| | 2; Biotin carboxylase | | | | | | |
| Q9D1E8 | 1-acyl-sn-glycerol-3- | Agpat5 | | 20.84 | 0.68 | 24.18 | 0.17 |
| | phosphate acyltransferase | | | | | | |
| | epsilon | | | | | | |
| Q9CZJ2 | Heat shock 70 kDa protein | Hspa12b | | 22.49 | 0.35 | 25.80 | 0.05 |
| | 12B | | | | | | |
| Q8BHS6 | Armadillo repeat- | Armcx3 | | 22.35 | 0.12 | 25.66 | 0.03 |
| | containing X-linked protein | | | | | | |
| | 3 | | | | | | |
| Q9ER41 | Torsin-1B | Tor1b | | 21.67 | 0.53 | 24.98 | 0.03 |
| Q91V01 | Lysophospholipid | Lpcat3 | | 22.24 | 0.38 | 25.53 | 0.33 |
| | acyltransferase 5 | | | | | | |
| Q80TL7 | Protein MON2 homolog | Mon2 | | 21.45 | 0.43 | 24.71 | 0.09 |
| Q8R1L4 | ER lumen protein-retaining | Kdelr3 | | 20.89 | 0.20 | 24.12 | 0.20 |
| | receptor 3 | | | | | | |
| Q9CPQ8 | ATP synthase subunit g, | Atp5l | | 26.15 | 0.38 | 29.37 | 0.05 |
| | mitochondrial | | | | | | |
| O70585 | Dystrobrevin beta | Dtnb | | 21.43 | 0.34 | 24.61 | 0.02 |
| P54116 | Erythrocyte band 7 | Stom | | 22.00 | 0.64 | 25.18 | 0.10 |
| | integral membrane protein | | | | | | |
| Q6P8H8 | Probable dolichyl | Alg8 | | 22.40 | 0.41 | 25.56 | 0.07 |
| | pyrophosphate | | | | | | |
| | Glc1Man9GlcNAc2 alpha- | | | | | | |
| | 1,3-glucosyltransferase | | | | | | |
| Q9QXB9 | Developmentally- | Drg2 | | 21.63 | 0.16 | 24.79 | 0.09 |
| | regulated GTP-binding | | | | | | |
| | protein 2 | | | | | | |
| Q04750 | DNA topoisomerase 1 | Top1 | | 22.98 | 0.36 | 26.08 | 0.08 |
| Q922P9 | Putative oxidoreductase | Glyr1 | | 22.44 | 0.13 | 25.53 | 0.07 |
| | GLYR1 | | | | | | |
| CON__ | Keratin, type I cytoskeletal | Krt20 | | 21.42 | 0.40 | 24.52 | 0.10 |
| Q9D312; | 20 | | | | | | |
| Q9D312 | | | | | | | |
| Q8K363 | ATP-dependent RNA | Ddx18 | | 21.77 | 0.36 | 24.85 | 0.16 |
| | helicase DDX18 | | | | | | |
| Q80UJ7 | Rab3 GTPase-activating | Rab3gap1 | | 21.67 | 0.38 | 24.74 | 0.06 |
| | protein catalytic subunit | | | | | | |
| Q9CQZ0 | ORM1-like protein 2 | Ormdl2 | | 21.55 | 0.65 | 24.61 | 0.10 |
| Q6A026 | Sister chromatid cohesion | Pds5a | | 22.68 | 0.62 | 25.72 | 0.08 |
| | protein PDS5 homolog A | | | | | | |
| Q569Z6 | Thyroid hormone | Thrap3 | | 22.61 | 0.49 | 25.65 | 0.11 |
| | receptor-associated | | | | | | |
| | protein 3 | | | | | | |
| P30999 | Catenin delta-1 | Ctnnd1 | | 24.58 | 0.37 | 27.62 | 0.11 |
| Q78IK4 | MICOS complex subunit | Apool | | 23.55 | 0.30 | 26.60 | 0.07 |
| | Mic27 | | | | | | |
| Q5XJY4 | Presenilins-associated | Parl | | 22.03 | 0.21 | 25.07 | 0.13 |
| | rhomboid-like protein, | | | | | | |
| | mitochondrial; P-beta | | | | | | |
| Q8CI11 | Guanine nucleotide- | Gnl3 | | 21.75 | 0.44 | 24.78 | 0.29 |
| | binding protein-like 3 | | | | | | |
| P57791 | CAAX prenyl protease 2 | Rce1 | | 21.69 | 0.29 | 24.73 | 0.09 |
| Q61595 | Kinectin | Ktn1 | | 23.73 | 0.18 | 26.75 | 0.09 |
| P55937 | Golgin subfamily A | Golga3 | | 22.68 | 0.15 | 25.70 | 0.60 |
| | member 3 | | | | | | |
| Q91XB7 | Protein YIF1A | Yif1a | | 22.23 | 0.55 | 25.24 | 0.07 |
| Q9ERG0 | LIM domain and actin- | Lima1 | | 21.70 | 0.62 | 24.71 | 0.10 |
| | binding protein 1 | | | | | | |
| Q8BHD7 | Polypyrimidine tract- | Ptbp3 | | 22.40 | 0.24 | 25.41 | 0.04 |
| | binding protein 3 | | | | | | |
| A2A5R2 | Brefeldin A-inhibited | Arfgef2 | | 21.24 | 0.15 | 24.24 | 0.51 |
| | guanine nucleotide- | | | | | | |
| | exchange protein 2 | | | | | | |
| E9PZJ8 | Activating signal | Ascc3 | | 22.55 | 0.29 | 25.52 | 0.22 |
| | cointegrator 1 complex | | | | | | |
| | subunit 3 | | | | | | |
| O54825 | Bystin | Bysl | | 21.26 | 0.37 | 24.23 | 0.06 |
| Q5SSZ5 | Tensin-3 | Tns3 | | 22.22 | 0.03 | 25.18 | 0.14 |
| P97742 | Carnitine O- | Cpt1a | | 24.14 | 0.25 | 27.09 | 0.07 |
| | palmitoyltransferase 1, | | | | | | |
| | liver isoform | | | | | | |
| Q6PHN9 | Ras-related protein Rab-35 | Rab35 | | 21.78 | 0.28 | 24.73 | 0.19 |
| P03911 | NADH-ubiquinone | Mtnd4 | | 22.99 | 0.15 | 25.86 | 0.18 |
| | oxidoreductase chain 4 | | | | | | |
| O35678 | Monoglyceride lipase | Mgll | | 21.79 | 0.42 | 24.61 | 0.15 |
| Q569Z5 | Probable ATP-dependent | Ddx46 | | 22.38 | 0.33 | 25.20 | 0.04 |
| | RNA helicase DDX46 | | | | | | |
| Q8R3C6 | Probable RNA-binding | Rbm19 | | 21.33 | 0.32 | 24.14 | 0.27 |
| | protein 19 | | | | | | |
| P59326 | YTH domain-containing | Ythdf1 | | 21.39 | 0.44 | 24.19 | 0.09 |
| | family protein 1 | | | | | | |
| Q3U821 | | Wdr75 | | 21.24 | 0.53 | 24.00 | 0.14 |
| Q9JIK5 | Nucleolar RNA helicase 2 | Ddx21 | | 24.33 | 0.29 | 27.09 | 0.25 |
| Q8C4J7 | Transducin beta-like | Tbl3 | | 21.35 | 0.47 | 24.06 | 0.28 |
| | protein 3 | | | | | | |
| Q7TPV4 | Myb-binding protein 1A | Mybbp1a | | 26.63 | 0.04 | 29.33 | 0.02 |
| Q6AW69 | Cingulin-like protein 1 | Cgnl1 | | 22.08 | 0.23 | 24.78 | 0.11 |
| Q9JHS4 | ATP-dependent Clp | Clpx | | 21.51 | 0.34 | 24.21 | 0.05 |
| | protease ATP-binding | | | | | | |
| | subunit clpX-like, | | | | | | |
| | mitochondrial | | | | | | |
| Q8BIG7 | Catechol O- | Comtd1 | | 21.79 | 0.29 | 24.49 | 0.49 |
| | methyltransferase domain- | | | | | | |
| | containing protein 1 | | | | | | |
| Q8C2Q3 | RNA-binding protein 14 | Rbm14 | | 22.09 | 0.28 | 24.77 | 0.13 |
| Q9CX30 | Protein YIF1B | Yif1b | | 20.91 | 0.43 | 23.58 | 0.30 |
| E9Q3L2 | | Pi4ka | | 21.25 | 0.03 | 23.91 | 0.24 |
| Q8CHK3 | Lysophospholipid | Mboat7 | | 21.60 | 0.27 | 24.27 | 0.11 |
| | acyltransferase 7 | | | | | | |
| Q99P58 | Ras-related protein Rab- | Rab27b | | 24.23 | 0.50 | 26.89 | 0.01 |
| | 27B | | | | | | |
| Q9Z1F9 | SUMO-activating enzyme | Uba2 | | 20.97 | 0.33 | 23.63 | 0.27 |
| | subunit 2 | | | | | | |
| Q9EPK7 | Exportin-7 | Xpo7 | | 21.27 | 0.40 | 23.90 | 0.23 |
| Q8BTX9 | Inactive hydroxysteroid | Hsdl1 | | 21.51 | 0.36 | 24.12 | 0.17 |
| | dehydrogenase-like | | | | | | |
| | protein 1 | | | | | | |
| Q9DBY1 | E3 ubiquitin-protein ligase | Syvn1 | | 24.43 | 0.53 | 27.04 | 0.12 |
| | synoviolin | | | | | | |
| Q8JZR0 | Long-chain-fatty-acid—CoA | Acsl5 | | 21.23 | 0.29 | 23.83 | 0.02 |
| | ligase 5 | | | | | | |
| Q9WV70 | Nucleolar complex protein | Noc2l | | 21.56 | 0.03 | 24.16 | 0.04 |
| | 2 homolog | | | | | | |
| Q9DBE8 | Alpha-1,3/1,6- | Alg2 | | 26.38 | 0.19 | 28.97 | 0.06 |
| | mannosyltransferase ALG2 | | | | | | |
| Q9D8M4 | 60S ribosomal protein L7- | Rpl7l1 | | 20.94 | 0.44 | 23.48 | 0.21 |
| | like 1 | | | | | | |
| Q8VDB2 | Dol-P- | Alg12 | | 21.49 | 0.16 | 24.01 | 0.05 |
| | Man:Man(7)GlcNAc(2)-PP- | | | | | | |
| | Dol alpha-1,6- | | | | | | |
| | mannosyltransferase | | | | | | |
| Q3TZM9 | GDP- | Alg11 | | 24.50 | 0.33 | 27.01 | 0.04 |
| | Man: Man(3)GlcNAc(2)-PP- | | | | | | |
| | Dol alpha-1,2- | | | | | | |
| | mannosyltransferase | | | | | | |
| Q9JIZ0; | Probable N- | Cml1; | | 24.56 | 0.12 | 27.07 | 0.09 |
| E0CYC6 | acetyltransferase CML1; N- | Nat8b | | | | | |
| | acetyltransferase 8B | | | | | | |
| Q8BP67 | 60S ribosomal protein L24 | Rpl24 | | 30.01 | 0.06 | 32.51 | 0.02 |
| Q99JY4 | TraB domain-containing | Trabd | | 24.61 | 0.26 | 27.04 | 0.12 |
| | protein | | | | | | |
| P58281 | Dynamin-like 120 kDa | Opa1 | | 24.34 | 0.17 | 26.76 | 0.04 |
| | protein, | | | | | | |
| | mitochondrial; Dynamin- | | | | | | |
| | like 120 kDa protein, form | | | | | | |
| | S1 | | | | | | |
| O09110 | Dual specificity mitogen- | Map2k3 | | 21.52 | 0.16 | 23.94 | 0.14 |
| | activated protein kinase 3 | | | | | | |
| Q9CR67 | Transmembrane protein | Tmem33 | | 25.71 | 0.21 | 28.13 | 0.07 |
| | 33 | | | | | | |
| Q9QYA2 | Mitochondrial import | Tomm40 | | 24.17 | 0.11 | 26.57 | 0.11 |
| | receptor subunit TOM40 | | | | | | |
| | homolog | | | | | | |
| Q99KI3 | ER membrane protein | Emc3 | | 22.58 | 0.26 | 24.98 | 0.22 |
| | complex subunit 3 | | | | | | |
| Q8CHJ2 | Aquaporin-12 | Aqp12 | | 26.86 | 0.01 | 29.25 | 0.01 |
| Q9D081 | UDP-N-acetylglucosamine | Alg14 | | 21.23 | 0.25 | 23.62 | 0.27 |
| | transferase subunit ALG14 | | | | | | |
| | homolog | | | | | | |
| P35282 | Ras-related protein Rab-21 | Rab21 | | 23.01 | 0.05 | 25.40 | 0.17 |
| P42227 | Signal transducer and | Stat3 | | 21.60 | 0.11 | 23.98 | 0.02 |
| | activator of transcription 3 | | | | | | |
| P70280 | Vesicle-associated | Vamp7 | | 21.90 | 0.35 | 24.26 | 0.07 |
| | membrane protein 7 | | | | | | |
| Q9CQU3 | Protein RER1 | Rer1 | | 25.50 | 0.12 | 27.85 | 0.11 |
| Q8VHE0 | Translocation protein | Sec63 | | 27.03 | 0.15 | 29.38 | 0.04 |
| | SEC63 homolog | | | | | | |
| Q925H6 | Keratin-associated protein | Krtap19-3 | | 21.47 | 0.10 | 23.81 | 0.09 |
| | 19-3 | | | | | | |
| Q91VK1; | Basic leucine zipper and | Bzw2 | | 20.75 | 0.09 | 23.08 | 0.09 |
| Q2L4X1 | W2 domain-containing | | | | | | |
| | protein 2 | | | | | | |
| P61620; | Protein transport protein | Sec61a1; | | 31.46 | 0.01 | 33.76 | 0.01 |
| Q9JLR1 | Sec61 subunit alpha | Sec61a2 | | | | | |
| | isoform 1; Protein | | | | | | |
| | transport protein Sec61 | | | | | | |
| | subunit alpha isoform 2 | | | | | | |
| Q8CFI7 | DNA-directed RNA | Polr2b | | 21.98 | 0.22 | 24.25 | 0.20 |
| | polymerase II subunit | | | | | | |
| | RPB2 | | | | | | |
| Q6PD26 | GPI transamidase | Pigs | | 21.05 | 0.14 | 23.32 | 0.23 |
| | component PIG-S | | | | | | |
| Q8BL03 | Mitochondrial basic amino | Slc25a29 | | 22.19 | 0.37 | 24.44 | 0.05 |
| | acids transporter | | | | | | |
| Q9D710 | Thioredoxin-related | Tmx2 | | 22.67 | 0.11 | 24.92 | 0.16 |
| | transmembrane protein 2 | | | | | | |
| O54962 | Barrier-to-autointegration | Banf1 | | 26.68 | 0.12 | 28.91 | 0.04 |
| | factor; Barrier-to- | | | | | | |
| | autointegration factor, N- | | | | | | |
| | terminally processed | | | | | | |
| Q91V04 | Translocating chain- | Tram1 | | 28.28 | 0.12 | 30.51 | 0.03 |
| | associated membrane | | | | | | |
| | protein 1 | | | | | | |
| Q99LG0 | Ubiquitin carboxyl- | Usp16 | | 21.63 | 0.32 | 23.86 | 0.08 |
| | terminal hydrolase 16 | | | | | | |
| Q921X9 | Protein disulfide- | Pdia5 | | 25.01 | 0.21 | 27.23 | 0.07 |
| | isomerase A5 | | | | | | |
| Q8BJM5 | Zinc transporter 6 | Slc30a6 | | 22.37 | 0.17 | 24.60 | 0.11 |
| Q62468 | Villin-1 | Vil1 | | 21.48 | 0.11 | 23.70 | 0.13 |
| P61514 | 60S ribosomal protein | Rpl37a | | 29.54 | 0.04 | 31.75 | 0.05 |
| | L37a | | | | | | |
| E9Q819 | Protein furry homolog | Fry | | 21.39 | 0.20 | 23.60 | 0.36 |
| Q8VCR2 | 17-beta-hydroxysteroid | Hsd17b13 | | 29.31 | 0.12 | 31.51 | 0.03 |
| | dehydrogenase 13 | | | | | | |
| O09167 | 60S ribosomal protein L21 | Rpl21 | | 30.13 | 0.06 | 32.33 | 0.04 |
| P25976 | Nucleolar transcription | Ubtf | | 21.79 | 0.18 | 23.99 | 0.15 |
| | factor 1 | | | | | | |
| P08752 | Guanine nucleotide- | Gnai2 | | 25.31 | 0.16 | 27.51 | 0.15 |
| | binding protein G(i) | | | | | | |
| | subunit alpha-2 | | | | | | |
| Q9CXK8 | 60S ribosome subunit | Nip7 | | 21.61 | 0.32 | 23.80 | 0.13 |
| | biogenesis protein NIP7 | | | | | | |
| | homolog | | | | | | |
| O35130 | Ribosomal RNA small | Emg1 | | 22.14 | 0.32 | 24.33 | 0.22 |
| | subunit methyltransferase | | | | | | |
| | NEP1 | | | | | | |
| P14115 | 60S ribosomal protein | Rpl27a | | 30.54 | 0.04 | 32.73 | 0.04 |
| | L27a | | | | | | |
| Q9CR57 | 60S ribosomal protein L14 | Rpl14 | | 31.06 | 0.04 | 33.25 | 0.02 |
| Q8C7H1 | Methylmalonic aciduria | Mmaa | | 21.49 | 0.36 | 23.67 | 0.15 |
| | type A homolog, | | | | | | |
| | mitochondrial | | | | | | |
| P62855 | 40S ribosomal protein S26 | Rps26 | | 29.62 | 0.16 | 31.80 | 0.08 |
| Q80U58; | Pumilio homolog 2; Pumilio | Pum2; | | 21.34 | 0.35 | 23.50 | 0.13 |
| Q80U78 | homolog 1 | Pum1 | | | | | |
| Q9EQC5 | N-terminal kinase-like | Scyl1 | | 23.51 | 0.14 | 25.66 | 0.20 |
| | protein | | | | | | |
| P62754 | 40S ribosomal protein S6 | Rps6 | | 30.48 | 0.02 | 32.63 | 0.03 |
| Q8BM55 | Transmembrane protein | Tmem214 | | 29.42 | 0.02 | 31.57 | 0.09 |
| | 214 | | | | | | |
| Q9R0Q9 | Mannose-P-dolichol | Mpdu1 | | 24.74 | 0.15 | 26.89 | 0.08 |
| | utilization defect 1 protein | | | | | | |
| Q80X95; | Ras-related GTP-binding | Rraga; | | 21.68 | 0.10 | 23.83 | 0.12 |
| Q6NTA4 | protein A; Ras-related GTP- | Rragb | | | | | |
| | binding protein B | | | | | | |
| Q60760 | Growth factor receptor- | Grb10 | | 22.65 | 0.19 | 24.79 | 0.25 |
| | bound protein 10 | | | | | | |
| Q3U1J0 | Sodium-coupled neutral | Slc38a5 | | 26.41 | 0.08 | 28.55 | 0.02 |
| | amino acid transporter 5 | | | | | | |
| Q91XE8 | Transmembrane protein | Tmem205 | | 24.54 | 0.30 | 26.68 | 0.09 |
| | 205 | | | | | | |
| Q6PGC1 | ATP-dependent RNA | Dhx29 | | 23.12 | 0.24 | 25.25 | 0.04 |
| | helicase Dhx29 | | | | | | |
| Q78XF5 | Oligosaccharyltransferase | Ostc | | 27.56 | 0.08 | 29.68 | 0.24 |
| | complex subunit OSTC | | | | | | |
| Q9D8W7 | OCIA domain-containing | Ociad2 | | 21.72 | 0.28 | 23.83 | 0.18 |
| | protein 2 | | | | | | |
| Q9D8T4 | Golgi apparatus | Tvp23b | | 21.40 | 0.31 | 23.51 | 0.02 |
| | membrane protein TVP23 | | | | | | |
| | homolog B | | | | | | |
| Q9D7S7 | 60S ribosomal protein L22- | Rpl22l1 | | 30.00 | 0.05 | 32.11 | 0.10 |
| | like 1 | | | | | | |
| P62331 | ADP-ribosylation factor 6 | Arf6 | | 25.24 | 0.13 | 27.35 | 0.03 |
| Q8BGS1 | Band 4.1-like protein 5 | Epb4115 | | 23.28 | 0.33 | 25.38 | 0.05 |
| Q09143 | High affinity cationic | Slc7a1 | | 24.66 | 0.10 | 26.76 | 0.13 |
| | amino acid transporter 1 | | | | | | |
| Q91ZN5 | Adenosine 3-phospho 5- | Slc35b2 | | 21.65 | 0.19 | 23.76 | 0.18 |
| | phosphosulfate | | | | | | |
| | transporter 1 | | | | | | |
| P70412 | CUB and zona pellucida- | Cuzd1 | | 29.15 | 0.07 | 31.25 | 0.02 |
| | like domain-containing | | | | | | |
| | protein 1 | | | | | | |
| Q8C3X8 | Lipase maturation factor 2 | Lmf2 | | 27.16 | 0.04 | 29.24 | 0.03 |
| Q6PFD9 | Nuclear pore complex | Nup98 | | 22.29 | 0.38 | 24.37 | 0.14 |
| | protein Nup98- | | | | | | |
| | Nup96; Nuclear pore | | | | | | |
| | complex protein | | | | | | |
| | Nup98; Nuclear pore | | | | | | |
| | complex protein Nup96 | | | | | | |
| O70152 | Dolichol-phosphate | Dpm1 | | 27.23 | 0.07 | 29.30 | 0.06 |
| | mannosyltransferase | | | | | | |
| | subunit 1 | | | | | | |
| Q9ERV1 | Probable E3 ubiquitin- | Mkrn2 | | 21.68 | 0.21 | 23.75 | 0.13 |
| | protein ligase makorin-2 | | | | | | |
| Q9QXX4 | Calcium-binding | Slc25a13 | | 25.60 | 0.18 | 27.67 | 0.13 |
| | mitochondrial carrier | | | | | | |
| | protein Aralar2 | | | | | | |
| P62911 | 60S ribosomal protein L32 | Rpl32 | | 30.40 | 0.04 | 32.47 | 0.08 |
| P28230 | Gap junction beta-1 | Gjb1 | | 21.26 | 0.20 | 23.33 | 0.07 |
| | protein | | | | | | |
| Q64310 | Surfeit locus protein 4 | Surf4 | | 28.82 | 0.11 | 30.88 | 0.06 |
| Q6ZWV3; | 60S ribosomal protein | Rpl10; | | 31.53 | 0.06 | 33.57 | 0.02 |
| P86048 | L10; 60S ribosomal protein | Rpl10l | | | | | |
| | L10-like | | | | | | |
| Q9CX86 | Heterogeneous nuclear | Hnrnpa0 | | 24.04 | 0.22 | 26.08 | 0.23 |
| | ribonucleoprotein A0 | | | | | | |
| Q9D1R9 | 60S ribosomal protein L34 | Rpl34 | | 31.01 | 0.03 | 33.05 | 0.06 |
| Q9CR89 | Endoplasmic reticulum- | Ergic2 | | 24.89 | 0.05 | 26.92 | 0.02 |
| | Golgi intermediate | | | | | | |
| | compartment protein 2 | | | | | | |
| P41105 | 60S ribosomal protein L28 | Rpl28 | | 31.08 | 0.10 | 33.10 | 0.06 |
| P62717 | 60S ribosomal protein | Rpl18a | | 31.31 | 0.06 | 33.33 | 0.01 |
| | L18a | | | | | | |
| Q62425 | Cytochrome c oxidase | Ndufa4 | | 28.78 | 0.03 | 30.80 | 0.03 |
| | subunit NDUFA4 | | | | | | |
| Q9D8V0 | Minor histocompatibility | Hm13 | | 27.51 | 0.08 | 29.52 | 0.02 |
| | antigen H13 | | | | | | |
| Q3UQ44 | Ras GTPase-activating-like | Iqgap2 | | 26.42 | 0.35 | 28.43 | 0.06 |
| | protein IQGAP2 | | | | | | |
| P62849 | 40S ribosomal protein S24 | Rps24 | | 29.08 | 0.08 | 31.06 | 0.03 |
| Q8K2C9 | Very-long-chain (3R)-3- | Hacd3 | | 27.06 | 0.16 | 29.04 | 0.07 |
| | hydroxyacyl-CoA | | | | | | |
| | dehydratase 3 | | | | | | |
| Q9CR62 | Mitochondrial 2- | Slc25a11 | | 26.97 | 0.03 | 28.95 | 0.04 |
| | oxoglutarate/malate | | | | | | |
| | carrier protein | | | | | | |
| E9Q7G0 | | Numa1 | | 25.31 | 0.08 | 27.29 | 0.09 |
| Q3U9G9 | Lamin-B receptor | Lbr | | 23.96 | 0.19 | 25.93 | 0.26 |
| Q99JW4 | LIM and senescent cell | Lims1 | | 22.81 | 0.20 | 24.78 | 0.03 |
| | antigen-like-containing | | | | | | |
| | domain protein 1 | | | | | | |
| Q923T9; | Calcium/calmodulin- | Camk2g; | | 22.09 | 0.29 | 24.05 | 0.11 |
| P28652 | dependent protein kinase | Camk2b | | | | | |
| | type II subunit | | | | | | |
| | gamma; Calcium/calmoduli | | | | | | |
| | n-dependent protein | | | | | | |
| | kinase type II subunit beta | | | | | | |
| Q9CQJ8 | NADH dehydrogenase | Ndufb9 | | 26.72 | 0.10 | 28.68 | 0.05 |
| | [ubiquinone] 1 beta | | | | | | |
| | subcomplex subunit 9 | | | | | | |
| Q9D6Z1 | Nucleolar protein 56 | Nop56 | | 27.28 | 0.05 | 29.24 | 0.04 |
| Q8BXZ1 | Protein disulfide- | Tmx3 | | 21.45 | 0.23 | 23.41 | 0.03 |
| | isomerase TMX3 | | | | | | |
| Q8R349 | Cell division cycle protein | Cdc16 | | 21.70 | 0.31 | 23.66 | 0.03 |
| | 16 homolog | | | | | | |
| O54692 | Centromere/kinetochore | Zw10 | | 24.16 | 0.03 | 26.12 | 0.15 |
| | protein zw10 homolog | | | | | | |
| P35293 | Ras-related protein Rab-18 | Rab18 | | 26.74 | 0.03 | 28.70 | 0.04 |
| Q60930 | Voltage-dependent anion- | Vdac2 | | 30.11 | 0.05 | 32.06 | 0.04 |
| | selective channel protein 2 | | | | | | |
| Q91VS8 | FERM, RhoGEF and | Farp2 | | 22.80 | 0.13 | 24.74 | 0.26 |
| | pleckstrin domain- | | | | | | |
| | containing protein 2 | | | | | | |
| Q9JIY5 | Serine protease HTRA2, | Htra2 | | 25.96 | 0.14 | 27.90 | 0.03 |
| | mitochondrial | | | | | | |
| Q8VDP6 | CDP-diacylglycerol-- | Cdipt | | 24.95 | 0.10 | 26.88 | 0.07 |
| | inositol 3- | | | | | | |
| | phosphatidyltransferase | | | | | | |
| Q3TDN2 | FAS-associated factor 2 | Faf2 | | 24.16 | 0.07 | 26.09 | 0.13 |
| P46978 | Dolichyl- | Stt3a | | 30.35 | 0.01 | 32.28 | 0.02 |
| | diphosphooligosaccharide— | | | | | | |
| | protein | | | | | | |
| | glycosyltransferase subunit | | | | | | |
| | STT3A | | | | | | |
| P68033; | Actin, alpha cardiac muscle | Actc1; Acta1 | | 28.88 | 0.14 | 30.79 | 0.07 |
| P68134 | 1; Actin, alpha skeletal | | | | | | |
| | muscle | | | | | | |
| P61804 | Dolichyl- | Dad1 | | 28.75 | 0.06 | 30.67 | 0.02 |
| | diphosphooligosaccharide— | | | | | | |
| | protein | | | | | | |
| | glycosyltransferase subunit | | | | | | |
| | DAD1 | | | | | | |
| Q9D8B3 | Charged multivesicular | Chmp4b | | 25.64 | 0.06 | 27.55 | 0.04 |
| | body protein 4b | | | | | | |
| P35980 | 60S ribosomal protein L18 | Rpl18 | | 31.42 | 0.05 | 33.32 | 0.05 |
| P62835 | Ras-related protein Rap-1A | Rapla | | 27.03 | 0.23 | 28.93 | 0.04 |
| Q99K01 | Pyridoxal-dependent | Pdxdc1 | | 24.67 | 0.13 | 26.54 | 0.17 |
| | decarboxylase domain- | | | | | | |
| | containing protein 1 | | | | | | |
| Q9CY27 | Very-long-chain enoyl-CoA | Tecr | | 27.72 | 0.05 | 29.59 | 0.01 |
| | reductase | | | | | | |
| P63011 | Ras-related protein Rab-3A | Rab3a | | 24.69 | 0.14 | 26.56 | 0.15 |
| Q9EPE9 | Manganese-transporting | Atp13a1 | | 28.11 | 0.00 | 29.97 | 0.04 |
| | ATPase 13A1 | | | | | | |
| Q8VCM8 | Nicalin | Ncln | | 26.77 | 0.06 | 28.62 | 0.02 |
| O08547 | Vesicle-trafficking protein | Sec22b | | 28.01 | 0.04 | 29.87 | 0.03 |
| | SEC22b | | | | | | |
| Q7TNC4 | Putative RNA-binding | Luc712 | | 24.49 | 0.17 | 26.35 | 0.32 |
| | protein Luc7-like 2 | | | | | | |
| P35279; | Ras-related protein Rab- | Rab6a; Rab6b | | 27.26 | 0.11 | 29.11 | 0.06 |
| P61294 | 6A;Ras-related protein | | | | | | |
| | Rab-6B | | | | | | |
| P56135 | ATP synthase subunit f, | Atp5j2 | | 26.66 | 0.08 | 28.51 | 0.04 |
| | mitochondrial | | | | | | |
| Q8CI04 | Conserved oligomeric | Cog3 | | 21.82 | 0.29 | 23.65 | 0.17 |
| | Golgi complex subunit 3 | | | | | | |
| P70245 | 3-beta-hydroxysteroid- | Ebp | | 25.05 | 0.10 | 26.88 | 0.04 |
| | Delta(8),Delta(7)- | | | | | | |
| | isomerase | | | | | | |
| O08912 | Polypeptide N- | Galnt1 | | 23.50 | 0.27 | 25.31 | 0.14 |
| | acetylgalactosaminyltransferase | | | | | | |
| | 1; Polypeptide N- | | | | | | |
| | acetylgalactosaminyltransferase | | | | | | |
| | 1 soluble form | | | | | | |
| Q6DFW4 | Nucleolar protein 58 | Nop58 | | 26.81 | 0.11 | 28.62 | 0.04 |
| Q91VC3 | Eukaryotic initiation factor | Eif4a3 | | 24.83 | 0.03 | 26.64 | 0.10 |
| | 4A-III; Eukaryotic initiation | | | | | | |
| | factor 4A-III, N-terminally | | | | | | |
| | processed | | | | | | |
| Q9CQY5 | Magnesium transporter | Magt1 | | 26.71 | 0.06 | 28.51 | 0.06 |
| | protein 1 | | | | | | |
| P41216 | Long-chain-fatty-acid—CoA | Acsl1 | | 24.38 | 0.30 | 26.17 | 0.19 |
| | ligase 1 | | | | | | |
| Q80X73 | Protein pelota homolog | Pelo | | 24.44 | 0.24 | 26.22 | 0.07 |
| Q9CQC7 | NADH dehydrogenase | Ndufb4 | | 26.40 | 0.04 | 28.17 | 0.05 |
| | [ubiquinone] 1 beta | | | | | | |
| | subcomplex subunit 4 | | | | | | |
| P16330 | 2,3-cyclic-nucleotide 3- | Cnp | | 24.45 | 0.05 | 26.21 | 0.21 |
| | phosphodiesterase | | | | | | |
| Q6ZWN5 | 40S ribosomal protein S9 | Rps9 | | 32.08 | 0.06 | 33.83 | 0.05 |
| Q63739 | Protein tyrosine | Ptp4a1 | | 23.59 | 0.09 | 25.34 | 0.11 |
| | phosphatase type IVA 1 | | | | | | |
| Q61102 | ATP-binding cassette sub- | Abcb7 | | 23.88 | 0.24 | 25.63 | 0.19 |
| | family B member 7, | | | | | | |
| | mitochondrial | | | | | | |
| O35129 | Prohibitin-2 | Phb2 | | 28.74 | 0.04 | 30.48 | 0.01 |
| P67778 | Prohibitin | Phb | | 28.68 | 0.13 | 30.42 | 0.07 |
| Q60931 | Voltage-dependent anion- | Vdac3 | | 28.07 | 0.09 | 29.81 | 0.03 |
| | selective channel protein 3 | | | | | | |
| P27659 | 60S ribosomal protein L3 | Rpl3 | | 32.58 | 0.01 | 34.31 | 0.03 |
| P62918 | 60S ribosomal protein L8 | Rpl8 | | 31.45 | 0.05 | 33.17 | 0.04 |
| Q9CQH3 | NADH dehydrogenase | Ndufb5 | | 25.75 | 0.11 | 27.46 | 0.03 |
| | [ubiquinone] 1 beta | | | | | | |
| | subcomplex subunit 5, | | | | | | |
| | mitochondrial | | | | | | |
| Q9EP69 | Phosphatidylinositide | Sacm1l | | 27.68 | 0.10 | 29.39 | 0.03 |
| | phosphatase SAC1 | | | | | | |
| Q7TMF3 | NADH dehydrogenase | Ndufa12 | | 26.35 | 0.01 | 28.05 | 0.01 |
| | [ubiquinone] 1 alpha | | | | | | |
| | subcomplex subunit 12 | | | | | | |
| Q60932 | Voltage-dependent anion- | Vdac1 | | 29.41 | 0.05 | 31.11 | 0.01 |
| | selective channel protein 1 | | | | | | |
| Q6DID7 | Protein wntless homolog | Wls | | 21.90 | 0.26 | 23.59 | 0.11 |
| P55012 | Solute carrier family 12 | Slc12a2 | | 21.71 | 0.08 | 23.40 | 0.06 |
| | member 2 | | | | | | |
| Q9CZM2 | 60S ribosomal protein L15 | Rpl15 | | 31.96 | 0.01 | 33.64 | 0.08 |
| Q9DC16 | Endoplasmic reticulum- | Ergic1 | | 27.51 | 0.06 | 29.18 | 0.01 |
| | Golgi intermediate | | | | | | |
| | compartment protein 1 | | | | | | |
| Q80XN0 | D-beta-hydroxybutyrate | Bdh1 | | 24.61 | 0.16 | 26.28 | 0.06 |
| | dehydrogenase, | | | | | | |
| | mitochondrial | | | | | | |
| Q9CR61 | NADH dehydrogenase | Ndufb7 | | 25.95 | 0.08 | 27.62 | 0.03 |
| | [ubiquinone] 1 beta | | | | | | |
| | subcomplex subunit 7 | | | | | | |
| P14148 | 60S ribosomal protein L7 | Rpl7 | | 31.92 | 0.09 | 33.58 | 0.05 |
| P19253 | 60S ribosomal protein | Rpl13a | | 31.62 | 0.05 | 33.27 | 0.03 |
| | L13a | | | | | | |
| Q6ZWU9 | 40S ribosomal protein S27 | Rps27 | | 29.78 | 0.01 | 31.43 | 0.04 |
| Q8VBZ3 | Cleft lip and palate | Clptm1 | | 22.50 | 0.29 | 24.15 | 0.05 |
| | transmembrane protein 1 | | | | | | |
| | homolog | | | | | | |
| P12970 | 60S ribosomal protein L7a | Rpl7a | | 32.38 | 0.01 | 34.03 | 0.02 |
| P47911 | 60S ribosomal protein L6 | Rpl6 | | 31.78 | 0.08 | 33.43 | 0.02 |
| Q9Z127 | Large neutral amino acids | Slc7a5 | | 25.04 | 0.07 | 26.68 | 0.03 |
| | transporter small subunit 1 | | | | | | |
| P47963 | 60S ribosomal protein L13 | Rpl13 | | 31.57 | 0.04 | 33.21 | 0.02 |
| Q6PB66 | Leucine-rich PPR motif- | Lrpprc | | 24.71 | 0.13 | 26.34 | 0.05 |
| | containing protein, | | | | | | |
| | mitochondrial | | | | | | |
| Q8BMG7 | Rab3 GTPase-activating | Rab3gap2 | | 21.78 | 0.07 | 23.41 | 0.14 |
| | protein non-catalytic | | | | | | |
| | subunit | | | | | | |
| Q6ZWY3 | 40S ribosomal protein S27- | Rps27l | | 28.34 | 0.09 | 29.96 | 0.06 |
| | like | | | | | | |
| Q8CC88 | von Willebrand factor A | Vwa8 | | 24.06 | 0.15 | 25.68 | 0.10 |
| | domain-containing protein | | | | | | |
| | 8 | | | | | | |
| O55143 | Sarcoplasmic/endoplasmic | Atp2a2 | | 30.33 | 0.01 | 31.95 | 0.02 |
| | reticulum calcium ATPase | | | | | | |
| | 2 | | | | | | |
| P62242 | 40S ribosomal protein S8 | Rps8 | | 31.98 | 0.04 | 33.60 | 0.04 |
| P84228 | Histone H3.2 | Hist1h3b | | 32.95 | 0.05 | 34.57 | 0.15 |
| Q9WUQ2 | Prolactin regulatory | Preb | | 28.00 | 0.01 | 29.62 | 0.01 |
| | element-binding protein | | | | | | |
| Q570Y9 | DEP domain-containing | Deptor | | 23.97 | 0.16 | 25.58 | 0.07 |
| | mTOR-interacting protein | | | | | | |
| Q9D8K3 | Derlin-3 | Derl3 | | 24.62 | 0.10 | 26.23 | 0.03 |
| Q8VEL0 | Motile sperm domain- | Mospd1 | | 23.64 | 0.10 | 25.24 | 0.08 |
| | containing protein 1 | | | | | | |
| Q8R2Y3 | Dolichol kinase | Dolk | | 21.31 | 0.20 | 22.91 | 0.24 |
| Q8VEM8 | Phosphate carrier protein, | Slc25a3 | | 30.15 | 0.03 | 31.75 | 0.04 |
| | mitochondrial | | | | | | |
| Q9DCS9 | NADH dehydrogenase | Ndufb10 | | 27.37 | 0.06 | 28.96 | 0.02 |
| | [ubiquinone] 1 beta | | | | | | |
| | subcomplex subunit 10 | | | | | | |
| P35550 | rRNA 2-O- | Fbl | | 27.96 | 0.12 | 29.54 | 0.08 |
| | methyltransferase | | | | | | |
| | fibrillarin | | | | | | |
| Q8BHY2 | Nucleolar complex protein | Noc4l | | 21.61 | 0.14 | 23.19 | 0.09 |
| | 4 homolog | | | | | | |
| P62702 | 40S ribosomal protein S4, | Rps4x | | 33.02 | 0.02 | 34.60 | 0.02 |
| | X isoform | | | | | | |
| Q9CQZ6 | NADH dehydrogenase | Ndufb3 | | 25.58 | 0.03 | 27.15 | 0.05 |
| | [ubiquinone] 1 beta | | | | | | |
| | subcomplex subunit 3 | | | | | | |
| Q3TDQ1 | Dolichyl- | Stt3b | | 27.60 | 0.12 | 29.17 | 0.04 |
| | diphosphooligosaccharide— | | | | | | |
| | protein | | | | | | |
| | glycosyltransferase subunit | | | | | | |
| | STT3B | | | | | | |
| Q566J8 | AarF domain-containing | Adck4 | | 21.41 | 0.30 | 22.94 | 0.05 |
| | protein kinase 4 | | | | | | |
| P62900 | 60S ribosomal protein L31 | Rpl31 | | 30.81 | 0.04 | 32.34 | 0.03 |
| Q99JR1 | Sideroflexin-1 | Sfxn1 | | 27.21 | 0.05 | 28.73 | 0.07 |
| P56382 | ATP synthase subunit | Atp5e | | 26.49 | 0.14 | 28.01 | 0.03 |
| | epsilon, mitochondrial | | | | | | |
| Q91VR2 | ATP synthase subunit | Atp5c1 | | 29.42 | 0.05 | 30.93 | 0.05 |
| | gamma, mitochondrial | | | | | | |
| Q8VCW8 | Acyl-CoA synthetase family | Acsf2 | | 27.62 | 0.11 | 29.13 | 0.02 |
| | member 2, mitochondrial | | | | | | |
| Q9DC69 | NADH dehydrogenase | Ndufa9 | | 28.10 | 0.02 | 29.61 | 0.01 |
| | [ubiquinone] 1 alpha | | | | | | |
| | subcomplex subunit 9, | | | | | | |
| | mitochondrial | | | | | | |
| Q791V5 | Mitochondrial carrier | Mtch2 | | 27.28 | 0.11 | 28.77 | 0.03 |
| | homolog 2 | | | | | | |
| P62821 | Ras-related protein Rab-1A | Rab1A | | 30.42 | 0.06 | 31.91 | 0.01 |
| Q9DBG7 | Signal recognition particle | Srpr | | 29.39 | 0.00 | 30.88 | 0.03 |
| | receptor subunit alpha | | | | | | |
| Q8BGH2 | Sorting and assembly | Samm50 | | 26.56 | 0.02 | 28.05 | 0.01 |
| | machinery component 50 | | | | | | |
| | homolog | | | | | | |
| Q9CQW2 | ADP-ribosylation factor- | Arl8b | | 25.00 | 0.08 | 26.49 | 0.02 |
| | like protein 8B | | | | | | |
| Q9JJI8 | 60S ribosomal protein L38 | Rpl38 | | 29.39 | 0.08 | 30.86 | 0.02 |
| P25444 | 40S ribosomal protein S2 | Rps2 | | 32.53 | 0.06 | 33.98 | 0.02 |
| P62737; | Actin, aortic smooth | Acta2; Actg2 | | 25.11 | 0.12 | 26.54 | 0.06 |
| P63268 | muscle; Actin, gamma- | | | | | | |
| | enteric smooth muscle | | | | | | |
| P46638; | Ras-related protein Rab- | Rab11b; | | 27.82 | 0.06 | 29.24 | 0.02 |
| P62492 | 11B; Ras-related protein | Rab11a | | | | | |
| | Rab-11A | | | | | | |
| Q6P4T2 | U5 small nuclear | Snrnp200 | | 24.09 | 0.13 | 25.51 | 0.14 |
| | ribonucleoprotein 200 kDa | | | | | | |
| | helicase | | | | | | |
| P61211 | ADP-ribosylation factor- | Arl1 | | 26.41 | 0.10 | 27.82 | 0.03 |
| | like protein 1 | | | | | | |
| Q8BFR5 | Elongation factor Tu, | Tufm | | 28.81 | 0.05 | 30.21 | 0.05 |
| | mitochondrial | | | | | | |
| B2RQC6 | CAD protein; Glutamine- | Cad | | 28.49 | 0.01 | 29.89 | 0.04 |
| | dependent carbamoyl- | | | | | | |
| | phosphate | | | | | | |
| | synthase;Aspartate | | | | | | |
| | carbamoyltransferase; | | | | | | |
| | Dihydroorotase | | | | | | |
| Q9DB25 | Dolichyl-phosphate beta- | Alg5 | | 26.28 | 0.25 | 27.68 | 0.14 |
| | glucosyltransferase | | | | | | |
| Q3TJD7 | PDZ and LIM domain | Pdlim7 | | 21.78 | 0.17 | 23.17 | 0.16 |
| | protein 7 | | | | | | |
| Q9D1Q4 | Dolichol-phosphate | Dpm3 | | 25.27 | 0.08 | 26.66 | 0.03 |
| | mannosyltransferase | | | | | | |
| | subunit 3 | | | | | | |
| P97351 | 40S ribosomal protein S3a | Rps3a | | 32.28 | 0.04 | 33.68 | 0.02 |
| Q04899 | Cyclin-dependent kinase | Cdk18 | | 23.23 | 0.07 | 24.62 | 0.23 |
| | 18 | | | | | | |
| Q9CQZ5 | NADH dehydrogenase | Ndufa6 | | 25.39 | 0.04 | 26.78 | 0.03 |
| | [ubiquinone] 1 alpha | | | | | | |
| | subcomplex subunit 6 | | | | | | |
| P35276 | Ras-related protein Rab-3D | Rab3d | | 28.02 | 0.08 | 29.41 | 0.04 |
| Q62186 | Translocon-associated | Ssr4 | | 31.60 | 0.04 | 32.99 | 0.02 |
| | protein subunit delta | | | | | | |
| P61255 | 60S ribosomal protein L26 | Rpl26 | | 31.76 | 0.04 | 33.14 | 0.08 |
| P84099 | 60S ribosomal protein L19 | Rpl19 | | 30.06 | 0.09 | 31.44 | 0.05 |
| Q9CXS4 | Centromere protein V | Cenpv | | 26.31 | 0.10 | 27.67 | 0.12 |
| Q9DC51 | Guanine nucleotide- | Gnai3 | | 27.49 | 0.07 | 28.86 | 0.05 |
| | binding protein G(k) | | | | | | |
| | subunit alpha | | | | | | |
| Q8R3L2 | Transcription factor 25 | Tcf25 | | 23.79 | 0.26 | 25.15 | 0.04 |
| Q99LE6 | ATP-binding cassette sub- | Abcf2 | | 26.21 | 0.12 | 27.53 | 0.05 |
| | family F member 2 | | | | | | |
| P53994 | Ras-related protein Rab-2A | Rab2a | | 28.42 | 0.08 | 29.74 | 0.04 |
| Q8CFE6 | Sodium-coupled neutral | Slc38a2 | | 23.75 | 0.09 | 25.07 | 0.03 |
| | amino acid transporter 2 | | | | | | |
| P36536 | GTP-binding protein SAR1a | Sar1a | | 27.97 | 0.03 | 29.29 | 0.04 |
| Q8BH59 | Calcium-binding | Slc25a12 | | 27.53 | 0.12 | 28.84 | 0.10 |
| | mitochondrial carrier | | | | | | |
| | protein Aralar1 | | | | | | |
| P62274 | 40S ribosomal protein S29 | Rps29 | | 28.63 | 0.09 | 29.94 | 0.04 |
| Q3TCT4 | Ectonucleoside | Entpd7 | | 21.48 | 0.10 | 22.78 | 0.11 |
| | triphosphate | | | | | | |
| | diphosphohydrolase 7 | | | | | | |
| P62818 | Protein S100-A3 | S100a3 | | 22.32 | 0.20 | 23.62 | 0.15 |
| P03921 | NADH-ubiquinone | Mtnd5 | | 24.50 | 0.06 | 25.79 | 0.08 |
| | oxidoreductase chain 5 | | | | | | |
| Q6ZWQ7 | | Spcs3 | | 29.41 | 0.04 | 30.70 | 0.01 |
| P63094; | Guanine nucleotide- | Gnas | | 25.20 | 0.07 | 26.49 | 0.03 |
| Q6R0H7 | binding protein G(s) | | | | | | |
| | subunit alpha isoforms | | | | | | |
| | short;Guanine nucleotide- | | | | | | |
| | binding protein G(s) | | | | | | |
| | subunit alpha isoforms | | | | | | |
| | XLas | | | | | | |
| Q9D7A6 | Signal recognition particle | Srp19 | | 25.49 | 0.10 | 26.77 | 0.02 |
| | 19 kDa protein | | | | | | |
| Q9D1G1 | Ras-related protein Rab-1B | Rab1b | | 25.11 | 0.22 | 26.39 | 0.08 |
| O09111 | NADH dehydrogenase | Ndufb11 | | 25.73 | 0.05 | 27.01 | 0.03 |
| | [ubiquinone] 1 beta | | | | | | |
| | subcomplex subunit 11, | | | | | | |
| | mitochondrial | | | | | | |
| P35278 | Ras-related protein Rab-5C | Rab5c | | 26.53 | 0.08 | 27.78 | 0.04 |
| Q9R0P6 | Signal peptidase complex | Sec11a | | 26.96 | 0.20 | 28.20 | 0.02 |
| | catalytic subunit SEC11A | | | | | | |
| O54774 | AP-3 complex subunit | Ap3d1 | | 25.30 | 0.07 | 26.54 | 0.01 |
| | delta-1 | | | | | | |
| Q921J2 | GTP-binding protein Rheb | Rheb | | 25.31 | 0.23 | 26.53 | 0.05 |
| Q62318 | Transcription intermediary | Trim28 | | 24.28 | 0.04 | 25.49 | 0.06 |
| | factor 1-beta | | | | | | |
| Q9R099 | Transducin beta-like | Tbl2 | | 27.84 | 0.05 | 29.05 | 0.03 |
| | protein 2 | | | | | | |
| Q8BK63 | Casein kinase I isoform | Csnk1a1 | | 24.09 | 0.09 | 25.29 | 0.09 |
| | alpha | | | | | | |
| Q60936 | Atypical kinase ADCK3, | Adck3 | | 24.38 | 0.12 | 25.57 | 0.07 |
| | mitochondrial | | | | | | |
| Q9DC70 | NADH dehydrogenase | Ndufs7 | | 26.97 | 0.03 | 28.15 | 0.06 |
| | [ubiquinone] iron-sulfur | | | | | | |
| | protein 7, mitochondrial | | | | | | |
| Q9QXW9 | Large neutral amino acids | Slc7a8 | | 26.95 | 0.06 | 28.13 | 0.07 |
| | transporter small subunit 2 | | | | | | |
| P61027 | Ras-related protein Rab-10 | Rab10 | | 26.83 | 0.17 | 27.99 | 0.06 |
| Q9QYG0 | Protein NDRG2 | Ndrg2 | | 21.90 | 0.19 | 23.06 | 0.04 |
| Q8CGK3 | Lon protease homolog, | Lonp1 | | 26.37 | 0.04 | 27.52 | 0.05 |
| | mitochondrial | | | | | | |
| P62267 | 40S ribosomal protein S23 | Rps23 | | 31.05 | 0.05 | 32.21 | 0.03 |
| Q9CPR4 | 60S ribosomal protein L17 | Rpl17 | | 31.80 | 0.06 | 32.95 | 0.05 |
| P03930 | ATP synthase protein 8 | Mtatp8 | | 26.72 | 0.02 | 27.87 | 0.06 |
| Q921L3 | Transmembrane and | Tmco1 | | 26.14 | 0.07 | 27.28 | 0.10 |
| | coiled-coil domain- | | | | | | |
| | containing protein 1 | | | | | | |
| Q8VD00 | Transmembrane protein | Tmem97 | | 30.90 | 0.05 | 32.03 | 0.06 |
| | 97 | | | | | | |
| P28571 | Sodium- and chloride- | Slc6a9 | | 24.41 | 0.16 | 25.54 | 0.03 |
| | dependent glycine | | | | | | |
| | transporter 1 | | | | | | |
| Q99LC3 | NADH dehydrogenase | Ndufa10 | | 28.34 | 0.16 | 29.46 | 0.14 |
| | [ubiquinone] 1 alpha | | | | | | |
| | subcomplex subunit 10, | | | | | | |
| | mitochondrial | | | | | | |
| P49718 | DNA replication licensing | Mcm5 | | 22.40 | 0.02 | 23.51 | 0.22 |
| | factor MCM5 | | | | | | |
| Q63932 | Dual specificity mitogen- | Map2k2 | | 23.91 | 0.12 | 25.02 | 0.02 |
| | activated protein kinase | | | | | | |
| | kinase 2 | | | | | | |
| Q8BMA6 | Signal recognition particle | Srp68 | | 29.10 | 0.02 | 30.21 | 0.01 |
| | subunit SRP68 | | | | | | |
| P83882 | 60S ribosomal protein | Rpl36a | | 30.83 | 0.05 | 31.93 | 0.08 |
| | L36a | | | | | | |
| Q9CQE7 | Endoplasmic reticulum- | Ergic3 | | 25.96 | 0.08 | 27.07 | 0.02 |
| | Golgi intermediate | | | | | | |
| | compartment protein 3 | | | | | | |
| Q6ZWV7 | 60S ribosomal protein L35 | Rpl35 | | 29.70 | 0.07 | 30.80 | 0.03 |
| Q9JKJ9 | 24-hydroxycholesterol 7- | Cyp39a1 | | 26.97 | 0.03 | 28.06 | 0.04 |
| | alpha-hydroxylase | | | | | | |
| Q9CWF2; | Tubulin beta-2B | Tubb2b; | | 21.61 | 0.12 | 22.69 | 0.07 |
| Q7TMM9 | chain; Tubulin beta-2A | Tubb2a | | | | | |
| | chain | | | | | | |
| E9PVA8 | | Gcn1l1 | | 28.94 | 0.00 | 30.01 | 0.03 |
| Q9QYC3 | Class A basic helix-loop- | Bhlha15 | | 23.03 | 0.07 | 24.08 | 0.19 |
| | helix protein 15 | | | | | | |
| Q9QXK3 | Coatomer subunit gamma-2 | Copg2 | | 25.32 | 0.12 | 26.36 | 0.09 |
| Q78IK2 | Up-regulated during | Usmg5 | | 26.43 | 0.06 | 27.47 | 0.04 |
| | skeletal muscle growth | | | | | | |
| | protein 5 | | | | | | |
| Q99MJ9 | ATP-dependent RNA | Ddx50 | | 21.61 | 0.16 | 22.64 | 0.10 |
| | helicase DDX50 | | | | | | |
| Q9Z110 | Delta-1-pyrroline-5- | Aldh18a1 | | 28.78 | 0.01 | 29.81 | 0.02 |
| | carboxylate | | | | | | |
| | synthase; Glutamate 5- | | | | | | |
| | kinase; Gamma-glutamyl | | | | | | |
| | phosphate reductase | | | | | | |
| P17892 | Pancreatic lipase-related | Pnliprp2 | | 30.09 | 0.02 | 31.11 | 0.01 |
| | protein 2 | | | | | | |
| Q9CQQ7 | ATP synthase F(0) complex | Atp5f1 | | 29.37 | 0.04 | 30.39 | 0.02 |
| | subunit B1, mitochondrial | | | | | | |
| O08786 | Cholecystokinin receptor | Cckar | | 26.52 | 0.15 | 27.53 | 0.05 |
| | type A | | | | | | |
| Q6ZQ58 | La-related protein 1 | Larp1 | | 26.89 | 0.07 | 27.89 | 0.07 |
| P00397 | Cytochrome c oxidase | Mtco1 | | 26.01 | 0.06 | 27.01 | 0.17 |
| | subunit 1 | | | | | | |

### Example 8: Bioengineering of Immune Checkpoint Ligand-Functionalized Mouse Cells (MSCs)

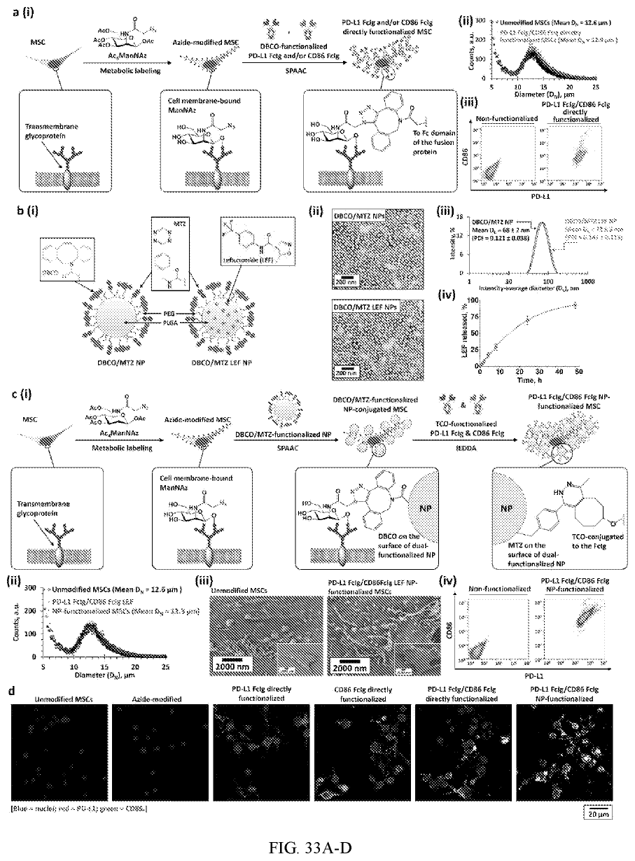

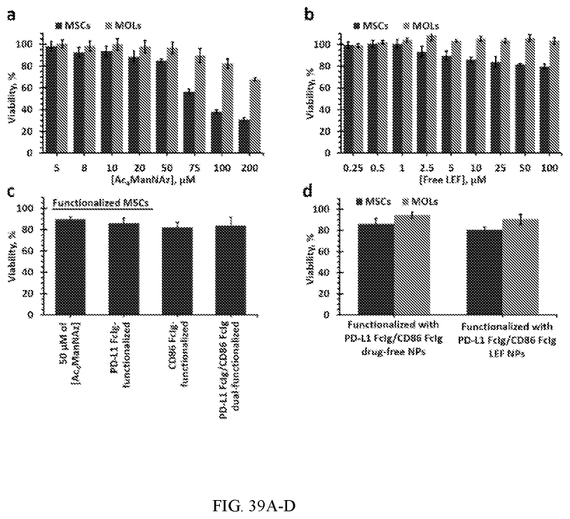

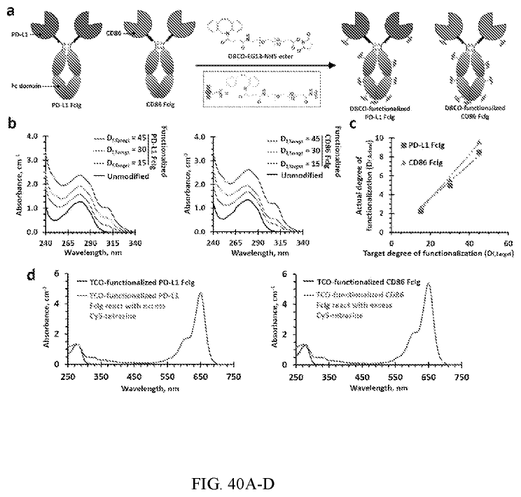

Immune checkpoint ligand-functionalized MSCs were bioengineered via metabolic glycoengineering followed by the bioorthogonal click reaction⁴⁸⁻⁵⁰. We evaluated direct bioconjugation (FIG. 33a) and NP pre-anchoring conjugation (FIG. 33b-c) strategies to functionalize the MSCs. These strategies employed azide-modified MSCs obtained by culturing MSCs with a subcytotoxic concentration of N-azidoacetylmannosamine tetraacylated (AC₄MaNAz; FIG. 39)⁴⁹. MSCs take up the ManNAz and convert it to azide-sialic acid derivatives to achieve N-linked glycosylate of cell surface proteins^{48, 50}. These azide-sialic acid derivatives on the surface of the glia provide sites for bioorthogonal strain-promoted azide-alkyne cycloaddition (SPAAC; FIG. 33a(i))^{48, 50}. In the direct functionalization method, dibenzocyclooctyne (DBCO)-functionalized PD-L1 Fc-fusion proteins (PD-L1-Ig) and CD86 Fc-fusion proteins (CD86-Ig)⁻⁵² (FIG. 40a-c) were directly conjugated to azide-modified MSCs through SPAAC⁴⁸⁻⁵⁰ at a target degree of conjugation of 5 μg of fusion protein per one million cells (FIG. 33a). The NP pre-anchoring conjugation strategy involved the preparation of drug-free and LEF-encapsulated DBCO- and methyltetrazine (MTZ)-functionalized NPs (DBCO/MTZ NPs) via the nanoprecipitation method (FIG. 33b)⁵². The encapsulated LEF DBCO/MTZ NPs (LEF NPs) were prepared using 3.3 wt/wt % of LEF⁵³, which controlled their release under physiological conditions (half-life 15.0±0.3 h) (FIG. 33b). We next conjugated DBCO/MTZ NPs to azide-modified MSCs via SPAAC at a target degree of conjugation of 500 μg NPs per one million cells (FIG. 33c). We then conjugated TCO-functionalized PD-L1-Ig and CD86-Ig to the NP-functionalized MSCs through the inverse electron-demand Diels-Alder (IEDDA) reaction⁵⁴ with the same target degree of functionalization as for the directly functionalized MSCs (FIG. 33c, and FIG. 40d). PD-L1-Ig/CD86-Ig LEF NP-functionalized mouse OLs (MOLs) were bioengineered using the same method with an identical degree of functionalization and LEF loading. Neither bioconjugation strategy significantly affected the size or viability of the MSCs and MOLs (FIG. 33a-c, and FIG. 39).

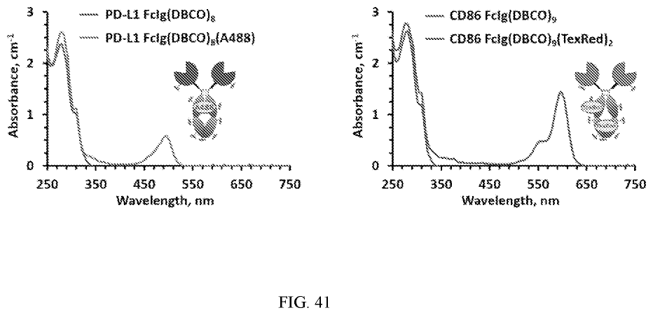

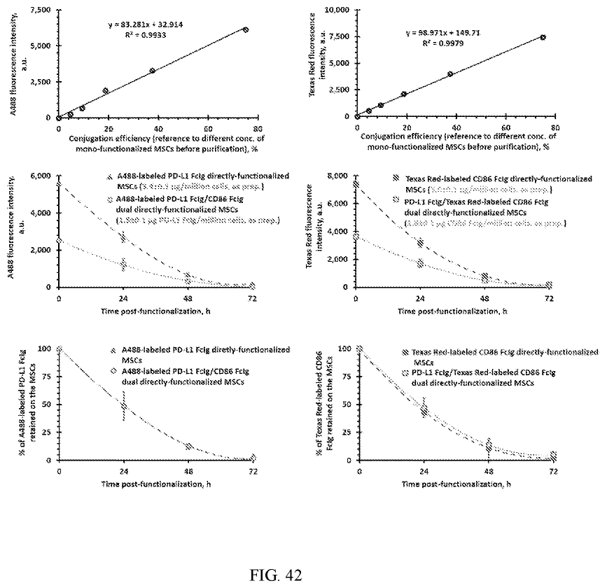

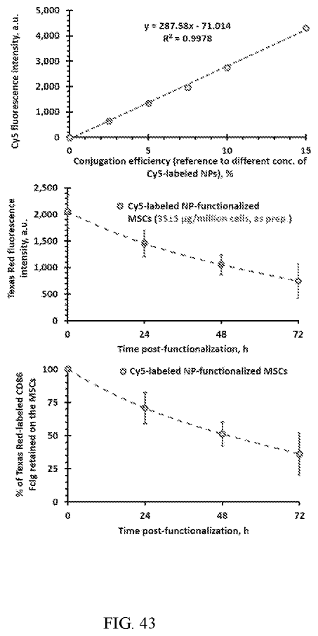

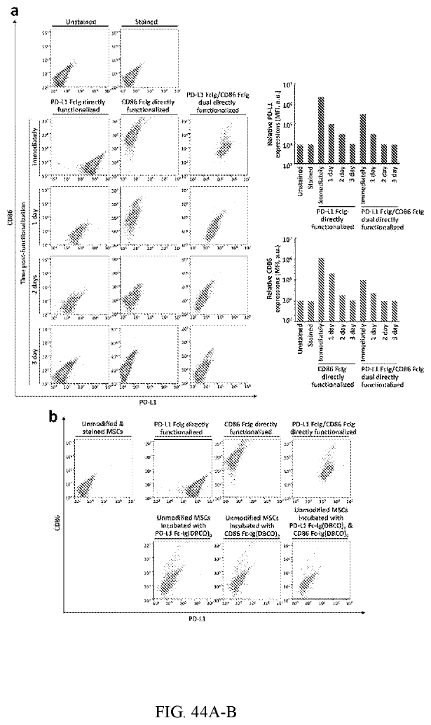

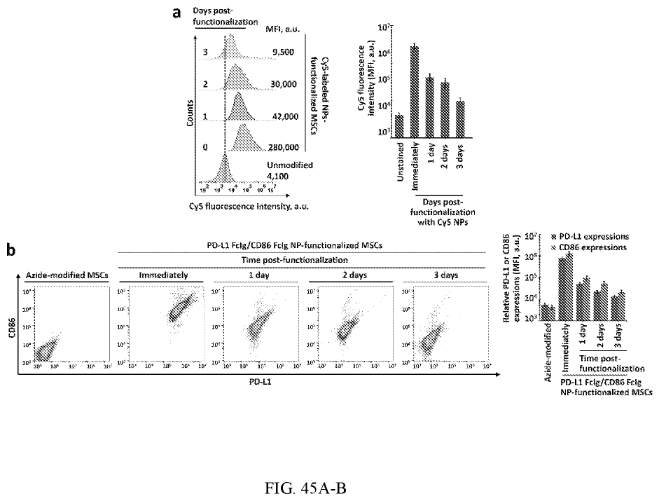

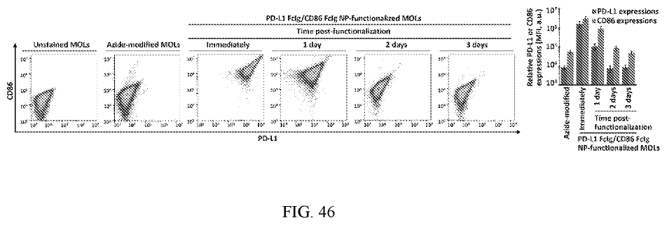

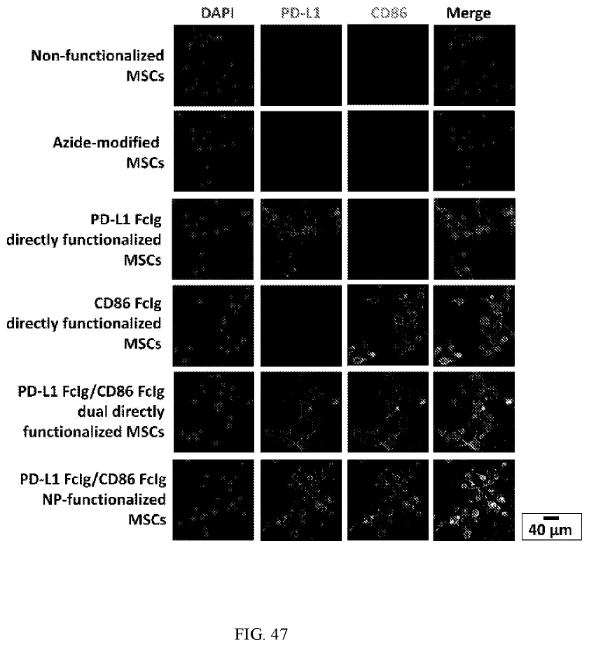

When we used A488-labeled PD-L1-Ig and Texas Red-labeled CD86-Ig (FIG. 41) for the bioconjugation, between 68% and 72% of the DBCO-functionalized fusion proteins were directly conjugated to the azide-modified MSCs (FIG. 42). When we functionalized using Cy5-labeled DBCO/MTZ NPs, 35±5 μg of the NPs were conjugated to one million of the MSCs (and thus 1.16 μg of encapsulated LEF for the LEF NP-functionalized MSCs; FIG. 43), which allowed a quantitative conjugation of TCO-functionalized fusion proteins (i.e., 5 μg of TCO-functionalized fusion protein per million cells). Fluorescence-activated cell sorting (FACS) assay further confirmed that PD-L1-Ig and CD86-Ig were conjugated to the MSCs (FIGS. 33a and c, and FIG. 44). The levels of PD-L1 and CD86 expressed by the directly functionalized MSCs declined much faster those functionalized through the NP pre-anchoring strategy because of cell proliferation and metabolic clearance (FIGS. 44 and 45)^{48, 50} A similar phenomenon was observed in the PD-L1-Ig/CD86-Ig NP-functionalized MOLs (FIG. 46). The functionalization of MSCs was further confirmed by confocal laser scanning microscopy (CLSM) staining with A488-labeled anti-PD-L1 and phycoerythrin (PE)-labeled anti-CD86 antibodies (FIG. 33d, and FIG. 47). Further, scanning electron microscopy indicated equal distribution of the conjugated PD-L1-Ig/CD86-Ig LEF NPs on the surface of the MSCs (FIG. 33c(iii)).

### Example 9: PD-L1- and CD86-Functionalized MSCs Downregulate Myelin-Specific T Cell Activation and Promote the Development of T_{reg} Cells In Vitro

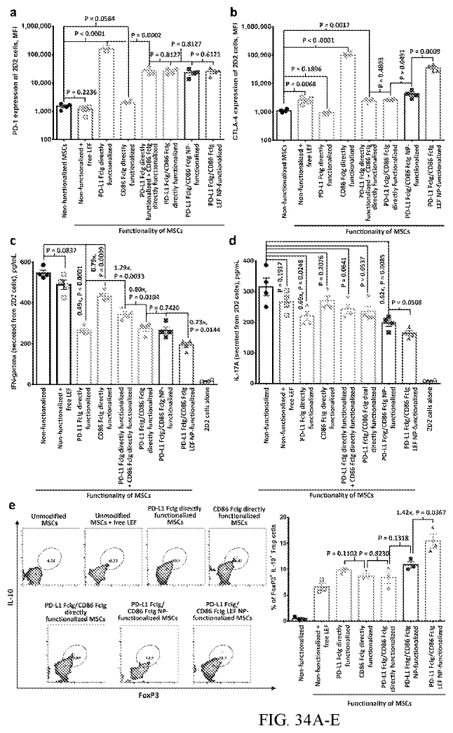

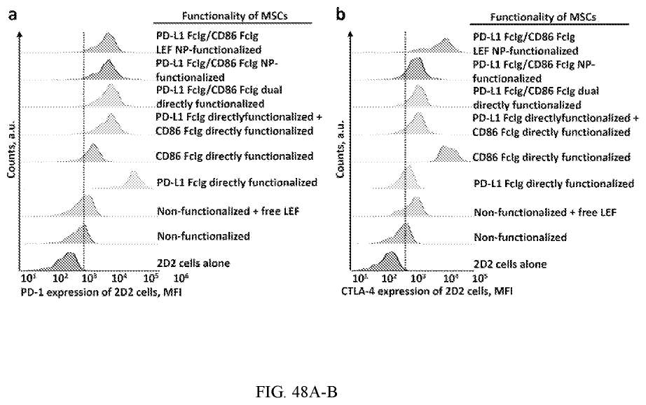

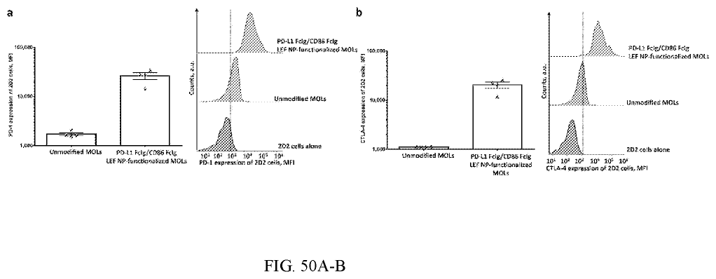

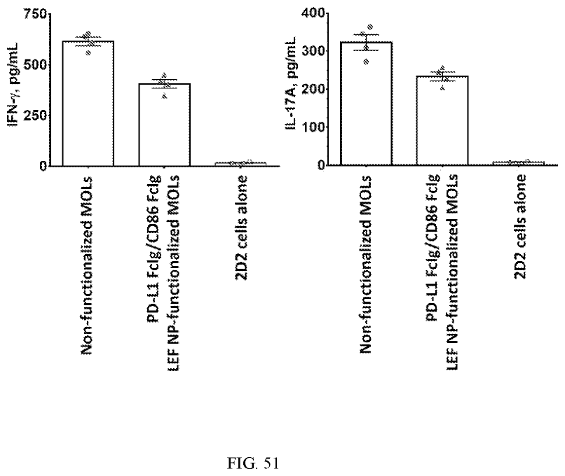

To evaluate the effects of MSC-conjugated PD-L1, CD86, and encapsulated LEF on antigen-specific CD4⁺ T cell activation, we cultured mono- and dual-functionalized MSCs with MOG-specific CD4⁺ T cells isolated from 2D2 mice (2D2 cells)^{55, 56} and quantified the PD-1 and CTLA-4 levels expressed by the 2D2 cells. Both types of directly monofunctionalized MSCs effectively upregulated the corresponding immune checkpoint pathway (FIG. 34a-b, and FIG. 48). A 11 combination of both monofunctionalized MSCs and dual-functionalized MSCs concurrently upregulated both immune checkpoint pathways in 2D2 cells (FIG. 34a-b, and FIG. 48), but the upregulations were less effective than they were with the same amount of monofunctionalized MSCs. The drug-free PD-L1-Ig/CD86-Ig NP-functionalized MSCs were as effective as the combination of two directly functionalized MSCs to upregulate the PD-1 and CTLA-4 expressions of 2D2 cells (FIG. 34a-b, and FIG. 48). Similar to the results of previous study⁵⁷, small-molecule LEF upregulated CD86 expression in the MSCs and therefore increased the expression of CTLA-4 in the co-cultured 2D2 cells (FIG. 34b, and FIG. 48). Thus, PD-L1-Ig/CD86-Ig LEF NP-functionalized MSCs were more effective than drug-free PD-L1-Ig/CD86-Ig NP-functionalized MSCs and dual directly functionalized MSCs in upregulating the CTLA-4 pathway (FIG. 34b, and FIG. 48). The remarkable upregulation of both inhibitory immune checkpoint pathways by the cocultured PD-L1 and CD86 dual-functionalized MSCs significantly reduced the level of effector molecules when evaluating the interferon gamma (IFN-γ, secreted from Tₕ1 cells)^{56, 58} and interleukin 17A (IL-17A, secreted from Tₕ17 cells)^{56, 59} secreted by the 2D2 cells through enzyme-linked immunosorbent assay (FIG. 34c-d). We observed a similar upregulation of PD-1 and CTLA-4 pathways in the 2D2 cells after culturing them with PD-L1-Ig/CD86-Ig LEF NP-functionalized MOLs (FIGS. 50 and 51).

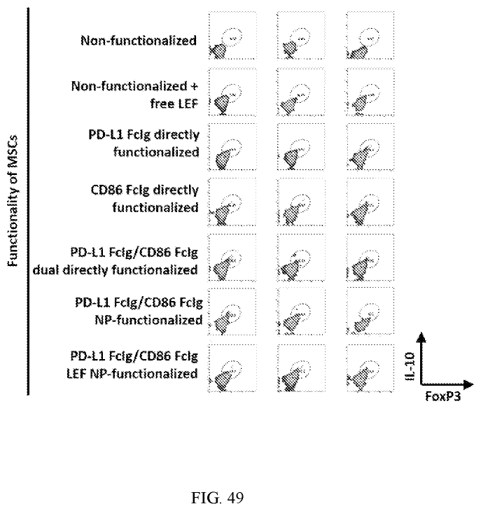

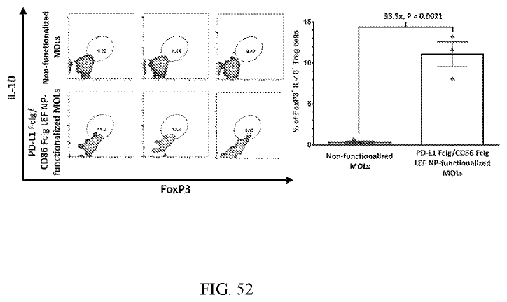

To determine whether PD-L1- and CD86-functionalized MSCs can promote the development of antigen-specific induced T_{reg} cells^{41, 56}, we quantified the population of FoxP3⁺ and IL10⁺ CD4⁺ T cells after culturing the 2D2 cells with different functionalized MSCs for 72 h. Incubation with unmodified MSCs in the presence of small-molecule LEF induced approximately 6% of 2D2 cells to develop into T_{reg} cells (FIG. 34e, and FIG. 49). All directly functionalized MSCs promoted the development of induced T_{reg} cells, as indicated by finding that 8-10% of the CD4⁺ expressed cells were FoxP3⁺ and IL10⁺ (FIG. 34e, and FIG. 49). The drug-free PD-L1-Ig/CD86-Ig NP-functionalized MSCs were as effective as the directly dual-functionalized MSCs in promoting native 2D2 cells to develop into induced T_{reg} cells. In contrast, the LEF-encapsulated NP-functionalized MSCs were 42% more effective than those of the drug-free NP-functionalized MSCs in their ability to transform native 2D2 cells into induced T_{reg} cells (FIG. 34e, and FIG. 49). Similarly, the PD-L1-Ig/CD86-Ig LEF NP-functionalized MOLs were 33.5 times more effective than unmodified MOLs with respect to their ability to promote the development of cocultured native 2D2 cells into myelin-specific T_{reg} cells (FIGS. 50 to 52).

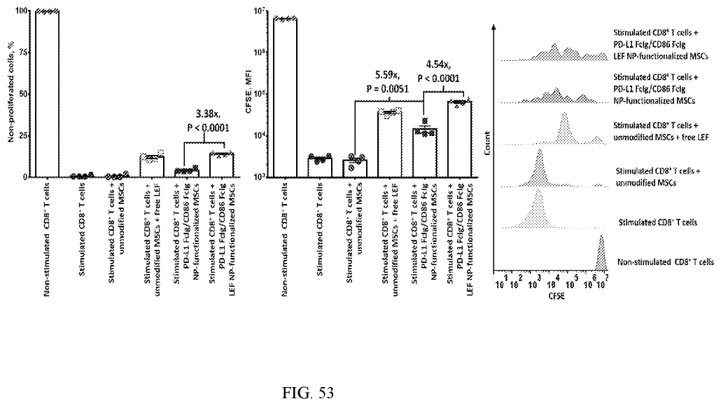

To demonstrate that PD-L1-Ig/CD86-Ig NP-functionalized MSCs can directly inhibit the activation of CD8⁺ T cells and thus reduce inflammation in the CNS, we performed a carboxyfluorescein succinimidyl ester (CFSE) assay to quantify the proliferation of stimulated CD8⁺ T cells (isolated from wild-type C57BL/6 mice) after culturing them with drug-free and LEF-encapsulated PD-L1-Ig/CD86-Ig NP-functionalized MSCs (FIG. 53). The mean fluorescence intensity (MFI) of CFSE-labeled CD8⁺ T cells cocultured with PD-L1-Ig/CD86-Ig NP-functionalized MSCs was 5.6 times higher than compared with that of these cells cultured with the unmodified MSCs (FIG. 53). These findings indicate that conjugated PD-L1⁶⁰ and CD86⁶¹ effectively inhibited the proliferation of stimulated CD8⁺ T cells, independent of the antigen. The MFI of CD8⁺ T cells cocultured with PD-L1-Ig/CD86-Ig LEF NP-functionalized MSCs was 4.5 times higher than compared with that of the MFI of cells cultured with drug-free functionalized MSCs (FIG. 53). These findings show that the encapsulated LEF released from the NPs inhibited the proliferation of activated CD8⁺ T cells in vitro.

### Example 10: PD-L1 and CD86 Directly Functionalized MSCs Prevent and Ameliorate Experimental Autoimmune Encephalomyelitis (EAE)

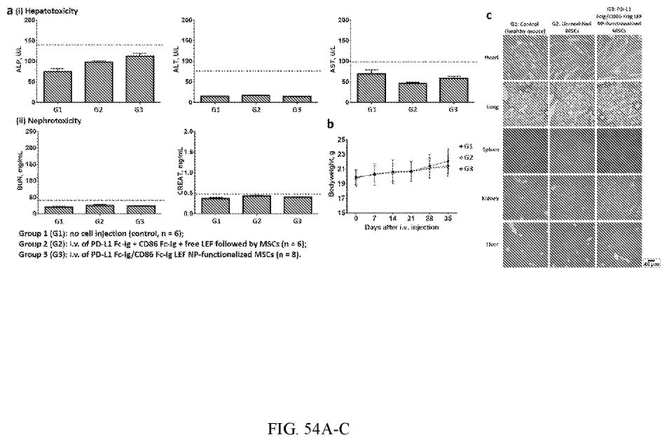

To determine whether i.v. administration of PD-L1- and CD86-functionalized MSCs can ameliorate CNS disorder, we used a monophasic chronic MOG₃₅₋₅₅-induced EAE model because it is the best-characterized model to develop therapies for MS⁶². When we performed an in vivo toxicity study, we found that i.v. administration of unmodified and PD-L1-Ig/CD86-Ig NP-functionalized MSCs (2×10⁶ cells per mouse) did not induce detectable pulmonary toxicity, hepatotoxicity, or nephrotoxicity in healthy C57BL/6 mice (FIG. 54).

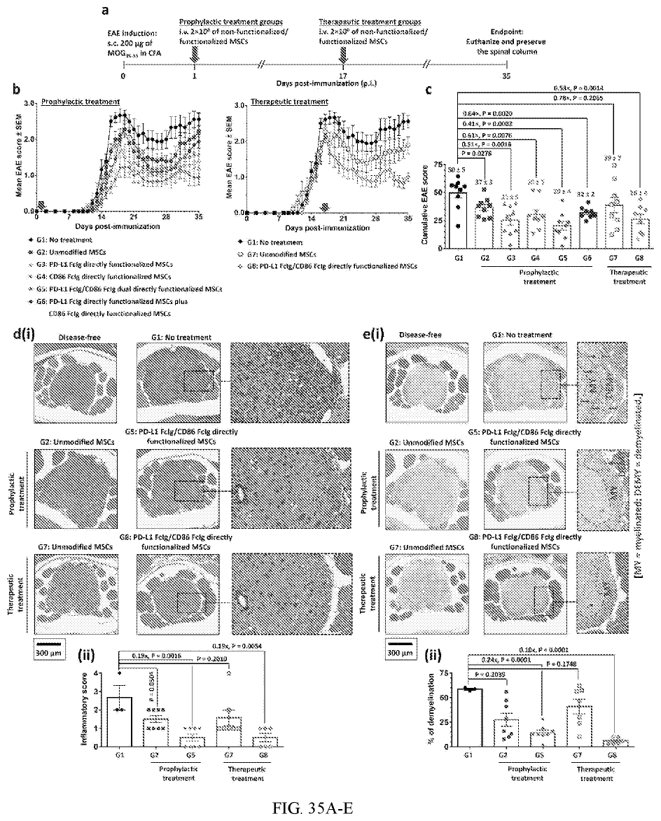

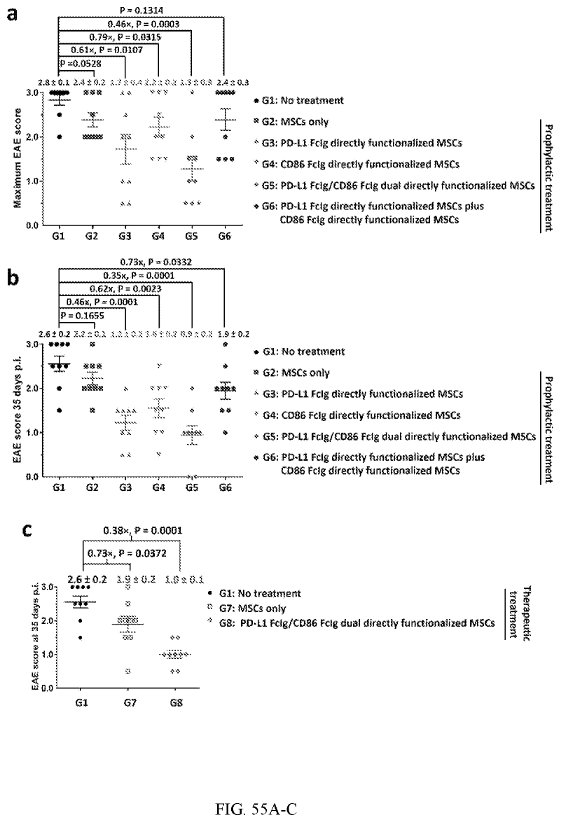

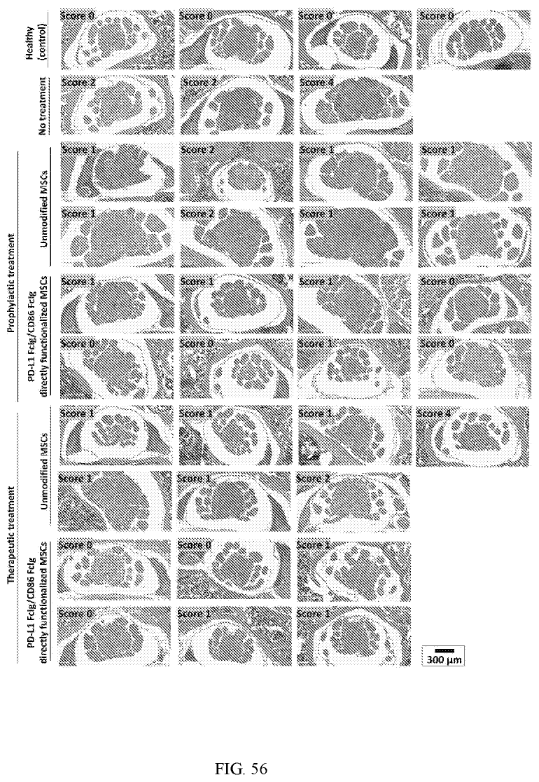

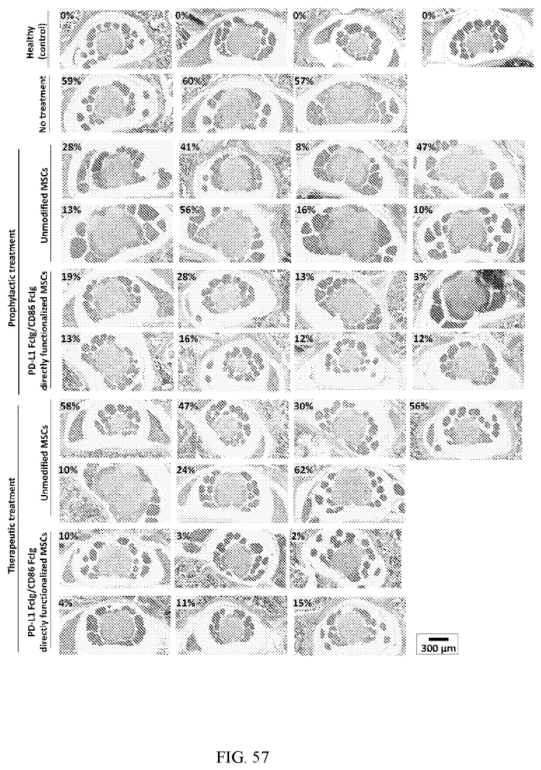

To demonstrate a prophylactic effect, we i.v. administered MSCs 24 h post-immunization (p.i.) with MOG₃₅₋₅₅. The administration of unmodified MSCs did not significantly affect disease progression or severity (FIG. 35a). Tail and hindlimb paralysis (EAE score≥2.5) were observed between 18 and 22 days p.i. Prophylactic treatments with PD-L1-Ig or CD86-Ig directly mono-functionalized MSCs did not significantly delay disease onset, though both treatments reduced severity as indicated by maximum EAE scores p.i. and cumulative EAE scores by 60% and 40% (FIG. 35b-c, and FIG. 55), respectively. Although prophylactic treatment with dual-functionalized MSCs did not completely prevent the development of EAE, its severity was significantly reduced (only 1 of 9 treated mice experienced partial hindlimb paralysis, EAE score≥2.0) (FIG. 35b-c, and FIG. 55). Spinal inflammation and demyelination in mice with EAE are marker of severity of clinical signs⁶³. Histological studies (FIG. 35d-e, and FIGS. 56 and 57) revealed that prophylactic treatment with PD-L1-Ig/CD86-Ig directly dual-functionalized MSCs reduced spinal inflammation by an average of 81% and demyelination by 76% compared with untreated mice at the study endpoint (36 or 37 days p.i.).

We therefore investigated the effects of treating EAE mice with PD-L1 and CD86 dual-functionalized MSCs after disease onset (FIG. 35b-c, and FIG. 55). Therapeutic treatment with PD-L1-Ig/CD86-Ig directly functionalized MSCs significantly reduced cumulative EAE scores by 50% (FIG. 35b-c, and FIG. 55). At the study endpoint (35 days p.i.), 7 of 9 treated mice no longer suffered detectable hindlimb weakness, whereas at least one hindlimb of the untreated mice was completely paralyzed (EAE score≥2.5; FIG. 35b-c, and FIG. 55). Histological studies showed that therapeutic treatment with dual-functionalized MSCs reduced spinal inflammation and demyelination by 81% and 90%, respectively, compared with those of untreated EAE mice 36 or 37 days p.i. (FIG. 35d-e, and FIGS. 56 and 57).

### Example 11: LEF-Encapsulated PD-L1-Ig CD86-Ig NP-Functionalized MSCs are More Effective than Directly Functionalized MSCs to Prevent and Treat EAE

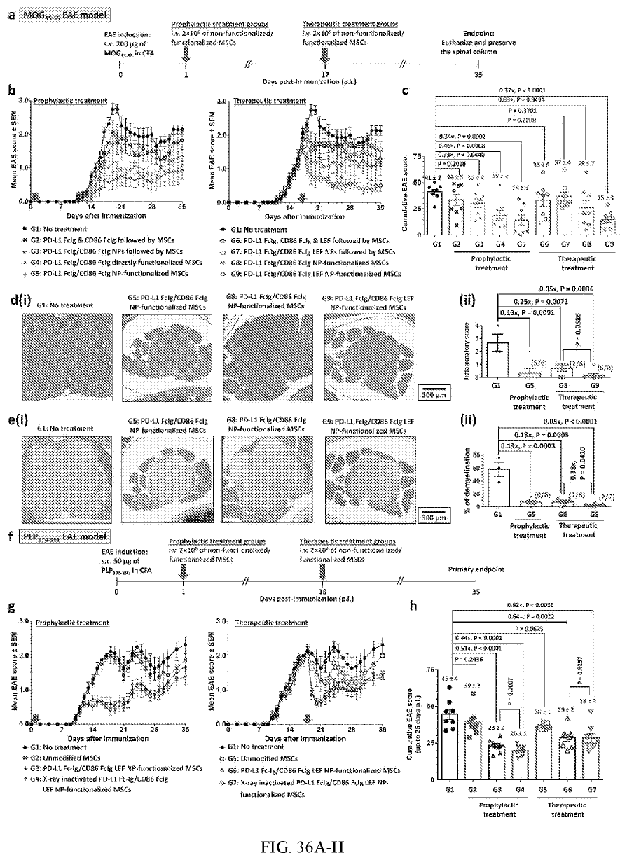

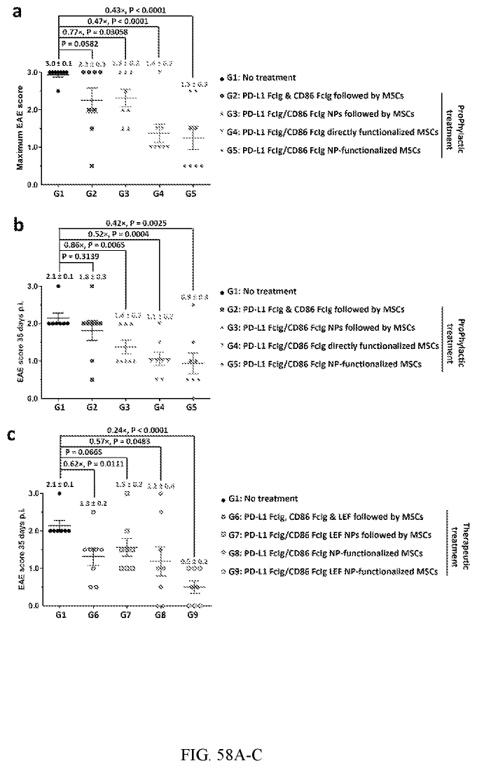

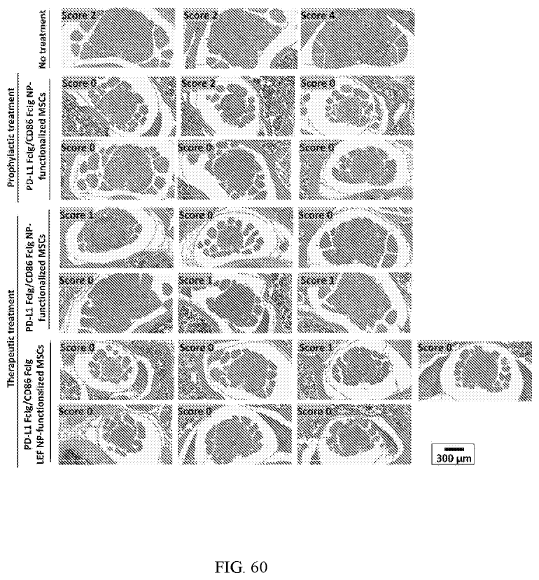

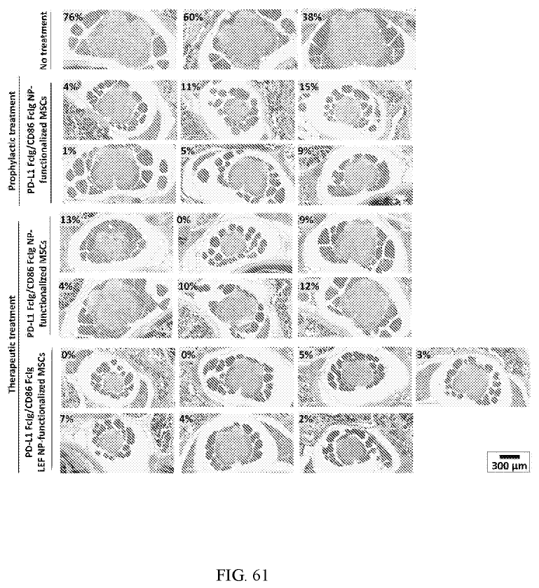

Considering the improved abilities of NP-functionalized MSCs to suppress pathogenic CD4⁺ T cell activation and to facilitate the development of antigen-specific T_{reg} cells in vitro (FIG. 34), we further investigated the abilities of drug-free and LEF-encapsulated PD-L1-Ig/CD86-Ig NP-functionalized MSCs to prevent the development and serve as a treatment for mice with EAE- (FIG. 36a). Prophylactic treatment with drug-free PD-L1-Ig/CD86-Ig NP-functionalized MSCs did not completely prevent the onset of disease, although such treatment was 12% more effective than PD-L1-Ig/CD86-Ig directly functionalized MSCs for reducing cumulative EAE scores upon completion of the study (4 of 8 treated mice suffered partial tail paresis (EAE score=0.5)) (FIG. 36b-c, and FIG. 58). However, prophylactic treatment with LEF-encapsulated PD-L1-Ig/CD86-Ig LEF NP-functionalized MSCs did not further reduce the severity of EAE symptoms than drug-free PD-L1-Ig/CD86-Ig NP-functionalized MSCs (FIG. 36b-c, and FIG. 58). Control prophylactic studies indicated that i.v. administration of small-molecule LEF with unconjugated PD-L1-Ig and CD86-Ig, or PD-L1-Ig/CD86-Ig LEF NPs followed by unmodified MSCs in treated mice did not inhibit the development of EAE or reduce the severity of the disease compared with untreated mice (FIG. 36b-c, and FIG. 58). Similarly, histological analysis showed that treatment with PD-L1-Ig/CD86-Ig LEF NP-functionalized MSCs was as effective as treatment with dual-functionalized MSCs, reducing spinal inflammation by 87% and demyelination by 89% compared with the results for untreated mice (FIG. 36d-e, and FIGS. 60 and 61).

Similar to the results of the prophylactic study, therapeutic treatment with drug-free PD-L1-Ig/CD86-Ig NP-functionalized MSCs was as effective as treatment with directly functionalized MSCs in inhibiting the progression of EAE and reversing certain associated symptoms. In contrast, the PD-L1-Ig/CD86-Ig LEF NP-functionalized MSCs were 29% more effective than the drug-free PD-L1-Ig/CD86-Ig NP-functionalized MSCs in reducing cumulative EAE scores (FIG. 36b-c, and FIG. 58). At 35 days p.i., all mice treated with PD-L1-Ig/CD86-Ig LEF NP-functionalized MSCs regained hindlimb strength (EAE score≤2.0; FIG. 36b-c, and FIG. 58), and 3 of 9 treated mice were symptom-free. This improved therapeutic efficiency shows that encapsulated LEF is required to control the proliferation of autoreactive T cells in the CNS. Consistent with the prophylactic study, treatment with small-molecule LEF, unconjugated PD-L1-Ig, and CD86-Ig or PD-L1-Ig/CD86-Ig LEF NPs followed by unmodified MSCs did not achieve significant therapeutic effects compared the result for untreated mice.

Histological analysis showed that therapeutic treatment with drug-free PD-L1-Ig/CD86-Ig NP-functionalized MSCs was as effective as treatment with dual-functionalized MSCs in reducing spinal cord inflammation by 75% and demyelination by 87% (compared with the results for untreated mice) at 36 or 37 days p.i. (FIG. 36d-e, and FIGS. 60 and 61). Treatment with LEF-encapsulated MSCs further reduced spinal inflammation by 95% (6 of 7 treated mice did not exhibit detectable spinal inflammation) and demyelination by 95% (2 of 7 treated mice did not exhibit detectable demyelination) compared with the results for untreated mice 36 or 37 days p.i. Although the degree of demyelination in EAE mice treated with PD-L1-Ig/CD86-Ig LEF NP-functionalized MSCs was similar in mice treated with drug-free PD-L1-Ig/CD86-Ig NP-functionalized MSCs (FIG. 61), LEF-encapsulated MSCs significantly reduced spinal inflammation (7 of 8 treated mice did not exhibit detectable inflammation) compared with the drug-free functionalized MSCs (3 of 8 treated mice did not exhibit detectable inflammation) (FIG. 60). Though treatment with drug-free PD-L1-Ig/CD86-Ig NP-functionalized MSCs also reduced EAE clinical signs, it less effectively reduced spinal cord inflammation and demyelination than with treatment with PD-L1-Ig/CD86-Ig LEF NP-functionalized MSCs. These findings support our hypothesis that the functionalized MSCs served as a vehicle for the therapeutic delivery of LEF into the spinal cord, thereby reducing the proliferation of autoreactive T cells in the CNS.

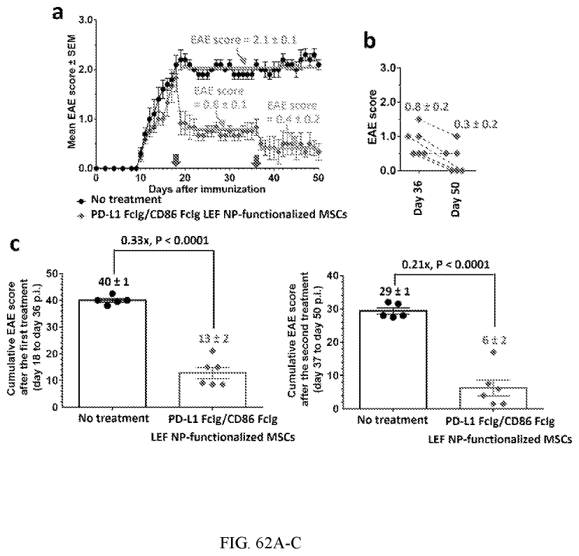

Recognizing that not all the EAE mice were cured after the first therapeutic treatment, we administered a second dose of PD-L1-Ig/CD86-Ig LEF NP-functionalized MSCs to the EAE mice 35 days p.i. In a separate therapeutic treatment study (FIG. 62), 4 of 6 mice responded to the second treatment with the PD-L1-Ig/CD86-Ig LEF NP-functionalized MSCs. The average EAE score significantly decreased by 50% (from 0.8 to 0.4) after the second treatment, and 3 of 6 of those mice were symptom-free at the study endpoint (50 days p.i.; FIG. 62).

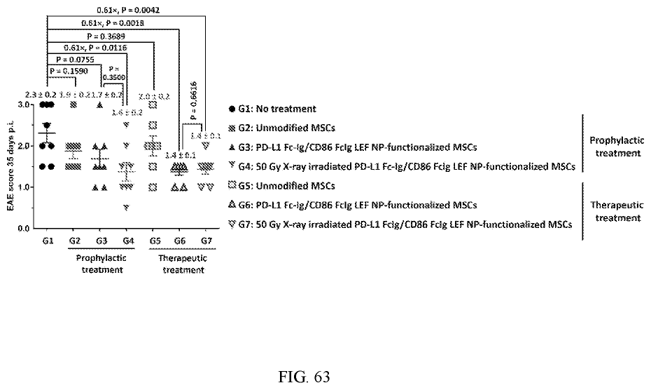

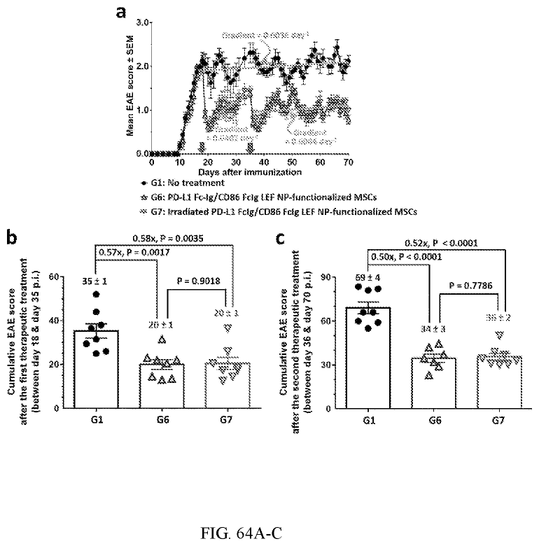

To demonstrate that PD-L1-Ig/CD86-Ig LEF NP-functionalized MSCs can treat relapsing-remitting MS, we used a PLP₁₇₈₋₁₉₁-induced EAE model⁶⁴ (FIG. 36f). Although prophylactic treatment with PD-L1-Ig/CD86-Ig NP-functionalized MSCs did not completely prevent the development of EAE symptoms in this model, it significantly ameliorated clinical symptoms as well as the cumulative EAE score (49% at up to 35 days p.i.) (FIG. 36g-h, and FIG. 63). Similar to the therapeutic effects of the MOG-induced model of EAE, therapeutic treatment with functionalized MSCs reduced the cumulative EAE score by 43% (FIG. 36g-h, and FIG. 63). Similar to the outcome of using the MOG₃₅₋₅₅-immunized model, a second therapeutic treatment, administered 17 days after the first treatment, significantly reduced disease progression from 0.0402 day⁻¹ to 0.0044 day⁻¹ (89% decrease; FIG. 64). These findings support the conclusion that a booster dose further improved the efficiency of therapy. In embodiments, the booster can be administered when the EAE score has plateaued, or when the rate of EAE score has stabilized.

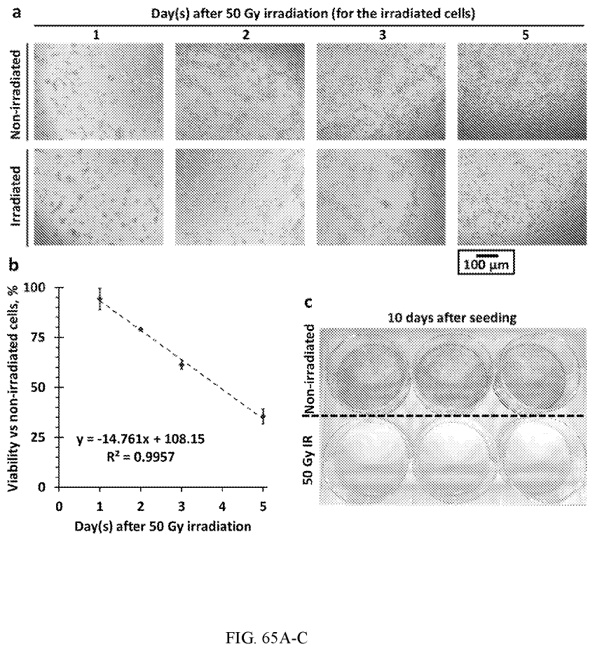

To prove that i.v. administered MSCs did not directly involve remyelination, we administered 50 Gy X ray-irradiated PD-L1-Ig/CD86-Ig LEF NP-functionalized MSCs for prophylactic and therapeutic treatments. The dying X ray-irradiated MSCs (FIG. 65) were as effective as the non-irradiated MSCs in reducing clinical signs and cumulative EAE scores, which indicates that bioengineered MSCs are not directly involved in myelin repair (FIG. 36g-h, and FIG. 63).

### Example 12: Bioengineered MOLs Effectively Ameliorate Active EAE

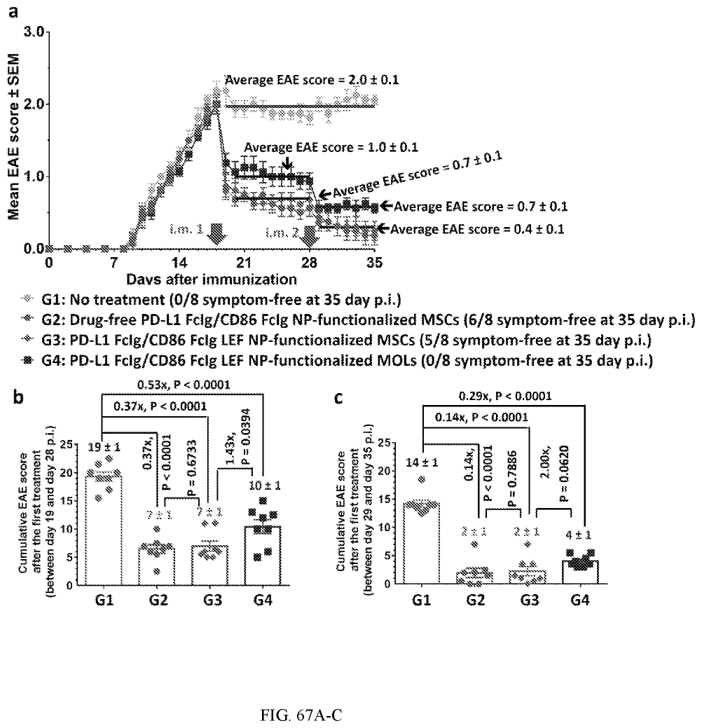

It has been demonstrated elsewhere herein that bioengineered SCs are useful for the treatment of MS. A further therapeutic study in MOG₃₅₋₅₅-immunized EAE mice with bioengineered MOLs demonstrates the ability of using myelin-expressing glial cells to induce antigen-specific immunotolerance and ameliorate active MS. In contrast to the unmodified MSCs, unmodified MOLs administered by i.v. rapidly reversed the hindlimb weakness symptoms within 24 h post-administration, but the EAE symptoms recurred 4 days later (FIG. 67). The therapeutic treatment with the unmodified MOLs did not significantly affect the overall clinical signs. The i.v. administration of PD-L1-Ig/CD86-Ig LEF NP-functionalized MOLs also rapidly reversed the EAE symptoms. Unlike the results for treatment with unmodified MOLs, the hindlimb weakness symptom (EAE score=2.0) completely disappeared in 6 out of 8 mice treated with the PD-L1-Ig/CD86-Ig LEF NP-functionalized MOLs (EAE score=1.3±0.4 at the study endpoint). The therapeutic studies confirmed the potential of using bioengineered myelin-expressing glial cells to treat active MS.

### Example 13: Intramuscular (i.m.) Administration of Bioengineered MSCs is as Effective as i.v. Administered Bioengineered MSCs and MOLs to Ameliorate Active EAE

Though our study focused on i.v. administration to allow functionalized cells to directly engage circulating autoreactive T cells and enter the CNS to resolve the EAE symptoms, we further investigated i.m. administration. Similar to i.v. administration, the first i.m. dose of drug-free PD-L1-Ig/CD86-Ig NP-functionalized MSCs reduced the average EAE score by 65% compared with the average EAE score of untreated mice (FIG. 67). All treated mice suffered only tail paralysis symptom 10 days after the therapeutic treatment. In contrast to i.v. administration method, i.m. administration of PD-L1-Ig/CD86-Ig LEF NP-functionalized MSCs did not achieve significant therapeutic improvement compared with drug-free PD-L1-Ig/CD86-Ig NP-functionalized MSCs (FIG. 67). These findings can be explained by the inability of i.m.-administered MSCs to deliver the encapsulated LEF to the CNS that inhibits the proliferation of autoreactive T cells. Similar to the results for i.v. administration, EAE mice responded well to a second i.m. treatment and exhibited further resolved EAE clinical signs. At the study endpoint (38 days p.i.), 6/8 and 5/8 of the mice treated with the drug-free/LEF-encapsulated LEG NP-functionalized MSCs were EAE symptom-free.

Mechanistic Insight: Immune Checkpoint Ligand-Bioengineered MSCs Prevent and Treat EAE Through the Induction of Antigen-Specific T_{reg} Cells

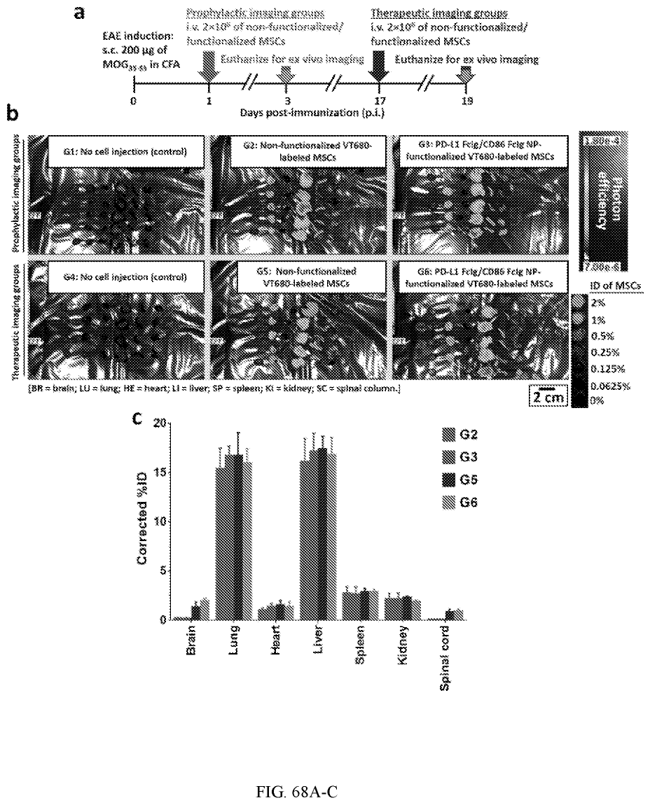

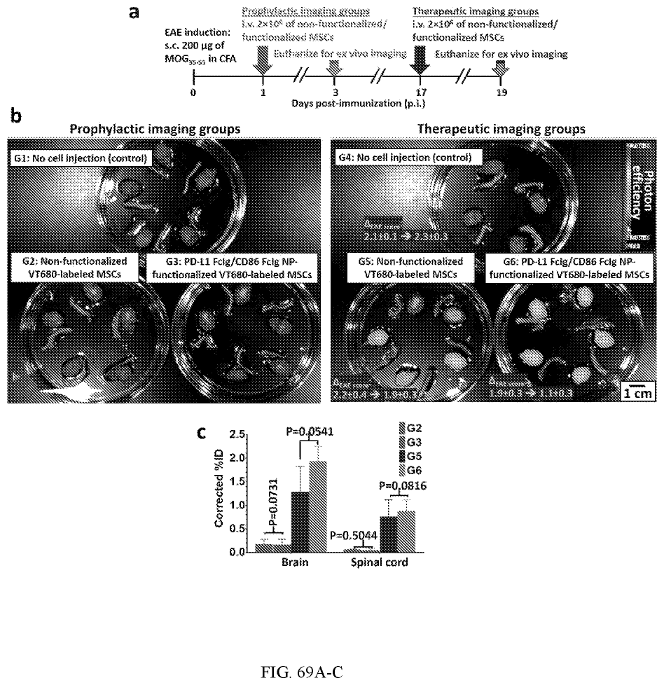

We next performed an ex vivo imaging study in the MOG₃₅₋₅₅-immunized EAE model to determine the biodistribution 48 h after the i.v. administration of VivoTag 680 (VT680)-labeled unmodified and PD-L1-Ig/CD86-Ig NP-functionalized MSCs (FIGS. 68 and 69). In the prophylactic imaging groups, the majority of the administered MSCs accumulated in peripheral organs, with ≤0.2% of the injected dose (ID) of the MSCs detected in the CNS (FIGS. 68 and 69), indicating that i.v. administered MSCs likely interacted with immune cells that infiltrated the CNS. In contrast, approximately 1.75% ID and 0.75% ID of MSCs accumulated in the brain and spinal cord (FIGS. 68 and 69), respectively. Although the majority of administered MSCs remained in peripheral organs, CNS-infiltrating MSCs may be required to maintain CNS-specific immunotolerance in MOG₃₅₋₅₅-immunized EAE mice.

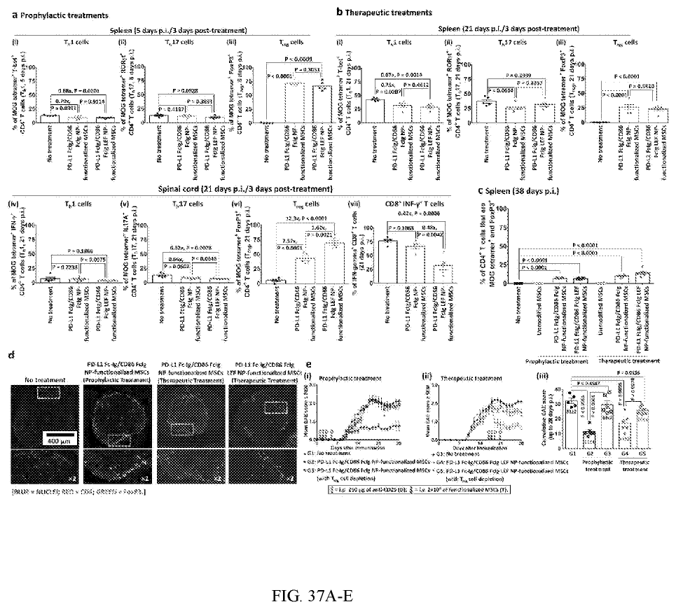

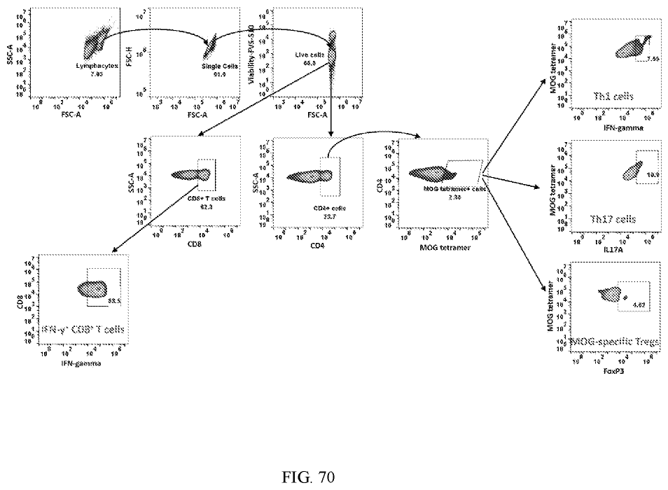

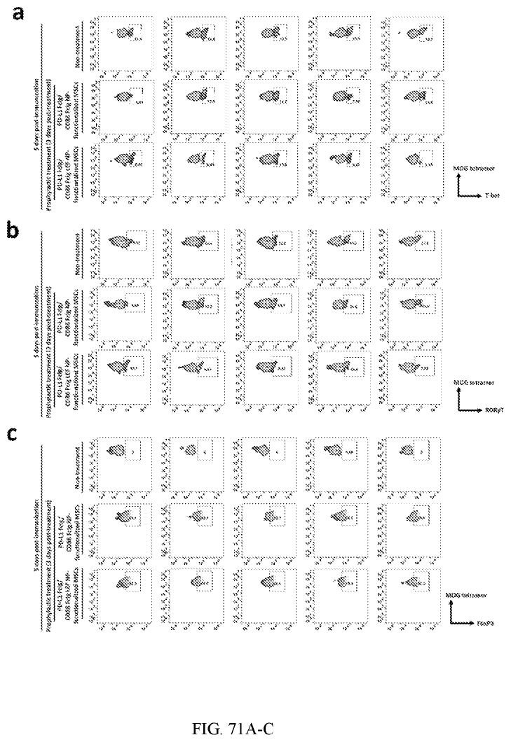

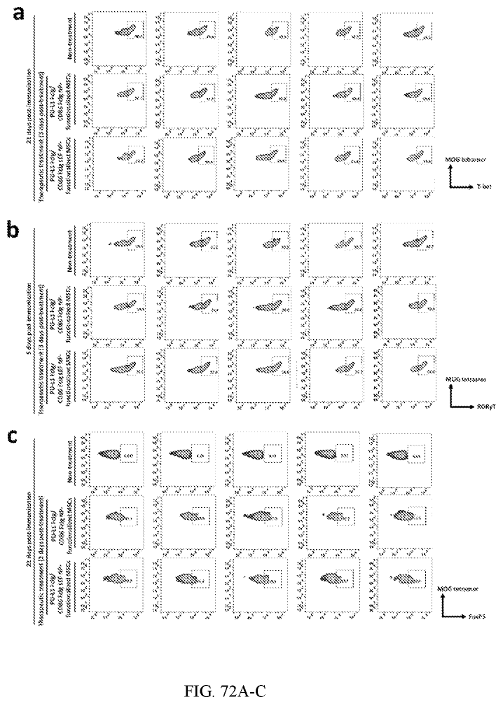

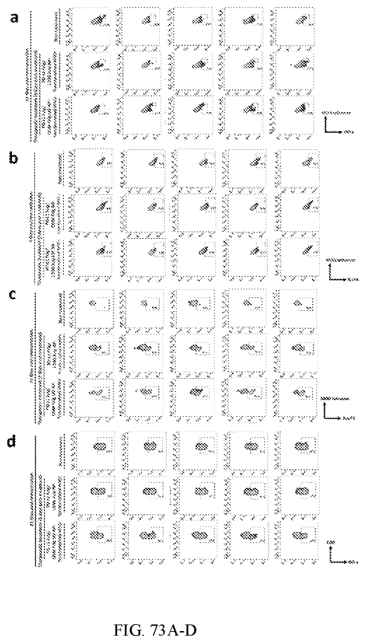

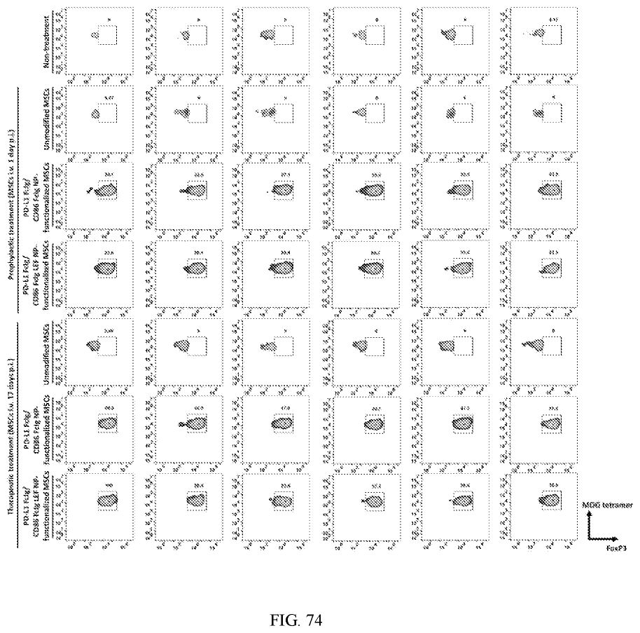

We next analyzed MOG₃₅₋₅₅-specific CD4⁺ T cell populations 3 days after prophylactic and therapeutic treatments with i.v. administered drug-free and LEF-encapsulated PD-L1-Ig/CD86-Ig NP-functionalized MSCs (FIG. 70). Prophylactic treatment with both functionalized MSCs were equally effective in promoting the development of MOG₃₅₋₅₅-specific splenic T_{reg} cells (approximately 70% of MOG₃₅₋₅₅⁺ CD4⁺ cells being FoxP3⁺) and slightly reduced the numbers of splenic MOG₃₅₋₅₅-specific Tₕ1 and Tₕ17 cells (FIG. 37a, and FIG. 71). Similarly, therapeutic treatment with both PD-L1-Ig/CD86-Ig NP-functionalized MSCs was equally effective in promoting the development of MOG₃₅₋₅₅-specific splenic T_{reg} cells (with approximately 25% of the splenic MOG₃₅₋₅₅⁺ CD4⁺ cells being FoxP3⁺) and slightly reducing the number of MOG-specific splenic Tₕ1 and Tₕ17 cells (FIG. 37b, and FIG. 72). In contrast, treatment with PD-L1-Ig/CD86-Ig LEF NP-functionalized MSCs induced 62% more MOG₃₅₋₅₅-specific spinal CD4⁺ T_{reg} cells than with treatment with drug-free PD-L1-Ig/CD86-Ig NP-functionalized MSCs (FIG. 37b, and FIG. 73). Thus, 32.2±7.6% of CD8⁺ T cells infiltrating the spinal cord expressed IFN-gamma (FIG. 37b, and FIG. 74), whereas 76.7±2.8% and 67.2±4.4% of the CD8⁺ T cells infiltrating the spinal cords of mice treated or not treated with the drug-free PD-L1-Ig/CD86-Ig NP-functionalized MSCs expressed IFN-gamma, respectively. Moreover, PD-L1-Ig/CD86-Ig NP-functionalized MSCs effectively inhibited the development of EAE and reversed certain early-onset symptoms by promoting the development of MOG₃₅₋₅₅-specific T_{reg} cells (FIG. 37c, and FIG. 74). Further, histopathological analysis of the spinal cord preserved 36 or 37 days p.i. revealed that prophylactic and therapeutic treatments with the PD-L1-Ig/CD86-Ig NP-functionalized MSCs promoted the development of suppressive CD4⁺ FoxP3⁺ T_{reg} cells in the spinal cord (FIG. 37d).

To confirm these findings, we performed T_{reg} cell depletion studies with CD25-specific antibodies in MOG₃₅₋₅₅-immunized mice (FIG. 37e)⁶⁵. Similar to the result for untreated mice, T_{reg} cell-depleted mice developed severe EAE symptoms after prophylactic treatment with PD-L1-Ig/CD86-Ig NP-functionalized MSCs (cumulative EAE score=31±2 versus 29±2 in the non-treatment control group) (FIG. 37e). The depletion of T_{reg} cells before treatment with PD-L1-Ig/CD86-Ig LEF NP-functionalized MSCs significantly reduced the therapeutic efficiency of the functionalized MSCs and increased the cumulative EAE scores by 88% (FIG. 37e). These findings indicate that T_{reg} cells induced by PD-L1-Ig/CD86-Ig LEF NP-functionalized MSCs are required to maintain immunotolerance to MOG₃₅₋₅₅-induced EAE.

### In Vivo Bioengineering—Examples 14-19

Materials: Biotin-poly(ethylene glycol)-b-poly(lactide-co-glycolide) (Bio-PEG-PLGA; molecular weight=2 kDa+10 kDa; catalog number: 909882), poly(lactide-co-glycolide) (PLGA, ester terminated; M_{w}=50-70 kDa), acetonitrile (HPLC grade, ≥99%), water (for molecular biology, sterile filtered) was purchased from Sigma. Methoxy ethylene glycol)-b-poly(lactide-co-glycolide) (mPEG-PLGA; molecular weight=2 kDa+15 kDa; AK027) and poly(lactide-co-glycolide)-cyanine 5 (Cy5-labeled PLGA; molecular weight=30-50 kDa; AV034) were purchased from Akina, Inc. (West Lafayette, IN). N-azidoacetylmannosamine tetraacylated (Ac₄ManNAz) and dibenzocyclooctyne-functionalized oligoethylene glycol N-hydroxysuccinimide ester (DBCO-PEG13-NHS ester; 95%) was purchased from Click Chemistry Tools (Scottsdale, AZ). Novex™ Avidin (catalog number: 43-440), biotin-Exendin 4 (AnaSpec; catalog number: NC1906171), and IGRP Catalytic Subunit-Related Protein (IGRP206-214; Eurogentec) were purchased from Fisher Scientific (Hampton, NH). Recombinant mouse PD-L1-Ig fusion protein (PD-L1-Ig; molecular weight=102 kDa; PR00112-1.9) was purchased from Absolute Antibody NA (Boston, MA). The fusion protein was supplied in sterilized 1×PBS.

Preparation of β cell-targeted NPs: Exendin 4-functionalized β cell-targeted NPs were prepared by a 2-step nanoprecipitation method, as previously reported. In the first step, biotin-functionalized Ac₄ManNAz NPs were prepared via nanoprecipitation with a 20 wt/wt % Ac₄ManNAz target loading. For the preparation of 20 mg biotin-functionalized Ac₄ManNAz NPs, 9.33 mg of biotin-PEG-PLGA, 4.67 mg of mPEG-PLGA, 6 mg of PLGA, and 4 mg of Ac₄ManNAz were dissolved in 2 mL of acetonitrile before being added slowly (1 ml/min) into 7 mL of stirring deionized water and stirred (1,000 rpm) under reduced pressure for 15 h. The nanoparticles were purified 3 times through Amicron Ultra ultrafiltration membrane filter (MWCO 100,000) as per the manufacturer's protocol. The purified NPs (suspended in deionized water) were concentrated to 40 mg/mL after purification. To prepare avidin-coated NPs, the purified Ac₄ManNAz NPs (20 mg, at a concentration of 40 mg/mL) were mixed with avidin (10 mg, at a concentration of 10 mg/mL in 0.1 M PBS) by vortex mix at 1,500 rpm for 1 min followed by incubation at 20° C. for 1 h under gentle mixing (100 rpm in a shaker). Unbound avidin was removed through 3 washes using an Amicron Ultra ultrafiltration membrane filter (MWCO 100,000) as per the manufacturer's protocol. The purified avidin-functionalized NPs were concentrated to 20 mg/mL (suspended in 0.1 M PBS) after purification. For the preparation of 20 mg of biotin-functionalized exendin 4-functionalized NPs, 60 μg of biotin-functionalized exendin 4 (60 μL, 1 mg/mL in deionized water) was added to the purified avidin NPs and incubated at 20° C. for 1 h under gentle mixing (100 rpm in a shaker). The NPs were washed twice through an Amicron Ultra ultrafiltration membrane filter (MWCO 100,000) as per the manufacturer's protocol. The purified NPs (suspended in 0.1 M PBS) were concentrated to 25 mg/mL and kept at 4° C. before further studies.

β cell-targeted Cy5-labeled (Ac₄ManNAz-free) NPs were prepared by the same method, except that 0.5 mg of Cy5-labeled PLGA was added to the polymer blend for each 10 mg of non-targeted NPs.

Preparation of non-targeted NPs: Non-targeted Ac₄ManNAz NPs were prepared through nanoprecipitation with a 20 wt/wt % Ac₄ManNAz target loading. For the preparation of 20 mg non-targeted Ac₄ManNAz NPs, 14 mg of mPEG-PLGA, 6 mg of PLGA, and 4 mg of Ac₄ManNAz were dissolved in 2 mL of acetonitrile before being added slowly (1 ml/min) into 7 mL of stirring deionized water. The mixture was allowed to stir at reduced pressure (1,000 rpm) for 15 h. The nanoparticles were purified 3 times via Amicron Ultra ultrafiltration membrane filter (MWCO 100,000) as per the manufacturer's protocol. The purified NPs (suspended in 0.1 M PBS) were concentrated to 25 mg/mL and kept at 4° C. before further studies.

Non-targeted Cy5-labeled (Ac₄ManNAz-free) NPs were prepared by the same method, except that 0.5 mg of Cy5-labeled PLGA was added to the polymer blend for each 10 mg of non-targeted NPs.

Characterization of NPs: Purified NPs were characterized by transmission electron microscopy (TEM) and the dynamic light scattering method. TEM images were recorded in a JEOL 1230 transmission electron microscope in Microscopy Services Laboratory (MSL) at the UNC School of Medicine. Before the imaging study, carbon-coated copper grids were glow discharged, and the samples were negatively stained with tungsten acetate (pH 7). The intensity-average diameter of both purified NPs (suspended in 1×PBS) was determined by a Zetasizer Nano ZSP Dynamic Light Scattering Instrument (Malvern). In vitro drug release studies were performed through Slide-A-Lyzer MINI Dialysis Devices (20K MWCO, Thermo Fisher) in the presence of a large excess of 1×PBS at 37° C. Unreleased Ac₄ManNAz from acetonitrile digested NP samples (1:9 1×PBS/acetonitrile; incubated at 4° C. for 72 h) were quantified by liquid chromatography-mass spectrometry in the Department of Chemistry Mass Spectrometry Core Laboratory at the UNC at Chapel Hill.

Preparation of DBCO-functionalized PD-L1-Ig: DBCO-functionalized PD-L1-Ig was functionalized by amine-NHS ester coupling reaction as previously reported. The target degree of functionalization was 60. Briefly, the PD-L1-Ig (1 mg/mL) was incubated with 60 molar equivalent of DBCO-EG13-NHS ester (25 mg/mL in DMSO) at 20° C. in dark for 2 h under gentle shaking (100 rpm). The PD-L1-Ig was purified by Zeba Spin 7K MWCO desalting column, according to the manufacturer's protocol. The concentrations and degrees of the DBCO incorporation of different purified DBCO-conjugated fusion proteins were determined spectroscopically using an absorption coefficient of DBCO at 310 nm (ε_{DBCO, 310 nm})=12,000 M⁻¹ mL cm⁻¹, an absorption coefficient of mouse immunoglobulin at 280 nm (ε_{280 nm})=1.26 mg⁻¹ mL cm⁻¹ (for PD-L1-Ig), and a DBCO correction factor at 280 nm (CF_{DBCO, 280 nm})=1.089 according to the manufacturer's instructions.

Texas Red-labeled DBCO-functionalized PD-L1-Ig was prepared by the same method. The target degree of functionalization was 60 for DBCO-EG13-NHS ester and 5 for Texas Red NHS ester. The concentration of purified PD-L1-Ig was determined by a Pierce™ BCA Protein Assay Kit (Thermo Fisher) and the number of conjugated Texas Red conjugated to PD-L1-Ig was calculated using a molar extinction at 595 nm of 80,000 M⁻¹ mL cm⁻¹.

In Vitro Studies-Cell lines: NIT-1 cells (murine β cell line established from non-diabetic NOD/Lt mice) were purchased from the American Type Culture Collection (Manassas, VA). NIT-1 cells were cultured in F-12 medium (Gibco) supplemented by 10% v/v fetal bovine serum (FBS, Seradigm), 2 mM GlutaMAX Supplement (Gibco), and antibiotic-antimycotic (Anti-Anti; 100 units of penicillin, 100 μg/mL of streptomycin and 0.25 μg/mL of amphotericin B; Gibco). MIN6 cells (murine β cell line established from non-diabetic C57BL\6 mice) were acquired from the American Type Culture Collection (Manassas, VA). MIN6 cells were cultured in DMEM (high glucose) medium (Gibco) supplemented by 15% v/v fetal bovine serum (FBS, Seradigm) and antibiotic-antimycotic (Anti-Anti; 100 units of penicillin, 100 μg/mL of streptomycin, and 0.25 μg/mL of amphotericin B; Gibco). Phenol red-free media were used for cell culture for in vitro binding studies.

In vitro binding assay: NIT-1 and MIN6 cells were seeded in a black 96-well plate at a density of 2×10⁴ cells/well (in phenol red-free media) at 37° C. for 18 h. Calculated amounts of targeted and non-targeted Cy5-labeled NPs were added to the plated cells and allowed to bind to the β cells at 37° C. for 1 h. Cells were washed 3 times with phenol red-free media before being imaged in an AMI HT Optical Imaging System (excitation wavelength=530±25 nm, emission wavelength=590±25 nm, exposure time=60 s) in the Biomedical Research Imaging Center at the UNC School of Medicine.

In vitro functionalization of NIT-1 cells through different pre-targeted strategies: NIT-1 cells were cultured with 50 μM of small-molecule or encapsulated Ac₄ManNAz in a complete culture medium for 1 h before being washed times to remove unbound Ac₄ManNAz or NPs. The Ac₄ManNAz-treated NIT-1 cells were allowed to culture in a complete cell culture medium for 4 days. After detachment of azide-modified NIT-1 cells through enzyme-free cell dissociation buffer (Gibco), cells (density=10×10⁶ cells/mL) were cultured with DBCO-functionalized PD-L1-Ig (or DBCO-functionalized TexRed-labeled PD-L1-Ig) at 37° C. for 1 h. After the removal of unbound DBCO-functionalized PD-L1-Ig, the cells were used for further FACS study or cultured in a complete cell culture medium for further time-dependent studies.

The amount of conjugated DBCO-functionalized TexRed-labeled PD-L1-Ig in the NIT-1 cells was quantified through an AMI HT Optical Imaging System (excitation wavelength=530±25 nm, emission wavelength=590±25 nm, exposure time=60 s) used in the Biomedical Research Imaging Center at the UNC School of Medicine.

A time-dependent FACS study was performed to quantify the PD-L1 on the surface of (non-labeled) PD-L1-Ig-functionalized NIT-1 cells at different time points after functionalization. For quantification, functionalized NIT-1 cells were detached by non-enzymatic cell dissociation buffer, before being stained with PE-labeled anti-mouse PD-L1 antibody (clone: MIH5, catalog number: 12-5982-82; Invitrogen) for the FACS study.

For CLSM study, NIT-1 cells were functionalized by the same method except that cells were seeded in a Nunc 154526 Chamber Slide System (1.5×10⁴ cells per chamber; Thermo Fisher) for 18 h before treated with Ac₄ManNAz for 1 h. The treated cells were washed and cultured in a complete cell culture medium for 4 days before being functionalized with (non-labeled) DBCO-functionalized PD-L1-Ig at the physiological conditions for 1 h. Cell wells were then washed with 1×PBS (containing 0.03% sodium azide, 10 mM of magnesium sulfate, and 5 wt/wt % bovine serum albumin) before being stained with PE-labeled anti-mouse PD-L1 antibody (clone: 10F.9G2; catalog number: MABF555; Sigma) in 1×PBS containing 0.03% sodium azide, 10 mM of magnesium sulfate and 5 wt/wt % bovine serum albumin. Cells were fixed with 4% paraformaldehyde (4% PFA; Sigma) before being imaged in a Zeiss LSM 710 Spectral Confocal Laser Scanning Microscope in the MSL at the UNC School of Medicine.

The viabilities of NIT-1 cells after incubated with different formulations of Ac₄ManNAz (50 μM) and PD-L1-Ig-functionalized NIT-1 cells were determined by MTS assay (CellTiter96@ Aqueous MTS Powder; Promega) according to the manufacturer's protocol 4 days after incubated at the physiological conditions.

T cell activation assay: IGRP-specific 8.3 T cells were isolated and expanded, as previously reported. Upon functionalization through the described method, functionalized NIT-1 cells were seeded in a 24 well plate (2×10⁴ cells/well; in 0.25 mL complete cell culture medium) in the presence of IGRP₂₀₆₋₂₁₄ peptide (5 μg per well) for 3 h. Expanded 8.3 T cells (2×10⁵ cells/well; effector:target=10:1; in 0.25 mL complete T cell culture medium) were added to the seeded functionalized NIT-1 cells, and cultured for 72 h. The non-adhesive cells were collected for the FACS study, as previously reported. Briefly, the non-adhesive cells were stained with anti-mouse CD8 antibody (clone: 37006; R&D System) and PE-labeled anti-mouse PD-1 antibody (clone: J43; Invitrogen) to quantify the cell surface T cell exhaustion marker PD-1 expressions. After cell surface marker staining, cells were fixed with 4% PFA and permeabilized using the intracellular staining permeabilization wash buffer (Biolegend), before being stained with Alexa Fluor 750-labeled anti-IFN gamma antibody (clone: 37895; catalog number: IC485S100UG; R&D System) for FACS study.

In vivo biodistribution studies—Mice: NOD/ShiLtJ mice (NOD mice, female, about eight weeks old), 8.3 TCR alpha/beta transgenic NOD mice (female, six weeks old), and BALB/c mice (female, seven to eight weeks old) were purchased from the Jackson Laboratory and housed in a sterilized clean room facility at the Animal Study Core, UNC Lineberger Comprehensive Cancer Center. CD-1 IGS mice (female, about eight weeks old) were purchased from the Charles River Laboratory. CD-1 IGS mice were maintained in the Division of Comparative Medicine (an AAALAC-accredited experimental animal facility) under a sterile environment at the University of North Carolina at Chapel Hill. All procedures involving experimental animals were performed as per the protocols approved by the UNC Institutional Animal Care and Use Committee. All in vivo therapeutic studies were performed and monitored independently by the Animal Study Core at the UNC Lineberger Comprehensive Cancer Center. Blood glucose, body weight, and body condition score of NOD mice were monitored twice a week (Monday morning and Thursday afternoon). Blood glucose was determined with a hand-held glucose meter (OneTouch Ultra 2 Blood Glucose Monitoring System).

In vivo toxicities of different pre-targeted treatment strategies: In vivo toxicities of different pretargeted treatment strategies were evaluated in healthy BALB/c mice. Mice were i.v. administrated with β cell-targeted Ac₄ManNAz NPs (180 μg of Ac₄ManNAz/mouse). DBCO-functionalized PD-L1-Ig (80 μg/mouse) was i.v. administered 3 days after the administration of β cell-targeted Ac₄ManNAz NPs. Circulation blood was collected 48 h after the administration of PD-LD1Ig. Blood samples were analyzed by the Animal Histopathology and Laboratory Medicine Core at UNC School of Medicine.

### Example 14: In Vivo Bioengineering of Immune Checkpoint Ligand-Functionalized Beta Cells

Preparation of Pre-Targeting and Effector Components for Pre-Targeted Bioengineering of β Cells

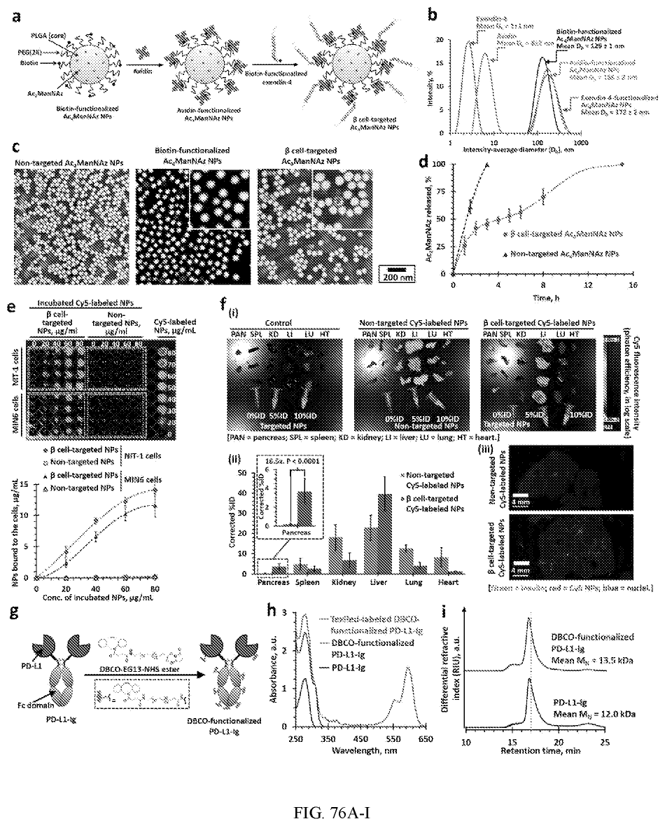

β cell-targeted Ac₄ManNAz NPs were prepared using a reported two-step biotin-avidin-based bioconjugation method (see FIG. 76a).⁷⁷ Briefly, Ac₄ManNAz-encapsulated biotin-functionalized poly(ethyleneglycol)-poly(lactic-co-glycolic acid) (PEG-PLGA) NPs were prepared via nanoprecipitation with a target Ac₄ManNAz loading of 20 wt/wt %. Avidin was conjugated to the purified biotin-functionalized Ac₄ManNAz NPs through the strong biotin-avidin interaction and physisorption in the presence of an excess amount of avidin. Upon removal of unbound avidin, biotin-functionalized exendin-4 was conjugated to the purified avidin-functionalized Ac₄ManNAz NPs through a strong biotin-avidin interaction in a 1:1 stoichiometry.

The bicinchoninic acid assay showed that 46±2 μg (681±30 pmol) of avidin was conjugated to each milligram of biotin-functionalized PEG-PLGA NPs, which allowed quantitative conjugation of 3 μg (680 pmol) biotin-functionalized exendin-4 for each milligram of PEG-PLGA NPs. The intensity-average diameter (Dₕ) of the Ac₄ManNAz NPs significantly increased from 129±1 nm (polydispersity index, PDI=0.072±0.020; see FIG. 76b) to 172±2 nm (PDI=0.182±0.020; see FIG. 76b) after functionalization with the avidin and biotin-functionalized exendin-4, as determined using dynamic light scattering method. A core-shell-like structure can be observed in the corresponding transmission electron microscopy (TEM) images due to the formation of a protein shell (see FIG. 76c). Each milligram of R cell-targeted Ac₄ManNAz NPs was encapsulated with 36±6 μg of Ac₄ManNAz (encapsulation efficiency=18%, as determined by liquid chromatography-mass spectrometry), and it underwent controlled release under physiological conditions (half-life=approximately 6 h; see FIG. 76d).

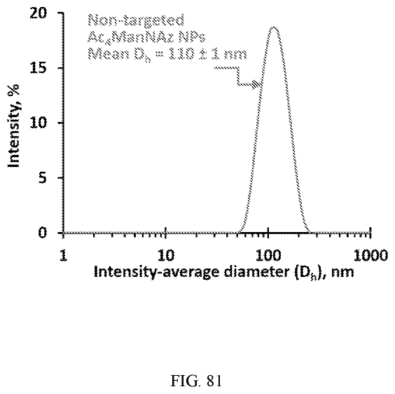

Non-targeted Ac₄ManNAz NPs of about 50 nm in diameter were prepared from methoxy-functionalized PEG-PLGA diblock copolymer via nanoprecipitation (see FIG. 76c; Supporting Information, FIG. 81). Each milligram of non-targeted NPs was encapsulated with 54±3 μg of Ac₄ManNAz (encapsulation efficiency=27%). Unlike the β cell-targeted Ac₄ManNAz NPs, all encapsulated Ac₄ManNAz were released from the NPs within 3 h under sink conditions (see FIG. 76d). The slower Ac₄ManNAz release kinetics that were recorded for the β cell-targeted NPs is due to the hydrophobic Ac₄ManNAz that binds non-specifically to the conjugated avidin.

Ac₄ManNAz-free Cy5-labeled β cell-targeted and non-targeted PEG-PLGA NPs were prepared via the same methods, with the exception that 1 wt/wt % of Cy5-labeled PLGA was added to the polymer blend to fabricate the core PEG-PLGA NPs.

### Example 15: In Vitro Assays of In Situ-Prepared Bioengineered Immune Checkpoint Ligand-Functionalized Beta Cells

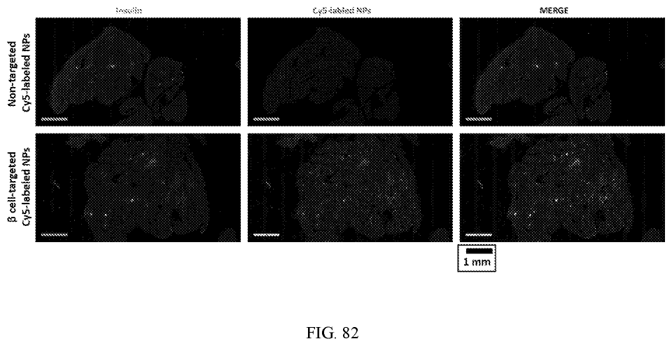

An in vitro binding assay that was performed using NIT-1 cells (insulinoma cells isolated from NOD mice⁷⁸) and MIN-6 cells (insulinoma cells isolated from C57BL/6 mice⁷⁹) confirmed that the β cell-targeted Cy5-labeled NPs bind selectively to the insulin-producing β cells in a concentration-dependent manner (see FIG. 76e). Insignificant non-specific binding was observed for the non-targeted NPs. An ex vivo biodistribution study that was performed in diabetic NOD mice (blood glucose level=300-450 mg/dL) revealed that 3.7±1.4% injected dose (ID) of the i.v. administered β cell-targeted NPs accumulated in the pancreas 3 h post-administration (see FIG. 76f(i), (ii)), which was 16.5 times more than the amount of non-targeted NPs that accumulated in the pancreas (see FIG. 76f(i), (ii)). An additional histopathological study confirmed that the β cell-targeted NPs accumulated mainly in the β cell-rich islets (see FIG. 76f(iii); Supporting Information, FIG. 82). The ex vivo biodistribution study also confirmed that the use of more immunogenic avidin⁸⁰ to functionalize the β cell-targeted NPs allows rapid clearance through the mononuclear phagocyte system (e.g., liver).⁸¹ This bioconjugation strategy effectively prevented the prolonged retention of NPs in the circulation system that non-specifically releases the encapsulated Ac₄ManNAz.

To improve the physiological stability of the therapeutic effector, we used PD-L1 immunoglobin Fc-fusion protein (PD-L1-Ig) for the pretargeted study. DBCO-functionalized N-hydroxysuccinimide (NHS) ester was conjugated to the primary amine-rich Fc component of PD-L1-Ig through an amine-N-hydroxysuccinimide ester coupling reaction (see FIG. 76g), as previously reported.⁸² UV-visible spectroscopy confirmed that each PD-L1-Ig conjugated to an average of 9 DBCO ligands (see FIG. 76h), and the Texas Red (TexRed)-labeled DBCO-functionalized PD-L1-Ig contained two additional conjugated TexRed molecules (see FIG. 76h). A size exclusion chromatography-multiple angle light scattering (SEC-MALS) study confirmed that the fusion protein maintains a uniform size distribution after functionalization (see FIG. 76i).

The biodistributions of β cell-targeted Cy5-labeled NPs and non-targeted Cy5-labeled NPs in diabetic NOD mice (blood glucose=300-450 mg/dL) were quantified by ex vivo fluorescent imaging method. Briefly, β cell-targeted and non-targeted Cy5-labeled NPs were i.v. administered to the diabetic NOD mice (5 mg of NPs/mouse). 3 h thereafter, the mice were euthanized. Pancreas and other key organs (liver, kidney, spleen, heart, and lung) were preserved for ex vivo imaging study in an AMI HT Optical Imaging System (excitation wavelength=530±25 nm, emission wavelength=590±25 nm, exposure time=60 s) in the Biomedical Research Imaging Center at the UNC School of Medicine. ID % in each organ was calculated by comparing the fluorescence efficiencies of different concentrations of standard Cy5-labeled NPs. The preserved pancreas samples were submitted to Pathology Services Core in the UNC Lineberger at the UNC School of Medicine for pathological study. Anti-insulin-stained pancreas sections were imaged in a Scan Scope FL (Leica Biosystems).

### Example 16: Evaluation of Different Pre-Targeted Strategies for Bioengineering β Cells In Vitro

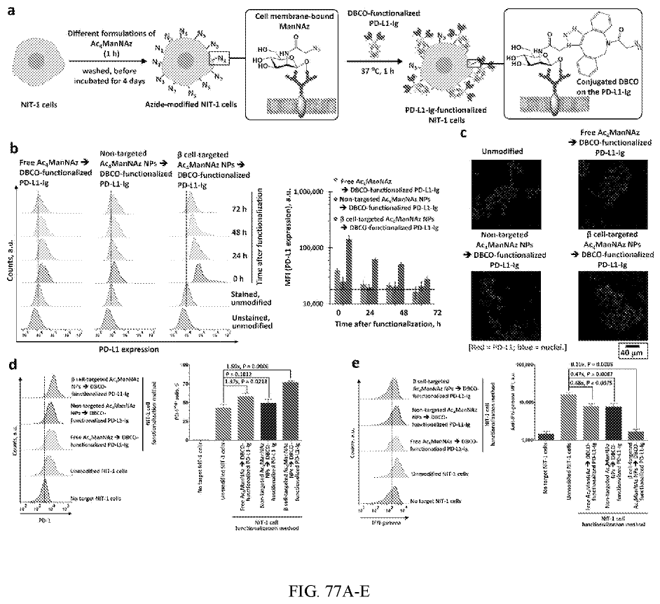

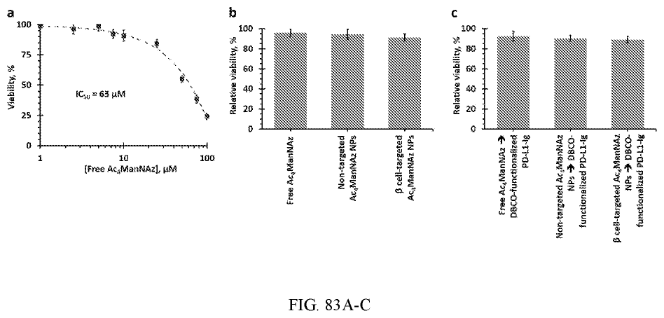

To validate the two-step two-component PD-L1 decoration strategy, we performed an in vitro functionalization study of NIT-1 cells (FIG. 77a). We first incubated the NIT-1 cells with small-molecule Ac₄ManNAz or different Ac₄ManNAz NPs (50 μM; see Supporting Information, FIG. 83a,b) at physiological conditions for 1 h (see FIG. 77a). The NIT-1 cells were washed to remove unbound Ac₄ManNAz before incubation in a complete cell culture medium for 4 days to allow the intracellular ManNAz to convert into an azide sialic acid derivative on the surface proteins of the cell (see FIG. 77a). The azide-modified NIT-1 cells were then incubated with DBCO-functionalized PD-L1-Ig at a target degree of functionalization of 5 μg fusion protein per 1×10⁶ cells at physiological conditions for 1 h to allow SPAAC between cell membrane-bound azide and conjugated DBCO on the PD-L1-Ig (see FIG. 77a). Using DBCO-functionalized TexRed-labeled PD-L1-Ig for biofunctionalization, the NIT-1 cells that were incubated with β cell-targeted Ac₄ManNAz NPs were functionalized with up to 4.3±0.2 μg of DBCO-functionalized PD-L1-Ig per 1×10⁶ cells, while the cells treated with small-molecule Ac₄ManNAz NPs and non-targeted Ac₄ManNAz NPs functionalized with less than 1 μg of PD-L1-Ig per 1×10⁶ cells. The in vitro functionalization did not affect the viability of the NIT-1 cells (see Supporting Information, FIG. 83b,c). Further study using fluorescence-activated cell sorting (FACS) confirmed that all 3 two-step pretargeted functionalization methods increased PD-L1 expression in the NIT-1 cells (see FIG. 77b). More specifically, PD-L1 expression the NIT-1 cells that were pretreated with the β cell-targeted Ac₄ManNAz NPs was 4 times higher than that of cells that were pretreated with small-molecule Ac₄ManNAz and 5.8 times higher than that of cells pretreated with non-targeted Ac₄ManNAz NPs immediately after being functionalized with the DBCO-functionalized PD-L1-Ig (see FIG. 77b). The higher initial conjugation efficiency can be explained by more of the azide group being decorated on the NIT-1 cells through pretreatment with β cell-targeted Ac₄ManNAz NPs. The PD-L1 expression of NIT-1 cells that were functionalized using all 3 different pretargeted functionalization strategies decrease over time after functionalization, due to cell proliferation and metabolic recycling.²¹ Functionalization of PD-L1-Ig on the NIT-1 cells was confirmed by a confocal laser scanning microscopy (CLSM) study after staining with phycoerythrin (PE)-labeled anti-PD-L1 antibody (see FIG. 77c).

We next performed islet-specific glucose-6-phosphatase catalytic subunit-related protein (IGRP)-specific cytotoxic T cell (8.3 T cell) assays (see FIG. 77d,e)^{49, 83} to investigate how different pretargeted strategies affect the functionalized NIT-1 cells in terms of inhibiting islet-specific T cell activation and cell killing. PD-L1-Ig-functionalized NIT-1 cells that were functionalized through the β cell-targeted Ac₄ManNAz NPs followed by DBCO-functionalized PD-L1-Ig were more effective than pretargeted conjugation using small-molecule Ac₄ManNAz and non-targeted Ac₄ManNAz NPs. More specifically, the PD-L1-Ig-functionalized NIT-1 cells that were functionalized through the β cell-targeted Ac₄ManNAz NPs upregulated PD-1 expression (T cell activation marker)⁸⁴ in the co-cultured 8.3 T cells by 80% (see FIG. 77d) and reduced antigen-specific T cell activation by 90% compared to non-functionalized NIT-1 cells (as evaluated by the reduction of intracellular IFN-gamma expression in the 8.3 T cells) (see FIG. 77e).

### Example 17: In Vivo Evaluation of Different Pre-Targeted Strategies for Bioengineering Pancreatic β Cells

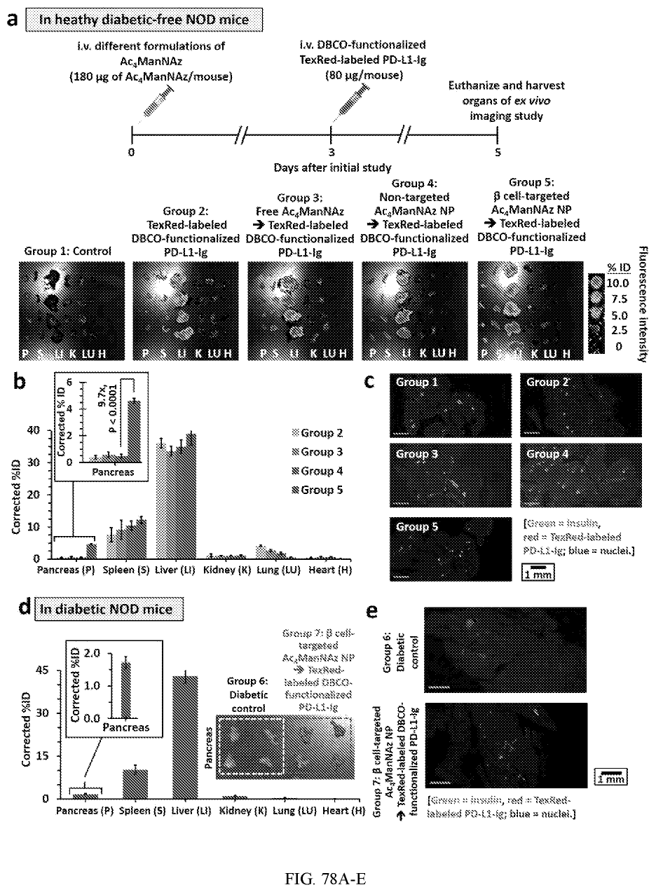

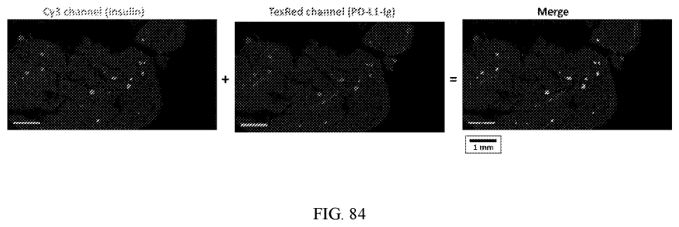

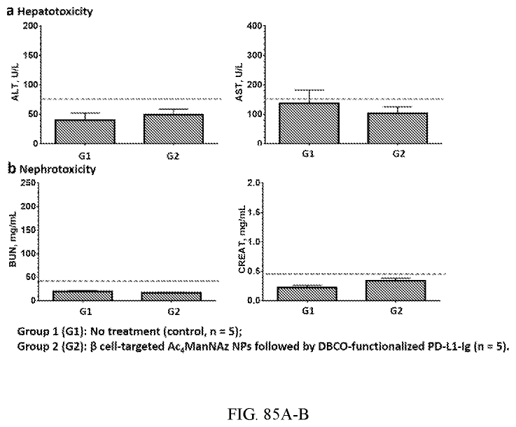

To demonstrate that the two-step, two-component pre-targeted strategies can decorate DBCO-functionalized PD-L1-Ig onto the insulin-producing β cells in vivo, we performed an ex vivo biodistribution study in non-diabetic NOD mice to quantify the accumulation of the DBCO-functionalized TexRed-labeled PD-L1-Ig (see FIG. 78a). In the pre-targeted biodistribution study, DBCO-functionalized TexRed-labeled PD-L1-Ig (80 μg/mouse) was administered i.v. 3 days post-administration of different Ac₄ManNAz formulations (180 μg/mouse). An ex vivo imaging study was performed 48 h post-administration of the PD-L1-Ig. Pretargeted functionalization with small-molecule Ac₄ManNAz and non-targeted Ac₄ManNAz NPs did not significantly affect the accumulation of TexRed-labeled PD-L1-Ig on the pancreas compared to the control group of i.v. administered DBCO-functionalized TexRed-labeled PD-L1-Ig (less than 0.5% ID accumulated in the pancreas; see FIG. 78a). However, pretargeted functionalization with the β cell-targeted Ac₄ManNAz NPs significantly increased the accumulation of the DBCO-functionalized PD-L1-Ig in the pancreas by about ten-fold (compared to the mice administered non-targeted Ac₄ManNAz NPs; see FIG. 78a). Further histopathological study confirmed that most of PD-L1-Ig that was administered using the pretargeted strategy with β cell-targeted Ac₄ManNAz NPs had accumulated in the β cell-rich islets (see FIG. 78b; Supporting Information, FIG. 84). None of the non-targeted and pretargeted strategies significantly affected the quantity of TexRed-labeled DBCO-functionalized PD-L1-Ig that accumulated in the spleen and liver. An additional toxicity study performed in healthy BALB/c mice confirmed that the pretargeted strategy with β cell-targeted Ac₄ManNAz NPs followed by DBCO-functionalized PD-L1-Ig did not induce significant hepatotoxicity and nephrotoxicity (see Supporting Information, FIG. 85), although most of β cell-targeted Ac₄ManNAz NPs (and thus Ac₄ManNAz) and DBCO-functionalized PD-L1-Ig accumulated in the liver.

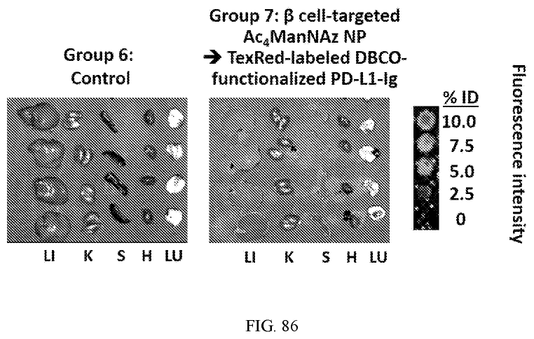

We next focused on investigating the pretargeted strategy using β cell-targeted Ac₄ManNAz NPs as the pretargeted component in diabetic NOD mice (see FIG. 78c). As in the biodistribution study that was performed in non-diabetic NOD mice, most of the i.v. administered TexRed-labeled PD-L1-Ig accumulated in the liver and spleen of diabetic NOD mice 5 days post-administration. Approximately 1.7±0.2% ID of the administered TexRed-labeled DBCO-functionalized PD-L1-Ig remained in the pancreas 5 days post-administration (see FIG. 78c; Supporting Information, FIG. 86). The smaller amount of PD-L1 that accumulated in the pancreas can be explained by the detachment of in vivo conjugated PD-L1 due to cell proliferation and metabolic recycling. A histopathological study confirmed that the islets in the preserved pancreas received the pretargeted treatment with R cell-targeted Ac₄ManNAz NPs followed by TexRed-labeled PD-L1-Ig expressing a higher level of PD-L1 than non-treated diabetic mice (see FIG. 78d).

The biodistributions of R cell-pretargeted TexRed-labeled DBCO-functionalized PD-L1-Ig in non-diabetic (9 weeks old, blood glucose<200 mg/mL) and diabetic NOD mice (blood glucose=350-450 mg/dL) were quantified by the ex vivo fluorescent imaging method. Briefly, mice were i.v. tail-vein administered different formulations of Ac₄ManNAz (180 μg of Ac₄ManNAz/mouse). Small-molecule Ac₄ManNAz was administered as Tween 20 formulation by first dissolving it in Tween 20 at a concentration of 25 mg/mL, before being diluted to 0.9 mg/mL with 0.1 M PBS for i.v. injection. TexRed-labeled DBCO-functionalized PD-L1-Ig (80 μg/mouse) was i.v. administered 3 days after the administration of Ac₄ManNAz. Mice were harvested 48 h after the administration of the TexRed-labeled DBCO-functionalized PD-L1-Ig. Pancreas and other key organs (liver, kidney, spleen, heart, and lung) were preserved for ex vivo imaging study in an AMI HT Optical Imaging System (excitation wavelength=530±25 nm, emission wavelength=590±25 nm, exposure time=60 s) used in the Biomedical Research Imaging Center at the UNC School of Medicine. ID % in each organ was calculated by comparing the fluorescence efficiencies of different concentrations of standard DBCO-functionalized TexRed-labeled NPs. The preserved pancreas samples were submitted to Pathology Services Core in the UNC Lineberger at the UNC School of Medicine for pathological study. Anti-insulin-stained pancreas sections were imaged in a Scan Scope FL (Leica Biosystems).

### Example 18: In Vivo Evaluation of Different Pre-Targeted Strategies to Reverse Early Onset TIDM in NOD Mice

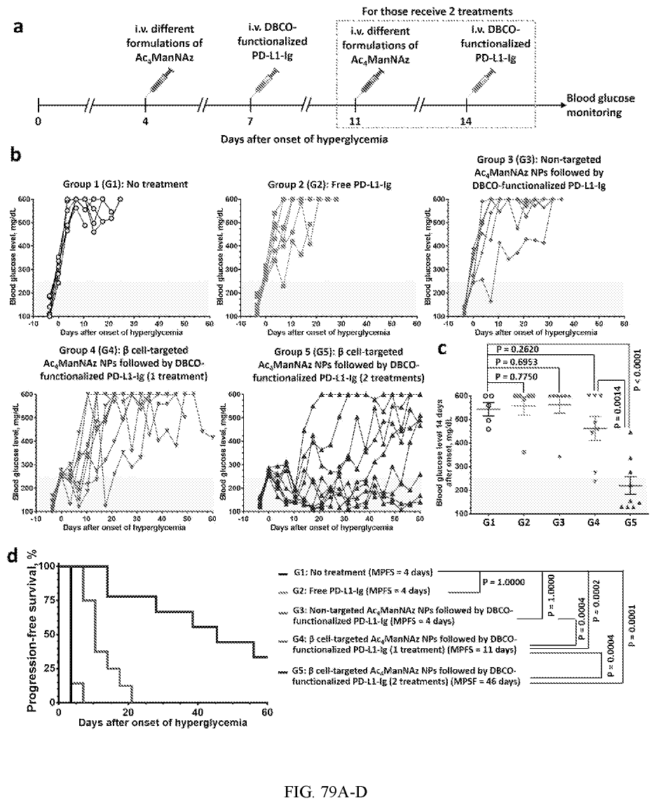

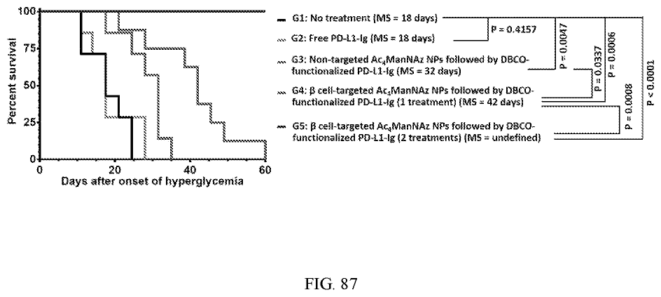

Guided by these findings, we performed a therapeutic efficacy treatment study of early onset hyperglycemia in NOD mice (blood glucose level>250 mg/dL) to demonstrate that the proposed pretargeted strategy can reverse early onset T1DM. In the therapeutic study, DBCO-functionalized PD-L1-Ig (80 μg/mouse) was administered i.v. 3 days after the administration of different Ac₄ManNAz NPs (180 μg of Ac₄ManNAz/mouse), which were administrated 4 days after the onset of T1DM (see FIG. 79a). Similar to the result that was observed in the biodistribution study, pretargeted treatment with the non-targeted Ac₄ManNAz NPs did not significantly affect blood glucose levels after the treatment compared to the non-treated mice and control group diabetic mice that were administrated DBCO-functionalized PD-L1-Ig (median progression-free survival (MPFS)=4 days; FIG. 79b-d). However, 6 of the 8 treated mice showed an initial response to pre-targeted treatment with the β cell-targeted Ac₄ManNAz NPs, and the treatment significantly prolonged the median survival (MS) from 18 days (for the non-treatment group) to 42 days (see Supporting Information, FIG. 87), although the MPFS increased only slightly to 11 days (see FIG. 79b,d). Recognizing that a single therapeutic treatment may not be sufficient to induce a robust immunotolerance due to the detachment of the in vivo conjugated PD-L1-Ig, we performed a dual pretargeted treatment study in which the mice received a second cycle of pretargeted treatment 4 days after the first cycle of pretargeted treatment (see FIG. 79a). In contrast to the results of the single pretargeted treatment, 7 out of the 9 treated mice showed a sustained response after two cycles of pretargeted treatments. The MPSF of the mice that received two rounds of pretargeted treatment increased significantly from 11 days (for mice that received a single pretargeted treatment) to 46 days (see FIG. 579). At the study's endpoint (60 days after the onset of T1DM), all NOD mice that received two cycles of pretargeted treatments survived (see Supporting Information, FIG. 87), with 3 out of 9 treated mice remaining normoglycemic.

In vivo therapeutic treatment was performed in early onset T1DM NOD mice (blood glucose=250-300 mg/dL; female). Mice in the pretargeted treatment group received i.v. administration of β cell-targeted or non-targeted Ac₄ManNAz NPs (180 μg of Ac₄ManNAz/mouse) 4 days after the onset of T1DM. DBCO-functionalized PD-L1-Ig (80 μg/mouse) was i.v. administered 3 days (day 7 after the onset of T1DM) after the administration of Ac₄ManNAz NPs. Mice in the control treatment group received a single i.v. administration of DBCO-functionalized PD-L1-Ig (80 μg/mouse) day 7 after the onset of T1DM. Mice that received two cycles of pretargeted treatment received the second i.v. administration of β cell-targeted Ac₄ManNAz NPs at day 11 after the onset of T1DM and DBCO-functionalized PD-L1-Ig at day 14 after the onset of T1DM. The blood glucose level of diabetic mice was measured twice a week (Tuesday morning and Friday afternoon) until it reached the desired experiment endpoint (death, 10% weight loss within 7 days, body condition score dropping below 2.0, or 60 days after the onset of T1DM).

### Example 19: Analyses of Pancreatic-Infiltrated T Cell Populations

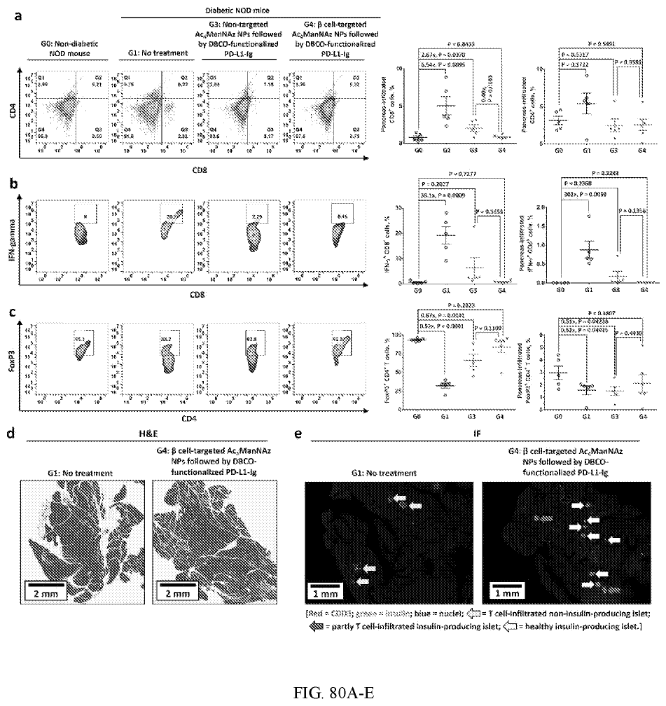

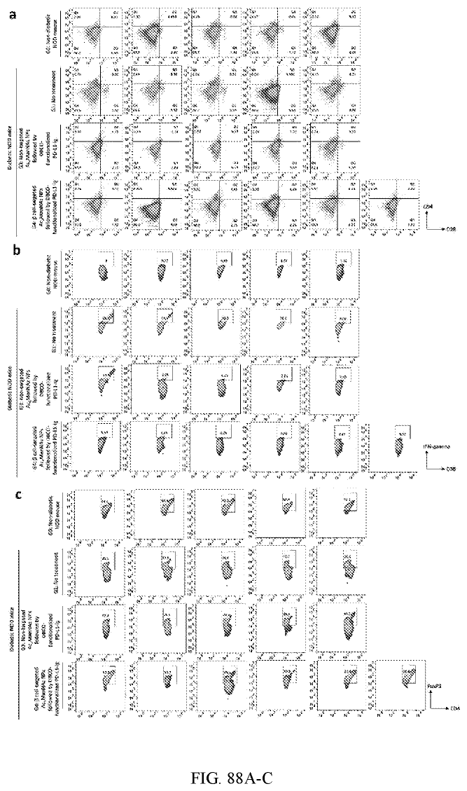

To obtain better insight into the therapeutic effect of the in vivo functionalized β cells, we analyzed the pancreas-infiltrated T cell populations 5 days after the pre-targeted treatments (or 12 days after onset of T1DM). Untreated diabetic NOD mice showed a 6.5-fold increase in the pancreas-infiltrated CD8⁺ T cells (with about 20% of them being IFN-γ⁺) compared to non-diabetic NOD mice of similar ages (see FIG. 80a,b; Supporting Information, FIG. 88a,b). Mice that received pre-targeted treatment with non-targeted Ac₄ManNAz NPs followed by DBCO-functionalized PD-L1-Ig showed a slight reduction in the number of pancreas-infiltrated CD8⁺ T cells, and the number of IFN-γ-expressing pancreas-infiltrated CD8⁺ T cells was comparable to that of healthy mice (see FIG. 80b; Supporting Information, FIG. 88a,b). Due to the increased amount of in vivo bioconjugated PD-L1-Ig, and, thus, stronger T cell exhaustion, mice that were treated with the β cell-targeted Ac₄ManNAz NPs followed by DBCO-functionalized PD-L1-Ig had a normal quantity of pancreas-infiltrated CD8⁺ T cells (and a normal level of IFN-γ-expressing pancreas-infiltrated CD8⁺ T cells) (see FIG. 80b; Supporting Information, FIG. 88a,b). Although the untreated diabetic mice and all treated NOD mice had numbers of CD4⁺ helper T cells that were comparable to those of healthy mice, untreated diabetic mice and mice treated with non-targeted Ac₄ManNAz NPs followed by DBCO-functionalized PD-L1-Ig had about 50% fewer FoxP3⁺ CD4⁺ Treg cells compared to healthy NOD mice and mice treated with β cell-targeted Ac₄ManNAz NPs followed by DBCO-functionalized PD-L1-Ig (see FIG. 80a,c; Supporting Information, FIG. 88a,c). Additionally, pathogenic helper T cells (e.g., IFN-γ⁺ CD4⁺ T cells) coexisted with Treg cells in the pancreas of diabetic NOD mice and mice that received non-targeted pretargeted treatment. Further histopathological studies confirmed that pretargeted treatment with Ac₄ManNAz NPs followed by DBCO-functionalized PD-L1-Ig significantly reduced the number of pancreas-infiltrated T cells (see FIG. 80d) and retained the insulin-producing islets (see FIG. 80e).

Pancreas-infiltrated T cell populations were analyzed by the FACS method, as previously reported. Briefly, diabetic NOD mice received treatment with β cell-targeted or non-targeted Ac₄ManNAz NPs (180 μg of Ac₄ManNAz/mouse) 4 days after the onset of T1DM. DBCO-functionalized PD-L1-Ig (80 μg/mouse) was i.v. administrated 3 days (day 7 after the onset of T1DM) after the administration of Ac₄ManNAz NPs. Mice were euthanized 5 days after the administration of DBCO-functionalized PD-L1-Ig (12 days post-onset of T1DM) for mechanistic study. The non-treatment group mice were euthanized 12 days after the onset of T1DM. Healthy non-diabetic NOD mice of similar age were used for the control study. Freshly preserved pancreas samples was digested with collagenase (2.5 mg/mL in HBBS buffer, 5 mL per pancreas; collagenase from Clostridium histolyticum; catalog number: C9407; Sigma) at 37° C. for 15 min, during which the pancreas suspensions were shaken 10 times every 4-5 min. Digestion was stopped by 10% FBS and isolated cells were mashed through a cell strainer (70 μm, Fisher). After washing the cell once with HBBS buffer, erythrocytes were lysed by ACK Lysis Buffer (Gibco) and washed before the FACS study. Isolated cells (suspended in 1×PBS) were first stained with Fixable Viability Stain 510 (catalog number: 564406; BD Bioscience), followed by A488-labeled anti-mouse CD8 antibody (clone: 37006; catalog number: FAB1509G100; R&D System) and PE-labeled anti-mouse CD4 antibody (clone: CT-CD4; catalog number: PIMA517450; Invitrogen). Cells were then fixed with 4% PFA, permeabilized, and stained with PE-Cyanine 7-labeled anti-IFN-γ antibody (clone: XMG1.2; catalog number: 25-7311-41, Invitrogen) and DyLight 650 anti-mouse FoxP3 polyclonal antibody (catalog number: PA5-22773, Invitrogen) before the FACS study. Data were acquired using a Thermo Fisher Attune NxT Analyzer or Intellicyt iQue Screener PLUS Analyzer in the Flow Cytometry Core Facility in the UNC School of Medicine.

Many modifications and other embodiments of the inventions set forth herein will come to mind to one skilled in the art to which the inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### BIBLIOGRAPHY

1. Atkinson, M. A., G. S. Eisenbarth, and A. W. Michels, Type 1 diabetes. Lancet, 2014. 383(9911): p. 69-82.
2. Van Belle, T. L., K. T. Coppieters, and M. G. Von Herrath, Type 1 Diabetes: Etiology, Immunology, and Therapeutic Strategies. Physiological Reviews, 2011. 91(1): p. 79-118.
3. Katsarou, A., et al., Type 1 diabetes mellitus. Nat Rev Dis Primers, 2017. 3: p. 17016.
4. Roglic, G. and World Health Organization, Global report on diabetes. 2016, Geneva, Switzerland: World Health Organization. 86 pages.
5. Ben Nasr, M., et al., PD-L1 genetic overexpression or pharmacological restoration in hematopoietic stem and progenitor cells reverses autoimmune diabetes. Sci Transl Med, 2017. 9(416).
6. Insel, R. A., et al., Staging presymptomatic type 1 diabetes: a scientific statement of JDRF, the Endocrine Society, and the American Diabetes Association. Diabetes Care, 2015. 38(10): p. 1964-74.
7. Raz, I., et al., Treatment of recent-onset type 1 diabetic patients with DiaPep277: results of a double-blind, placebo-controlled, randomized phase 3 trial. Diabetes Care, 2014. 37(5): p. 1392-400.
8. Buzzetti, R., et al., C-peptide response and HLA genotypes in subjects with recent-onset type 1 diabetes after immunotherapy with DiaPep277: an exploratory study. Diabetes, 2011. 60(11): p. 3067-72.
9. Liu, Y.-F., M. Peakman, and C. M. Dayan, Safely targeting autoimmunity in type 1 diabetes: the MonoPepfT1De trial. Practical Diabetes, 2013. 30(4): p. 148-150a.
10. Smith, E. L. and M. Peakman, Peptide Immunotherapy for Type 1 Diabetes—Clinical Advances. Frontiers in Immunology, 2018. 9.
11. Luo, X., S. D. Miller, and L. D. Shea, Immune Tolerance for Autoimmune Disease and Cell Transplantation. Annu Rev Biomed Eng, 2016. 18: p. 181-205.
12. Romagnani, S., Immunological tolerance and autoimmunity. Intern Emerg Med, 2006. 1(3): p. 187-96.
13. Schwartz, R. H., T cell anergy. Annu Rev Immunol, 2003. 21: p. 305-34.
14. Sinha, A. A., M. T. Lopez, and H. O. McDevitt, Autoimmune diseases: the failure of self tolerance. Science, 1990. 248(4961): p. 1380-8.
15. Sakaguchi, S., F. Powrie, and R. M. Ransohoff, Re-establishing immunological self-tolerance in autoimmune disease. Nat Med, 2012. 18(1): p. 54-8.
16. Wen, X., et al., Transplantation of NIT-1 cells expressing pD-L1 for treatment of streptozotocin-induced diabetes. Transplantation, 2008. 86(11): p. 1596-602.
17. Ansari, M. J., et al., The programmed death-1 (PD-1) pathway regulates autoimmune diabetes in nonobese diabetic (NOD) mice. J Exp Med, 2003. 198(1): p. 63-9.
18. Dahlen, E., G. Hedlund, and K. Dawe, Low CD86 expression in the nonobese diabetic mouse results in the impairment of both T cell activation and CTLA-4 up-regulation. J Immunol, 2000. 164(5): p. 2444-56.
19. Kanzaki, M., et al., Galectin-9 and T cell immunoglobulin mucin-3 pathway is a therapeutic target for type 1 diabetes. Endocrinology, 2012. 153(2): p. 612-20.
20. Agatemor, C., et al., Exploiting metabolic glycoengineering to advance healthcare. Nat Rev Chem, 2019. 3(10): p. 605-620.
21. Du, J., et al., Metabolic glycoengineering: sialic acid and beyond. Glycobiology, 2009. 19(12): p. 1382-401.
22. Kim, E. and H. Koo, Biomedical applications of copper-free click chemistry: in vitro, in vivo, and ex vivo. Chemical Science, 2019. 10(34): p. 7835-7851.
23. Chang, P. V., et al., Copper-free click chemistry in living animals. Proc Natl Acad Sci USA, 2010. 107(5): p. 1821-6.
24. Baskin, J. M., et al., Copper-free click chemistry for dynamic in vivo imaging. Proc Natl Acad Sci USA, 2007. 104(43): p. 16793-7.
25. Tian, X., et al., Organ-specific metastases obtained by culturing colorectal cancer cells on tissue-specific decellularized scaffolds. Nat Biomed Eng, 2018. 2: p. 443-452.
28. Sakaguchi, S., Yamaguchi, T., Nomura, T. & Ono, M. Regulatory T cells and immune tolerance. Cell 133, 775-787 (2008).
29. Goverman, J. M. Immune tolerance in multiple sclerosis. Immunol Rev 241, 228-240 (2011).
30. Vandenbark, A. A. & Offner, H. Critical evaluation of regulatory T cells in autoimmunity: are the most potent regulatory specificities being ignored? Immunology 125, 1-13 (2008).
31. Goldenberg, M. M. Multiple sclerosis review. P T 37, 175-184 (2012).
32. Torkildsen, O., Myhr, K. M. & Bo, L. Disease-modifying treatments for multiple sclerosis—a review of approved medications. Eur J Neurol 23 Suppl 1, 18-27 (2016).
33. Mendes, A. & Sa, M. J. Classical immunomodulatory therapy in multiple sclerosis: how it acts, how it works. Arq Neuropsiquiatr 69, 536-543 (2011).
34. Kammona, O. & Kiparissides, C. Recent Advances in Antigen-Specific Immunotherapies for the Treatment of Multiple Sclerosis. Brain Sci 10 (2020).
35. Jyothi, M. D., Flavell, R. A. & Geiger, T. L. Targeting autoantigen-specific T cells and suppression of autoimmune encephalomyelitis with receptor-modified T lymphocytes. Nat Biotechnol 20, 1215-1220 (2002).
36. Duffy, S. S., Keating, B. A. & Moalem-Taylor, G. Adoptive Transfer of Regulatory T Cells as a Promising Immunotherapy for the Treatment of Multiple Sclerosis. Front Neurosci 13, 1107 (2019).
37. Joller, N., Peters, A., Anderson, A. C. & Kuchroo, V. K. Immune checkpoints in central nervous system autoimmunity. Immunol Rev 248, 122-139 (2012).
38. Chitnis, T. & Khoury, S. J. Role of costimulatory pathways in the pathogenesis of multiple sclerosis and experimental autoimmune encephalomyelitis. J Allergy Cin Immunol 112, 837-849; quiz 850 (2003).
39. Trabattoni, D. et al. Costimulatory pathways in multiple sclerosis: distinctive expression of PD-1 and PD-L1 in patients with different patterns of disease. J Immunol 183, 4984-4993 (2009).
40. Gerdes, L. A. et al. CTLA4 as Immunological Checkpoint in the Development of Multiple Sclerosis. Ann Neurol 80, 294-300 (2016).
41. Bilate, A. M. & Lafaille, J. J. Induced CD4+Foxp3+ regulatory T cells in immune tolerance. Annu Rev Immunol 30, 733-758 (2012).
42. Aly, L., Hemmer, B. & Korn, T. From Leflunomide to Teriflunomide: Drug Development and Immunosuppressive Oral Drugs in the Treatment of Multiple Sclerosis. Curr Neuropharmacol 15, 874-891 (2017).
43. Klotz, L. et al. Teriflunomide treatment for multiple sclerosis modulates T cell mitochondrial respiration with affinity-dependent effects. Sci Transl Med 11 (2019).
44. Duncan, I. D. et al. The adult oligodendrocyte can participate in remyelination. Proc Natl Acad Sci USA 115, E11807-E11816 (2018).
45. Mosahebi, A., Fuller, P., Wiberg, M. & Terenghi, G. Effect of allogeneic Schwann cell transplantation on peripheral nerve regeneration. Exp Neurol 173, 213-223 (2002).
46. Oudega, M. & Xu, X. M. Schwann cell transplantation for repair of the adult spinal cord. J Neurotrauma 23, 453-467 (2006).
47. Baron-Van Evercooren, A., Avellana-Adalid, V., Lachapelle, F. & Liblau, R. Schwann cell transplantation and myelin repair of the CNS. Mult Scler 3, 157-161 (1997).
49. Au, K. M., Medik, Y., Ke, Q., Tisch, R. & Wang, A. Z. Immune Checkpoint-Bioengineered Beta Cell Vaccine Reverses Early-Onset Type 1 Diabetes. Adv Mater, e2101253 (2021).
51. Au, K. M. et al. Bespoke Pretargeted Nanoradioimmunotherapy for the Treatment of Non-Hodgkin Lymphoma. ACS Nano 12, 1544-1563 (2018).
52. Au, K. M., Wang, A. Z. & Park, S. I. Pretargeted delivery of PI3K/mTOR small-molecule inhibitor-loaded nanoparticles for treatment of non-Hodgkin's lymphoma. Sci Adv 6, eaaz9798 (2020).
53. Sharma, P., Gangopadhyay, D., Mishra, P. C., Mishra, H. & Singh, R. K. Detection of in Vitro Metabolite Formation of Leflunomide: A Fluorescence Dynamics and Electronic Structure Study. J Med Chem 59, 3418-3426 (2016).
54. Oliveira, B. L., Guo, Z. & Bernardes, G. J. L. Inverse electron demand Diels-Alder reactions in chemical biology. Chem Soc Rev 46, 4895-4950 (2017).
55. Bettelli, E. et al. Myelin oligodendrocyte glycoprotein-specific T cell receptor transgenic mice develop spontaneous autoimmune optic neuritis. J Exp Med 197, 1073-1081 (2003).
56. Roberts, R. A. et al. Towards programming immune tolerance through geometric manipulation of phosphatidylserine. Biomaterials 72, 1-10 (2015).
57. Korn, T., Magnus, T., Toyka, K. & Jung, S. Modulation of effector cell functions in experimental autoimmune encephalomyelitis by leflunomide—mechanisms independent of pyrimidine depletion. J Leukoc Biol 76, 950-960 (2004).
58. Bradley, L. M., Dalton, D. K. & Croft, M. A direct role for IFN-gamma in regulation of Th1 cell development. J Immunol 157, 1350-1358 (1996).
59. Tsai, H. C., Velichko, S., Hung, L. Y. & Wu, R. IL-17A and Th17 cells in lung inflammation: an update on the role of Th17 cell differentiation and IL-17R signaling in host defense against infection. Clin Dev Immunol 2013, 267971 (2013).
60. Karwacz, K. et al. PD-L1 co-stimulation contributes to ligand-induced T cell receptor down-modulation on CD8+ T cells. EMBO Mol Med 3, 581-592 (2011).
61. Jeannin, P. et al. Soluble CD86 is a costimulatory molecule for human T lymphocytes. Immunity 13, 303-312 (2000).
62. Mendel, I., Kerlero de Rosbo, N. & Ben-Nun, A. A myelin oligodendrocyte glycoprotein peptide induces typical chronic experimental autoimmune encephalomyelitis in H-2b mice: fine specificity and T cell receptor V beta expression of encephalitogenic T cells. Eur J Immunol 25, 1951-1959 (1995).
63. Constantinescu, C. S., Farooqi, N., O'Brien, K. & Gran, B. Experimental autoimmune encephalomyelitis (EAE) as a model for multiple sclerosis (MS). Br J Pharmacol 164, 1079-1106 (2011).
64. Tompkins, S. M. et al. De novo central nervous system processing of myelin antigen is required for the initiation of experimental autoimmune encephalomyelitis. J Immunol 168, 4173-4183 (2002).
65. Setiady, Y. Y., Coccia, J. A. & Park, P. U. In vivo depletion of CD4+FOXP3+ Treg cells by the PC61 anti-CD25 monoclonal antibody is mediated by FcgammaRIII+ phagocytes. Eur J Immunol 40, 780-786 (2010).
66. Arellano, B., Graber, D. J. & Sentman, C. L. Regulatory T cell-based therapies for autoimmunity. Discov Med 22, 73-80 (2016).
67. Getts, D. R. et al. Microparticles bearing encephalitogenic peptides induce T-cell tolerance and ameliorate experimental autoimmune encephalomyelitis. Nat Biotechnol 30, 1217-1224 (2012).
68. Getts, D. R. et al. Tolerance induced by apoptotic antigen-coupled leukocytes is induced by PD-L1+ and IL-10-producing splenic macrophages and maintained by T regulatory cells. J Immunol 187, 2405-2417 (2011).
69. Lutterotti, A. et al. Antigen-specific tolerance by autologous myelin peptide-coupled cells: a phase 1 trial in multiple sclerosis. Sci Transl Med 5, 188ra175 (2013).
70. Smith, C. E., Eagar, T. N., Strominger, J. L. & Miller, S. D. Differential induction of IgE-mediated anaphylaxis after soluble vs. cell-bound tolerogenic peptide therapy of autoimmune encephalomyelitis. Proc Natl Acad Sci USA 102, 9595-9600 (2005).
71. Au, K. M., Park, S. I. & Wang, A. Z. Trispecific natural killer cell nanoengagers for targeted chemoimmunotherapy. Science Advances 6, eaba8564 (2020).
72. Li, Y. & Kurlander, R. J. Comparison of anti-CD3 and anti-CD28-coated beads with soluble anti-CD3 for expanding human T cells: differing impact on CD8 T cell phenotype and responsiveness to restimulation. J Transl Med 8, 104 (2010).
73. Okuda, Y., Okuda, M. & Bernard, C. C. The suppression of T cell apoptosis influences the severity of disease during the chronic phase but not the recovery from the acute phase of experimental autoimmune encephalomyelitis in mice. J Neuroimmunol 131, 115-125 (2002).
74. Wang, P.; Yoo, B.; Yang, J.; Zhang, X.; Ross, A.; Pantazopoulos, P.; Dai, G.; Moore, A., GLP-1R-targeting magnetic nanoparticles for pancreatic islet imaging. Diabetes 2014, 63 (5), 1465-74.
75. Widmann, C.; Dolci, W.; Thorens, B., Agonist-induced internalization and recycling of the glucagon-like peptide-1 receptor in transfected fibroblasts and in insulinomas. Biochem J 1995, 310 (Pt 1), 203-14.
76. Zhang, X.; Kang, Y.; Wang, J.; Yan, J.; Chen, Q.; Cheng, H.; Huang, P.; Gu, Z., Engineered PD-L1-Expressing Platelets Reverse New-Onset Type 1 Diabetes. Adv Mater 2020, 32 (26), e1907692.
77. Townsend, S. A.; Evrony, G. D.; Gu, F. X.; Schulz, M. P.; Brown, R. H., Jr.; Langer, R., Tetanus toxin C fragment-conjugated nanoparticles for targeted drug delivery to neurons. Biomaterials 2007, 28 (34), 5176-84.
78. Hamaguchi, K.; Gaskins, H. R.; Leiter, E. H., NIT-1, a pancreatic beta-cell line established from a transgenic NOD/Lt mouse. Diabetes 1991, 40 (7), 842-9.
79. Ishihara, H.; Asano, T.; Tsukuda, K.; Katagiri, H.; Inukai, K.; Anai, M.; Kikuchi, M.; Yazaki, Y.; Miyazaki, J. I.; Oka, Y., Pancreatic beta cell line MIN6 exhibits characteristics of glucose metabolism and glucose-stimulated insulin secretion similar to those of normal islets. Diabetologia 1993, 36 (11), 1139-45.
80. Scott, D.; Nitecki, D. E.; Kindler, H.; Goodman, J. W., Immunogenicity of biotinylated hapten-avidin complexes. Mol Immunol 1984, 21 (11), 1055-60.
81. Sinitsyn, V. V.; Mamontova, A. G.; Checkneva, Y. Y.; Shnyra, A. A.; Domogatsky, S. P., Rapid blood clearance of biotinylated IgG after infusion of avidin. J Nucl Med 1989, 30 (1), 66-9.
82. Au, K. M.; Tripathy, A.; Lin, C. P.; Wagner, K.; Hong, S.; Wang, A. Z.; Park, S. I., Bespoke Pretargeted Nanoradioimmunotherapy for the Treatment of Non-Hodgkin Lymphoma. ACS Nano 2018, 12 (2), 1544-1563.
83. Krishnamurthy, B.; Mariana, L.; Gellert, S. A.; Colman, P. G.; Harrison, L. C.; Lew, A. M.; Santamaria, P.; Thomas, H. E.; Kay, T. W., Autoimmunity to both proinsulin and IGRP is required for diabetes in nonobese diabetic 8.3 TCR transgenic mice. J Immunol 2008, 180 (7), 4458-64.
84. Yi, J. S.; Cox, M. A.; Zajac, A. J., T-cell exhaustion: characteristics, causes and conversion. Immunology 2010, 129 (4), 474-81.

What is claimed is:

1. A functionalized cell comprising, a cell comprising a decorated cell surface, wherein the decorated cell surface comprises at least one covalently attached immune checkpoint molecule.

2. The functionalized cell of claim 1, wherein the immune checkpoint molecule is selected from the group consisting of PD-L1, CD86, Gal-9, PD-L2, TIGIT, TIM-1, TIM-3, TNFR1, VISTA, BTLA, NKG2A, CTLA-4, B7-H3, B7-H4, B7-H5, B7-H6, B7-H7, ICOS, NKp30, LAG3, CD137, and CD96.

3. The functionalized cell of claim 1, wherein the cell is a beta cell, a Schwann cell, oligodendrocytes, a pneumocyte, a platelet, a epithelial cell, a hepatocyte, or a synovial cell.

4. The functionalized cell of claim 1, wherein the at least one covalently attached immune checkpoint molecule is attached through a glycoengineered moiety or through a thiol-maleimide conjugation.

5. The functionalized cell of claim 1, wherein the at least one covalently attached immune checkpoint molecule is an immune checkpoint molecule-functionalized nanoparticle or polymer.

6. The functionalized cell of claim 4, wherein the glycoengineered moiety comprises a residue of an amide of mannosamine or galactosamine.

7. The functionalized cell of claim 6, wherein the glycoengineered moiety further comprises a residue of an azide, a dibenzocyclooctyne, or a tetrazine covalently attached to the residue of an amide of mannosamine or galactosamine.

8. The functionalized cell of claim 7, wherein the dibenzocyclooctyne is DBCO.

9. The functionalized cell of claim 4, 6, 7 or 8, wherein the glycoengineered moiety further comprises a residue of a dendrimer, a linear polymer, a nanoparticle, or a Fc fusion protein.

10. The functionalized cell of claim 9, wherein the dendrimer is a multivalent dendrimer.

11. The functionalized cell of claim 10, wherein the multivalent dendrimer is a polyamidoamine dendrimer.

12. The functionalized cell of claim 11, wherein the polyamidoamine dendrimer has a MW of from about 500 to about 1,000,000.

13. The functionalized cell of claim 12, wherein the polyamidoamine dendrimer has a MW of from about 25,000 to about 30,000.

14. The functionalized cell of claim 1, comprising from about 0.5 μg to about 50.0 μg of the at least one covalently attached immune checkpoint molecule per about 1 million functionalized cells.

15. The functionalized cell of claim 1, comprising at least one PD-L1, at least one CD86, and at least one Gal-9.

16. The functionalized cell of claim 1, comprising at least one PD-L1 and at least one CD86.

17. The functionalized cell of claim 5, having one of the following structures:

[symbol]

18. The functionalized cell of claim 17, wherein the nanoparticle comprises a cargo.

19. The functionalized cell of claim 18, wherein the cargo is an immunosuppressive agent.

20. The functionalized cell of claim 19, wherein the immunosuppressive agent is selected from the group consisting of leflunomide azathioprine, lenalidomide, pomalidomide, methotrexate, azathioprine and thalidomide.

21. The functionalized cell of claim 2, wherein the immune checkpoint molecule is selected from the group consisting of PD-L1, CD86, and Gal-9.

22. The functionalized cell of claim 1, wherein the cell is viable for about 1 day to about 7 days under physiological conditions.

23. The functionalized cell of claim 1, wherein the cell is viable for about 2 days to about 6 days under physiological conditions.

24. The functionalized cell of claim 1, wherein the cell is viable for about 3 days to about 4 days under physiological conditions.

25. The functionalized cell of claim 1, wherein the cell is viable for about 5 days to about 21 days under physiological conditions.

26. The functionalized cell of claim 4, comprising:
a glycoengineered moiety having the structure: (a transmembrane glycoprotein)-(a residue of an azide-containing molecule)-(a residue of a cyclooctyne)-(a linker 1)-(a residue of a functionalized dendrimer)_{q}-(a residue of an immune checkpoint molecule),
wherein,
q is one or zero; and,
the dash represents a covalent bond.

27. The functionalized cell of claim 4, comprising:
a glycoengineered moiety having the structure: (a transmembrane glycoprotein)-(a residue of an cyclooctyne-containing molecule)-(a residue of a azide)-(a linker 1)-(a residue of a functionalized dendrimer)_{q}-(a residue of an immune checkpoint molecule),
wherein,
q is one or zero; and,
the dash represents a covalent bond.

28. The functionalized cell of any one of claims 26-27, wherein q is one.

29. The functionalized cell of any one of claim 26-27, wherein q is zero.

30. The functionalized cell of claim 4, comprising:
a glycoengineered moiety having the structure: (a transmembrane glycoprotein)-(a residue of an azide-containing molecule)-(a residue of a cyclooctyne)-(a linker 1)-(immune checkpoint molecule FcIg fusion protein),
wherein, the dash represents a covalent bond.

31. The functionalized cell of claim 4, comprising:
a glycoengineered moiety having the structure: (a transmembrane glycoprotein)-(a residue of a cycoloctyne-containing molecule)-(a residue of a azide)-(a linker 1)-(immune checkpoint molecule FcIg fusion protein),
wherein, the dash represents a covalent bond.

32. The functionalized cell of claim 26, wherein the residue of a functionalized dendrimer has the structure: -(dendrimer)-(a linker 2)-(a residue of a cyclooctyne)-(a residue of an azide-containing molecule).

33. The functionalized cell of claim 32, wherein the linker 2 has the structure. 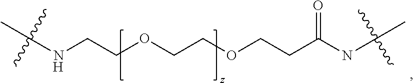
wherein, z is an integer from 0 to 10.

34. The functionalized cell of claim 33, wherein z is 3.

35. An acellular pancreatic extracellular matrix comprising, a functionalized cell of claim 1; and decellularized pancreatic-derived proteins.

36. The acellular pancreatic extracellular matrix of claim 35, wherein the functionalized cells form three-dimensional spheroid colonies.

37. The acellular pancreatic extracellular matrix of claim 35, wherein the acellular pancreatic extracellular matrix is in the form of an injectable.

38. The acellular pancreatic extracellular matrix of claim 37, wherein the acellular pancreatic extracellular matrix is in the form of an injectable that is not a gel.

39. A pharmaceutical composition comprising, a functionalized cell of claim 1 or an acellular pancreatic extracellular matrix of claim 35, and a pharmaceutically acceptable excipient.

40. A vaccine comprising a functionalized cell of claim 1 or an acellular pancreatic extracellular matrix of claim 35, and a pharmaceutically acceptable liquid vehicle.

41. A method of treating or delaying onset of an autoimmune disease in a subject, comprising:
administering to the subject, a functionalized cell of claim 1 or an acellular pancreatic extracellular matrix of claim 35 or a pharmaceutical composition of claim 39 or a vaccine of claim 40.

42. The method of claim 41, wherein the autoimmune disease is type 1 diabetes, multiple sclerosis, autoimmune colitis, arthritis, lupus, or psoriasis.

43. The method of claim 42, wherein the autoimmune colitis is ulcerative colitis or crohn's disease.

44. The method of claim 42, wherein the arthritis is rheumatoid arthritis.

45. The method of claim 41, wherein the autoimmune disease is early-onset type 1 diabetes or early-onset hyperglycemia.

46. The method of claim 45, wherein the functionalized cell is a beta cell.

47. The method of claim 46, wherein the subject is at risk of developing diabetes or has diabetes.

48. The method of claim 41, wherein the autoimmune disease is multiple sclerosis.

49. The method of claim 48, wherein the functionalized cell is a cell associated with myelin sheath.

50. The method of claim 49, wherein the subject is at risk of developing multiple sclerosis or has multiple sclerosis.

51. The method of claim 49, wherein the subject has relapsing multiple sclerosis.

52. The method of claim 41, wherein treating an autoimmune disease is reducing the severity of symptoms of the autoimmune disease.

53. The method of claim 50, wherein treating the subject with multiple sclerosis is reducing the severity of multiple sclerosis symptoms.

54. The method of claim 41, further comprising administering a booster dose.

55. A method of delivery of a cargo into the CNS of a subject, comprising: administering to the subject, the functionalized cell of claim 5.

56. The method of claim 55, wherein the administering is intravenous.

57. A method of reducing inflammation in a CNS microenvironment, comprising: administering to the subject, the functionalized cell of claim 5, wherein systemic immunosuppression is not induced.

58. A method of reversing early-onset type 1 diabetes in a subject, comprising: administering to the subject, a functionalized cell of claim 1 or an acellular pancreatic extracellular matrix of claim 35 or a pharmaceutical composition of claim 39 or a vaccine of claim 40.

59. A method of modulating the T_{reg}:T_{eff} ratio in a subject, comprising:
administering to the subject, a functionalized cell of claim 1 or an acellular pancreatic extracellular matrix of claim 35 or a pharmaceutical composition of claim 39 or a vaccine of claim 40.

60. A method of exhausting autoreactive effector T-cells in a subject, comprising administering to the subject, a functionalized cell of claim 1 or an acellular pancreatic extracellular matrix of claim 35 or a pharmaceutical composition of claim 39 or a vaccine of claim 40.

61. A method of protecting pancreatic beta cells in a subject, comprising administering to the subject, a functionalized cell of claim 1 or an acellular pancreatic extracellular matrix of claim 35 or a pharmaceutical composition of claim 39 or a vaccine of claim 40.

62. The method of claim 42, 59, 60, or 61, further comprising a second administration at a time period after the administering.

63. A method of preparing a functionalized cell of claim 1, comprising:
glycoengineering a cell to express a glycoengineered moiety comprising an azide moiety, a cyclooctyne moiety, or a tetrazine moiety; and
covalently linking an immune checkpoint molecule through the azide moiety, cyclooctyne moiety, or tetrazine moiety,
to prepare a functionalized cell.

64. The method of claim 63, further comprising, prior to the glycoengineering, harvesting the cell from a subject.

65. The method of claim 63 or 64, further comprising, after the linking, preserving the functionalized cell.

66. The functionalized cell of claim 1, wherein the cell is a living cell.

67. A method of preparing a functionalized cell, comprising:
covalently attaching an immune checkpoint molecule through a thiol maleimide conjugation, to prepare a functionalized cell.

68. An in vivo method of preparing a functionalized cell in an organism, comprising:
administering to the organism in any order:
a cell labeling agent comprising a ligand reactive group, and
one or more active agents comprising a covalently bound ligand that reacts with the ligand reactive group,
wherein the functionalized cell is prepared in vivo.

69. The method of claim 68, wherein the ligand reactive group comprises an azide moiety.

70. The method of claim 68, wherein the cell is a beta cell, a Schwann cell, oligodendrocytes, a pneumocyte, a platelet, a epithelial cell, a hepatocyte, or a synovial cell.

71. A method of treating an autoimmune disease in a subject, comprising:
administering to the subject in any order:
a cell labeling agent comprising a ligand reactive group, and
one or more active agents comprising a covalently bound ligand that reacts with the ligand reactive group, wherein a functionalized cell is prepared in vivo, and
wherein the autoimmune disease is treated.

72. The method of claim 71, wherein the autoimmune disease is Type 1 diabetes mellitus.

73. A method of anergizing an autoreactive T-cell in a subject, comprising:
contacting the autoreactive T-cell with a functionalized cell, wherein the functionalized cell is prepared by administering to the subject in any order:
a cell labeling agent comprising a ligand reactive group, and
one or more active agents comprising a covalently bound ligand that reacts with the ligand reactive group, wherein the functionalized cell is prepared in vivo, and wherein the functionalized cell contacts the autoreactive T-cell, and wherein the T-cell is anergized.

74. The method of claim 73, wherein the T-cell is anergized and systemic immunosuppression is not induced.

75. The method of claim 74, wherein the systemic immunosuppression is long-term.

76. The method of claim 74, wherein the systemic immunosuppression is long-term and irreversible.
